(19) United States
(12) Patent Application Publication
(10) Pub. No.: US 2026/0217651 A1
(43) Pub. Date: Jul. 30, 2026

(54) SUBSTITUTED PHENOL DERIVATIVE AND USE THEREOF

(71) Applicant: CHENGDU MFS PHARMA. CO., LTD., Chengdu, Sichuan (CN)

(72) Inventors: Guangwu CHEN, Chengdu, Sichuan (CN); Jin LIU, Chengdu, Sichuan (CN); Wensheng ZHANG, Chengdu, Sichuan (CN); Hancheng ZHANG, Chengdu, Sichuan (CN); Zhenbiao XIE, Chengdu, Sichuan (CN); Haijun MA, Chengdu, Sichuan (CN)

(21) Appl. No.: 19/144,721

(22) PCT Filed: Dec. 26, 2023
(86) PCT No.: PCT/CN2023/141870

§ 371 Date: Jun. 30, 2025

(30) Foreign Application Priority Data
Dec. 29, 2022 (CN) 202211709368.6
Jul. 28, 2023 (CN) 202310941721.1

(51) Int. Cl. C07D 211/64; A61K 31/4525; A61K 31/4535; A61K 31/4545; A61K 31/495; C07D 211/58; C07D 241/04; C07D 401/04; C07D 405/04; C07D 405/12; C07D 409/04; C07D 417/04

(52) U.S. Cl. CPC C07D 211/64; A61K 31/4525; A61K 31/4535; A61K 31/4545; A61K 31/495; C07D 211/58; C07D 241/04; C07D 401/04; C07D 405/04; C07D 405/12; C07D 409/04; C07D 417/04

(57) ABSTRACT

A substituted phenol derivative and a use thereof, are in the field of medicinal chemistry. The substituted phenol derivative is a compound as represented by formula IA, or a stereoisomer thereof, or a pharmaceutically acceptable salt thereof, or a solvate thereof, or a prodrug thereof, or a metabolite thereof, or a deuterated derivative thereof. The derivative not only has sedative, hypnotic and/or anesthetic effects and/or can control status epilepticus, but also has an analgesic effect. The compound can be used in the preparation of a drug that has both an analgesic effect and sedative, hypnotic and/or anesthetic effects and a drug capable of controlling status epilepticus.

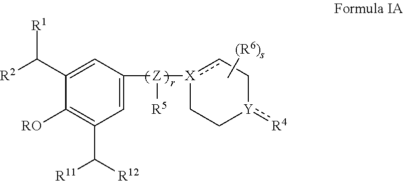

### TECHNICAL FIELD

The present invention relates to the field of medicinal chemistry, and more particular to a class of novel substituted phenol derivatives, their synthetic methods, and their applications in the preparation of drugs that exhibit both analgesic effects and sedative, hypnotic, and/or anesthetic effects, and/or are effective in controlling status epilepticus.

### BACKGROUND ART

Propofol is a rapid, short-acting intravenous general anesthetic widely used clinically for the induction and maintenance of general anesthesia and sedation of critically ill patients in ICUs. It offers advantages such as rapid induction of anesthesia, quick recovery with complete functional restoration, and low incidence of postoperative nausea and vomiting.

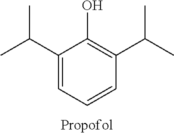

However, clinical intravenous general anesthetic drugs including propofol, etomidate, fospropofol disodium, and ciprofol do not have analgesic effects. Therefore, opioid analgesics such as fentanyl, alfentanil, sufentanil, or remifentanil must be administered simultaneously during clinical anesthesia.

In response to the aforementioned issues, there is an urgent need to develop a drug that integrates sedative, hypnotic, and/or anesthetic properties, status epilepticus control, and analgesic effects. A compound has sedative, hypnotic, and/or anesthetic effects, can control status epilepticus, and also has analgesic effects, it can achieve more complete analgesia, significantly reduce the dosage of opioid analgesics, reduce the adverse reactions of opioid analgesics, and make the sedative, hypnotic, and/or anesthetic process more stable. At the same time, it can also reduce the dosage of other drugs during combined anesthesia, accelerate the patient's recovery from sedative, hypnotic, and/or anesthetic states, and improve patient safety.

### SUMMARY

The objective of the present invention is to provide a novel series of substituted phenol derivatives of formula IA, or a stereoisomer thereof, a pharmaceutically acceptable salt thereof, a solvate thereof, a prodrug thereof, a metabolite thereof, or a deuterated derivative thereof, pharmaceutical composition thereof, their synthetic methods, and their use in the preparation of drugs inducing sedative, hypnotic, and/or anesthetic effects, as well as for controlling status epilepticus.

The present invention provides a compound of formula IA, or a stereoisomer thereof, a pharmaceutically acceptable salt thereof, a solvate thereof, a prodrug thereof, a metabolite thereof, or a deuterated derivative thereof:

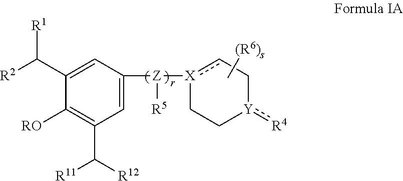

wherein, R is selected from hydrogen, COR^{a}, COCH(NH₂)R^{a1}, COOR^{h1}, (CH₂COO)ᵥR^{h1}, PO(OR^{h1})(OR^{h2}) or CH₂OPO(OR^{h1})(OR^{h2}); R^{a} and R^{a1} are each independently selected from hydrogen, C₁₋₄ alkyl, C₂₋₄ alkenyl, C₂₋₄ alkynyl, 3-6 membered cycloalkyl, 3-6 membered heterocyclyl, aryl or heteroaryl; R^{h1}, R^{h2} are each independently selected from hydrogen, C₁₋₄ alkyl, 3-6 membered cycloalkyl, 3-6 membered heterocyclyl, aryl or heteroaryl; v is an integer from 0-2;

The bond [symbol] connecting X can be a double bond or a single bond; when the bond [symbol] connecting X is a double bond, X is selected from C;
when the bond [symbol] connecting X is a single bond, X is selected from CR³ and N;

The bond [symbol] connecting Y can be a double bond or a single bond; when the bond [symbol] connecting Y is a double bond, Y is selected from C;
when the bond [symbol] connecting Y is a single bond, Y is selected from CR³ and N;
Z is selected from none, CR⁵′, N, O;
R¹, R², R¹¹ and R¹² are each independently selected from hydrogen, halogen, C₁₋₄ alkyl, C₁₋₄ alkenyl or 3-6 membered cycloalkyl;
s is selected from an integer from 0 to 8, and each R⁶ is independently selected from hydrogen, C₁₋₄ alkyl, halogenated C₁₋₄ alkyl, C₂₋₄ alkenyl, C₂₋₄ alkynyl, 3-6 membered cycloalkyl, 3-6 membered heterocyclyl, halogen, C₁₋₄ alkoxy, CN, NO₂, OR^{f}, SR^{f}, NR^{d1}R^{d2}, CORⁱ, (CR^{a1}R^{a2})ₘCOR^{b}; or, s is selected from an integer from 2 to 8, wherein two R⁶ bounded are connected to form a ring, and the remaining R⁶ is each independently selected from hydrogen, C₁₋₄ alkyl, halogenated C₁₋₄ alkyl, halogen, C₁₋₄ alkoxy, CN, NO₂, OR^{f}, SR^{f}, NR^{d1}R^{d2}, CORⁱ, (CR^{a1}R^{a2})ₘCOR^{b};
R⁵ and R^{5′} are each independently selected from none, hydrogen, C₁₋₈ alkyl, (CR^{a1}R^{a2})ₘCOR^{b};
R³ is hydrogen, (CR^{a1}R^{a2})ₘCOOR^{b1}, or groups including C₁₋₄ alkyl, C₂₋₄ alkenyl, C₂₋₄ alkynyl, 3-6 membered cycloalkyl, 3-6 membered heterocyclyl, aryl or heteroaryl unsubstituted or optionally substituted by one or more R^{x}; wherein R^{x} is each independently selected from cyano, nitro, hydroxyl, carboxyl, halogen, C₁₋₄ alkyl, C₁₋₄ alkoxy, (CR^{a1}R^{a2})ₘNR^{d1}R^{d2}, 3-8 membered cycloalkyl or its halogenated derivatives, 3-8 membered heterocyclyl or its halogenated derivatives, aryl or its halogenated or deuterated derivatives, heteroaryl or its halogenated or deuterated derivatives;
R⁴ is hydrogen,

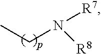

(CR^{a1}R^{a2})ₘCOOR^{b1}, (CR^{a1}R^{a2})ₘOCOR^{b}, (CR^{a1}R^{a2})ₙCOR^{c}, CONR^{d1}R^{d2}, (CR^{a1}R^{a2})ₙOR^{f};
when Z is selected from none, X is N, Y is N, R⁴ is

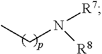

R⁷ is hydrogen or groups consisting of C₁₋₄ alkyl, 3-6 membered cycloalkyl, 3-6 membered heterocyclyl, aryl or heteroaryl unsubstituted or optionally substituted by one or more R^{y}; wherein R^{y} is each independently selected from cyano, nitro, hydroxyl, carboxyl, halo, C₁₋₄ alkyl, alkoxy, C₁₋₄ alkylthio, O(CR^{a1}R^{a2})ₘNR^{d1}R^{d2}, (CR^{a1}R^{a2})ₘNR^{d1}R^{d2}, 3-8 membered cycloalkyl or its halogenated derivatives, 3-8 membered heterocyclyl or its halogenated derivatives, aryl or its halogenated or deuterated derivatives, heteroaryl or its halogenated or deuterated derivatives;
R⁸ is hydrogen, C₁₋₄ alkyl, (CR^{a1}R^{a2})ₘCOOR^{b1}, (CR^{a1}R^{a2})ₘOCOR^{b}, (CR^{a1}R^{a2})ₙCOR^{c}, CONR^{d1}R^{d2}, (CR^{a1}R^{a2})ₙOR^{f}, (CR^{a1}R^{a2})ₘSO₂OR^{b1}, (CR^{a1}R^{a2})ₘSOOR^{b1};
wherein, R^{a1}, R^{a2} are each independently selected from hydrogen, halogen, C₁₋₄ alkyl, halogenated C₁₋₄ alkyl, C₂₋₄ alkenyl, C₂₋₄ alkynyl, 3-6 membered cycloalkyl, 3-6 membered heterocyclyl, aryl, heteroaryl, CN, OR^{f}, SR^{f}, or NR^{d1}R^{d2}; or, R^{a1} and R^{a2}, together with the carbon atom to which they are attached, form a 3-6 membered cyclic structure containing 0 or 1 heteroatom selected from N, O, or S;
wherein, R^{b1} is hydrogen, C₁₋₄ alkyl, C₁₋₄ alkoxy, halogenated C₁₋₄ alkyl, 3-6 membered cycloalkyl, 3-6 membered heterocyclyl, aryl, or heteroaryl;
R^{b} is hydrogen, C₁₋₄ alkyl, C₁₋₄ alkoxy, halogenated C₁₋₄ alkyl, C₂₋₄ alkenyl, C₂₋₄ alkynyl, 3-6 membered cycloalkyl, 3-6 membered heterocyclyl, aryl, or heteroaryl;
R^{c} is hydrogen, C₁₋₄ alkyl, C₁₋₄ alkoxy, C₂₋₄ alkenyl, C₂₋₄ alkynyl, 3-6 membered cycloalkyl, 3-6 membered heterocyclyl, aryl, or heteroaryl;
R^{d1} and R^{d2} are each independently selected from hydrogen, C₁₋₄ alkyl, C₁₋₄ alkoxy, 3-6 membered cycloalkyl, 3-6 membered heterocyclyl; or, R^{d1} and R^{d2} taken together with the nitrogen atom to which they are attached form a 3-6 membered ring containing 1 or 2 heteroatom selected from N, O, or S;
wherein the alkyl, alkenyl, alkynyl, cycloalkyl, heterocyclyl, aryl, or heteroaryl in R^{a1}, R^{a2}, R^{d1}, R^{d2}, R^{b}, R^{b1}, R^{c}, R⁵ and R^{5′} are unsubstituted or optionally substituted by one or more groups selected from halogen, C₁₋₄ alkyl, C₁₋₄ alkoxy, halogenated C₁₋₄ alkyl, C₂₋₄ alkenyl, C₂₋₄ alkynyl, 3-6 membered cycloalkyl, 3-6 membered heterocyclyl, CN, NO₂, OR^{f}, SR^{f}, NR^{d1}R^{d2}, CORⁱ, COOR^{f}, OCORⁱ, CONR^{d1}R^{d2}, NR^{d1}CORⁱ, NR^{d1}SO₂Rⁱ, or SO₂Rⁱ;
R^{f} is selected from hydrogen, C₁₋₄ alkyl, or halogenated C₁₋₄ alkyl;
Rⁱ is selected from hydrogen, C₁₋₄ alkyl, C₂₋₄ alkenyl, C₂₋₄ alkynyl, 3-6 membered cycloalkyl, or 3-6 membered heterocyclyl;
m is 0, 1, 2;
n is 0, 1, 2;
r is 0, 1, 2, 3;
p is 0, 1, 2, 3.

Further, said compound has a structure of formula I:

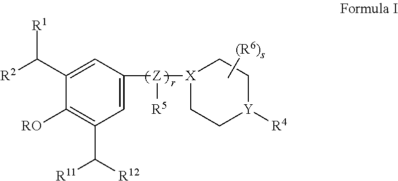

wherein, R is selected from hydrogen, COR^{a}, COCH(NH₂)R^{a1}, COOR^{h1}, (CH₂COO)ᵥR^{h1}, PO(OR^{h1})(OR^{h2}) or CH₂OPO(OR^{h1})(OR^{h2}); R^{a} and R^{a1} are each independently selected from hydrogen, C₁₋₄ alkyl, C₂₋₄ alkenyl, C₂₋₄ alkynyl, 3-6 membered cycloalkyl, 3-6 membered heterocyclyl, aryl or heteroaryl; R^{h1}, R^{h2} are each independently selected from hydrogen, C₁₋₄ alkyl, 3-6 membered cycloalkyl, 3-6 membered heterocyclyl, aryl or heteroaryl; v is an integer from 1 to 2;
X is selected from CR³, N;
Y is selected from CR³, N;
Z is selected from none, CR⁵′, N, O;
R¹, R², R¹¹ and R¹² are each independently selected from hydrogen, halogen, C₁₋₄ alkyl, C₂₋₄ alkenyl, or 3-6 membered cycloalkyl;
s is selected from an integer from 0 to 8, and each R⁶ is independently selected from hydrogen, C₁₋₄ alkyl, halogenated C₁₋₄ alkyl, C₂₋₄ alkenyl, C₂₋₄ alkynyl, 3-6 membered cycloalkyl, 3-6 membered heterocyclyl, halogen, C₁₋₄ alkoxy, CN, NO₂, OR^{f}, SR^{f}, NR^{d1}R^{d2}, CORⁱ, (CR^{a1}R^{a2})ₘCOR^{b}; or, s is selected from an integer from 2 to 8, wherein two R⁶ groups are bounded form a ring, and the remaining Reis each independently selected from hydrogen, C₁₋₄ alkyl, halogenated C₁₋₄ alkyl, halogen, C₁₋₄ alkoxy, CN, NO₂, OR^{f}, SR^{f}, NR^{d1}R^{d2}, CORⁱ, (CR^{a1}R^{a2})ₘCOR^{b};
R⁵ and R^{5′} are each independently selected from none, hydrogen, C₁₋₈ alkyl, (CR^{a1}R^{a2})ₘCOR^{b};
R³ is hydrogen, (CR^{a1}R^{a2})ₘCOOR^{b1}, or unsubstituted or optionally substituted by one or more R^{x} groups consisting of C₁₋₄ alkyl, C₂₋₄ alkenyl, C₂₋₄ alkynyl, 3-6 membered cycloalkyl, 3-6 membered heterocyclyl, aryl or heteroaryl; wherein R^{x} is each independently selected from cyano, nitro, hydroxyl, carboxy, halogen, C₁₋₄ alkyl, C₁₋₄ alkoxy, (CR^{a1}R^{a2})ₘNR^{d1}R^{d2}, 3-8 membered cycloalkyl or its halogenated derivatives, 3-8 membered heterocyclyl or its halogenated derivatives, aryl or its halogenated or deuterated derivatives, heteroaryl or its halogenated or deuterated derivatives;
R⁴ is hydrogen,

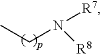

(CR^{a1}R^{a2})ₘCOOR^{b1}, (CR^{a1}R^{a2})ₘOCOR^{b}, (CR^{a1}R^{a2})ₙCOR^{c}, CONR^{d1}R^{d2}, (CR^{a1}R^{a2}), OR^{f};
when Z is selected from none, X is N, Y is N, R⁴ is

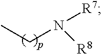

R⁷ is hydrogen, or groups consisting of C₁₋₄ alkyl, 3-6 membered cycloalkyl, 3-6 membered heterocyclyl, aryl or heteroaryl unsubstituted or optionally substituted with one or more R^{y}; wherein R^{y} is each independently selected from cyano, nitro, hydroxyl, carboxy, halogen, C₁₋₄ alkyl, alkoxy, C₁₋₄ alkylthio, O(CR^{a1}R^{a2})ₘNR^{d1}R^{d2}, (CR^{a1}R^{a2})ₘNR^{d1}R^{d2}, 3-8 membered cycloalkyl or its halogenated derivatives, 3-8 membered heterocyclyl or its halogenated derivatives, aryl or its halogenated or deuterated derivatives, heteroaryl or its halogenated or deuterated derivatives;
R⁸ is hydrogen, C₁₋₄ alkyl, (CR^{a1}R^{a2})ₘCOOR^{b1}, (CR^{a1}R^{a2})ₘOCOR^{b}, (CR^{a1}R^{a2})ₙCOR^{c}, CONR^{d1}R^{d2}, (CR^{a1}R^{a2})ₙOR^{f}, (CR^{a1}R^{a2})ₘSO₂OR^{b1}, (CR^{a1}R^{a2})ₘSOOR^{b1};
wherein, R^{a1}, R^{a2} are each independently selected from hydrogen, halogen, C₁₋₄ alkyl, halogenated C₁₋₄ alkyl, C₂₋₄ alkenyl, C₂₋₄ alkynyl, 3-6 membered cycloalkyl, 3-6 membered heterocyclyl, aryl, heteroaryl, CN, OR^{f}, SR^{f}, or NR^{d1}R^{d2}; or, R^{a1} and R^{a2} taken together with the carbon atom to which they are attached, form a 3-6 membered ring containing 0 or 1 heteroatom selected from N, O, or S;
R^{b1} is hydrogen, C₁₋₄ alkyl, C₁₋₄ alkoxy, halogenated C₁₋₄ alkyl, 3-6 membered cycloalkyl, 3-6 membered heterocyclyl, aryl, or heteroaryl;
R^{b} is hydrogen, C₁₋₄ alkyl, C₁₋₄ alkoxy, halogenated C₁₋₄ alkyl, C₂₋₄ alkenyl, C₂₋₄ alkynyl, 3-6 membered cycloalkyl, 3-6 membered heterocyclyl, aryl, or heteroaryl;
R^{c} is hydrogen, C₁₋₄ alkyl, C₁₋₄ alkoxy, C₂₋₄ alkenyl, C₂₋₄ alkynyl, 3-6 membered cycloalkyl, 3-6 membered heterocyclyl, aryl, or heteroaryl;
R^{d1}, R^{d2} are each independently selected from hydrogen, C₁₋₄ alkyl, C₁₋₄ alkoxy, 3-6 membered cycloalkyl, 3-6 membered heterocyclyl; or, R^{d1} and R^{d2} taken together with the nitrogen atom to which they are attached, form a 3-6 membered ring containing 1 or 2 heteroatom selected from N, O, or S;

Wherein the alkyl, alkenyl, alkynyl, cycloalkyl, heterocyclyl, aryl, or heteroaryl in R^{a1}, R^{a2}, R^{d1}, R^{d2}, R^{b}, R^{b1}, R^{c}, R⁵ and R^{5′} are unsubstituted or optionally substituted by one or more groups selected from halogen, C₁₋₄ alkyl, C₁₋₄ alkoxy, halogenated C₁₋₄ alkyl, C₂₋₄ alkenyl, C₂₋₄ alkynyl, 3-6 membered cycloalkyl, 3-6 membered heterocyclyl, CN, NO₂, OR^{f}, SR^{f}, NR^{d1}R^{d2}, CORⁱ, COOR^{f}, OCORⁱ, CONR^{d1}R^{d2}, NR^{d1}CORⁱ, NR^{d1}SO₂Rⁱ, or SO₂Rⁱ;
R^{f} is selected from hydrogen, C₁₋₄ alkyl, or halogenated C₁₋₄ alkyl;
Rⁱ is selected from hydrogen, C₁₋₄ alkyl, C₂₋₄ alkenyl, C₂₋₄ alkynyl, 3-6 membered cycloalkyl, or 3-6 membered heterocyclyl;
m is 0, 1, 2;
n is 0, 1, 2;
r is 0, 1, 2, 3;
p is 0, 1, 2, 3.

Further, said compound has a structure of formula II:

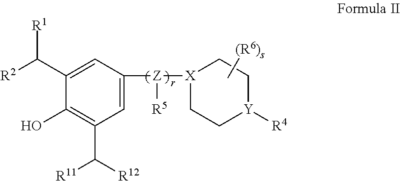

wherein, X is selected from CR³, N;
Y is selected from CR³, N;
Z is selected from none, CR⁵′, N, O;
R¹, R², R¹¹, R¹² are each independently selected from hydrogen, halogen, C₁₋₄ alkyl, C₂₋₄ alkenyl, or 3-6 membered cycloalkyl;
s is selected from an integer from 0 to 8, and each R⁶ is independently selected from hydrogen, C₁₋₄ alkyl, halogenated C₁₋₄ alkyl, C₂₋₄ alkenyl, C₂₋₄ alkynyl, 3-6 membered cycloalkyl, 3-6 membered heterocyclyl, halogen, C₁₋₄ alkoxy, CN, NO₂, OR^{f}, SR^{f}, NR^{d1}R^{d2}, CORⁱ, (CR^{a1}R^{a2})ₘCOR^{b}; or, s is selected from an integer from 2 to 8, wherein two Re groups are bounded to form a ring, and the remaining R⁶ each independently selected from hydrogen, C₁₋₄ alkyl, halogenated C₁₋₄ alkyl, halogen, C₁₋₄ alkoxy, CN, NO₂, OR^{f}, SR^{f}, NR^{d1}R^{d2}, CORⁱ, (CR^{a1}R^{a2})ₘCOR^{b};
R⁵ and R^{5′} are each independently selected from none, hydrogen, C₁₋₈ alkyl, (CR^{a1}R^{a2})ₘCOR^{b};
R³ is hydrogen, (CR^{a1}R^{a2})ₘCOOR^{b1}, unsubstituted or optionally substituted by one or more R^{x} groups consisting of C₁₋₄ alkyl, C₂₋₄ alkenyl, C₂₋₄ alkynyl, 3-6 membered cycloalkyl, 3-6 membered heterocyclyl, aryl or heteroaryl; wherein R^{a} is each independently selected from cyano, nitro, hydroxyl, carboxy, halogen, C₁₋₄ alkyl, C₁₋₄ alkoxy, (CR^{a1}R^{a2})ₘNR^{d1}R^{d2}, 3-8 membered cycloalkyl or its halogenated derivatives, 3-8 membered heterocyclyl or its halogenated derivatives, aryl or its halogenated or deuterated derivatives, heteroaryl or its halogenated or deuterated derivatives;
R⁴ is hydrogen,

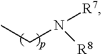

(CR^{a1}R^{a2})ₘCOOR^{b1}, (CR^{a1}R^{a2})ₘOCOR^{b}, (CR^{a1}R^{a2})ₙCOR^{c}, CONR^{d1}R^{d2}, (CR^{a1}R^{a2})ₙOR^{f};
when Z is selected from none, X is N, Y is N, R⁴ is

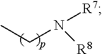

R⁷ is hydrogen, groups consisting of C₁₋₄ alkyl, 3-6 membered cycloalkyl, 3-6 membered heterocyclyl, aryl or heteroaryl unsubstituted or optionally substituted by one or more R^{y}; R^{y} is each independently selected from cyano, nitro, hydroxyl, carboxy, halogen, C₁₋₄ alkyl, alkoxy, C₁₋₄ Alkylthio, O(CR^{a1}R^{a2})ₘNR^{d1}R^{d2}, (CR^{a1}R^{a2})ₘNR^{d1}R^{d2}, 3-8 membered cycloalkyl or its halogen-substituted derivatives, 3-8 membered heterocyclyl or its halogen-substituted derivatives, aryl or its halogen-substituted or deuterated derivatives, heteroaryl or its halogen-substituted or deuterated derivatives;
R⁸ is hydrogen, C₁₋₄ alkyl, (CR^{a1}R^{a2})ₘCOOR^{b1}, (CR^{a1}R^{a2})ₘOCOR^{b}, (CR^{a1}R^{a2})ₙCOR^{c}, CONR^{d1}R^{d2}, (CR^{a1}R^{a2})ₙOR^{f}, (CR^{a1}R^{a2})ₘSO₂OR^{b1}, (CR^{a1}R^{a2})ₘSOOR^{b1};
wherein, R^{a1}, R^{a2} are each independently selected from hydrogen, halogen, C₁₋₄ alkyl, halogenated C₁₋₄ alkyl, C₂₋₄ alkenyl, C₂₋₄ alkynyl, 3-6 membered cycloalkyl, 3-6 membered heterocyclyl, aryl, heteroaryl, CN, OR^{f}, SR^{f}, or NR^{d1}R^{d2}; or, R^{a1} and R^{a2} taken together with the carbon atom to which they are attached, form a 3-6 membered ring containing 0 or 1 heteroatom selected from N, O, or S;
R^{b1} is hydrogen, C₁₋₄ alkyl, C₁₋₄ alkoxy, halogenated C₁₋₄ alkyl, 3-6 membered cycloalkyl, 3-6 membered heterocyclyl, aryl, or heteroaryl;
R^{b} is hydrogen, C₁₋₄ alkyl, C₁₋₄ alkoxy, halogenated C₁₋₄ alkyl, C₂₋₄ alkenyl, C₂₋₄ alkynyl, 3-6 membered cycloalkyl, 3-6 membered heterocyclyl, aryl, or heteroaryl;
R^{c} is hydrogen, C₁₋₄ alkyl, C₁₋₄ alkoxy, C₂₋₄ alkenyl, C₂₋₄ alkynyl, 3-6 membered cycloalkyl, 3-6 membered heterocyclyl, aryl, or heteroaryl;
R^{d1}, R^{d2} are each independently selected from hydrogen, C₁₋₄ alkyl, C₁₋₄ alkoxy, 3-6 membered cycloalkyl, 3-6 membered heterocyclyl; or, R^{d1} and R^{d2} taken together with the nitrogen atom to which they are attached, form a 3-6 membered ring containing 1 or 2 heteroatom selected from N, O, or S;

Wherein the alkyl, alkenyl, alkynyl, cycloalkyl, heterocyclyl, aryl, or heteroaryl in R^{a1}, R^{a2}, R^{d1}, R^{d2}, R^{b}, R^{b1}, R^{c}, R⁵ and R^{5′} are unsubstituted or optionally substituted by one or more groups consisting of halogen, C₁₋₄ alkyl, C₁₋₄ alkoxy, halogenated C₁₋₄ alkyl, C₂₋₄ alkenyl, C₂₋₄ alkynyl, 3-6 membered cycloalkyl, 3-6 membered heterocyclyl, CN, NO₂, OR^{f}, SR^{f}, NR^{d1}R^{d2}, CORⁱ, COOR^{f}, OCORⁱ, CONR^{d1}R^{d2}, NR^{d1}CORⁱ, NR^{d1}SO₂Rⁱ, or SO₂Rⁱ;
R^{f} is selected from hydrogen, C₁₋₄ alkyl, or halogenated C₁₋₄ alkyl;
Rⁱ is selected from hydrogen, C₁₋₄ alkyl, C₂₋₄ alkenyl, C₂₋₄ alkynyl, 3-6 membered cycloalkyl, or 3-6 membered heterocyclyl;
m is selected from 0, 1, 2;
n is selected from 0, 1, 2;
r is selected from 0, 1, 2, 3;
p is selected from 0, 1, 2, 3.

Further, said compound has a structure of formula Ila:

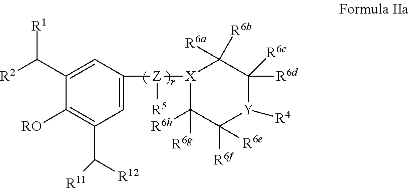

wherein, X is selected from CR³, N;
Y is selected from CR³, N;
Z is selected from none, CR⁵′, N, O;
R¹, R², R¹, R¹² are each independently selected from hydrogen, halogen, C₁₋₄ alkyl, C₂₋₄ alkenyl, or 3-6 membered cycloalkyl;
R^{6a}, R^{6b}, R^{6c}, R^{6d}, R^{6e}, R^{6f}, R^{6g}, R^{6h} are each independently selected from hydrogen, C₁₋₄ alkyl, halogenated C₁₋₄ alkyl, C₂₋₄ alkenyl, C₂₋₄ alkynyl, 3-6 membered cycloalkyl, 3-6 membered heterocyclyl, halogen, C₁₋₄ alkoxy, CN, NO₂, OR^{f}, SR^{f}, NR^{d1}R^{d2}, CORⁱ, (CR^{a1}R^{a2})ₘCOR^{b}; or, any two groups among R^{6a}, R^{6b}, R^{6c}, R^{6d}, R^{6e}, R^{6f}, R^{6g}, R^{6h} cyclize to form a spiro ring, bridged ring, or fused ring, when the remaining groups are each independently selected from hydrogen, C₁₋₄ alkyl, halogenated C₁₋₄ alkyl, halogen, C₁₋₄ alkoxy, CN, NO₂, OR^{f}, SR^{f}, NR^{d1}R^{d2}, CORⁱ, (CR^{a1}R^{a2})ₘCOR^{b};
R⁵ and R^{5′} are each independently selected from none, hydrogen, C₁₋₈ alkyl, (CR^{a1}R^{a2})ₘCOR^{b};
R³ is hydrogen, (CR^{a1}R^{a2})ₘCOOR^{b1}, or groups consisting of C₁₋₄ alkyl, C₂₋₄ alkenyl, C₂₋₄ alkynyl, 3-6 membered cycloalkyl, 3-6 membered heterocyclyl, aryl or heteroaryl unsubstituted or optionally substituted by one or more R^{x}; wherein R^{x} is each independently selected from cyano, nitro, hydroxyl, carboxy, halogen, C₁₋₄ alkyl, C₁₋₄ alkoxy, (CR^{a1}R^{a2})ₘNR^{d1}R^{d2}, 3-8 membered cycloalkyl or its halogenated derivatives, 3-8 membered heterocyclyl or its halogenated derivatives, aryl or its halogenated or deuterated derivatives, heteroaryl or its halogenated or deuterated derivatives;
R⁴ is hydrogen,

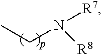

(CR^{a1}R^{a2})ₘCOOR^{b1}, (CR^{a1}R^{a2})ₘOCOR^{b}, (CR^{a1}R^{a2})ₙCOR^{c}, CONR^{d1}R^{d2}, (CR^{a1}R^{a2})ₙOR^{f};
when Z is selected from none, X is N, Y is N, R⁴ is

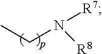

R⁷ is hydrogen, or groups consisiting of C₁₋₄ alkyl, 3-6 membered cycloalkyl, 3-6 membered heterocyclyl, aryl or heteroaryl unsubstituted or optionally substituted with one or more R^{y}; wherein R^{y} is each independently selected from cyano, nitro, hydroxyl, carboxy, halogen, C₁₋₄ alkyl, C₁₋₄ alkoxy, C₁₋₄ alkylthio, O(CR^{a1}R^{a2})ₘNR^{d1}R^{d2}, (CR^{a1}R^{a2})ₘNR^{d1}R^{d2}, 3-8 membered cycloalkyl or its halogenated derivatives, 3-8 membered heterocyclyl or its halogenated derivatives, aryl or its halogenated or deuterated derivatives, heteroaryl or its halogenated or deuterated derivatives;
R⁸ is hydrogen, C₁₋₄ alkyl, (CR^{a1}R^{a2})ₘCOOR^{b1}, (CR^{a1}R^{a2})ₘOCOR^{b}, (CR^{a1}R^{a2})ₙCOR^{c}, CONR^{d1}R^{d2}, (CR^{a1}R^{a2})ₙOR^{f}, (CR^{a1}R^{a2})ₘSO₂OR^{b1}, (CR^{a1}R^{a2})ₘSOOR^{b1};
wherein, R^{a1}, R^{a2} are each independently selected from hydrogen, halogen, C₁₋₄ alkyl, halogenated C₁₋₄ alkyl, C₂₋₄ alkenyl, C₂₋₄ alkynyl, 3-6 membered cycloalkyl, 3-6 membered heterocyclyl, aryl, heteroaryl, CN, OR^{f}, SR^{f}, or NR^{d1}R^{d2}; or, R^{a1} and R^{a2} taken together with the carbon atom to which they are attached, form a 3-6 membered ring containing 0 or 1 heteroatom selected from N, O, or S;
R^{b1} is hydrogen, C₁₋₄ alkyl, C₁₋₄ alkoxy, halogenated C₁₋₄ alkyl, 3-6 membered cycloalkyl, 3-6 membered heterocyclyl, aryl, or heteroaryl;
R^{b} is hydrogen, C₁₋₄ alkyl, C₁₋₄ alkoxy, halogenated C₁₋₄ alkyl, C₂₋₄ alkenyl, C₂₋₄ alkynyl, 3-6 membered cycloalkyl, 3-6 membered heterocyclyl, aryl, or heteroaryl;
R^{c} is hydrogen, C₁₋₄ alkyl, C₁₋₄ alkoxy, C₂₋₄ alkenyl, C₂₋₄ alkynyl, 3-6 membered cycloalkyl, 3-6 membered heterocyclyl, aryl, or heteroaryl;
R^{d1}, R^{d2} are each independently selected from hydrogen, C₁₋₄ alkyl, C₁₋₄ alkoxy, 3-6 membered cycloalkyl, 3-6 membered heterocyclyl; or, R^{d1} and R^{d2} taken together with the nitrogen atom to which they are attached, form a 3-6 ring containing 1 or 2 heteroatom selected from N, O, or S;

Wherein the alkyl, alkenyl, alkynyl, cycloalkyl, heterocyclyl, aryl, or heteroaryl in R^{a1}, R^{a2}, R^{d1}, R^{d2}, R^{b}, R^{b1}, R^{c}, R⁵ and R^{5′} are unsubstituted or optionally substituted by one or more groups selected from halogen, C₁₋₄ alkyl, C₁₋₄ alkoxy, halogenated C₁₋₄ alkyl, C₂₋₄ alkenyl, C₂₋₄ alkynyl, 3-6 membered cycloalkyl, 3-6 membered heterocyclyl, CN, NO₂, OR^{f}, SR^{f}, NR^{d1}R^{d2}, CORⁱ, COOR^{f}, OCORⁱ, CONR^{d1}R^{d2}, NR^{d1}CORⁱ, NR^{d1}SO₂Rⁱ, or SO₂Rⁱ;

R^{f} is selected from hydrogen, C₁₋₄ alkyl, or halogenated C₁₋₄ alkyl;

Rⁱ is selected from hydrogen, C₁₋₄ alkyl, C₂₋₄ alkenyl, C₂₋₄ alkynyl, 3-6 membered cycloalkyl, or 3-6 membered heterocyclyl;
m is selected from 0, 1, 2;
n is selected from 0, 1, 2;
r is selected from 0, 1, 2, 3;
p is selected from 0, 1, 2, 3.
   said

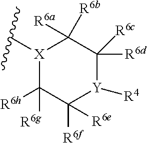

structural fragment is one of the following structures:

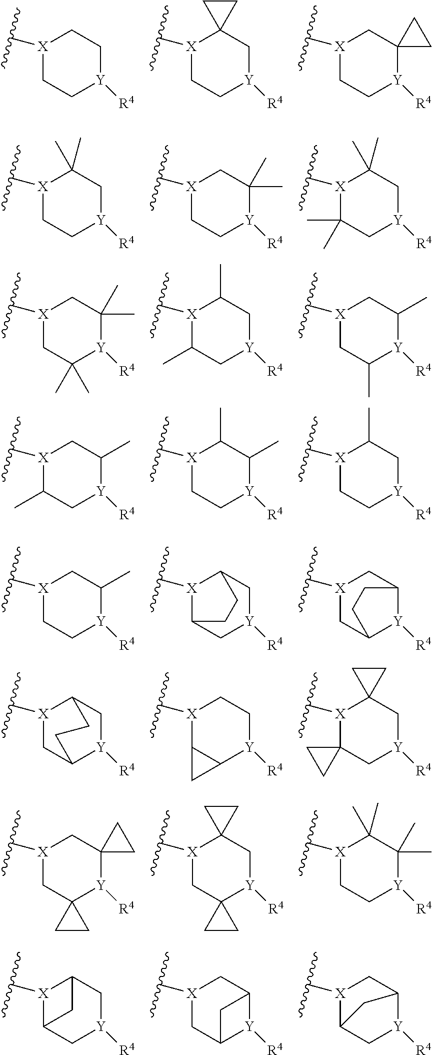

X, Y, R⁴ are as previously described.

Further, said compound has a structure of formula III:

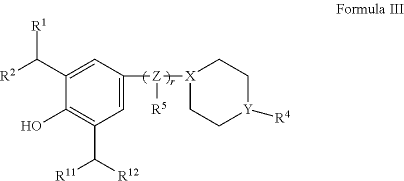

wherein,
X is selected from CR³, N;
Y is selected from CR³, N;
Z is selected from none, CR⁵′, N, O;
R¹, R², R¹¹, R¹² are each independently selected from hydrogen, halogen, C₁₋₄ alkyl, C₂₋₄ alkenyl, or 3-6 membered cycloalkyl;
R⁵ and R^{5′} are each independently selected from none, hydrogen, C₁₋₈ alkyl, (CR^{a1}R^{a2})ₘCOR^{b};
R³ is hydrogen, (CR^{a1}R^{a2})ₘCOOR^{b1}, u or groups consisiting of C₁₋₄ alkyl, C₂₋₄ alkenyl, C₂₋₄ alkynyl, 3-6 membered cycloalkyl, 3-6 membered heterocyclyl, aryl or heteroaryl unsubstituted or optionally substituted by one or more R^{x}; wherein R^{x} is each independently selected from cyano, nitro, hydroxyl, carboxy, halogen, C₁₋₄ alkyl, C₁₋₄ alkoxy, (CR^{a1}R^{a2})ₘNR^{d1}R^{d2}, 3-8 membered cycloalkyl or its halogenated derivatives, 3-8 membered heterocyclyl or its halogenated derivatives, aryl or its halogenated or deuterated derivatives, heteroaryl or its halogenated or deuterated derivatives;
R⁴ is hydrogen,

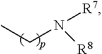

(CR^{a1}R^{a2})ₘCOOR^{b1}, (CR^{a1}R^{a2})ₘOCOR^{b}, (CR^{a1}R^{a2})ₙCOR^{c}, CONR^{d1}R^{d2}, (CR^{a1}R^{a2})ₙOR^{f};
when Z is selected from none, X is N, Y is N, R⁴ is

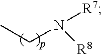

R⁷ is hydrogen, or groups consisting of C₁₋₄ alkyl, 3-6 membered cycloalkyl, 3-6 membered heterocyclyl, aryl or heteroaryl unsubstituted or optionally substituted by one or more R^{y}; wherein R^{y} is each independently selected from cyano, nitro, hydroxyl, carboxy, halogen, C₁₋₄ alkyl, C₁₋₄ alkoxy, C₁₋₄ alkylthio, O(CR^{a1}R^{a2})ₘNR^{d1}R^{d2}, (CR^{a1}R^{a2})ₘNR^{d1}R^{d2}, 3-8 membered cycloalkyl or its halogenated derivatives, 3-8 membered heterocyclyl or its halogenated derivatives, aryl or its halogenated or deuterated derivatives, heteroaryl or its halogenated or deuterated derivatives;
R⁸ is hydrogen, C₁₋₄ alkyl, (CR^{a1}R^{a2})ₘCOOR^{b1}, (CR^{a1}R^{a2})ₘOCOR^{b}, (CR^{a1}R^{a2})ₙCOR^{c}, CONR^{d1}R^{d2}, (CR^{a1}R^{a2})ₙOR^{f}, (CR^{a1}R^{a2})ₘSO₂OR^{b1}, (CR^{a1}R^{a2})ₘSOOR^{b1};
wherein, R^{a1}, R^{a2} are each independently selected from hydrogen, halogen, C₁₋₄ alkyl, halogenated C₁₋₄ alkyl, C₂₋₄ alkenyl, C₂₋₄ alkynyl, 3-6 membered cycloalkyl, 3-6 membered heterocyclyl, aryl, heteroaryl, CN, OR^{f}, SR^{f}, or NR^{d1}R^{d2}; or, R^{a1} and R^{a2} taken together with the carbon atom to which they are attached, form a 3-6 membered ring containing 0 or 1 heteroatom selected from N, O, or S;
R^{b1} is hydrogen, C₁₋₄ alkyl, C₁₋₄ alkoxy, halogenated C₁₋₄ alkyl, 3-6 membered cycloalkyl, 3-6 membered heterocyclyl, aryl, or heteroaryl;
R^{b} is hydrogen, C₁₋₄ alkyl, C₁₋₄ alkoxy, halogenated C₁₋₄ alkyl, C₂₋₄ alkenyl, C₂₋₄ alkynyl, 3-6 membered cycloalkyl, 3-6 membered heterocyclyl, aryl, or heteroaryl;
R^{c} is hydrogen, C₁₋₄ alkyl, C₁₋₄ alkoxy, C₂₋₄ alkenyl, C₂₋₄ alkynyl, 3-6 membered cycloalkyl, 3-6 membered heterocyclyl, aryl, or heteroaryl;
R^{d1}, R^{d2} are each independently selected from hydrogen, C₁₋₄ alkyl, C₁₋₄ alkoxy, 3-6 membered cycloalkyl, 3-6 membered heterocyclyl; or, R^{d1} and R^{d2} taken together with the nitrogen atom to which they are attached, form a 3-6 membered ring containing 1 or 2 heteroatom selected from N, O, or S;

Wherein the alkyl, alkenyl, alkynyl, cycloalkyl, heterocyclyl, aryl, or heteroaryl in R^{a1}, R^{a2}, R^{d1}, R^{d2}, R^{b}, R^{b1}, R^{c}, R⁵ and R^{5′} are unsubstituted or optionally substituted by one or more groups selected from halogen, C₁₋₄ alkyl, C₁₋₄ alkoxy, halogenated C₁₋₄ alkyl, C₂₋₄ alkenyl, C₂₋₄ alkynyl, 3-6 membered cycloalkyl, 3-6 membered heterocyclyl, CN, NO₂, OR^{f}, SR^{f}, NR^{d1}R^{d2}, CORⁱ, COOR^{f}, OCORⁱ, CONR^{d1}R^{d2}, NR^{d1}CORⁱ, NR^{d1}SO₂Rⁱ, or SO₂Rⁱ;
R^{f} is selected from hydrogen, C₁₋₄ alkyl, or halogenated C₁₋₄ alkyl;
Rⁱ is selected from hydrogen, C₁₋₄ alkyl, C₂₋₄ alkenyl, C₂₋₄ alkynyl, 3-6 membered cycloalkyl, or 3-6 membered heterocyclyl;
m is selected from 0, 1, 2;
n is selected from 0, 1, 2;
r is selected from 0, 1, 2, 3;
p is selected from 0, 1, 2, 3.

Further, said compound has a structure of formula IV:

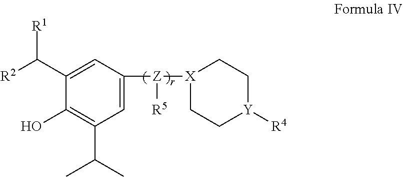

wherein, X is selected from CR³, N;
Y is selected from CR³, N;
Z is selected from none, CR⁵′, N, O;
R¹, R² are each independently selected from hydrogen, halogen, C₁₋₄ alkyl, C₂₋₄ alkenyl, or 3-6 membered cycloalkyl;
R⁵ and R^{5′} are each independently selected from none, hydrogen, C₁₋₈ alkyl, (CR^{a1}R^{a2})ₘCOR^{b};
R³ is hydrogen, (CR^{a1}R^{a2})ₘCOOR^{b1}, or groups consisiting of C₁₋₄ alkyl, C₂₋₄ alkenyl, C₂₋₄ alkynyl, 3-6 membered cycloalkyl, 3-6 membered heterocyclyl, aryl or heteroaryl unsubstituted or optionally substituted by one or more R^{x}; wherein R^{x} is each independently selected from cyano, nitro, hydroxyl, carboxy, halogen, C₁₋₄ alkyl, C₁₋₄ alkoxy, (CR^{a1}R^{a2})ₘNR^{d1}R^{d2}, 3-8 membered cycloalkyl or its halogenated derivatives, 3-8 membered heterocyclyl or its halogenated derivatives, aryl or its halogenated or deuterated derivatives, heteroaryl or its halogenated or deuterated derivatives;
R⁴ is hydrogen,

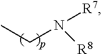

(CR^{a1}R^{a2})ₘCOOR^{b1}, (CR^{a1}R^{a2})ₘOCOR^{b}, (CR^{a1}R^{a2})ₙCOR^{c}, CONR^{d1}R^{d2}, (CR^{a1}R^{a2})ₙOR^{f};
when Z is selected from none, X is N, Y is N, R⁴ is

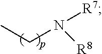

R⁷ is hydrogen, uor groups consisting of C₁₋₄ alkyl, 3-6 membered cycloalkyl, 3-6 membered heterocyclyl, aryl or heteroaryl unsubstituted or optionally substituted by one or more R^{y}; wherein R^{y} is each independently selected from cyano, nitro, hydroxyl, carboxy, halogen, C₁₋₄ alkyl, C₁₋₄ alkoxy, C₁₋₄ alkylthio, O(CR^{a1}R^{a2})ₘNR^{d1}R^{d2}, (CR^{a1}R^{a2})ₘNR^{d1}R^{d2}, 3-8 membered cycloalkyl or its halogenated derivatives, 3-8 membered heterocyclyl or its halogenated derivatives, aryl or its halogenated or deuterated derivatives, heteroaryl or its halogenated or deuterated derivatives;
R⁸ is hydrogen, C₁₋₄ alkyl, (CR^{a1}R^{a2})ₘCOOR^{b1}, (CR^{a1}R^{a2})ₘOCOR^{b}, (CR^{a1}R^{a2})ₙCOR^{c}, CONR^{d1}R^{d2}, (CR^{a1}R^{a2})ₙOR^{f}, (CR^{a1}R^{a2})ₘSO₂OR^{b1}, (CR^{a1}R^{a2})ₘSOOR^{b1};
wherein, R^{a1}, R^{a2} are each independently selected from hydrogen, halogen, C₁₋₄ alkyl, halogenated C₁₋₄ alkyl, C₂₋₄ alkenyl, C₂₋₄ alkynyl, 3-6 membered cycloalkyl, 3-6 membered heterocyclyl, aryl, heteroaryl, CN, OR^{f}, SR^{f}, or NR^{d1}R^{d2}; or, R^{a1} and R^{a2} taken together with the carbon atom to which they are attached, form a 3-6 membered ring containing 0 or 1 heteroatom selected from N, O, or S;
R^{b1} is hydrogen, C₁₋₄ alkyl, C₁₋₄ alkoxy, halogenated C₁₋₄ alkyl, 3-6 membered cycloalkyl, 3-6 membered heterocyclyl, aryl, or heteroaryl;
R^{b} is hydrogen, C₁₋₄ alkyl, C₁₋₄ alkoxy, halogenated C₁₋₄ alkyl, C₂₋₄ alkenyl, C₂₋₄ alkynyl, 3-6 membered cycloalkyl, 3-6 membered heterocyclyl, aryl, or heteroaryl;
R^{c} is hydrogen, C₁₋₄ alkyl, C₁₋₄ alkoxy, C₂₋₄ alkenyl, C₂₋₄ alkynyl, 3-6 membered cycloalkyl, 3-6 membered heterocyclyl, aryl, or heteroaryl;
R^{d1}, R^{d2} are each independently selected from hydrogen, C₁₋₄ alkyl, C₁₋₄ alkoxy, 3-6 membered cycloalkyl, 3-6 membered heterocyclyl; or, R^{d1} and R^{d2} taken together with the nitrogen atom to which they are attached, form a 3-6 membered ring structure containing 1 or 2 heteroatom selected from N, O, or S;

Wherein the alkyl, alkenyl, alkynyl, cycloalkyl, heterocyclyl, aryl, or heteroaryl in R^{a1}, R^{a2}, R^{d1}, R^{d2}, R^{b}, R^{b1}, R^{c}, R⁵ and R^{5′} are unsubstituted or optionally substituted by one or more groups selected from halogen, C₁₋₄ alkyl, C₁₋₄ alkoxy, halogenated C₁₋₄ alkyl, C₂₋₄ alkenyl, C₂₋₄ alkynyl, 3-6 membered cycloalkyl, 3-6 membered heterocyclyl, CN, NO₂, OR^{f}, SR^{f}, NR^{d1}R^{d2}, CORⁱ, COOR^{f}, OCORⁱ, CONR^{d1}R^{d2}, NR^{d1}CORⁱ, NR^{d1}SO₂Rⁱ, or SO₂Rⁱ;
R^{f} is selected from hydrogen, C₁₋₄ alkyl, or halogenated C₁₋₄ alkyl;
Rⁱ is selected from hydrogen, C₁₋₄ alkyl, C₂₋₄ alkenyl, C₂₋₄ alkynyl, 3-6 membered cycloalkyl, or 3-6 membered heterocyclyl;
m is selected from 0, 1, 2;
n is selected from 0, 1, 2;
r is selected from 0, 1, 2, 3;
p is selected from 0, 1, 2, 3.

Further, said compound has a structure of formula V-1 or formula V-2:

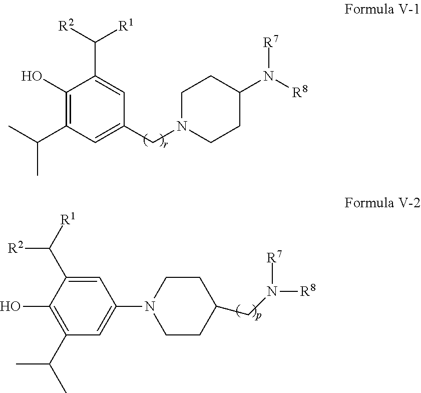

wherein,
R¹, R² are each independently selected from hydrogen, halogen, C₁₋₄ alkyl, C₂₋₄ alkenyl, or 3-6 membered cycloalkyl;
R⁷ is hydrogen, or groups consisting of C₁₋₄ alkyl, 3-6 membered cycloalkyl, 3-6 membered heterocyclyl, aryl or heteroaryl unsubstituted or optionally substituted by one or more R^{y}; wherein R^{y} is each independently selected from cyano, nitro, hydroxyl, carboxy, halogen, C₁₋₄ alkyl, C₁₋₄ alkoxy, C₁₋₄ alkylthio, O(CR^{a1}R^{a2})ₘNR^{d1}R^{d2}, (CR^{a1}R^{a2})ₘNR^{d1}R^{d2}, 3-8 membered cycloalkyl or its halogenated derivatives, 3-8 membered heterocyclyl or its halogenated derivatives, aryl or its halogenated or deuterated derivatives, heteroaryl or its halogenated or deuterated derivatives;
R⁸ is hydrogen, C₁₋₄ alkyl, (CR^{a1}R^{a2})ₘCOOR^{b1}, (CR^{a1}R^{a2})ₘOCOR^{b}, (CR^{a1}R^{a2})ₙCOR^{c}, CONR^{d1}R^{d2}, (CR^{a1}R^{a2})ₙOR^{f}, (CR^{a1}R^{a2})ₘSO₂OR^{b1}, (CR^{a1}R^{a2})ₘSOOR^{b1};
wherein, R^{a1}, R^{a2} are each independently selected from hydrogen, halogen, C₁₋₄ alkyl, halogenated C₁₋₄ alkyl, C₂₋₄ alkenyl, C₂₋₄ alkynyl, 3-6 membered cycloalkyl, 3-6 membered heterocyclyl, aryl, heteroaryl, CN, OR^{f}, SR^{f}, or NR^{d1}R^{d2}; or, R^{a1} and R^{a2} taken together with the carbon atom to which they are attached, form a 3-6 membered ring containing 0 or 1 heteroatom selected from N, O, or S;
R^{b1} is hydrogen, C₁₋₄ alkyl, C₁₋₄ alkoxy, halogenated C₁₋₄ alkyl, 3-6 membered cycloalkyl, 3-6 membered heterocyclyl, aryl, or heteroaryl;
R^{b} is hydrogen, C₁₋₄ alkyl, C₁₋₄ alkoxy, halogenated C₁₋₄ alkyl, C₂₋₄ alkenyl, C₂₋₄ alkynyl, 3-6 membered cycloalkyl, 3-6 membered heterocyclyl, aryl, or heteroaryl;
R^{c} is hydrogen, C₁₋₄ alkyl, C₁₋₄ alkoxy, C₂₋₄ alkenyl, C₂₋₄ alkynyl, 3-6 membered cycloalkyl, 3-6 membered heterocyclyl, aryl, or heteroaryl;
R^{d1}, R^{d2} are each independently selected from hydrogen, C₁₋₄ alkyl, C₁₋₄ alkoxy, 3-6 membered cycloalkyl, 3-6 membered heterocyclyl; or, R^{d1} and R^{d2} taken together with the nitrogen atom to which they are attached, form a 3-6 membered ring containing 1 or 2 heteroatom selected from N, O, or S;

Wherein the alkyl, alkenyl, alkynyl, cycloalkyl, heterocyclyl, aryl, or heteroaryl in R^{a1}, R^{a2}, R^{d1}, R^{d2}, R^{b}, R^{b1} and R^{c} are unsubstituted or optionally substituted by one or more groups selected from halogen, C₁₋₄ alkyl, C₁₋₄ alkoxy, halogenated C₁₋₄ alkyl, C₂₋₄ alkenyl, C₂₋₄ alkynyl, 3-6 membered cycloalkyl, 3-6 membered heterocyclyl, CN, NO₂, OR^{f}, SR^{f}, NR^{d1}R^{d2}, CORⁱ, COOR^{f}, OCORⁱ, CONR^{d1}R^{d2}, NR^{d1}CORⁱ, NR^{d1}SO₂Rⁱ, or SO₂Rⁱ;
R^{f} is selected from hydrogen, C₁₋₄ alkyl or halogenated C₁₋₄ alkyl;
Rⁱ is selected from hydrogen, C₁₋₄ alkyl, C₂₋₄ alkenyl, C₂₋₄ alkynyl, 3-6 membered cycloalkyl, or 3-6 membered heterocyclyl;
m is selected from 0, 1, 2;
n is selected from 0, 1, 2;
r is selected from 0, 1, 2, 3;
p is selected from 0, 1, 2, 3.

Further, said compound has a structure of formula V-3:

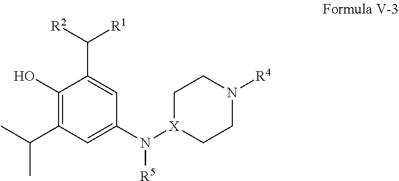

wherein,
X is selected from CR³, N;
R¹, R² are each independently selected from hydrogen, halogen, C₁₋₄ alkyl, C₂₋₄ alkenyl, or 3-6 membered cycloalkyl;
R⁵ is selected from none, hydrogen, C₁₋₈ alkyl, (CR^{a1}R^{a2})ₘCOR^{b};
R³ is hydrogen, (CR^{a1}R^{a2})ₘCOOR^{b1}, or groups consisting of C₁₋₄ alkyl, C₂₋₄ alkenyl, C₂₋₄ alkynyl, 3-6 membered cycloalkyl, 3-6 membered heterocyclyl, aryl or heteroaryl unsubstituted or optionally substituted by one or more R^{x}, wherein R^{x} is each independently selected from cyano, nitro, hydroxyl, carboxy, halogen, C₁₋₄ alkyl, C₁₋₄ alkoxy, (CR^{a1}R^{a2})ₘNR^{d1}R^{d2}, 3-8 membered cycloalkyl or its halogenated derivatives, 3-8 membered heterocyclyl or its halogenated derivatives, aryl or its halogenated or deuterated derivatives, heteroaryl or its halogenated or deuterated derivatives;
R⁴ is hydrogen, (CR^{a1}R^{a2})ₘCOOR^{b1}, (CR^{a1}R^{a2})ₘOCOR^{b}, (CR^{a1}R^{a2})ₙCOR^{c}, CONR^{d1}R^{d2}, (CR^{a1}R^{a2})ₙOR^{f};
wherein, R^{a1}, R^{a2} are each independently selected from hydrogen, halogen, C₁₋₄ alkyl, halogenated C₁₋₄ alkyl, C₂₋₄ alkenyl, C₂₋₄ alkynyl, 3-6 membered cycloalkyl, 3-6 membered heterocyclyl, aryl, heteroaryl, CN, OR^{f}, SR^{f} or NR^{d1}R^{d2}; or, R^{a1} and R^{a2} taken together with the carbon atom to which they are attached, form a 3-6 membered ring containing 0 or 1 heteroatom selected from N, O, or S;
R^{b1} is hydrogen, C₁₋₄ alkyl, C₁₋₄ alkoxy, halogenated C₁₋₄ alkyl, 3-6 membered cycloalkyl, 3-6 membered heterocyclyl, aryl, or heteroaryl;
R^{b} is hydrogen, C₁₋₄ alkyl, C₁₋₄ alkoxy, halogenated C₁₋₄ alkyl, C₂₋₄ alkenyl, C₂₋₄ alkynyl, 3-6 membered cycloalkyl, 3-6 membered heterocyclyl, aryl, or heteroaryl;
R^{c} is hydrogen, C₁₋₄ alkyl, C₁₋₄ alkoxy, C₂₋₄ alkenyl, C₂₋₄ alkynyl, 3-6 membered cycloalkyl, 3-6 membered heterocyclyl, aryl, or heteroaryl;
R^{d1}, R^{d2} are each independently selected from hydrogen, C₁₋₄ alkyl, C₁₋₄ alkoxy, 3-6 membered cycloalkyl, 3-6 membered heterocyclyl; or, R^{d1} and R^{d2} taken together with the nitrogen atom to which they are attached, form a 3-6 membered ring containing 1 or 2 heteroatom selected from N, O, or S;

Wherein, the alkyl, alkenyl, alkynyl, cycloalkyl, heterocyclyl, aryl, or heteroaryl in R^{a1}, R^{a2}, R^{d1}, R^{d2}, R^{b}, R^{b1}, R^{c} and R⁵ are unsubstituted or optionally substituted by one or more groups selected from halogen, C₁₋₄ alkyl, C₁₋₄ alkoxy, halogenated C₁₋₄ alkyl, C₂₋₄ alkenyl, C₂₋₄ alkynyl, 3-6 membered cycloalkyl, 3-6 membered heterocyclyl, CN, NO₂, OR^{f}, SR^{f}, NR^{d1}R^{d2}, CORⁱ, COOR^{f}, OCORⁱ, CONR^{d1}R^{d2}, NR^{d1}CORⁱ, NR^{d1}SO₂Rⁱ, or SO₂Rⁱ;
R^{f} is selected from hydrogen, C₁₋₄ alkyl or halogenated C₁₋₄ alkyl;
Rⁱ is selected from hydrogen, C₁₋₄ alkyl, C₂₋₄ alkenyl, C₂₋₄ alkynyl, 3-6 membered cycloalkyl, or 3-6 membered heterocyclyl;
m is selected from 0, 1, 2;
n is selected from 0, 1, 2.

Further, said compound has a structure of formula V-4:

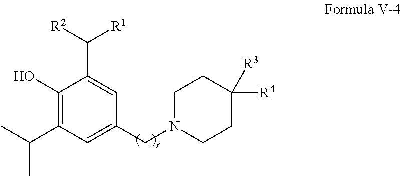

wherein,
R¹, R² are each independently selected from hydrogen, halogen, C₁₋₄ alkyl, C₂₋₄ alkenyl or 3-6 membered cycloalkyl;
R³ is hydrogen, (CR^{a1}R^{a2})ₘCOOR^{b1}, or groups consisting of C₁₋₄ alkyl, C₂₋₄ alkenyl, C₂₋₄ alkynyl, 3-6 membered cycloalkyl, 3-6 membered heterocyclyl, aryl or heteroaryl unsubstituted or optionally substituted by one or more R^{x}; wherein R^{x} is each independently selected from cyano, nitro, hydroxyl, carboxy, halogen, C₁₋₄ alkyl, C₁₋₄ alkoxy, (CR^{a1}R^{a2})ₘNR^{d1}R^{d2}, 3-8 membered cycloalkyl or its halogenated derivatives, 3-8 membered heterocyclyl or its halogenated derivatives, aryl or its halogenated or deuterated derivatives, heteroaryl or its halogenated or deuterated derivatives;
R⁴ is hydrogen,

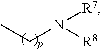

(CR^{a1}R^{a2})ₘCOOR^{b1}, (CR^{a1}R^{a2})ₘOCOR^{b}, (CR^{a1}R^{a2})ₙCOR^{c}, CONR^{d1}R^{d2}, (CR^{a1}R^{a2})ₙOR^{f};
R⁷ is hydrogen, or groups consisiting of C₁₋₄ alkyl, 3-6 membered cycloalkyl, 3-6 membered heterocyclyl, aryl or heteroaryl unsubstituted or optionally substituted by one or more R^{y}; wherein R^{y} is each independently selected from cyano, nitro, hydroxyl, carboxy, halogen, C₁₋₄ alkyl, C₁₋₄ alkoxy, C₁₋₄ alkylthio, O(CR^{a1}R^{a2})ₘNR^{d1}R^{d2}, (CR^{a1}R^{a2})ₘNR^{d1}R^{d2}, 3-8 membered cycloalkyl or its halogenated derivatives, 3-8 membered heterocyclyl or its halogenated derivatives, aryl or its halogenated or deuterated derivatives, heteroaryl or its halogenated or deuterated derivatives;
R⁸ is hydrogen, C₁₋₄ alkyl, (CR^{a1}R^{a2})ₘCOOR^{b1}, (CR^{a1}R^{a2})ₘOCOR^{b}, (CR^{a1}R^{a2})ₙCOR^{c}, CONR^{d1}R^{d2}, (CR^{a1}R^{a2})ₙOR^{f}, (CR^{a1}R^{a2})ₘSO₂OR^{b1}, (CR^{a1}R^{a2})ₘSOOR^{b1};
wherein, R^{a1}, R^{a2} are each independently selected from hydrogen, halogen, C₁₋₄ alkyl, halogenated C₁₋₄ alkyl, C₂₋₄ alkenyl, C₂₋₄ alkynyl, 3-6 membered cycloalkyl, 3-6 membered heterocyclyl, aryl, heteroaryl, CN, OR^{f}, SR^{f}, or NR^{d1}R^{d2}; or, R^{a1} and R^{a2} taken together with the carbon atom to which they are attached, form a 3-6 membered ring containing 0 or 1 heteroatom selected from N, O, or S;
R^{b1} is hydrogen, C₁₋₄ alkyl, C₁₋₄ alkoxy, halogenated C₁₋₄ alkyl, 3-6 membered cycloalkyl, 3-6 membered heterocyclyl, aryl, or heteroaryl;
R^{b} is hydrogen, C₁₋₄ alkyl, C₁₋₄ alkoxy, halogenated C₁₋₄ alkyl, C₂₋₄ alkenyl, C₂₋₄ alkynyl, 3-6 membered cycloalkyl, 3-6 membered heterocyclyl, aryl, or heteroaryl;
R^{c} is hydrogen, C₁₋₄ alkyl, C₁₋₄ alkoxy, C₂₋₄ alkenyl, C₂₋₄ alkynyl, 3-6 membered cycloalkyl, 3-6 membered heterocyclyl, aryl, or heteroaryl;
R^{d1}, R^{d2} are each independently selected from hydrogen, C₁₋₄ alkyl, C₁₋₄ alkoxy, 3-6 membered cycloalkyl, 3-6 membered heterocyclyl; or, R^{d1} and R^{d2} taken together with the nitrogen atom to which they are attached, form a 3-6 membered ring containing 1 or 2 heteroatom selected from N, O, or S;

Wherein, the alkyl, alkenyl, alkynyl, cycloalkyl, heterocyclyl, aryl, or heteroaryl in R^{a1}, R^{a2}, R^{d1}, R^{d2}, R^{b}, R^{b1} and R^{c} are unsubstituted or optionally substituted by one or more groups selected from halogen, C₁₋₄ alkyl, C₁₋₄ alkoxy, halogenated C₁₋₄ alkyl, C₂₋₄ alkenyl, C₂₋₄ alkynyl, 3-6 membered cycloalkyl, 3-6 membered heterocyclyl, CN, NO₂, OR^{f}, SR^{f}, NR^{d1}R^{d2}, CORⁱ, COOR^{f}, OCORⁱ, CONR^{d1}R^{d2}, NR^{d1}CORⁱ, NR^{d1}SO₂Rⁱ, or SO₂Rⁱ;
R^{f} is selected from hydrogen, C₁₋₄ alkyl, or halogenated C₁₋₄ alkyl;
Rⁱ is selected from hydrogen, C₁₋₄ alkyl, C₂₋₄ alkenyl, C₂₋₄ alkynyl, 3-6 membered cycloalkyl, or 3-6 membered heterocyclyl;
m is selected from 0, 1, 2;
n is selected from 0, 1, 2;
r is selected from 0, 1, 2, 3;
p is selected from 0, 1, 2, 3.

Further, said compound has a structure of formula V-5 or V-6:

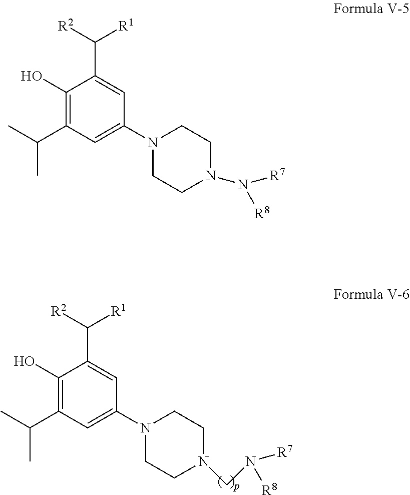

wherein,
R¹, R² are each independently selected from hydrogen, halogen, C₁₋₄ alkyl, C₂₋₄ alkenyl or 3-6 membered cycloalkyl;
R⁷ is hydrogen, or groups consisiting of C₁₋₄ alkyl, 3-6 membered cycloalkyl, 3-6 membered heterocyclyl, aryl or heteroaryl unsubstituted or optionally substituted by one or more R^{y}; R^{y} is each independently selected from cyano, nitro, hydroxyl, carboxy, halogen, C₁₋₄ alkyl, C₁₋₄ alkoxy, C₁₋₄ alkylthio, O(CR^{a1}R^{a2})ₘNR^{d1}R^{d2}, (CR^{a1}R^{a2})ₘNR^{d1}R^{d2}, 3-8 membered cycloalkyl or its halogenated derivatives, 3-8 membered heterocyclyl or its halogenated derivatives, aryl or its halogenated or deuterated derivatives, heteroaryl or its halogenated or deuterated derivatives;
R⁸ is hydrogen, C₁₋₄ alkyl, (CR^{a1}R^{a2})ₘCOOR^{b1}, (CR^{a1}R^{a2})ₘOCOR^{b}, (CR^{a1}R^{a2})ₙCOR^{c}, CONR^{d1}R^{d2}, (CR^{a1}R^{a2})ₙOR^{f}, (CR^{a1}R^{a2})ₘSO₂OR^{b1}, (CR^{a1}R^{a2})ₘSOOR^{b1};
wherein, R^{a1}, R^{a2} are each independently selected from hydrogen, halogen, C₁₋₄ alkyl, halogenated C₁₋₄ alkyl, C₂₋₄ alkenyl, C₂₋₄ alkynyl, 3-6 membered cycloalkyl, 3-6 membered heterocyclyl, aryl, heteroaryl, CN, OR^{f}, SR^{f}, or NR^{d1}R^{d2}; or, R^{a1} and R^{a2} taken together with the carbon atom to which they are attached, form a 3-6 membered ring containing 0 or 1 heteroatom selected from N, O, or S;
R^{b1} is hydrogen, C₁₋₄ alkyl, C₁₋₄ alkoxy, halogenated C₁₋₄ alkyl, 3-6 membered cycloalkyl, 3-6 membered heterocyclyl, aryl, or heteroaryl;
R^{b} is hydrogen, C₁₋₄ alkyl, C₁₋₄ alkoxy, halogenated C₁₋₄ alkyl, C₂₋₄ alkenyl, C₂₋₄ alkynyl, 3-6 membered cycloalkyl, 3-6 membered heterocyclyl, aryl, or heteroaryl;
R^{c} is hydrogen, C₁₋₄ alkyl, C₁₋₄ alkoxy, C₂₋₄ alkenyl, C₂₋₄ alkynyl, 3-6 membered cycloalkyl, 3-6 membered heterocyclyl, aryl, or heteroaryl;
R^{d1}, R^{d2} are each independently selected from hydrogen, C₁₋₄ alkyl, C₁₋₄ alkoxy, 3-6 membered cycloalkyl, 3-6 membered heterocyclyl; or, R^{d1} and R^{d2} taken together with the nitrogen atom to which they are attached, form a 3-6 membered ring containing 1 or 2 heteroatom selected from N, O, or S;
Wherein, the alkyl, alkenyl, alkynyl, cycloalkyl, heterocyclyl, aryl, or heteroaryl in R^{a1}, R^{a2}, R^{d1}, R^{d2}, R^{b}, R^{b1} and R^{c} are unsubstituted or optionally substituted by one or more groups selected from halogen, C₁₋₄ alkyl, C₁₋₄ alkoxy, halogenated C₁₋₄ alkyl, C₂₋₄ alkenyl, C₂₋₄ alkynyl, 3-6 membered cycloalkyl, 3-6 membered heterocyclyl, CN, NO₂, ORE, SR^{f}, NR^{d1}R^{d2}, CORⁱ, COOR^{f}, OCORⁱ, CONR^{d1}R^{d2}, NR^{d1}CORⁱ, NR^{d1}SO₂Rⁱ, or SO₂Rⁱ,
R^{f} is selected from hydrogen, C₁₋₄ alkyl, or halogenated C₁₋₄ alkyl;
Rⁱ is selected from hydrogen, C₁₋₄ alkyl, C₂₋₄ alkenyl, C₂₋₄ alkynyl, 3-6 membered cycloalkyl, or 3-6 membered heterocyclyl;
m is selected from 0, 1, 2;
n is selected from 0, 1, 2;
p is selected from 0, 1, 2, 3.

Further, said compound has a structure of formula VI-1 or VI-2:

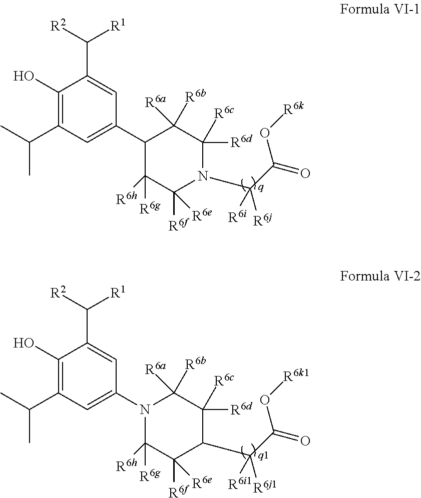

R¹, R², R¹¹, R¹² R^{6a}, R^{6b}, R^{6c}, R^{6d}, R^{6e}, R^{6f}, R^{6g} and R^{6h} are as described in claim 4;
R⁶ⁱ, R^{6j}, R⁶ⁱ¹, R^{6j1} are each independently selected from hydrogen, halogen, C₁₋₆ alkyl, halogenated C₁₋₆ alkyl, C₂₋₆ alkenyl, C₂₋₆ alkynyl, 3-6 membered cycloalkyl, 3-6 membered heterocyclyl, aryl, heteroaryl, CN, OR^{f}, SR^{f} or NR^{d1}R^{d2}, or, R⁶ⁱ and R^{6j}, or, R⁶ⁱ¹ and R^{6j1} selected taken together with the carbon atom to which they are attached, form a 3-6 menbered ring containing 0 or 1 heteroatom selected from N, O, or S;
R^{6k}, R^{6k1} are each independently selected from hydrogen, C₁₋₆ alkyl, halogenated C₁₋₆ alkyl, C₂₋₆ alkenyl, C₂₋₆ alkynyl, 3-6 membered cycloalkyl, 3-6 membered heterocyclyl, aryl, heteroaryl;
R^{f} is selected from hydrogen, C₁₋₄ alkyl or halogenated C₁₋₄ alkyl;
R^{d1}, R^{d2} are each independently selected from hydrogen, C₁₋₄ alkyl, C₁₋₄ alkoxy, 3-6 membered cycloalkyl, 3-6 membered heterocyclyl; or, R^{d1} and R^{d2} taken together with the nitrogen atom to which they are attached, form a 3-6 membered ring containing 1 or 2 heteroatom selected from N, O, or S;
q is selected from 0, 1, 2, 3, 4;
q1 is selected from 0, 1, 2, 3, 4.

Further, the structure of said compound is selected from:

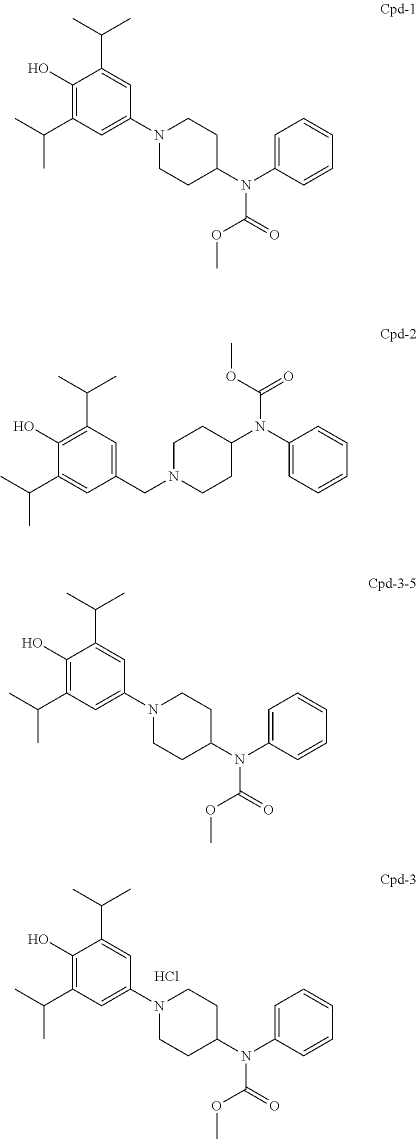 [SMILES: COC(=O)N(c1ccccc1)C1CCN(c2cc(C(C)C)c(O)c(C(C)C)c2)CC1 ; COC(=O)N(c1ccccc1)C1CCN(Cc2cc(C(C)C)c(O)c(C(C)C)c2)CC1 ; COC(=O)N(c1ccccc1)C1CCN(c2cc(C(C)C)c(O)c(C(C)C)c2)CC1 ; COC(=O)N(c1ccccc1)C1CCN(c2cc(C(C)C)c(O)c(C(C)C)c2)CC1 ; Cl]

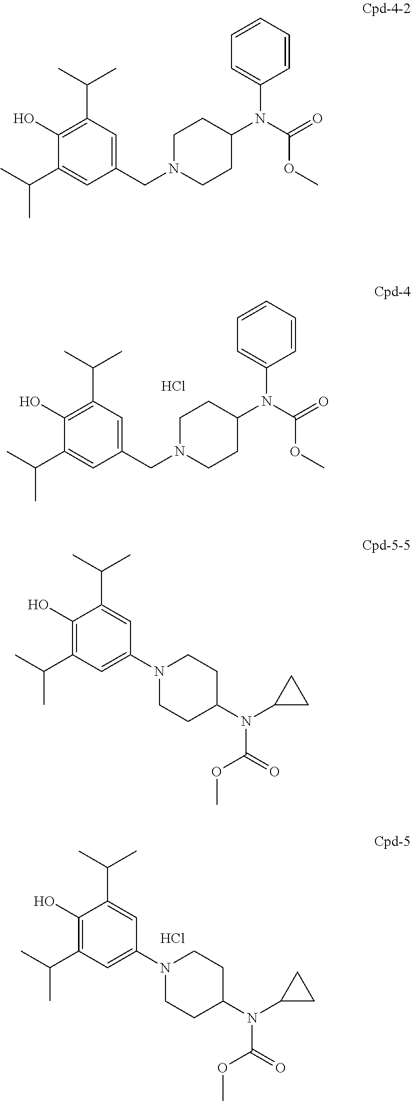

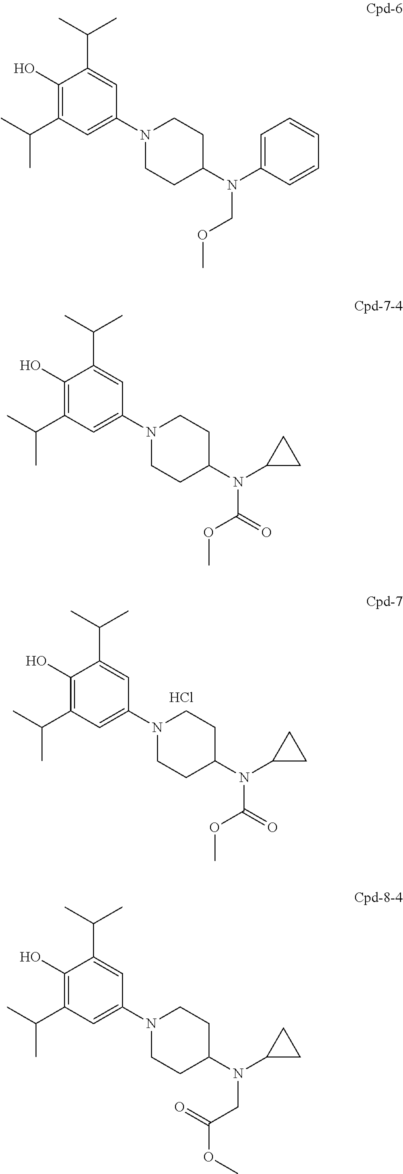 [SMILES: COCN(c1ccccc1)C1CCN(c2cc(C(C)C)c(O)c(C(C)C)c2)CC1 ; COC(=O)N(C1CC1)C1CCN(c2cc(C(C)C)c(O)c(C(C)C)c2)CC1 ; COC(=O)N(C1CC1)C1CCN(c2cc(C(C)C)c(O)c(C(C)C)c2)CC1 ; Cl ; COC(=O)CN(C1CC1)C1CCN(c2cc(C(C)C)c(O)c(C(C)C)c2)CC1]

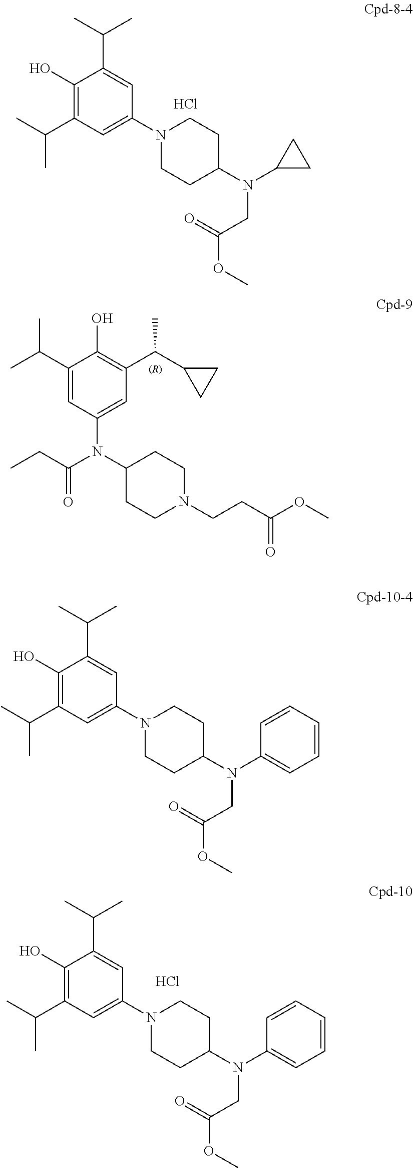

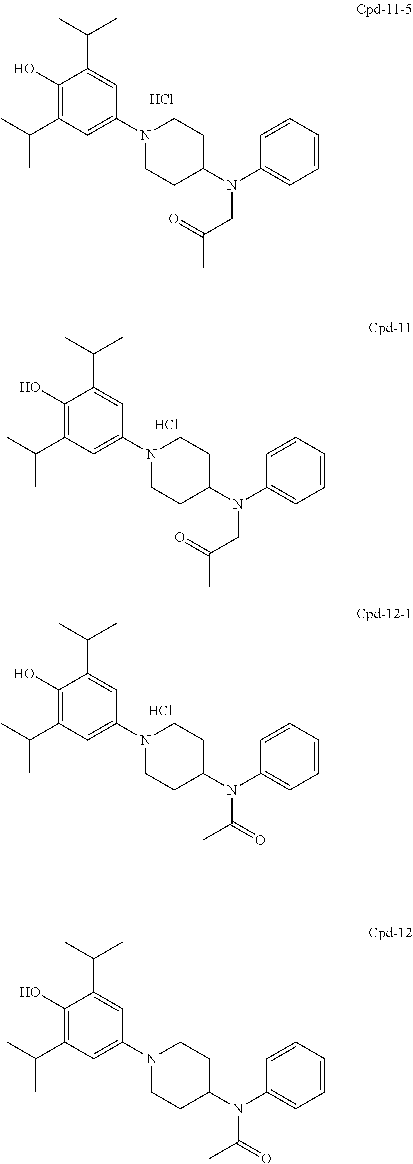

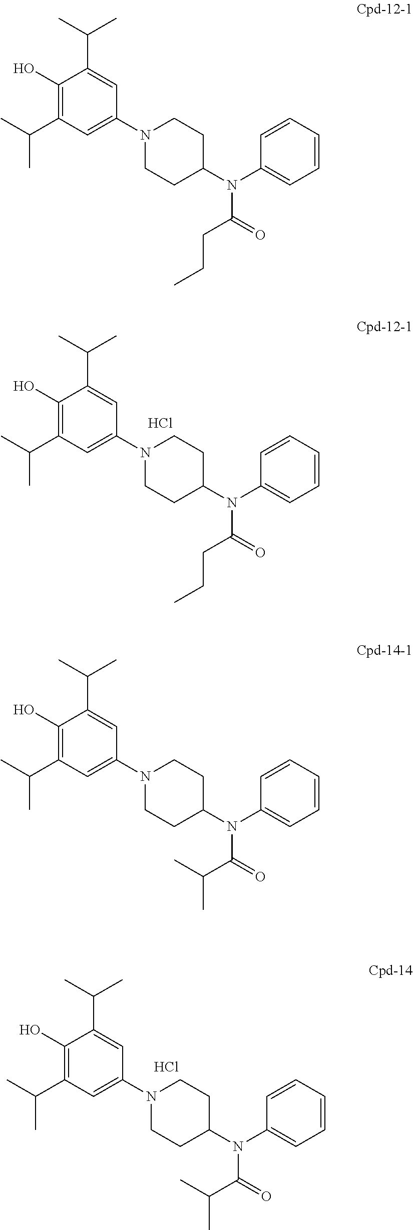 [SMILES: CCCC(=O)N(c1ccccc1)C1CCN(c2cc(C(C)C)c(O)c(C(C)C)c2)CC1 ; CCCC(=O)N(c1ccccc1)C1CCN(c2cc(C(C)C)c(O)c(C(C)C)c2)CC1 ; Cl ; CC(C)C(=O)N(c1ccccc1)C1CCN(c2cc(C(C)C)c(O)c(C(C)C)c2)CC1 ; CC(C)C(=O)N(c1ccccc1)C1CCN(c2cc(C(C)C)c(O)c(C(C)C)c2)CC1 ; Cl]

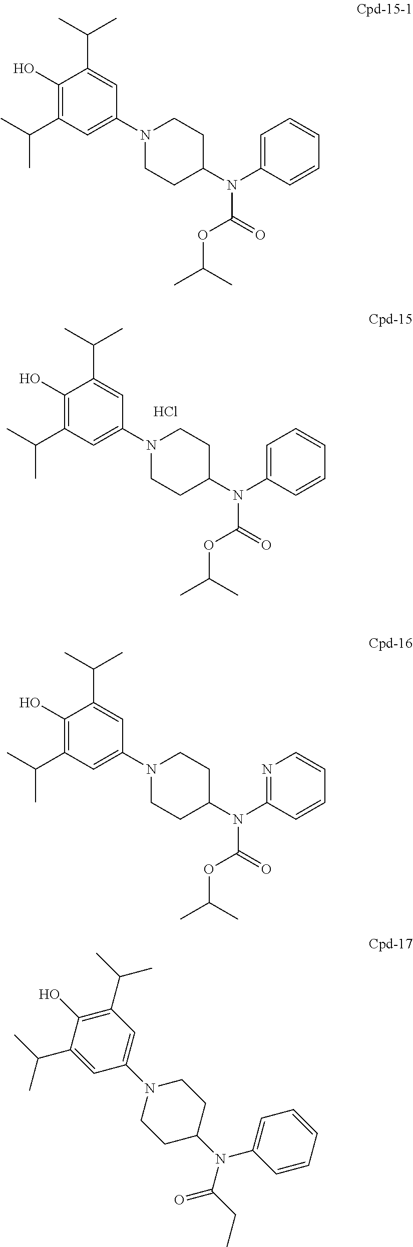 [SMILES: CC(C)OC(=O)N(c1ccccc1)C1CCN(c2cc(C(C)C)c(O)c(C(C)C)c2)CC1 ; CC(C)OC(=O)N(c1ccccc1)C1CCN(c2cc(C(C)C)c(O)c(C(C)C)c2)CC1 ; Cl ; CC(C)OC(=O)N(c1ccccn1)C1CCN(c2cc(C(C)C)c(O)c(C(C)C)c2)CC1 ; CCC(=O)N(c1ccccc1)C1CCN(c2cc(C(C)C)c(O)c(C(C)C)c2)CC1]

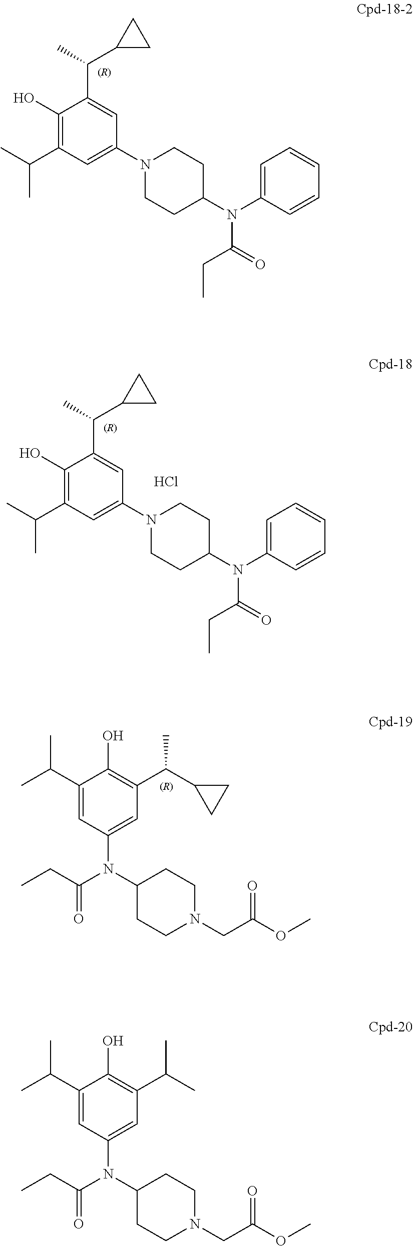

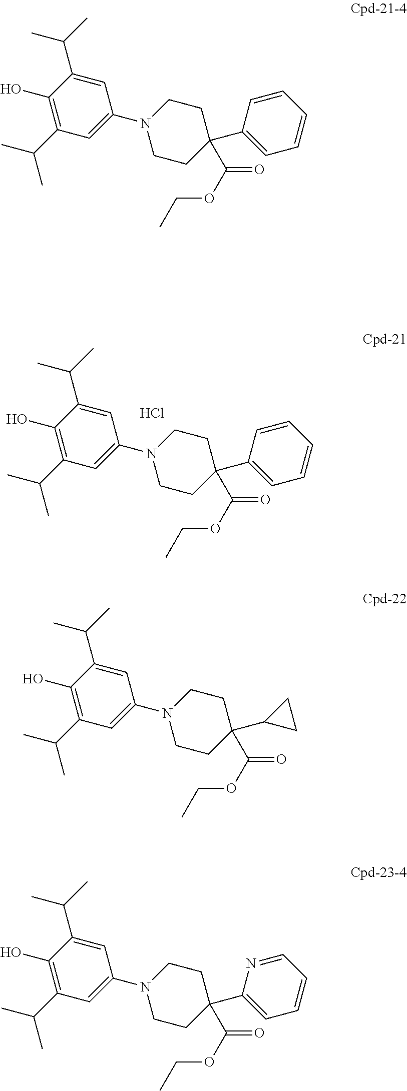 [SMILES: CCOC(=O)C1(c2ccccc2)CCN(c2cc(C(C)C)c(O)c(C(C)C)c2)CC1 ; CCOC(=O)C1(c2ccccc2)CCN(c2cc(C(C)C)c(O)c(C(C)C)c2)CC1 ; Cl ; CCOC(=O)C1(C2CC2)CCN(c2cc(C(C)C)c(O)c(C(C)C)c2)CC1 ; CCOC(=O)C1(c2ccccn2)CCN(c2cc(C(C)C)c(O)c(C(C)C)c2)CC1]

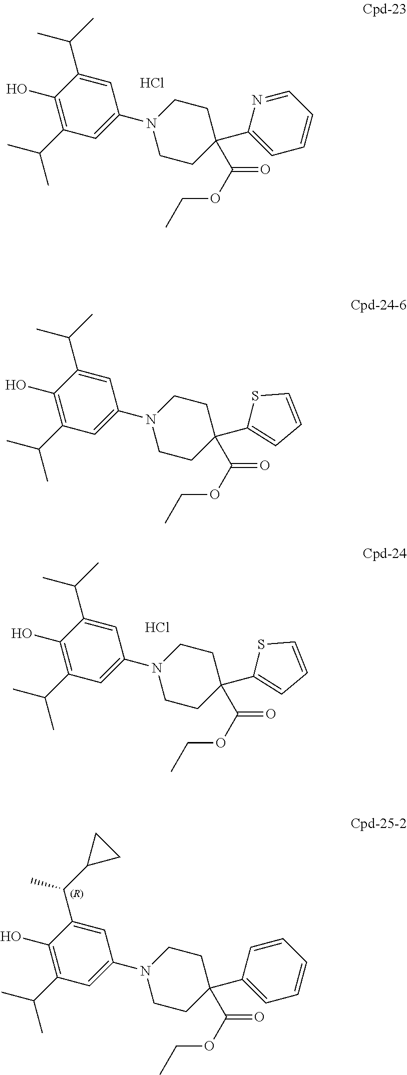

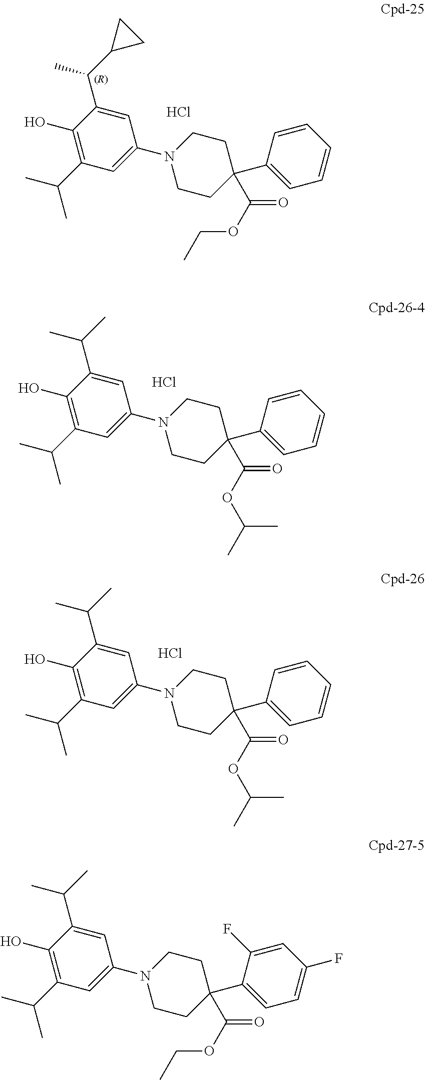

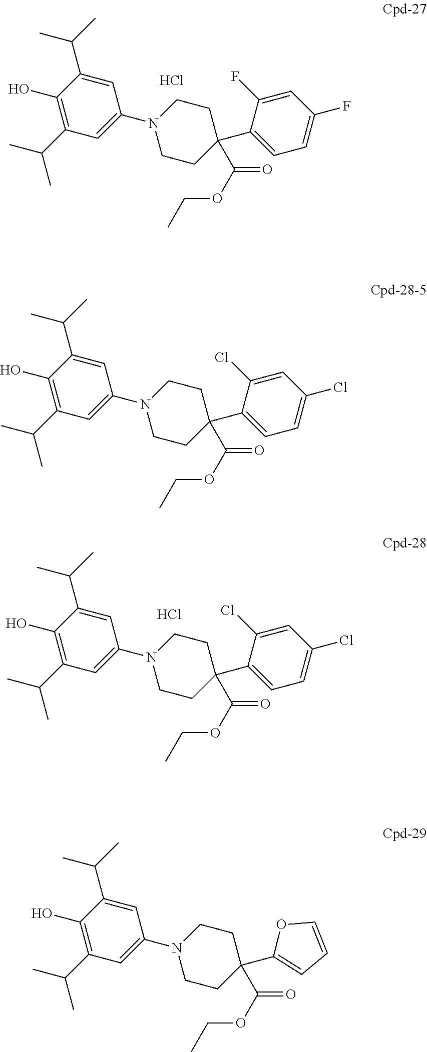 [SMILES: CCOC(=O)C1(c2ccc(F)cc2F)CCN(c2cc(C(C)C)c(O)c(C(C)C)c2)CC1 ; Cl ; CCOC(=O)C1(c2ccc(Cl)cc2Cl)CCN(c2cc(C(C)C)c(O)c(C(C)C)c2)CC1 ; CCOC(=O)C1(c2ccc(Cl)cc2Cl)CCN(c2cc(C(C)C)c(O)c(C(C)C)c2)CC1 ; Cl ; CCOC(=O)C1(c2ccco2)CCN(c2cc(C(C)C)c(O)c(C(C)C)c2)CC1]

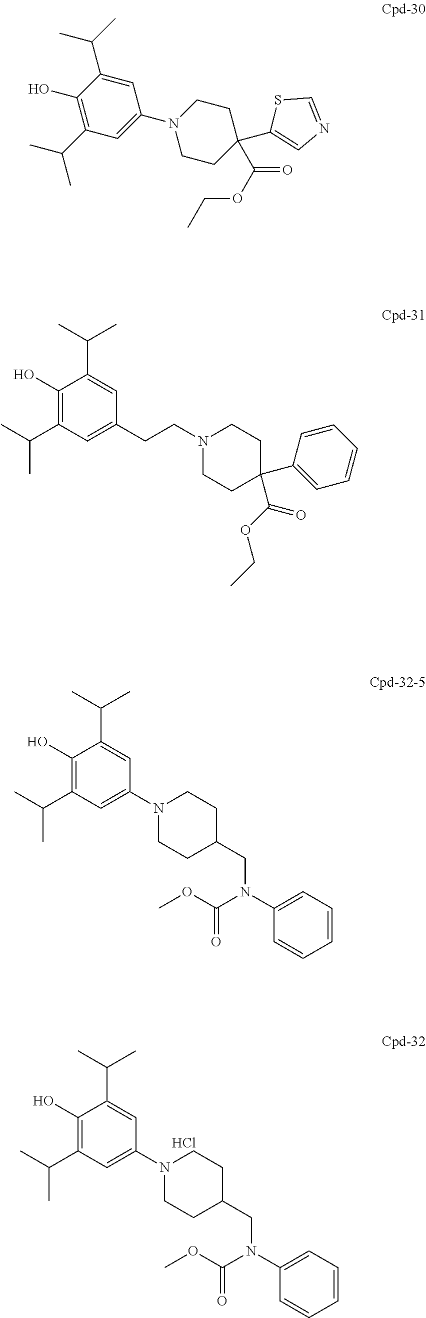 [SMILES: CCOC(=O)C1(c2cncs2)CCN(c2cc(C(C)C)c(O)c(C(C)C)c2)CC1 ; CCOC(=O)C1(c2ccccc2)CCN(CCc2cc(C(C)C)c(O)c(C(C)C)c2)CC1 ; COC(=O)N(CC1CCN(c2cc(C(C)C)c(O)c(C(C)C)c2)CC1)c1ccccc1 ; COC(=O)N(CC1CCN(c2cc(C(C)C)c(O)c(C(C)C)c2)CC1)c1ccccc1 ; Cl]

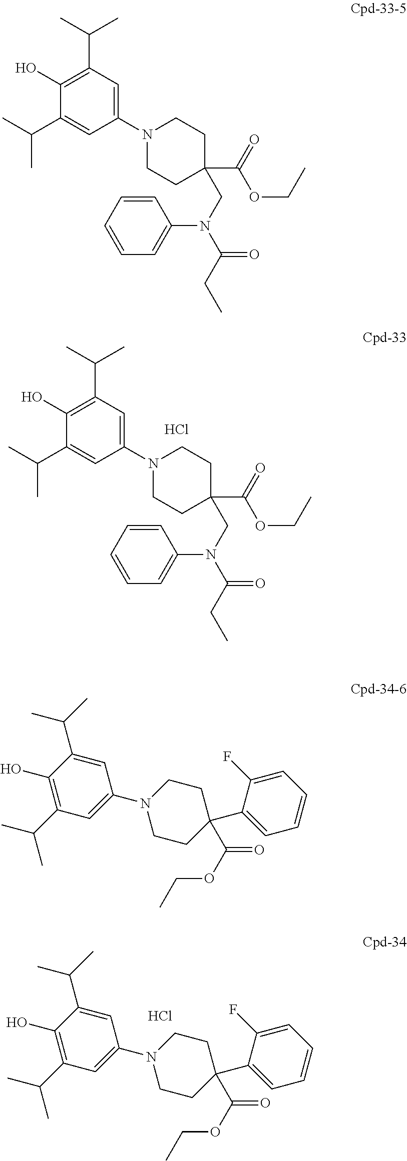

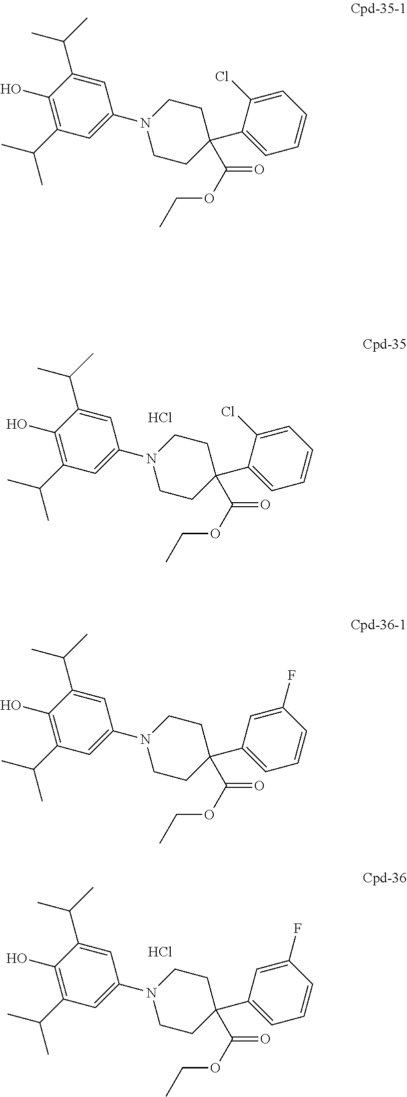 [SMILES: CCOC(=O)C1(c2ccccc2Cl)CCN(c2cc(C(C)C)c(O)c(C(C)C)c2)CC1 ; CCOC(=O)C1(c2ccccc2Cl)CCN(c2cc(C(C)C)c(O)c(C(C)C)c2)CC1 ; Cl ; CCOC(=O)C1(c2cccc(F)c2)CCN(c2cc(C(C)C)c(O)c(C(C)C)c2)CC1 ; CCOC(=O)C1(c2cccc(F)c2)CCN(c2cc(C(C)C)c(O)c(C(C)C)c2)CC1 ; Cl]

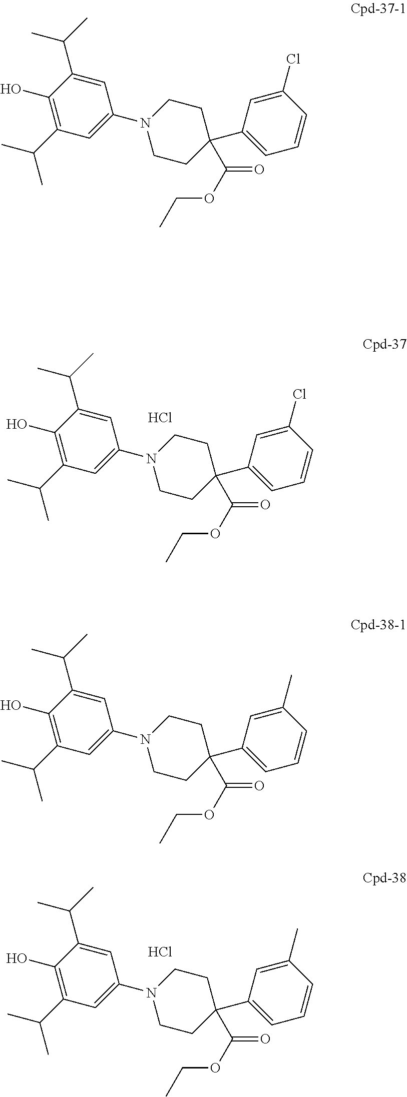 [SMILES: CCOC(=O)C1(c2cccc(Cl)c2)CCN(c2cc(C(C)C)c(O)c(C(C)C)c2)CC1 ; CCOC(=O)C1(c2cccc(Cl)c2)CCN(c2cc(C(C)C)c(O)c(C(C)C)c2)CC1 ; Cl ; CCOC(=O)C1(c2cccc(C)c2)CCN(c2cc(C(C)C)c(O)c(C(C)C)c2)CC1 ; CCOC(=O)C1(c2cccc(C)c2)CCN(c2cc(C(C)C)c(O)c(C(C)C)c2)CC1 ; Cl]

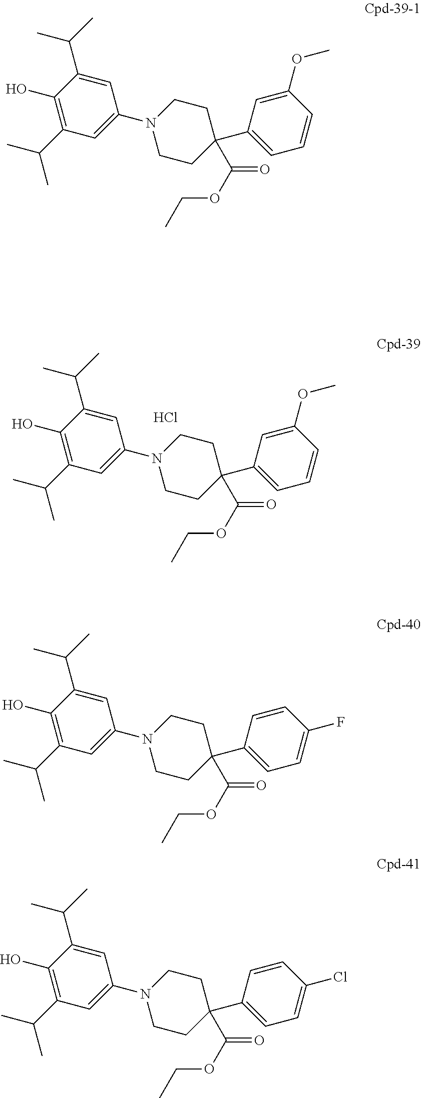

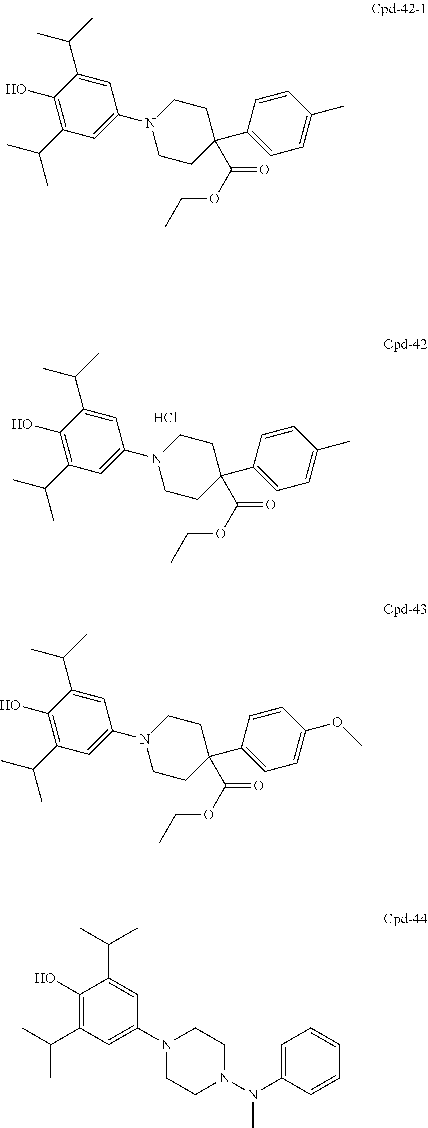

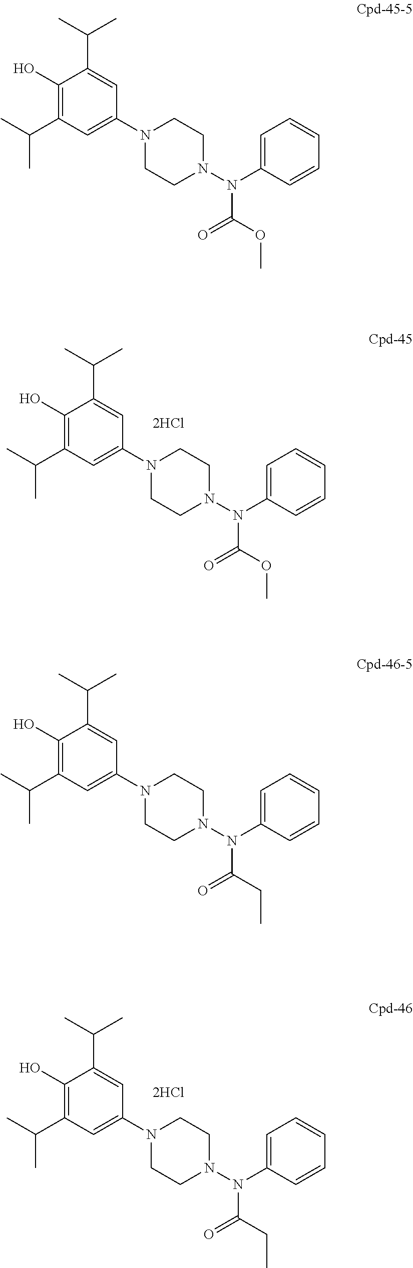 [SMILES: COC(=O)N(c1ccccc1)N1CCN(c2cc(C(C)C)c(O)c(C(C)C)c2)CC1 ; COC(=O)N(c1ccccc1)N1CCN(c2cc(C(C)C)c(O)c(C(C)C)c2)CC1 ; CCC(=O)N(c1ccccc1)N1CCN(c2cc(C(C)C)c(O)c(C(C)C)c2)CC1 ; CCC(=O)N(c1ccccc1)N1CCN(c2cc(C(C)C)c(O)c(C(C)C)c2)CC1]

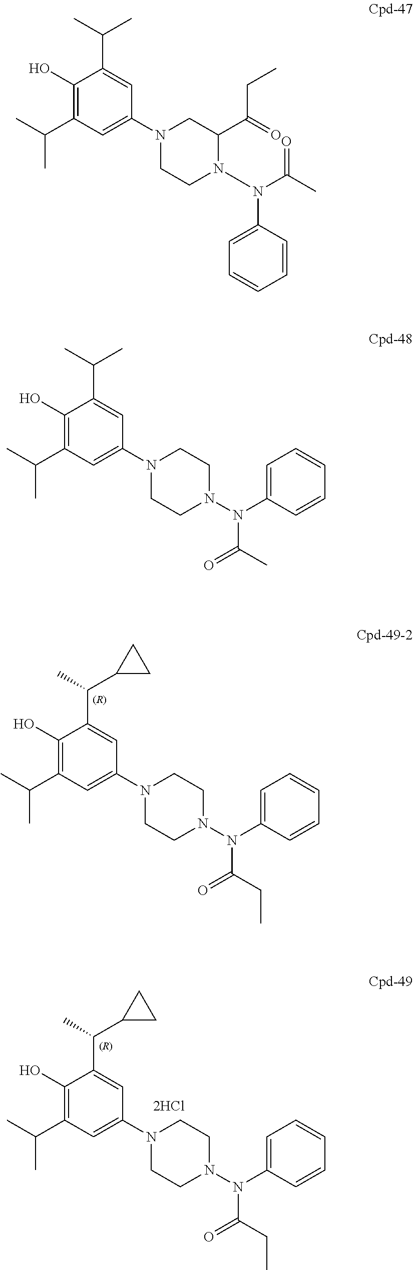

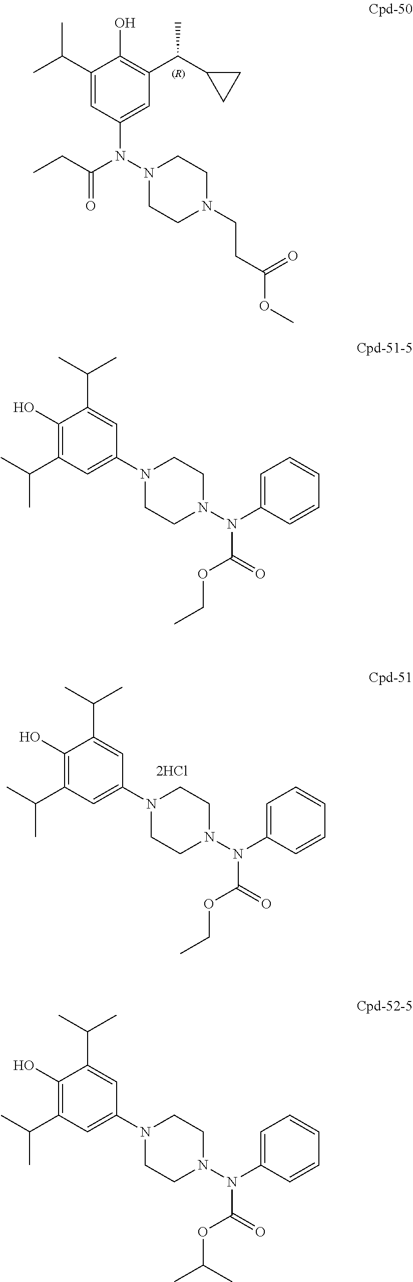

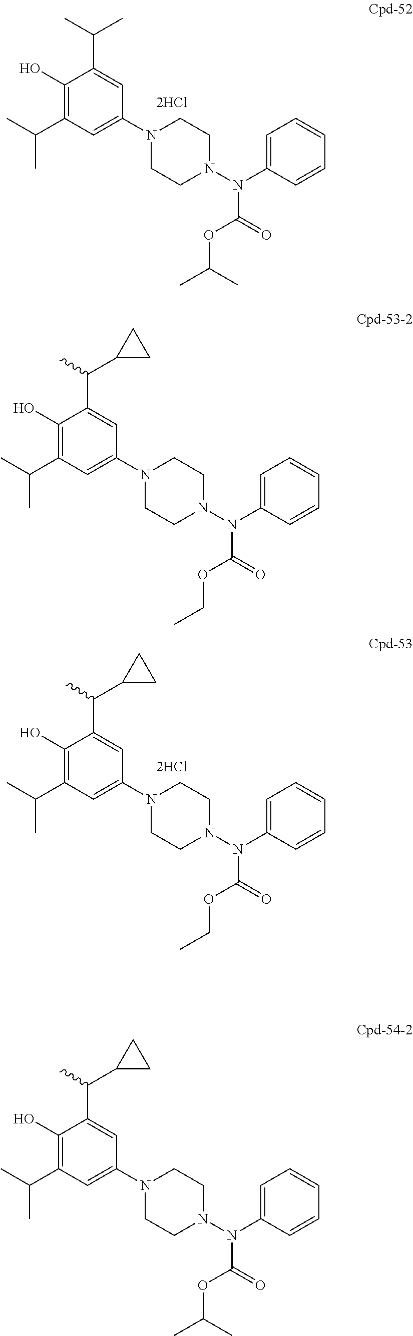

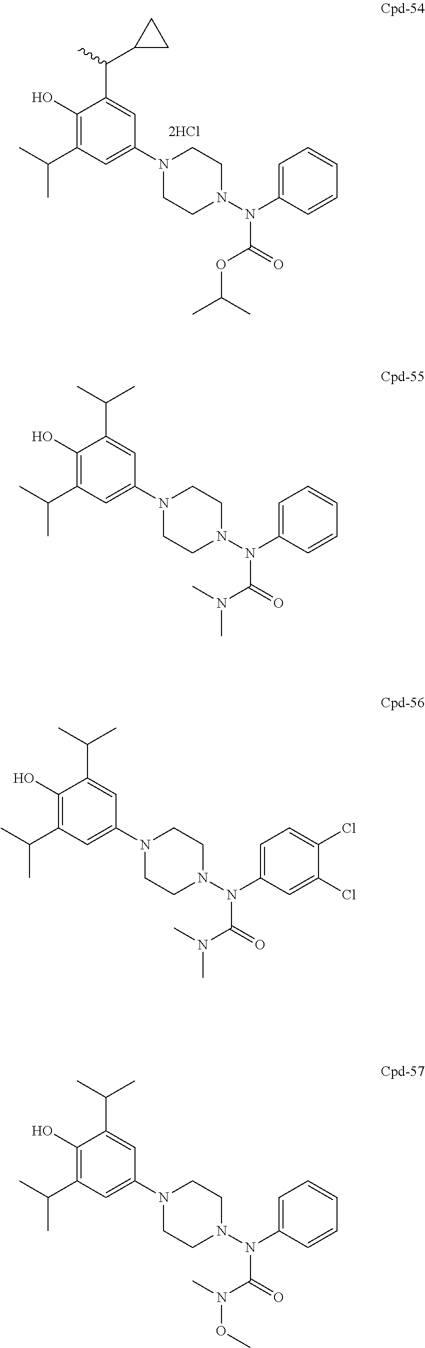 [SMILES: CC(C)OC(=O)N(c1ccccc1)N1CCN(c2cc(C(C)C)c(O)c(C(C)C3CC3)c2)CC1 ; CC(C)c1cc(N2CCN(N(C(=O)N(C)C)c3ccccc3)CC2)cc(C(C)C)c1O ; CC(C)c1cc(N2CCN(N(C(=O)N(C)C)c3ccc(Cl)c(Cl)c3)CC2)cc(C(C)C)c1O ; CON(C)C(=O)N(c1ccccc1)N1CCN(c2cc(C(C)C)c(O)c(C(C)C)c2)CC1]

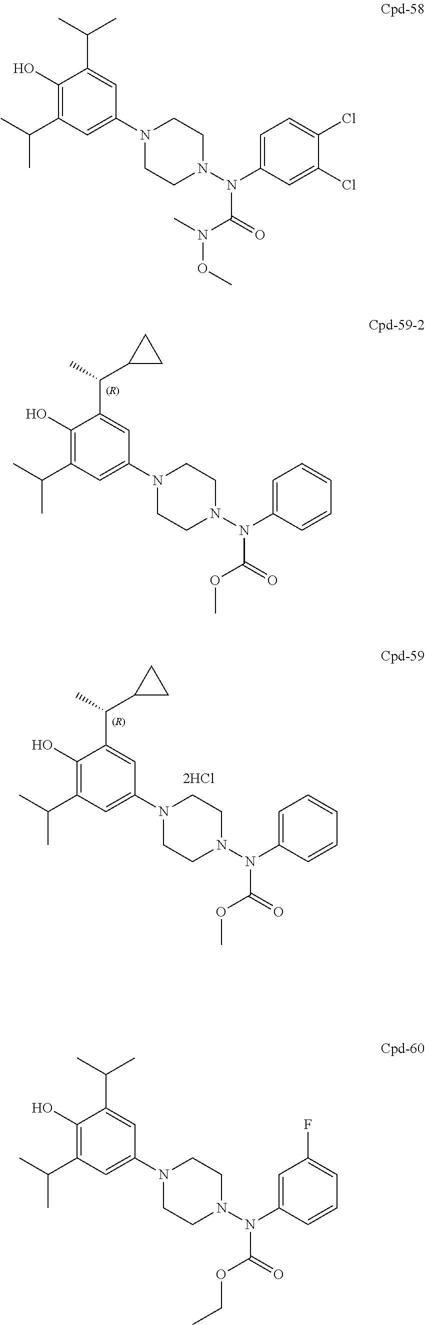

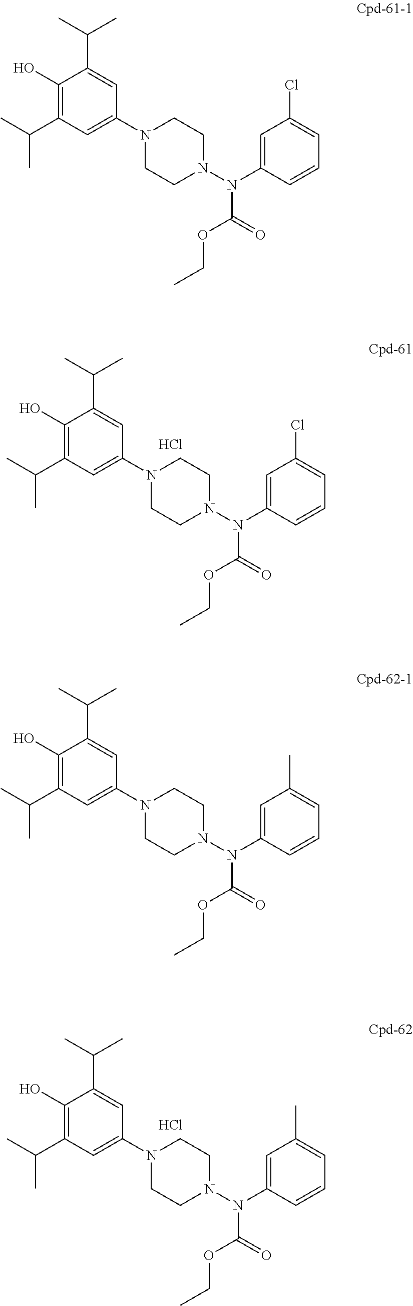 [SMILES: CCOC(=O)N(c1cccc(Cl)c1)N1CCN(c2cc(C(C)C)c(O)c(C(C)C)c2)CC1 ; CCOC(=O)N(c1cccc(Cl)c1)N1CCN(c2cc(C(C)C)c(O)c(C(C)C)c2)CC1 ; Cl ; CCOC(=O)N(c1cccc(C)c1)N1CCN(c2cc(C(C)C)c(O)c(C(C)C)c2)CC1 ; CCOC(=O)N(c1cccc(C)c1)N1CCN(c2cc(C(C)C)c(O)c(C(C)C)c2)CC1 ; Cl]

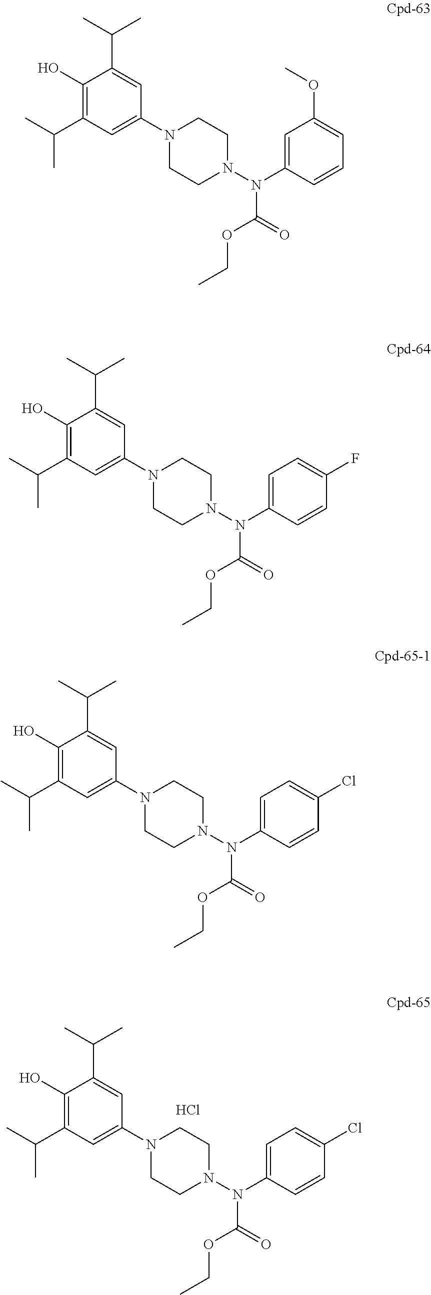 [SMILES: CCOC(=O)N(c1cccc(OC)c1)N1CCN(c2cc(C(C)C)c(O)c(C(C)C)c2)CC1 ; CCOC(=O)N(c1ccc(F)cc1)N1CCN(c2cc(C(C)C)c(O)c(C(C)C)c2)CC1 ; CCOC(=O)N(c1ccc(Cl)cc1)N1CCN(c2cc(C(C)C)c(O)c(C(C)C)c2)CC1 ; CCOC(=O)N(c1ccc(Cl)cc1)N1CCN(c2cc(C(C)C)c(O)c(C(C)C)c2)CC1 ; Cl]

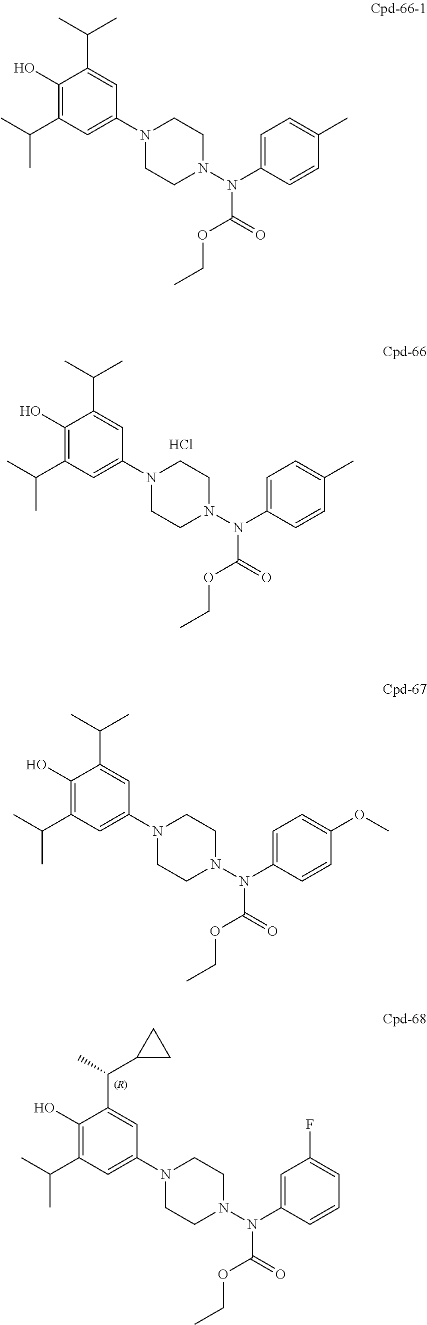

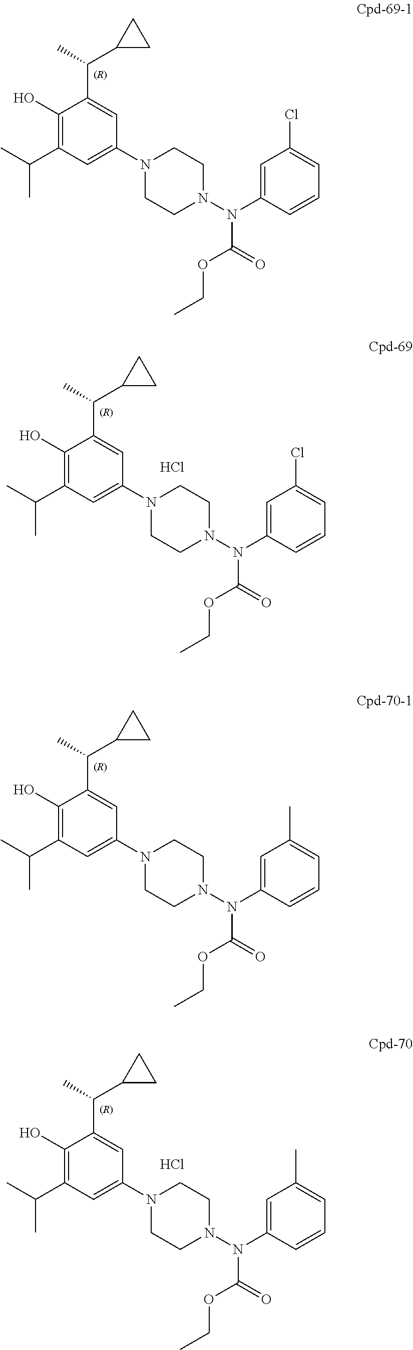

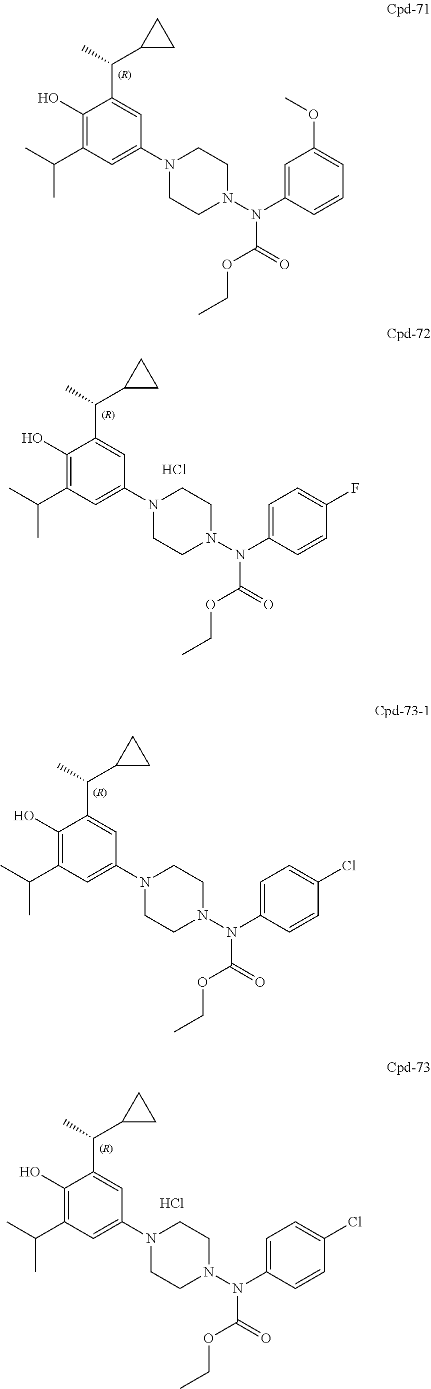

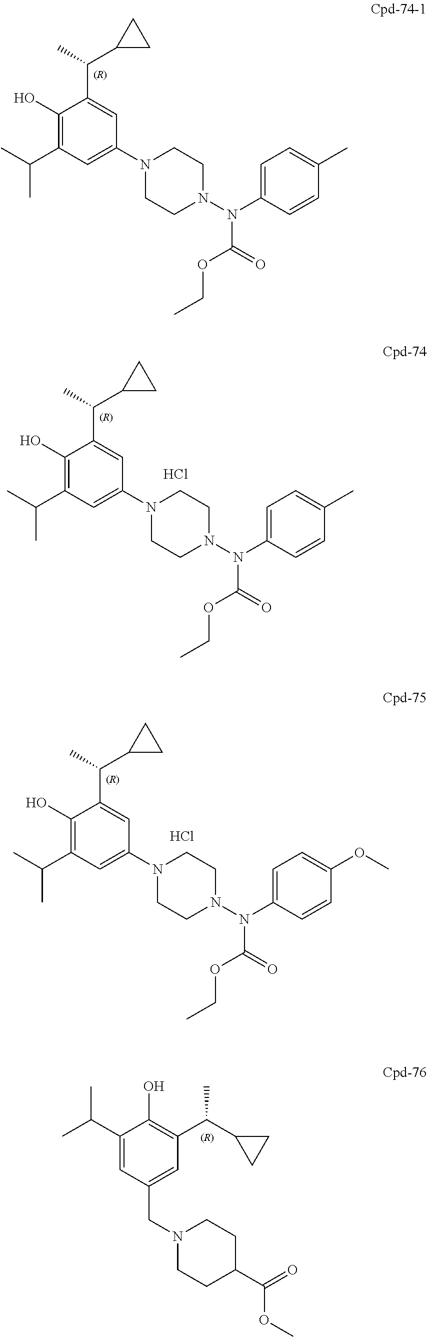

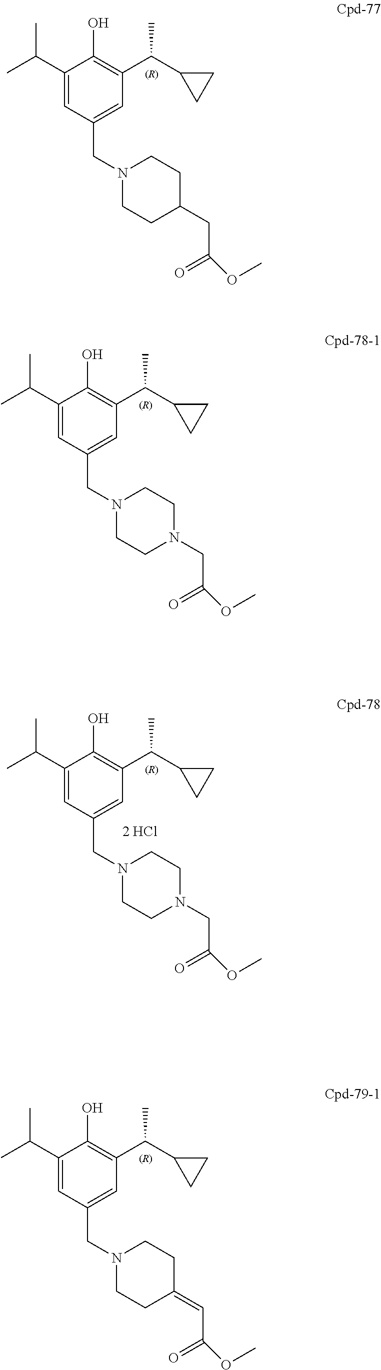

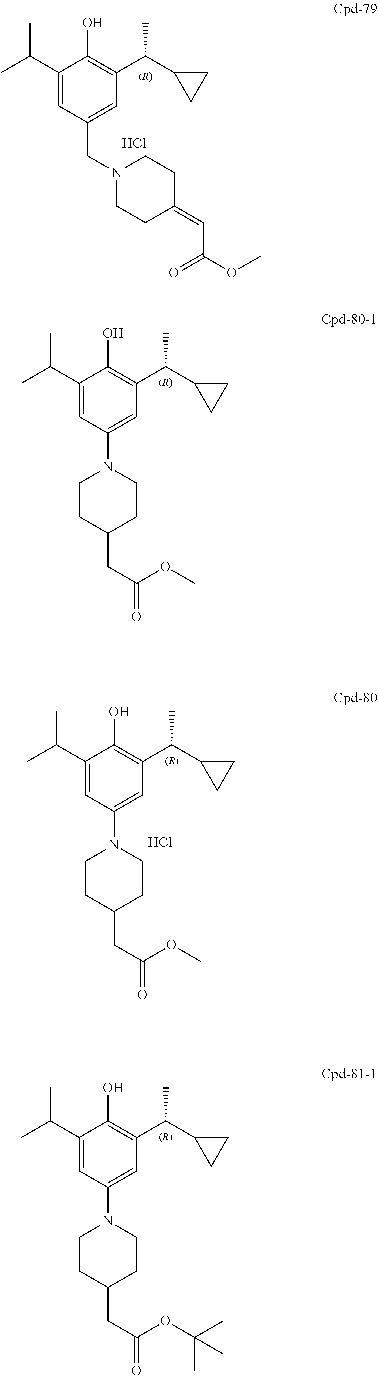

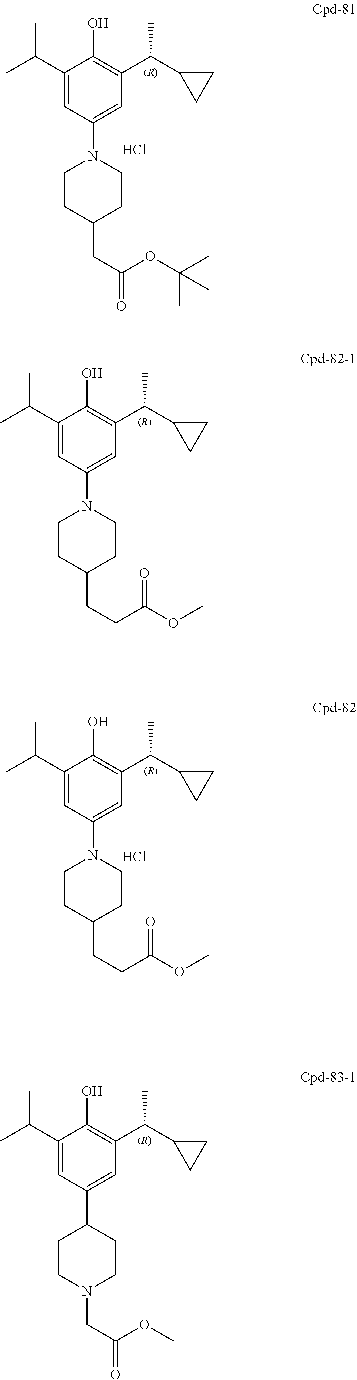

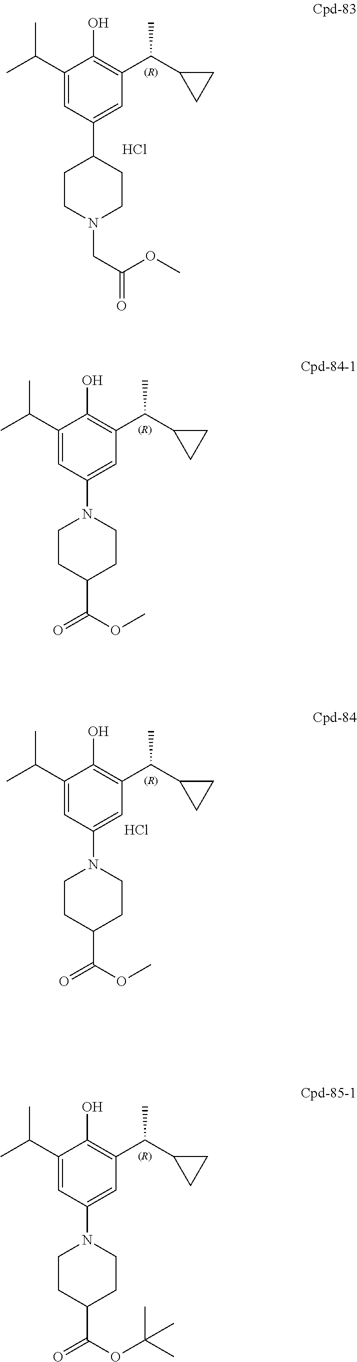

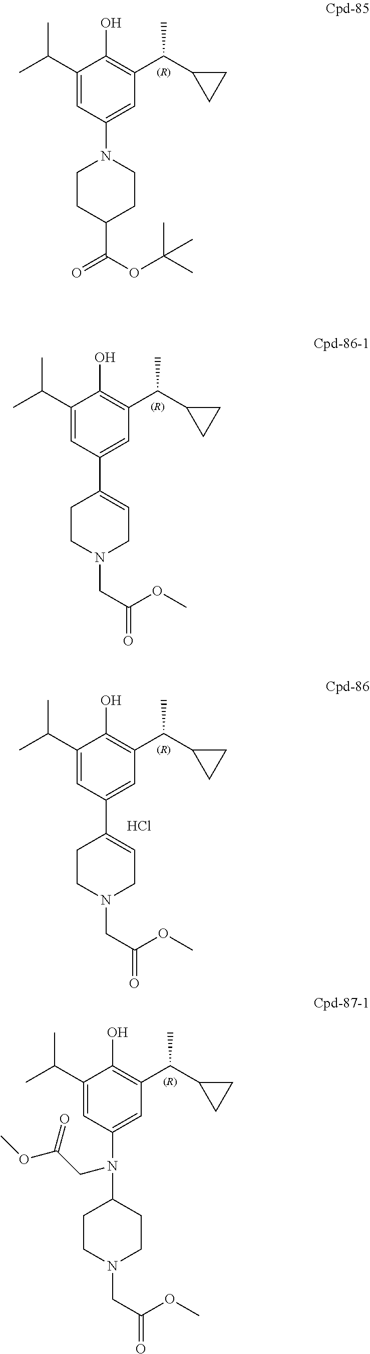

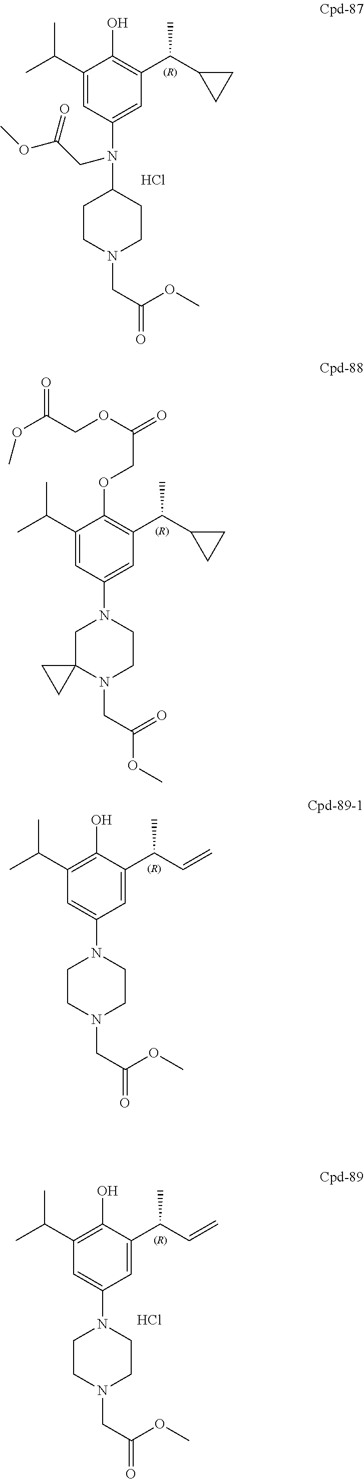

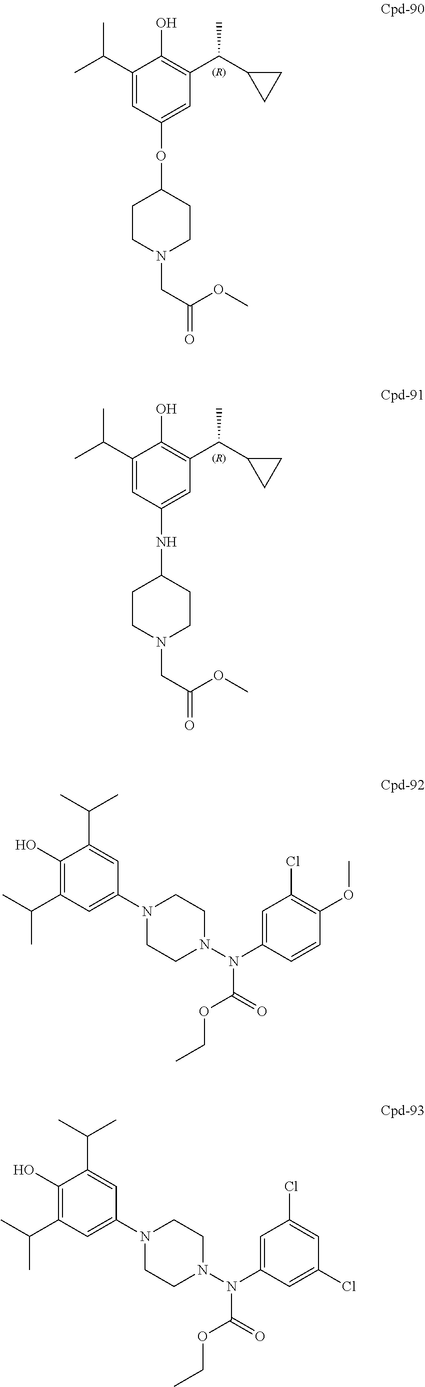

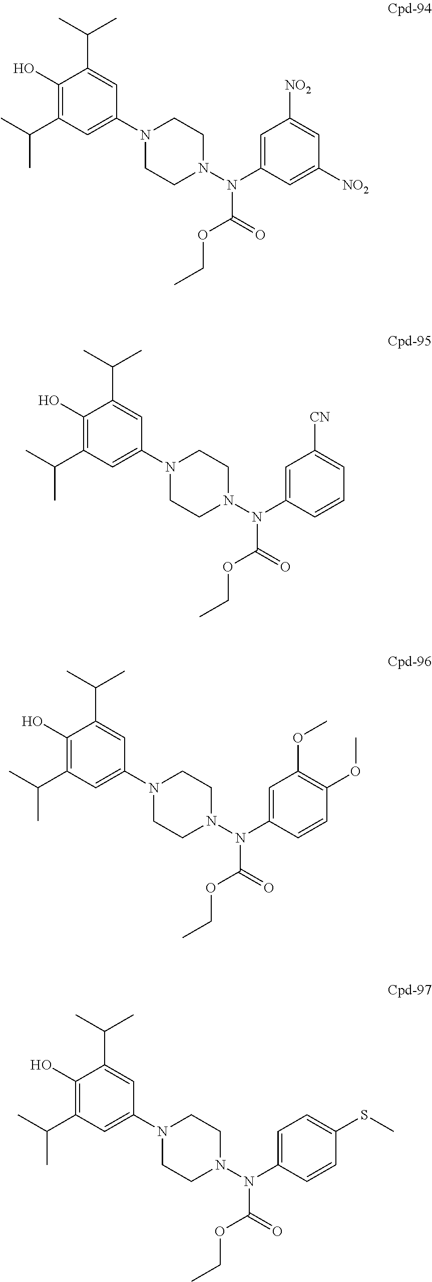

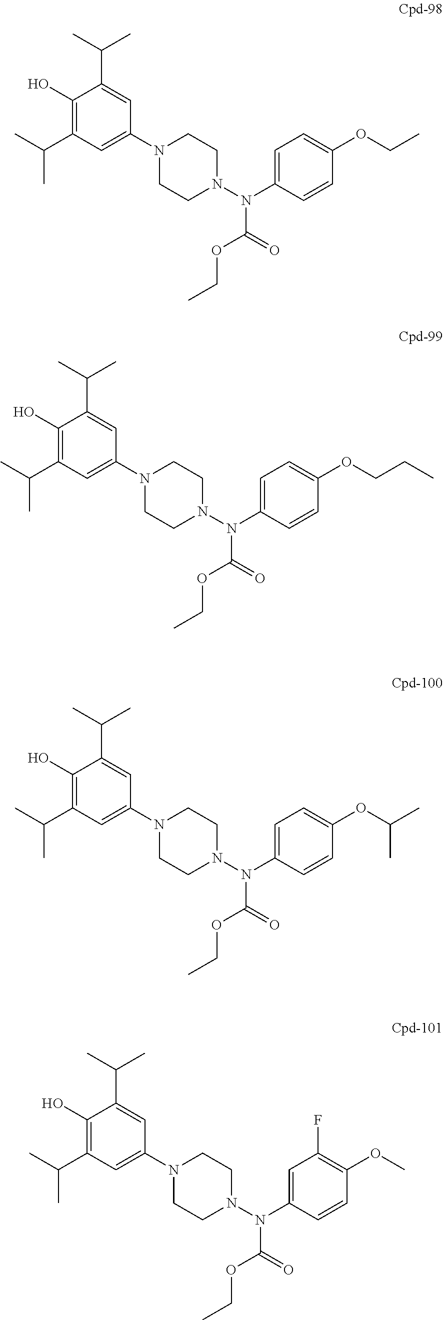

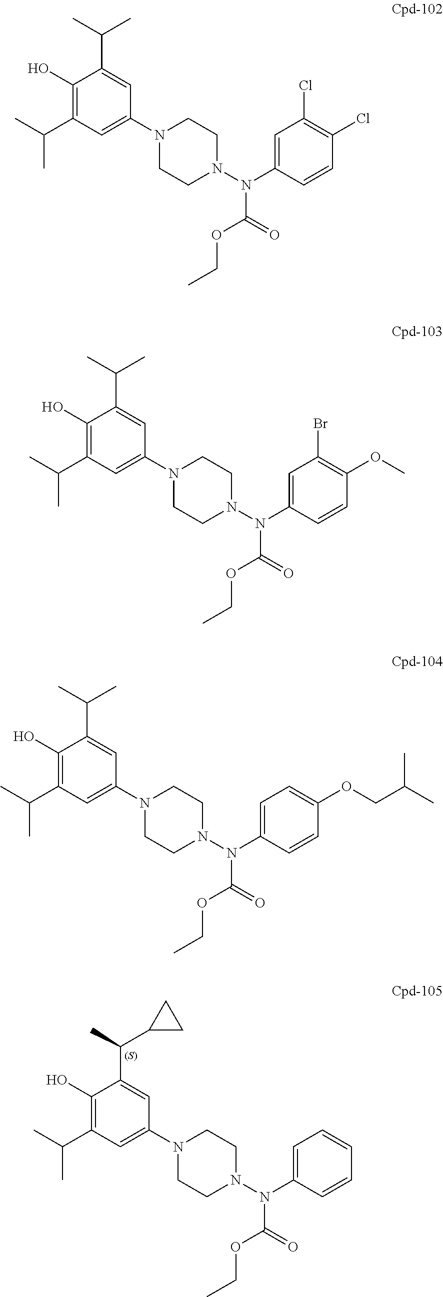

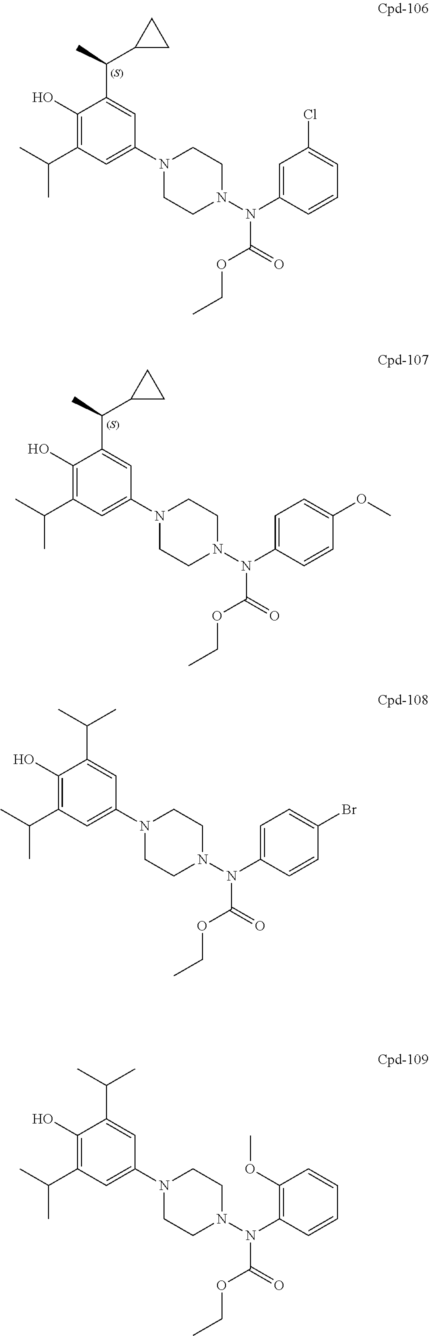

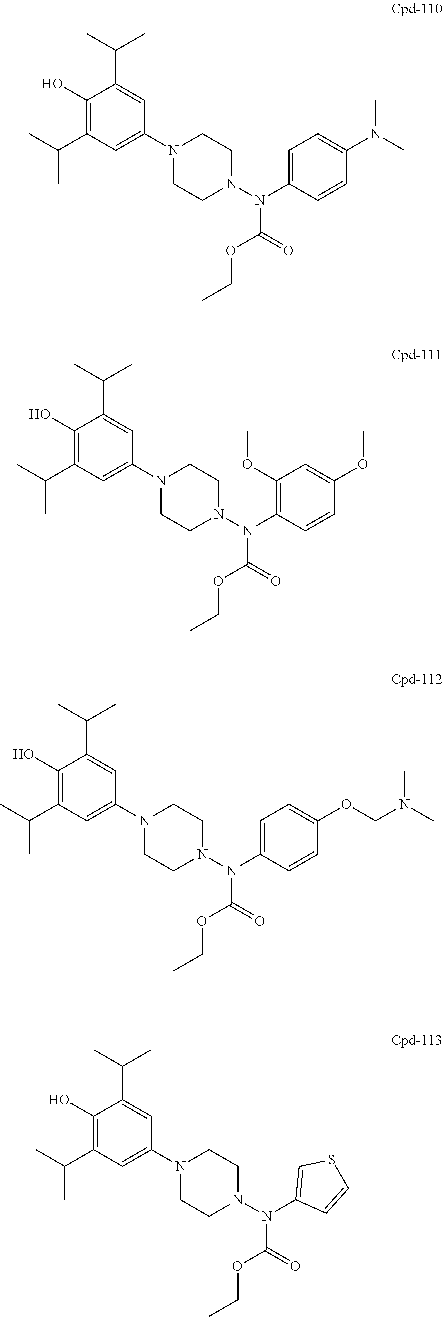

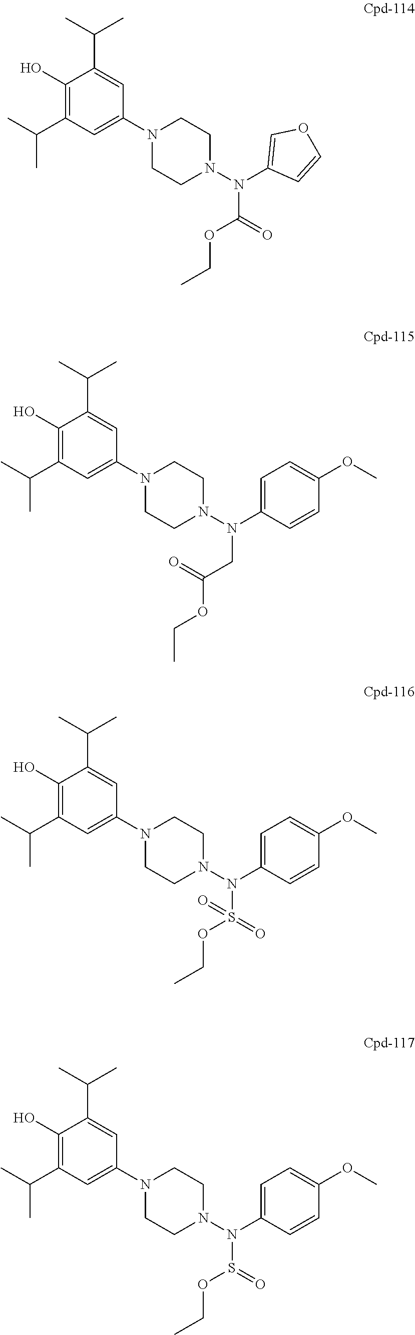

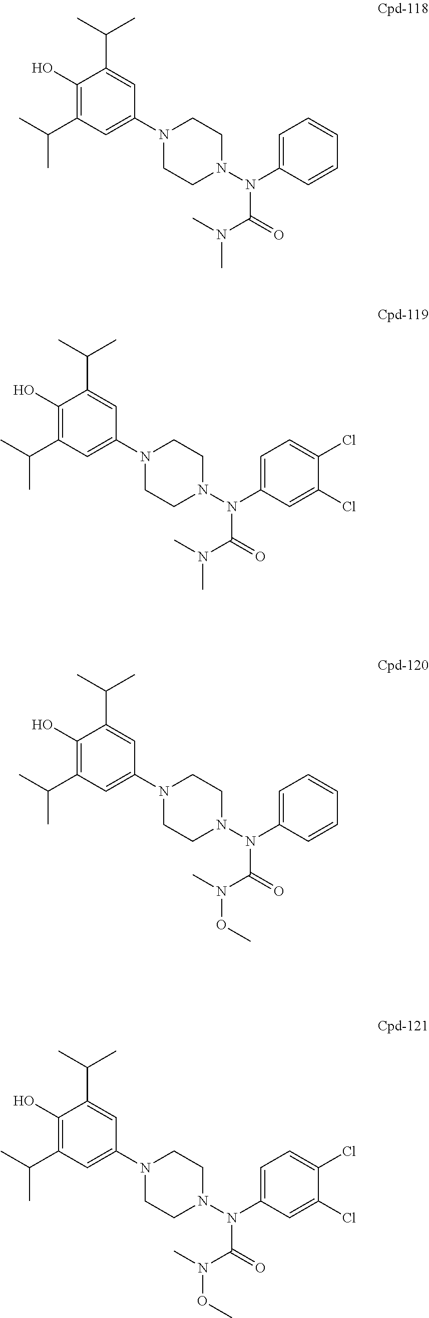 [SMILES: CC(C)c1cc(N2CCN(N(C(=O)N(C)C)c3ccccc3)CC2)cc(C(C)C)c1O ; CC(C)c1cc(N2CCN(N(C(=O)N(C)C)c3ccc(Cl)c(Cl)c3)CC2)cc(C(C)C)c1O ; CON(C)C(=O)N(c1ccccc1)N1CCN(c2cc(C(C)C)c(O)c(C(C)C)c2)CC1 ; CON(C)C(=O)N(c1ccc(Cl)c(Cl)c1)N1CCN(c2cc(C(C)C)c(O)c(C(C)C)c2)CC1]

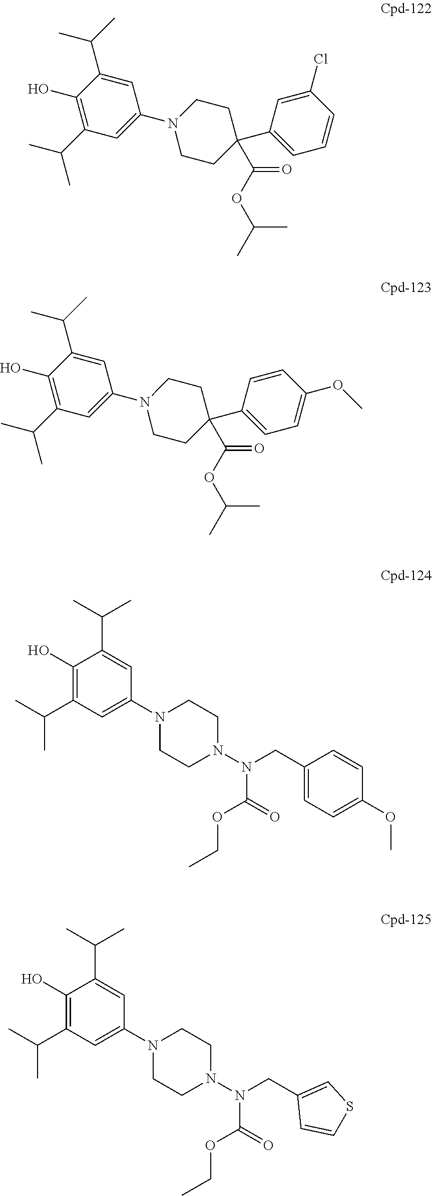

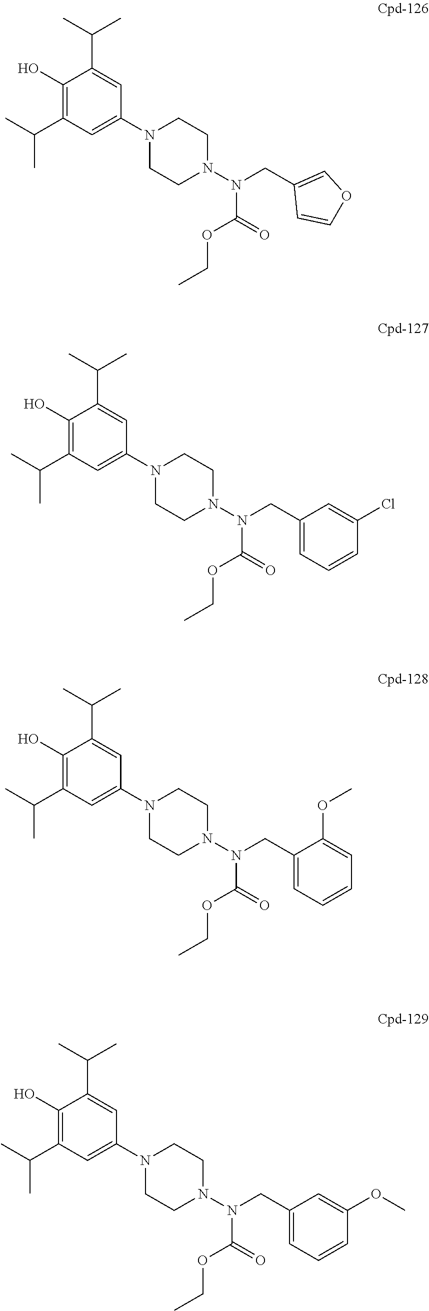

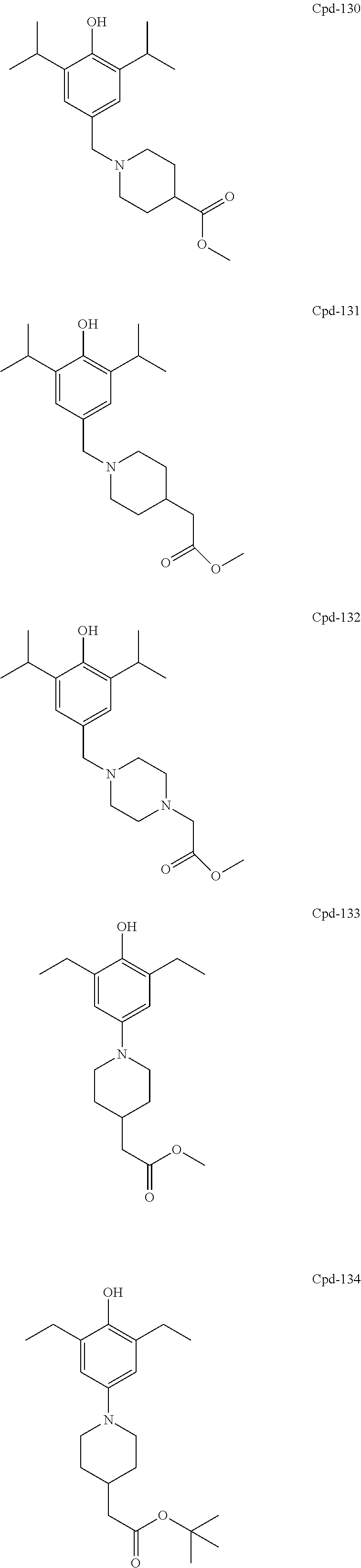

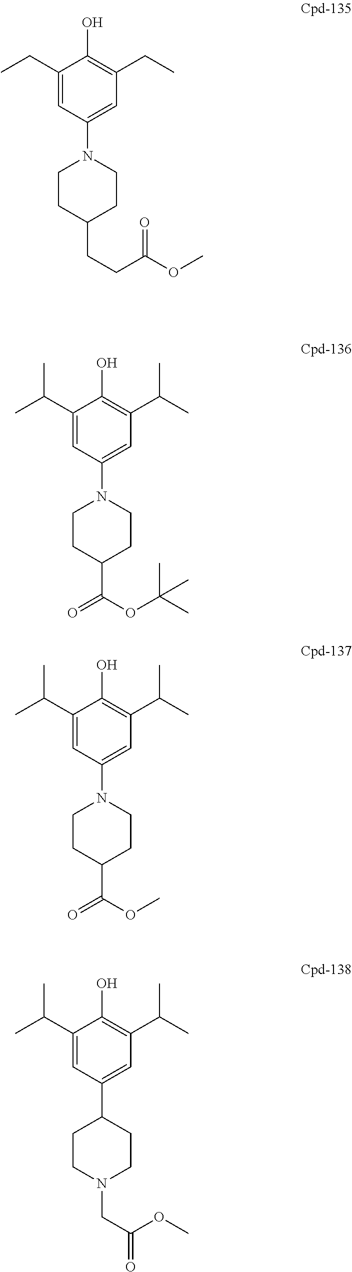

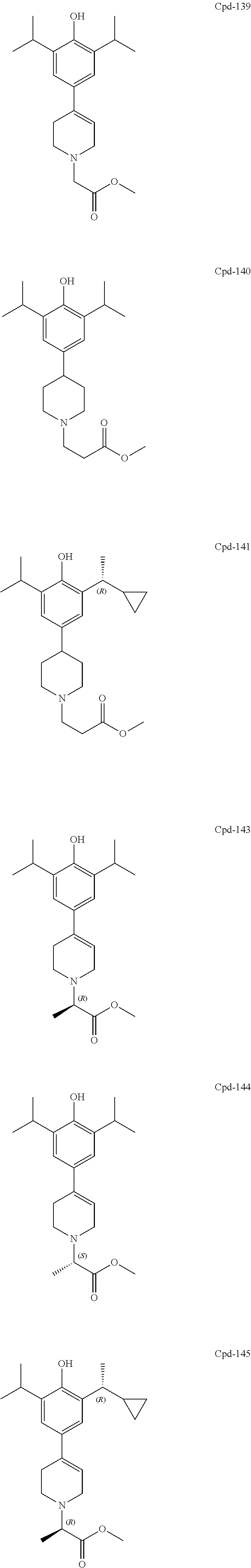

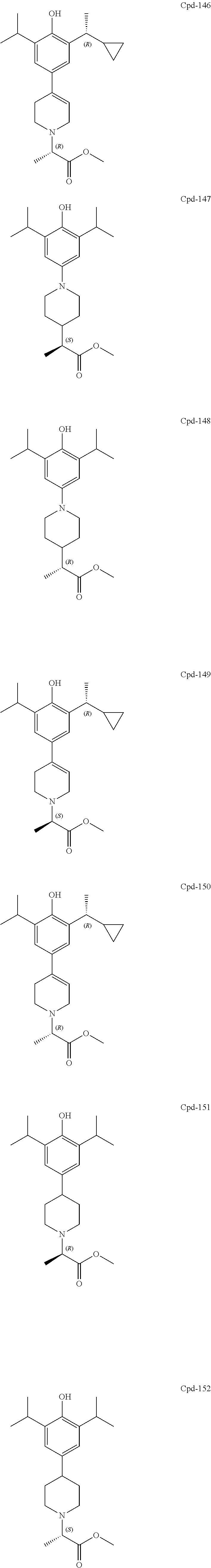

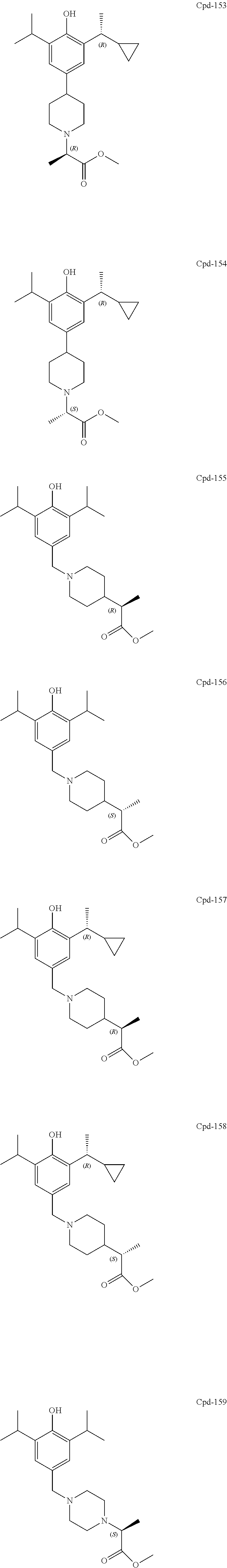

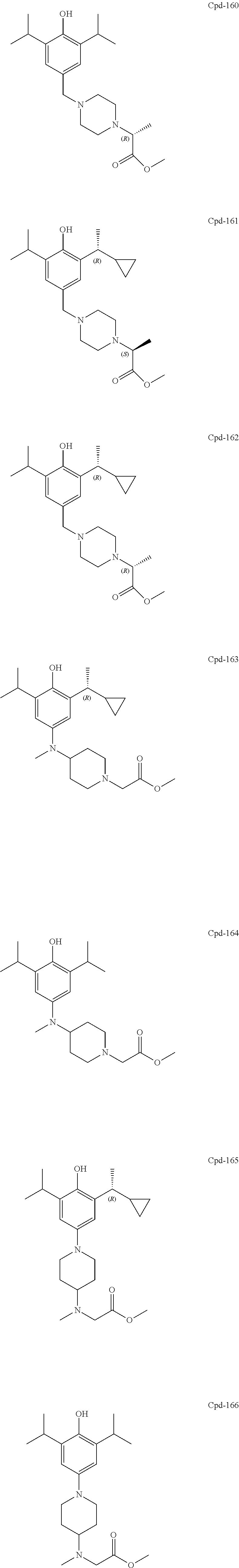

Further, the pharmaceutically acceptable salt is selected from acetate, adipate, aspartate, benzoate, benzene sulfonate, bicarbonate, carbonate, bisulfate, sulfate, borate, camphorsulfonate, citrate, cyclamate, edisylate, ethanesulfonate, formate, fumarate, glucoheptonate, gluconate, glucuronate, hexafluorophosphate, hydrochloride, hydrobromide, hydroiodide, hydroxyethylsulfonate, lactate, malate, maleate, malonate, mesylate, methylsulfate, naphthylate, napsylate, nicotinate, nitrate, orotate, oxalate, palmitate, pamoate, phosphate, hydrogen phosphate, dihydrogen phosphate, pyroglutamate, saccharate, stearate, succinate, tannate, tartrate, tosylate, trifluoroacetate, xinafoate, methanesulfonate or p-toluenesulfonate.

Further, the present invention also provides a drug, it is prepared by using the compound according to any one of the above, or a stereoisomer thereof, or a pharmaceutically acceptable salt thereof, or a solvate thereof, or a prodrug thereof, or a metabolite thereof, or a deuterated derivative thereof as active ingredients, with addition of pharmaceutically acceptable excipients.

The present invention also provides the use of compounds according to anyone of the above, or a stereoisomer thereof, or a pharmaceutically acceptable salt thereof, or a solvate thereof, or a prodrug thereof, or a metabolite thereof, or a deuterated derivative thereof, in preparation of a drug having an analgesic effect.

The present invention also provides the use of compounds according to anyone of the above, or a stereoisomer thereof, or a pharmaceutically acceptable salt thereof, or a solvate thereof, or a prodrug thereof, or a metabolite thereof, or a deuterated derivative thereof, in preparation of a drug for inducing sedation, hypnosis, and/or anesthesia, and/or for controlling status epilepticus.

The present invention also provides the use of compounds according to anyone of the above, or a stereoisomer thereof, or a pharmaceutically acceptable salt thereof, or a solvate thereof, or a prodrug thereof, or a metabolite thereof, or a deuterated derivative thereof, in preparation of a drug having an anesthetic effect, or having both an analgesic effect and sedative, hypnotic and/or anesthetic, and/or being capable of controlling status epilepticus.

The “drug with analgesic effect” according to the present invention denotes a drug that no response to noxious stimuli or an increased response threshold to noxious stimuli when the compounds of the present invention produce sedative, hypnotic and/or anesthetic effects.

The “drug with sedative effects” according to the present invention denotes a drug that effectively aids sleep and improves sleep quality, thereby mitigating the severe harm caused by insomnia, treating insomnia, and enhancing sleep outcomes.

The “drug with hypnotic effects” according to the present invention denotes a drug that induces drowsiness and promotes sleep. It suppresses the central nervous system, causing sedation at low doses and general anesthesia at excessive doses.

The “drug with anesthetic effects” according to the present invention denotes to a drug that induces reversible functional inhibition of the central and/or peripheral nervous systems, characterized primarily by the loss of sensation, particularly pain perception.

Preferably, the anesthesia is general anesthesia.

The “general anesthesia” mentioned in the present invention denotes the temporary inhibition of the central nervous system caused by anesthetics entering the body. Clinically, it is characterized by loss of consciousness, absence of pain perception throughout the body, amnesia, reflex inhibition, and skeletal muscle relaxation.

The “status epilepticus.” mentioned in the present invention denotes recurrent epileptic seizures without full recovery of consciousness between episodes, or a single seizure lasting over 30 minutes. Prolonged seizures can cause irreversible brain damage due to hyperthermia, circulatory failure, or neuronal excitotoxicity, leading to high morbidity and mortality. Thus, status epilepticus is a common medical emergency in internal medicine.

The compounds and derivatives provided in the present invention can be named according to the IUPAC (International Union of Pure and Applied Chemistry) or CAS (Chemical Abstracts Service, Columbus, OH) nomenclature systems.

Unless otherwise specified, definitions of groups or terms provided herein apply throughout the entire specification. Terms not explicitly defined shall be interpreted according to their generally accepted meanings in the field, consistent with the disclosure and context.

In this context, “substituted” means that hydrogen atoms in a molecule are replaced by other different atoms or groups, or more substitutions on the same or different atoms within the molecule.

The minimum and maximum number of carbon atoms in a hydrocarbon group are indicated by prefixes. For example, the prefix C_{a-b} alkyl represents an alkyl group containing “a” to “b” carbon atoms. For example, C₁₋₆ alkyl refers to a straight or branched chain alkyl group containing 1 to 6 carbon atoms.

“Alkyl” refers to a saturated hydrocarbon chain with a specified number of carbon atoms. For example, a C₁-C₆ alkyl group means an alkyl group having 1 to 6 carbon atoms, i.e., 1, 2, 3, 4, 5, or 6 carbon atoms. alkyl groups may be straight-chain or branched. Representative branched alkyl groups have one, two, or three branches. Alkyl includes methyl, ethyl, propyl (n-propyl and isopropyl), butyl (n-butyl, isobutyl, and tert-butyl), pentyl (n-pentyl, isopentyl, and neopentyl), hexyl, and their various branched isomers, etc.

“Alkenyl” refers to an unsaturated hydrocarbon chain containing at least one carbon-carbon double bond. For example, a C₂-C₆ alkenyl group means an alkenyl group having 2 to 6 carbon atoms, i.e., 2, 3, 4, 5, or 6 carbon atoms. Alkenyl groups may be straight-chain or branched. Included:

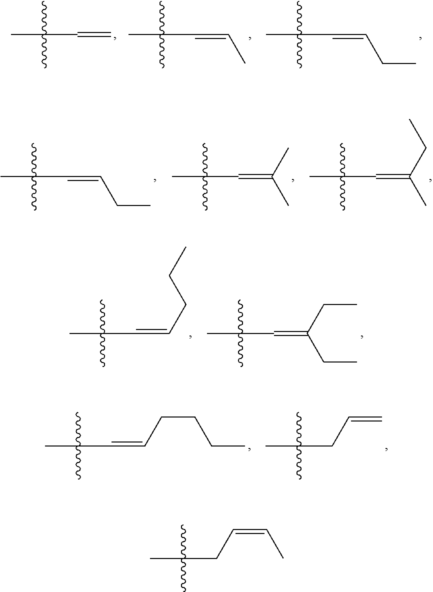

etc.

“Alkynyl” refers to an unsaturated hydrocarbon chain containing at least one carbon-carbon triple bond. For example, a C₂-C₆ alkynyl group means an alkynyl group having 2 to 6 carbon atoms, i.e., 2, 3, 4, 5, or 6 carbon atoms. Alkynyl groups may be straight-chain or branched. Included:

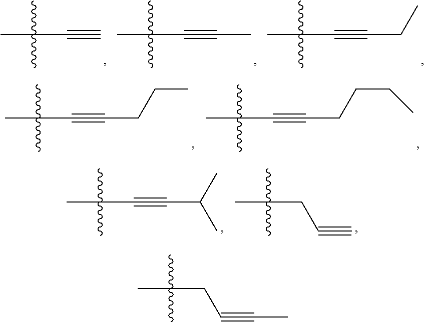

etc.

“Halogen” is fluorine, chlorine, bromine, or iodine.

As used in the present invention, “forming a 3- to 6-membered ring structure” means forming a 3- to 6-membered cycloalkyl, heterocyclic group, aryl, or heteroaryl group.

“Cycloalkyl” refers to a saturated or unsaturated all-carbon monocyclic or polycyclic system (including fused, spiro, or bridged rings) without a conjugated x-electron system, encompassing but not limited to:

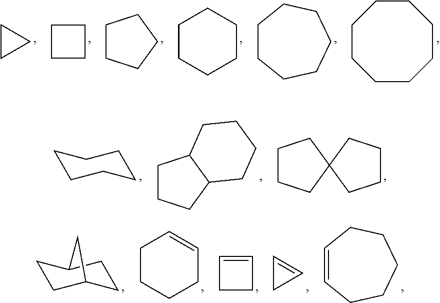

“Alkoxy” refers to an —O-alkyl group. Non-limiting examples include methoxy, ethoxy, n-propoxy, isopropoxy, n-butoxy, sec-butoxy, tert-butoxy, n-pentoxy, n-hexoxy, cyclopropoxy, and cyclobutoxy.

“Heterocyclyl” refers to a cycloalkyl group in which at least one ring carbon atom is replaced by a heteroatom selected from O, N, or S, including but not limited to:

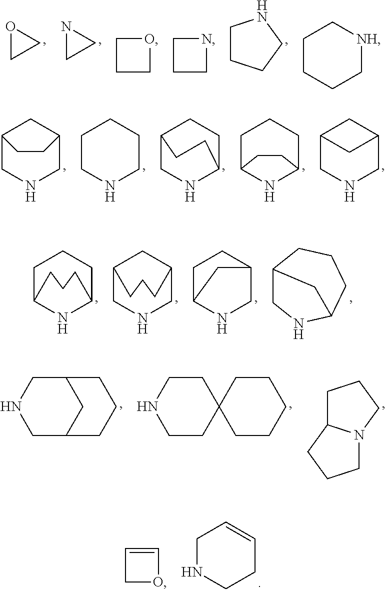

“Aryl” refers to an all-carbon monocyclic or polycyclic system (including fused, spiro, or bridged rings) with a conjugated n-electron system, including but not limited to: phenyl, naphthyl, phenanthrenyl, anthracenyl, fluorenyl, and indenyl.

The aryl ring may be fused to other cyclic groups (either saturated or unsaturated rings), but must not contain heteroatoms such as O, N, or S. Additionally, the attachment point to the parent structure must be on a carbon atom within the conjugated x-electron system ring, including but not limited to:

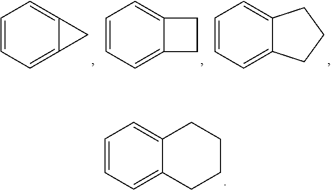

“Heteroaryl” refers to an aryl group in which at least one ring carbon atom is replaced by a heteroatom selected from O, N, or S, while maintaining a conjugated π-electron system, including but not limited to:

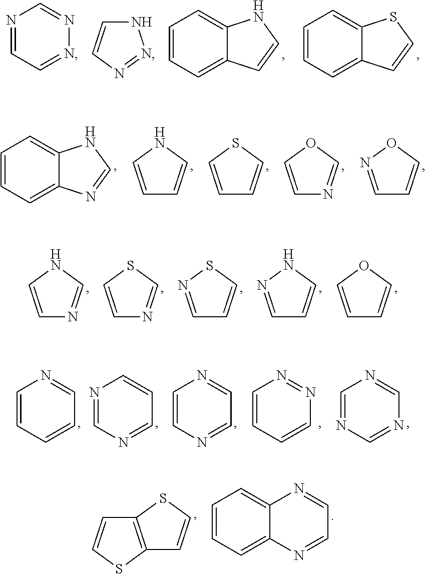

“Deuterated derivative” refers to a compound in which one or more hydrogen atoms are replaced by deuterium.

“Pharmaceutically acceptable” means that a carrier, vehicle, diluent, excipient, and/or the formed salt is generally chemically or physically compatible with other components constituting a pharmaceutical dosage form and physiologically compatible with the recipient.

“Salt” refers to an acid and/or base salt formed by the compound or its stereoisomer with inorganic and/or organic acids and/or bases, including zwitterions (internal salts) and quaternary ammonium salts, such as alkylammonium salts. These salts can be obtained directly during the final isolation and purification of the compound, or by appropriately mixing the compound or its stereoisomer with a certain amount of acid or base (e.g., in equivalent amounts). These salts may form precipitates in solution and be collected by filtration, recovered after solvent evaporation, or lyophilized from an aqueous medium.

In the present invention, the pharmaceutically acceptable salts of the compound may include, but are not limited to: acetate, adipate, aspartate, benzoate, besylate, bicarbonate, carbonate, bisulfate, sulfate, borate, camphorsulfonate, citrate, cyclamate, edisylate, esylate, formate, fumarate, gluceptate, gluconate, glucuronate, hexafluorophosphate, hydrochloride, hydrobromide, hydroiodide, hydroxyethanesulfonate, lactate, malate, maleate, malonate, mesylate, methylsulfate, naphthoate, napsylate, nicotinate, nitrate, orotate, oxalate, palmitate, pamoato, phosphate, hydrogen phosphate, dihydrogen phosphate, pyroglutamate, saccharate, stearate, succinate, tannate, tartrate, tosylate, trifluoroacetate, xinafoate, methanesulfonate, p-toluenesulfonate, or quaternary ammonium salts.

The compound of the present invention or composition thereof, as well as the use method thereof:

The compounds of the present invention, and various crystal forms, pharmaceutically acceptable inorganic or organic salts, hydrates or solvates thereof, as well as the pharmaceutical composition containing the compound of the present invention as the main active ingredients can be used for sedation, hypnosis and/or general anesthesia. The compound of the present invention can also be used for controlling epileptic persistent state and the like.

The pharmaceutical composition of the present invention includes a compound of the present invention or a pharmaceutically acceptable salt thereof within a safe and effective amount, as well as a pharmaceutically acceptable excipient or carrier thereof.

The administration ways for the compound or pharmaceutical composition of the present invention include (but not limited to) intragastric, intraintestinal, extragastrointestinal (intravenous, intramuscular or subcutaneous), oral and various local administration.

The composition for extragastrointestinal injection (intravenous, intramuscular, subcutaneous) may contain physiologically acceptable sterile aqueous or anhydrous solutions, dispersions, suspensions or emulsions, and sterile powder used for reconstitution into sterile injectable solutions or dispersions. Suitable aqueous and non-aqueous carriers, diluents, solvents or excipients include water, ethanol, polyols and their suitable mixtures.

Solid dosage forms for oral administration include capsules, tablets, pills, powders and granules. In these solid dosage forms, the active compound is mixed with at least one conventional inert excipient (or carrier), such as sodium citrate or dicalcium phosphate, or mixed with following ingredients: (a) bulking agent or compatibilizer, such as starch, lactose, sucrose, glucose, mannitol and silicic acid; (b) binding agent, such as hydroxymethylcellulose, alginate, gelatin, polyvinylpyrrolidone, sucrose and gum arabic; (c) moisturing agent, such as glycerin; (d) disintegrating agent, such as agar, calcium carbonate, potato starch or cassava starch, alginate, some complex silicates, and sodium carbonate; (e) solvents, such as paraffin; (f) absorption accelerators, such as quaternary amine compounds; (g) wetting agents, such as cetyl alcohol and glycerin monostearate; (h) adsorbents, such as kaolin; and (i) lubricants, such as talc, calcium stearate, magnesium stearate, solid polyethylene glycol, sodium dodecyl sulfate, or mixtures thereof. In capsules, tablets and pills, the dosage form may also include buffers.

The liquid dosage forms used for oral administration include pharmaceutically acceptable emulsion, solution, suspension, syrup or tincture. In addition to the active compounds, the liquid dosage form may comprise inert diluents commonly used in the art, such as water or other solvents, solubilizers and emulsifiers, for example, ethanol, isopropanol, ethyl carbonate, ethyl acetate, propylene glycol, 1,3-butanediol, dimethylformamide and oil, especially cottonseed oil, peanut oil, corn germ oil, olive oil, castor oil and sesame oil or the mixture thereof, etc.

Solid dosage forms such as tablets, sugar pills, capsules, pills and granules can be prepared by coating and shell materials, such as casing and other materials known in the art. They may comprise an opaque agent, and the release of the active compound or compound in the composition may be delayed in a certain part of the digestive tract. Examples of embedding components that can be used are polymers and waxes. If necessary, the active compound may also form a microcapsule form with one or more of above excipients.

The dosage form of the compound of the present invention for local administration includes ointment, powder, patch, spray and inhalant. The active ingredient is mixed in sterile conditions with a biologically acceptable carrier and any preservatives, buffers, or propellants that may be required if necessary.

Except for these inert diluents, the composition may also include auxiliaries such as wetting agents, emulsifiers and suspensions, sweeteners, flavouring agents and perfumes.

Except for the active compounds, the suspension may contain a suspending agent, such as ethoxylated isooctadecanol, polyoxyethylene sorbitol and dehydrated sorbitol ester, microcrystalline cellulose, aluminum methoxide and agar or the mixture thereof, etc.

The compound of the present invention can be administered alone or in combination with other pharmaceutically acceptable compounds.

When the pharmaceutical composition is used, the safe and effective amount of the compound of the present invention is administrated to the mammal (such as human) in need thereof, in which the dosage is the pharmaceutically acceptable safe and effective dosage.

When the pharmaceutical composition is used, the safe and effective amount of the compound of the present invention is administrated to the mammal (such as human) that need to be treated, in which the pharmaceutically effective dosage is given.

Compared with the technology currently available, the compounds provided by the present invention have achieved the following beneficial effects:

1. It is well-known to those skilled in the art that clinically used intravenous general anesthetics such as propofol, etomidate, fospropofol disodium, and ciprofol do not have analgesic effects. However, the compounds of the present invention have analgesic effects while exhibiting sedative, hypnotic, and/or anesthetic effects.
2. Due to the analgesic effects of the compounds of the present invention, opioid analgesics such as fentanyl, alfentanil, sufentanil, or remifentanil can be reduced or avoided in clinical application, thereby reducing the occurrence of adverse reactions of opioid analgesics such as circulatory depression, respiratory depression, urinary retention, and skin pruritus.

In summary, the compounds provided by the present invention not only have sedative, hypnotic, and/or anesthetic effects and can control status epilepticus, but also have analgesic effects while producing the above effects. Therefore, the compounds provided by the present invention have broad application prospects in the preparation of drugs that have both analgesic effects and sedative, hypnotic, and/or anesthetic effects, as well as drugs that can control status epilepticus, providing a new choice for clinically preparing drugs that have both analgesic effects and sedative, hypnotic, and/or anesthetic effects and can control status epilepticus.

Obviously, according to the above content of the present invention, other various forms of modifications, substitutions, or changes can be made without departing from the basic technical idea of the present invention according to the ordinary technical knowledge and customary means in the art.

The following further details the above content of the present invention through specific embodiments in the form of examples. However, it should not be understood that the scope of the above subject matter of the present invention is limited to the following examples. All technologies implemented based on the above content of the present invention belong to the scope of the present invention.

### EXAMPLES

The raw materials and equipment used in the implementation of this invention are commercially available products.

The structure of the compounds was determined by nuclear magnetic resonance (NMR) and/or mass spectrometry (MS). NMR shifts (8) are given in units of 10-6 (ppm). NMR measurements were performed using a Bruker Avance III 400 spectrometer, with deuterated dimethyl sulfoxide (d₆-DMSO) or deuterated methanol (CD₃OD) as the solvent and tetramethylsilane (TMS) as the internal standard.

Liquid chromatography-mass spectrometry (LCMS) was performed on an Agilent 1260-6110 LCMS system (ESI) with a Waters X-Bridge C18 column (50 mm×4.6 mm×3.5 μm). The column temperature was maintained at 40° C., and the flow rate was 2.0 mL/min. The mobile phase consisted of a gradient program: starting with 95% [water+0.05% trifluoroacetic acid (TFA)] and 5% [acetonitrile (CH₃CN)+0.05% TFA], transitioning to 0% [water+0.05% TFA] and 100% [CH₃CN+0.05% TFA] over 3 minutes, holding for 1 minute, then returning to the initial conditions (95% [water+0.05% TFA] and 5% [CH₃CN+0.05% TFA]) over 0.05 minutes, and maintaining these conditions for an additional 0.7 minutes.

### 1) Materials and Reagents

The silica gel plate (HSGF254) for thin layer chromatography was bought from Yantai Xinnuo Chemical Co., Ltd, with the thickness of 1 mm.

Thin layer chromatography (TLC) was bought from Yantai Jiangyou silicone Development Co., Ltd., with the thickness of 0.2±0.03 mm.

Silica gel used for column chromatography was mostly made by Rushan Sun Desiccant Co., Ltd. (Weihai, Shandong) with 100-200 meshes or 200-300 meshes.

### 2) The Main Instruments

Electronic Balance Sartorius BSA124S (manufactured by Sartorius Scientific Instruments (Beijing) Co., Ltd.);

MS-H-PRO⁺ digital control heating type magnetic stirrer (Dragon Lab Instruments Beijing Co., Ltd.)

Contact Voltage Regulator (manufacturer: Zhejiang Tianzheng Electric Co., Ltd);

Temperature Controller (made by Shanghai Lulin Electric Co., Ltd);

Three-function Ultraviolet Analysis (model: ZF-2, manufactured by Shanghai Anting Electronic Instrument Factory);

Rotary Evaporator R-201 (manufactured by Shanghai Shenshun Biological Technology Co., Ltd)

Constant Temperature Water Bath (model: W201D, manufactured by Shanghai Shenshun Biological Technology Co., Ltd)

Circulating Water Vacuum Pump SHB-III (manufactured by Zhengzhou Huicheng Technology Industry and Trade Co., Ltd)

Mobile Water Pump SHB-B95 (manufactured by Zhengzhou Huicheng Technology Industry and Trade Co., Ltd)

Low-temperature Cooling Liquid Circulating Pump (manufactured by Gongyi Yuhua Instrument Co., Ltd)

2XZ-2 vane-type vacuum pump (Linhai Yongwu Vacuum Equipment Co., Ltd.);

VRD-16 bipolar vane-type vacuum pump (Zhejiang Feiji Electromechanical Co., Ltd.);

DGJ-10C vacuum freeze-dryer (Shanghai Biorise Biotechnology Co., Ltd.); Biotage Isolera One flash preparative liquid-phase chromatograph (Biotage Sweden AB).

### Example 1 Preparation of Compound Cpd-1

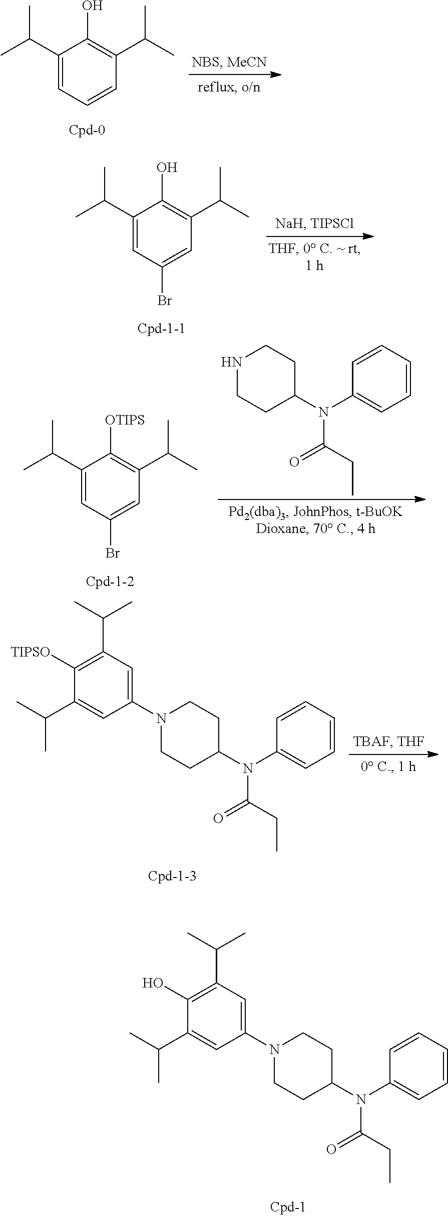

### 1. Preparation of Compound Cpd-1-1

At 0° C., NBS (30.0 g, 168.54 mmol) was added dropwise into a solution of Cpd-0 (25.0 g, 140.45 mmol) in acetonitrile (300 mL), and the mixture was refluxed with stirring overnight. The reaction was monitored by TLC until completion, H₂O (200 mL) was added, the mixture was extracted with ethyl acetate (3×100 mL). The combined organic layers were washed with saturated brine, dried over anhydrous Na₂SO₄, filtered, and concentrated under reduced pressure to obtain a crude product. The crude product was purified by silica gel column chromatography (dichloromethane/petroleum ether (v/v)=1/200-1/50), with TLC (dichloromethane/petroleum ether (v/v)=1/20) momnitoring, and fractions with Rf=0.4-0.5 were collected to yield compound Cpd-1-1 (19.49 g, yield 54.2%) as yellow oil. ESI[M+H]⁺=257.1.

### 2. Preparation of Compound Cpd-1-2

At 0° C., NaH (4.57 g, 60%, 114.18 mmol) was added in portions to a solution of Cpd-1-1 (19.49 g, 76.12 mmol) in dry tetrahydrofuran (120 mL), after the mixture was stirred for 30 minutes, TIPSCl (16.14 g, 83.73 mmol) was added to the mixture. The reaction system was warmed to room temperature and stirred for 30 minutes. The reaction was monitored by TLC until completion, ice-water (100 mL) was added to the reaction system, the organic phase was extracted with ethyl acetate (3×100 mL), washed with saturated brine, dried over anhydrous Na₂SO₄, filtered, and concentrated under reduced pressure to obtain a crude product. The crude product was purified by silica gel column chromatography (heptane as eluent), with TLC (ethyl acetate/petroleum ether (v/v)=1/50) monitoring, and the fractions with Rf=0.5-0.6 were collected to yield compound Cpd-1-2 (30.18 g, yield 96.2%) as white solid. ESI[M+H]⁺=413.3.

### 3. Preparation of Compound Cpd-1-3

At room temperature, compound Cpd-1-2 (138.8 mg, 0.34 mmol), N-phenyl-N-(piperidin-4-yl)propionamide (236.6 mg, 1.02 mmol), Pd₂(dba)₃ (31.1 mg, 0.034 mmol), t-BuOK (76.3 mg, 0.68 mmol), and JohnPhos (22 mg, 0.072 mmol) were dissolved in 1,4-dioxane (6 mL), the reaction system was purged with nitrogen three times, and then stirred at 70° C. for 4 hours under nitrogen protection. The reaction was monitored by TLC until completion, the crude product was obtained by concentration under reduced pressure and purified by silica gel column chromatography (ethyl acetate/petroleum ether (v/v)=1/100-1/10), with TLC (ethyl acetate/petroleum ether (v/v)=1/10) monitoring, and the fractions with Rf=0.3-0.4 were collected to yield compound Cpd-1-3 (160.0 mg, yield 83.4%) as white solid. ESI[M+H]⁺=565.4.

### 4. Preparation of Compound Cpd-1

At 0° C., TBAF (0.56 mL, 1 mol/L in THE, 0.56 mmol) was added to a solution of Cpd-1-3 (160.0 mg, 0.28 mmol) in dry tetrahydrofuran (5 mL), and the mixture was stirred at 0° C. for 1 hour. The reaction was monitored by TLC until completion, water (10 mL) was added to the reaction system, and extracted with ethyl acetate (3×10 mL), the combined organic layers were dried over anhydrous Na₂SO₄, filtered and concentrated under reduced pressure to obtain a crude produc. The crude product was purified by silica gel column chromatography (ethyl acetate/petroleum ether (v/v)=1/100-1/5), with TLC (ethyl acetate/petroleum ether (v/v)=1/5) monitoring, and the fractions with Rf=0.3-0.4 were collected to yield compound Cpd-1 (86.0 mg, yield 74.5%) as white solid. ESI[M+H]⁺=409.3.

¹H NMR (400 MHz, d₆-DMSO) δ 7.57-7.39 (m, 4H), 7.24 (d, J=7.1 Hz, 2H), 6.50 (s, 2H), 4.63-4.48 (m, 1H), 3.38 (d, J=11.9 Hz, 2H), 3.26-3.17 (m, 2H), 2.71-2.55 (m, 2H), 1.95-1.69 (m, 4H), 1.46-1.18 (m, 2H), 1.09 (d, J=6.9 Hz, 12H), 0.89 (t, J=7.4 Hz, 3H).

### Example 2 Preparation of Compound Cpd-2

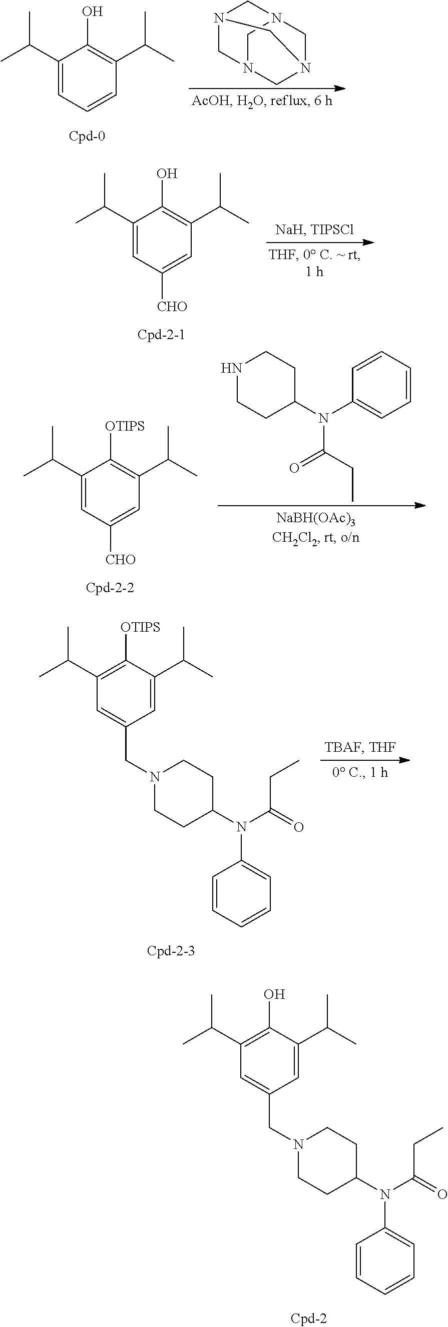

### 1. Preparation of Compound Cpd-2-1

At 0° C., Cpd-0 (5.4 g, 30.1 mmol), urotropine (8.43 g, 60.2 mmol), acetic acid (25.0 mL, 0.83 mL/mmol) and water (5.08 mL, 0.17 mL/mmol) were added to a round-bottom flask, and the mixture was refluxed for 6 hours. The reaction was monitored by TLC until completion, ice-water (100 mL) was added to the reaction system, and the solid was precipitated to obtain the crude product. The crude product was purified by silica gel column chromatography (dichloromethane/petroleum ether (v/v)=1/200-1/30), with TLC (dichloromethane/petroleum ether (v/v)=1/30) monitorin, and fractions with Rf=0.4-0.5 were collected to yield Compound Cpd-2-1 (6.2 g, yield 99.9%) as yellow oil. ESI[M+H]⁺=207.1.

### 2. Preparation of Compound Cpd-2-2

At 0° C., NaH (1.81 g, 60%, 45.1 mmol) was added in portions to a solution of Cpd-2-1 (6.2 g, 30.09 mmol) in dry tetrahydrofuran (100 mL), after the mixture was stirred for 30 minutes, TIPSCl (6.38 g, 33.1 mmol) was added to the mixture. The reaction system was warmed to room temperature and stirred for 1 hour. The reaction was monitored by TLC until completion, ice-water (100 mL) was added to the reaction system. The organic phase was extracted with ethyl acetate (3×100 mL), washed with saturated brine, dried over anhydrous Na₂SO₄, filtered, and concentrated under reduced pressure to obtain a crude product. The crude product was purified by silica gel column chromatography (heptane as eluent), with TLC (ethyl acetate/petroleum ether (v/v)=1/20) monitoring, and the fractions with Rf=0.5-0.6 were collected to yield compound Cpd-2-2 (10.78 g, yield 98.9%) as white solid. ESI[M+H]⁺=363.2.

### 3. Preparation of Compound Cpd-2-3

At room temperature, compound Cpd-2-2 (145 mg, 0.40 mmol), N-phenyl-N-(piperidin-4-yl) propionamide (185 mg, 0.80 mmol) were dissolved in dichloromethane (10 mL), and then stirred at room temperature for 2 hours. NaBH(OAc) 3 (254 mg, 1.20 mmol) was added to the above reaction system, and the mixture was stirred at room temperature overnight. The reaction was monitored by TLC until completion, saturated aqueous sodium bicarbonate (10 mL) was added to the reaction system, and the mixture was extracted with ethyl acetate (3×10 mL), the combined organic layers were dried over anhydrous Na₂SO₄, filtered and concentrated under reduced pressure to obtain a crude produc. The crude product was purified by silica gel column chromatography (ethyl acetate/petroleum ether (v/v)=1/100-1/3), with TLC (ethyl acetate/petroleum ether (v/v)=1/3) monitoring, and the fractions with Rf=0.3-0.4 were collected to yield compound Cpd-2-3 (184 mg, yield 79.5%) as white solid. ESI[M+H]⁺=579.4.

### 4. Preparation of Compound Cpd-2

At 0° C., TBAF (0.64 mL, 1 mol/L in THF, 0.64 mmol) was added to a solution of Cpd-2-3 (184 mg, 0.32 mmol) in dry tetrahydrofuran (5 mL), and the mixture was stirred at 0° C. for 1 hour. The reaction was monitored by TLC until completion, water (10 mL) was added to the reaction system, and extracted with ethyl acetate (3×10 mL), the combined organic layers were dried over anhydrous Na₂SO₄, filtered and concentrated under reduced pressure to obtain a crude produc. The crude product was purified by silica gel column chromatography (ethyl acetate/petroleum ether (v/v)=1/100-1/2), with TLC (ethyl acetate/petroleum ether (v/v)=1/2) monitoring, and the fractions with Rf=0.3-0.4 were collected to yield compound Cpd-2 (125 mg, yield 92.5%) as white solid. ESI[M+H]⁺=423.3.

¹H NMR (400 MHz, d₆-DMSO) δ 7.90 (s, 1H), 7.50-7.39 (m, 3H), 7.19 (d, J=6.7 Hz, 2H), 6.78 (s, 2H), 4.42 (s, 1H), 3.30 (s, 2H), 3.23 (dd, J=13.7, 6.9 Hz, 2H), 2.92-2.71 (m, 2H), 2.07-1.87 (m, 2H), 1.85-1.75 (m, 2H), 1.74-1.60 (m, 2H), 1.28-1.14 (m, 2H), 1.09 (d, J=6.8 Hz, 12H), 0.86 (t, J=7.4 Hz, 3H).

### Example 3 Preparation of Compound Cpd-3

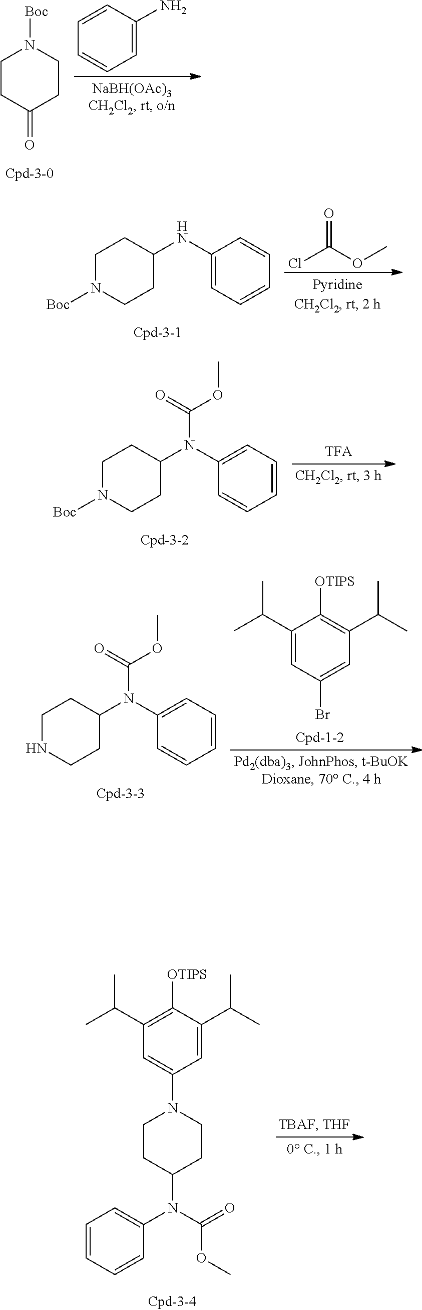

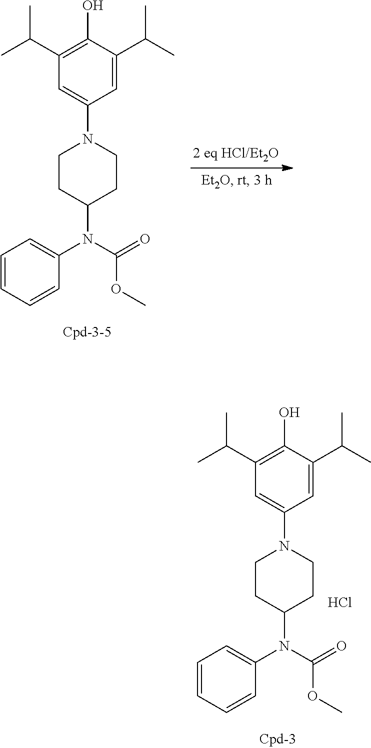 [SMILES: COC(=O)N(c1ccccc1)C1CCN(c2cc(C(C)C)c(O)c(C(C)C)c2)CC1 ; COC(=O)N(c1ccccc1)C1CCN(c2cc(C(C)C)c(O)c(C(C)C)c2)CC1 ; Cl]

### 1. Preparation of Compound Cpd-3-1

At room temperature, Compound Cpd-3-0 (10.0 g, 50.0 mmol) and aniline (5.6 g, 60.0 mmol) were dissolved in dichloromethane (150 mL), and the mixture was stirred at room temperature for 2 hours. NaBH(OAc)₃ (21.2 g, 100.0 mmol) was added to the above reaction system, and the mixture was stirred at room temperature overnight. The reaction was monitored by TLC until completion, saturated aqueous sodium bicarbonate (100 mL) was added to the reaction system, and extracted with ethyl acetate (3×100 mL), the combined organic layers were dried over anhydrous Na₂SO₄, filtered and concentrated under reduced pressure to obtain a crude produc. The crude product was purified by silica gel column chromatography (ethyl acetate/petroleum ether (v/v)=1/100-1/2), with TLC (ethyl acetate/petroleum ether (v/v)=1/2) monitoring, and the fractions with Rf=0.3-0.4 were collected to yield compound Cpd-3-1 (12.1 mg, yield 87.7%) as white solid. ESI[M+H]⁺=277.1.

### 2. Preparation of Compound Cpd-3-2

At room temperature, Pyridine (1.97 g, 25 mmol) and methyl carbonochloridate (930 mg, 10.0 mmol) were added to a solution of Cpd-3-1 (1.38 g, 5.0 mmol) in dry dichloromethane (25 mL), and the mixture was stirred at room temperature for 2 hours. The reaction was monitored by TLC until completion, ice-water (50 mL) was added to the reaction system, the organic phase was extracted with ethyl acetate (3×50 mL), the combined organic layers were dried over anhydrous Na₂SO₄, filtered and concentrated under reduced pressure to obtain a crude produc, the crude product was purified by silica gel column chromatography (ethyl acetate/petroleum ether (v/v)=1/100-1/3), with TLC (ethyl acetate/petroleum ether (v/v)=1/3) monitoring, and the fractions with Rf=0.3-0.4 were collected to yield compound Cpd-3-2 (1.35 g, yield 81.3%) as white solid. ESI[M+H]⁺=335.1.

### 3. Preparation of Compound Cpd-3-3

At room temperature, Compound Cpd-3-2 (1.35 g, 4.06 mmol) was dissolved in dichloromethane (10 mL), and trifluoroacetic acid (2.31 g, 20.3 mmol) was added to the above reaction system, and the mixture was stirred at room temperature for 3 hours. The reaction was monitored by TLC until completion, saturated aqueous sodium bicarbonate was added to adjust the pH to 7-8. Concentrated under reduced pressure to obtain a crude product, the crude product was purified by silica gel column chromatography (methanol/dichloromethane (v/v)=1/100-1/10), with TLC (methanol/dichloromethane (v/v)=1/10) monitoring, and the fractions with Rf=0.3-0.4 were collected to yield compound Cpd-3-3 (755 mg, yield 79.5%) as white solid. ESI[M+H]⁺=235.3.

### 4. Preparation of Compound Cpd-3-4

At room temperature, Compound Cpd-3-3 (238.7 mg, 1.02 mmol), Cpd-1-2 139 mg, 0.34 mmol), Pd₂(dba)₃ (31.1 mg, 0.034 mmol), t-BuOK (76.3 mg, 0.68 mmol) and JohnPhos (22 mg, 0.072 mmol) were dissolved in 1,4-dioxane (5 mL), the reaction system was purged with nitrogen three times, and then stirred at 70° C. for 4 hours under nitrogen protection. The reaction was monitored by TLC until completion, the crude product was obtained by concentration under reduced pressure and purified by silica gel column chromatography (ethyl acetate/petroleum ether (v/v)=1/100-1/10), with TLC (ethyl acetate/petroleum ether (v/v)=1/10) monitoring, and the fractions with Rf=0.3-0.4 were collected to yield compound Cpd-3-4 (120 mg, yield 62.3%) as white solid. ESI[M+H]⁺=567.3.

### 5. Preparation of Compound Cpd-3-5

At 0° C., TBAF (0.42 mL, 1 mol/L in THE, 0.42 mmol) was added to a solution of Cpd-3-4 (120 mg, 0.21 mmol) in dry tetrahydrofuran (5 mL), and the mixture was stirred at 0° C. for 1 hour. The reaction was monitored by TLC until completion, water (10 mL) was added to the reaction system, and extracted with ethyl acetate (3×10 mL), the combined organic layers were dried over anhydrous Na₂SO₄, filtered and concentrated under reduced pressure to obtain a crude product. The crude product was purified by silica gel column chromatography (ethyl acetate/petroleum ether (v/v)=1/100-1/4), with TLC (ethyl acetate/petroleum ether (v/v)=1/4) monitoring, and the fractions with Rf=0.3-0.4 were collected to yield compound Cpd-3-5 (74.3 mg, yield 86.2%) as white solid. ESI[M+H]⁺=411.2.

### 6. Preparation of Compound Cpd-3

At room temperature, a solution of HCl/EtO (0.18 mL, 2 mol/L in Et₂O, 0.36 mmol) was slowly added to a solution of Cpd-3-5 (74.3 mg, 0.18 mmol) in diethyl ether (5 mL). The mixture was stirred at room temperature for 3 h, and the crude product was obtained by concentration under reduced pressure at low temperature. washed with diethyl ether and dried to yield compound Cpd-3 (60 mg, yield 74.7%). ESI[M+H]⁺=411.2.

¹H NMR (400 MHz, d₆-DMSO) δ 8.72 (s, 1H), 7.46 (d, J=7.6 Hz, 3H), 7.33 (s, 2H), 7.24 (d, J=7.2 Hz, 2H), 4.63 (s, 1H), 3.96-3.71 (m, 2H), 3.56 (s, 3H), 3.48 (dd, J=11.3, 7.5 Hz, 2H), 3.34-3.25 (m, 2H), 2.17-2.00 (m, 2H), 1.86 (d, J=8.6 Hz, 2H), 1.13 (d, J=6.8 Hz, 12H).

### Example 4 Preparation of Compound Cpd-4

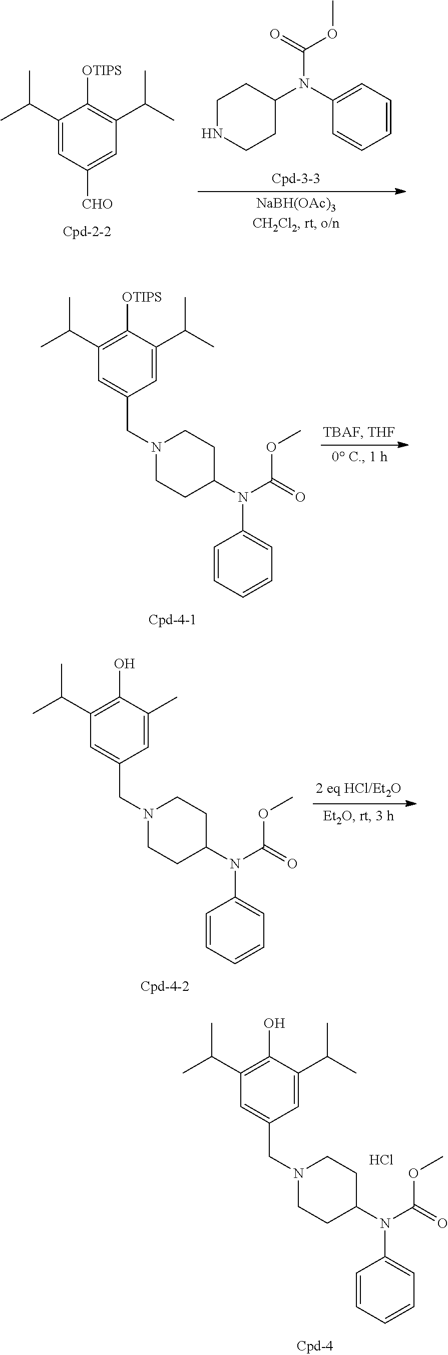

According with the preparation of the Cpd-2, the target compound Cpd-4 was obtained starting from Cpd-2-2 and Cpd-3-3.
Compound Cpd-4: white solid, 60.4 mg, ESI[M+H]⁺=425.2.
¹H NMR (400 MHz, d₆-DMSO) δ 8.42 (s, 1H), 7.48-7.33 (m, 3H), 7.20-7.12 (m, 2H), 7.04 (s, 2H), 4.29 (dt, J=14.1, 9.6 Hz, 1H), 4.06 (d, J=5.0 Hz, 2H), 3.52 (s, 3H), 3.28 (dd, J=13.6, 6.8 Hz, 4H), 3.04 (dd, J=23.3, 11.4 Hz, 2H), 2.00 (d, J=12.2 Hz, 2H), 1.69-1.51 (m, 2H), 1.13 (t, J=7.5 Hz, 12H).

### Example 5 Preparation of Compound Cpd-5

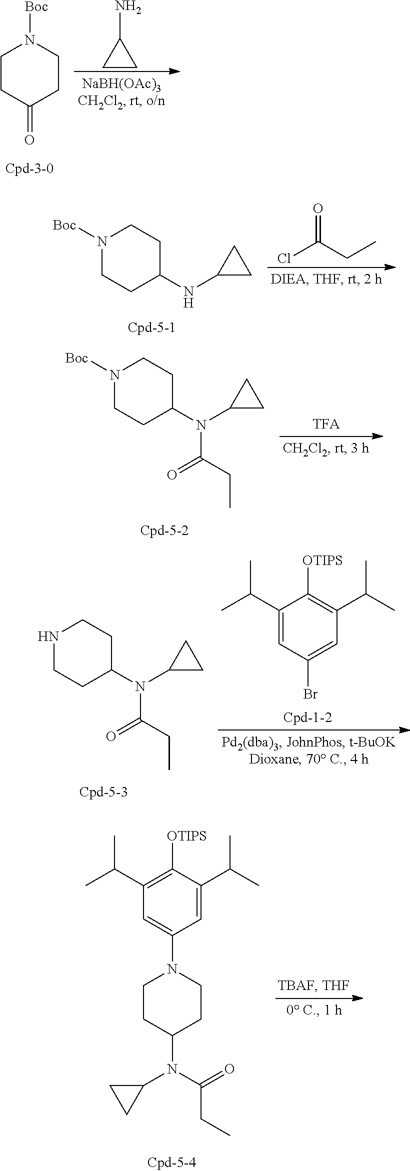

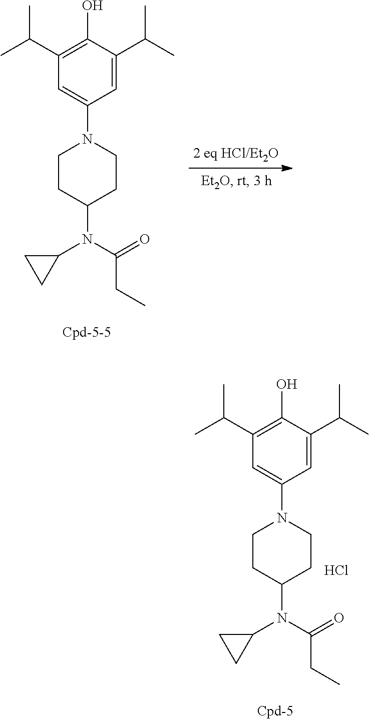 [SMILES: CCC(=O)N(C1CC1)C1CCN(c2cc(C(C)C)c(O)c(C(C)C)c2)CC1 ; CCC(=O)N(C1CC1)C1CCN(c2cc(C(C)C)c(O)c(C(C)C)c2)CC1 ; Cl]

### 1. Preparation of Compound Cpd-5-1

At room temperature, Compound Cpd-3-0 (10.0 g, 50.2 mmol) and Cyclopropylamine (5.73 g, 100.4 mmol) were dissolved in dichloromethane (150 mL), and the mixture was stirred at room temperature for 2 hours. NaBH(OAc) 3 (31.9 g, 150.6 mmol) was added to the above reaction system, and the mixture was stirred at room temperature overnight. The reaction was monitored by TLC until completion, saturated aqueous sodium bicarbonate (100 mL) was added to the reaction system, and extracted with ethyl acetate (3×100 mL), the combined organic layers were dried over anhydrous Na₂SO₄, filtered and concentrated under reduced pressure to obtain a crude produc. The crude product was purified by silica gel column chromatography (ethyl acetate/petroleum ether (v/v)=1/100-1/2), with TLC (ethyl acetate/petroleum ether (v/v)=1/2) monitoring, and the fractions with Rf=0.3-0.4 were collected to yield compound Cpd-5-1 (9.9 g, yield 83.3%) as white solid. ESI[M+H]⁺=241.1.

### 2. Preparation of Compound Cpd-5-2

At room temperature, DIEA (1.94 g, 15 mmol) and propionyl chloride (920 mg, 10.0 mmol) were added to a solution of Cpd-5-1 (1.20 g, 5.0 mmol) in dry dichloromethane (25 mL), and the mixture was stirred at room temperature for 2 hours. The reaction was monitored by TLC until completion, ice-water (50 mL) was added to the reaction system, the organic phase was extracted with Ethyl acetate (3×50 mL), the combined organic layers were dried over anhydrous Na₂SO₄, filtered and concentrated under reduced pressure to obtain a crude produc, the crude product was purified by silica gel column chromatography (ethyl acetate/petroleum ether (v/v)=1/100-1/3), with TLC (ethyl acetate/petroleum ether (v/v)=1/3) monitoring, and the fractions with Rf=0.3-0.4 were collected to yield compound Cpd-5-2 (1.33 g, yield 90.3%) as white solid. ESI[M+H]⁺=297.2.

### 3. Preparation of Compound Cpd-5-3

At room temperature, Compound Cpd-5-2 (1.33 g, 4.50 mmol) was dissolved in dichloromethane (10 mL), and trifluoroacetic acid (2.57 g, 22.5 mmol) was added to the above reaction system, and the mixture was stirred at room temperature for 3 hours, The reaction was monitored by TLC until completion, saturated aqueous sodium bicarbonate was added to adjust the pH to 7-8. Concentrated under reduced pressure to obtain a crude product, the crude product was purified by silica gel column chromatography (methanol/dichloromethane (v/v)=1/100-1/10), with TLC (methanol/dichloromethane (v/v)=1/10) monitoring, and the fractions with Rf=0.3-0.4 were collected to yield compound Cpd-5-3 (684 mg, yield 77.2%) as white solid. ESI[M+H]⁺=197.1.

### 4. Preparation of Compound Cpd-5-4

At room temperature, Compound Cpd-5-3 (210 mg, 1.08 mmol), Cpd-1-2 (207.6 mg, 0.54 mmol), Pd₂(dba)₃ (49.5 mg, 0.054 mmol), t-BuOK (242.4 mg, 2.16 mmol) and JohnPhos (32.2 mg, 0.108 mmol) were dissolved in 1,4-dioxane (5 mL), the reaction system was purged with nitrogen three times, and then stirred at 70° C. for 4 hours under nitrogen protection. The reaction was monitored by TLC until completion, the crude product was obtained by concentration under reduced pressure and purified by silica gel column chromatography (ethyl acetate/petroleum ether (v/v)=1/100-1/10), with TLC (ethyl acetate/petroleum ether (v/v)=1/10) monitoring, and the fractions with Rf=0.3-0.4 were collected to yield compound Cpd-5-4 (246 mg, yield 86.2%) as white solid. ESI[M+H]⁺=529.1.

### 5. Preparation of Compound Cpd-5-5

At 0° C., TBAF (0.94 mL, 1 mol/L in THE, 0.94 mmol) was added to a solution of Cpd-5-4 (246 mg, 0.47 mmol) in dry tetrahydrofuran (5 mL), and the mixture was stirred at 0° C. for 1 hour. The reaction was monitored by TLC until completion, water (10 mL) was added to the reaction system, and extracted with ethyl acetate (3×10 mL), the combined organic layers were dried over anhydrous Na₂SO₄, filtered and concentrated under reduced pressure to obtain a crude product. The crude product was purified by silica gel column chromatography (ethyl acetate/petroleum ether (v/v)=1/100-1/4), with TLC (ethyl acetate/petroleum ether (v/v)=1/4) monitoring, and the fractions with Rf=0.3-0.4 were collected to yield compound Cpd-5-5 (167.4 mg, yield 95.7%) as white solid. ESI[M+H]⁺=373.2.

### 6. Preparation of Compound Cpd-5

At room temperature, a solution of HCl/EtO (0.15 mL, 2 mol/L in Et₂O, 0.30 mmol) was slowly added to a solution of Cpd-5-5 (167.4 mg, 0.45 mmol) in diethyl ether (5 mL). The mixture was stirred at room temperature for 3 hours, and the crude product was obtained by concentration under reduced pressure at low temperature. Washed with diethyl ether and dried to yield compound Cpd-5 (144.7 mg, yield 77.2%). ESI[M+H]⁺=373.3.

¹H NMR (400 MHz, d₆-DMSO) δ 7.54 (s, 2H), 4.37 (t, J=12.5 Hz, 1H), 3.76 (dd, J=20.4, 10.5 Hz, 2H), 3.44 (d, J=11.0 Hz, 2H), 3.38-3.25 (m, 2H), 2.76-2.57 (m, 3H), 2.57-2.51 (m, 2H), 1.85 (d, J=12.3 Hz, 2H), 1.16 (d, J=6.8 Hz, 12H), 1.01 (t, J=7.4 Hz, 3H), 0.93-0.83 (m, 4H).

### Example 6 Preparation of Compound Cpd-7 to Cpd-8

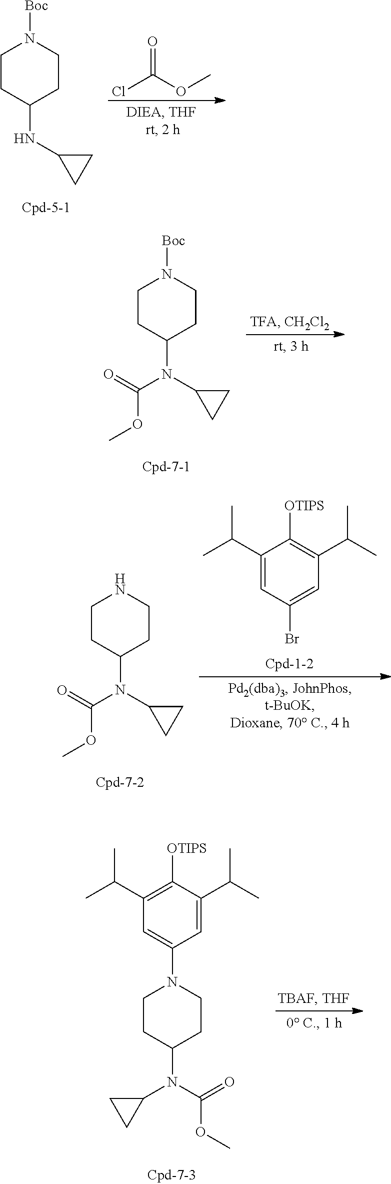

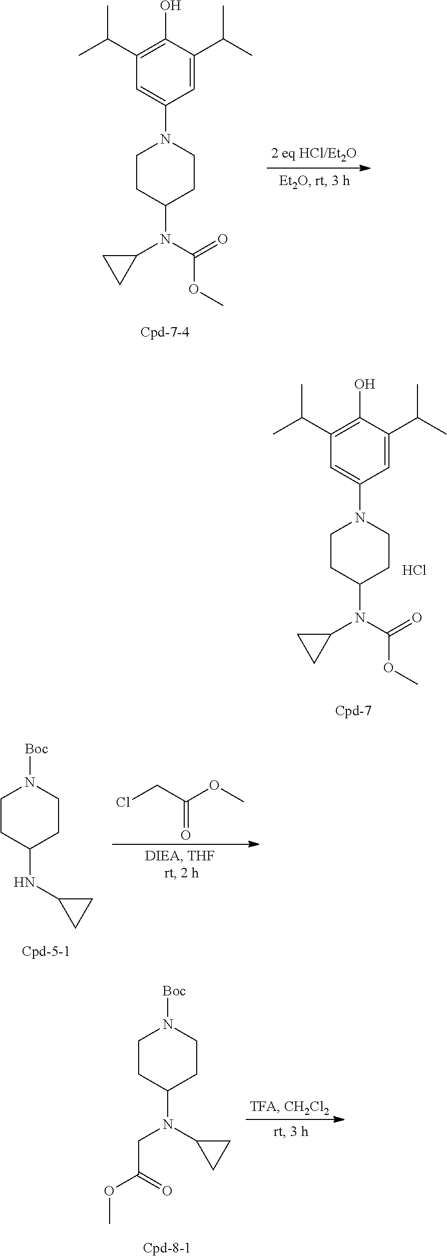

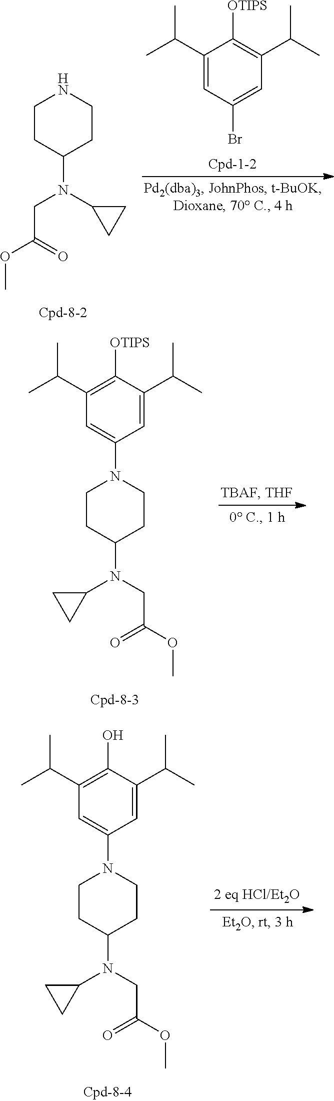

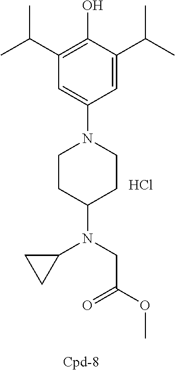 [SMILES: COC(=O)CN(C1CC1)C1CCN(c2cc(C(C)C)c(O)c(C(C)C)c2)CC1 ; Cl]

According with the preparation of the Cpd-5, the target compound Cpd-7 to Cpd-8 were obtained starting from Cpd-5-1.

Compound Cpd-7: white solid, 118.7 mg, ESI[M+H]⁺=375.3.

¹H NMR (400 MHz, d₆-DMSO) δ 8.71 (s, 1H), 7.45 (s, 2H), 4.06 (s, 1H), 3.86-3.68 (m, 2H), 3.63 (s, 4H), 3.49 (d, J=11.4 Hz, 2H), 3.35-3.26 (m, 2H), 2.71-2.52 (m, 2H), 1.92 (d, J=12.7 Hz, 2H), 1.16 (d, J=6.8 Hz, 12H), 0.84-0.75 (m, 2H), 0.74-0.65 (m, 2H).

Compound Cpd-8: white solid, 184.8 mg, ESI[M+H]⁺=389.2.

¹H NMR (400 MHz, d₆-DMSO) δ 8.70 (s, 1H), 7.52 (s, 2H), 4.62 (s, 4H), 4.04 (s, 2H), 3.73-3.64 (m, 2H), 3.54 (d, J=10.8 Hz, 2H), 3.32 (dt, J=13.6, 6.8 Hz, 2H), 2.79 (s, 1H), 2.46-2.35 (m, 2H), 2.29 (d, J=11.5 Hz, 2H), 1.15 (d, J=6.8 Hz, 12H), 1.01-0.82 (m, 2H), 0.79-0.62 (m, 2H).

### Example 7 Preparation of Compound Cpd-9

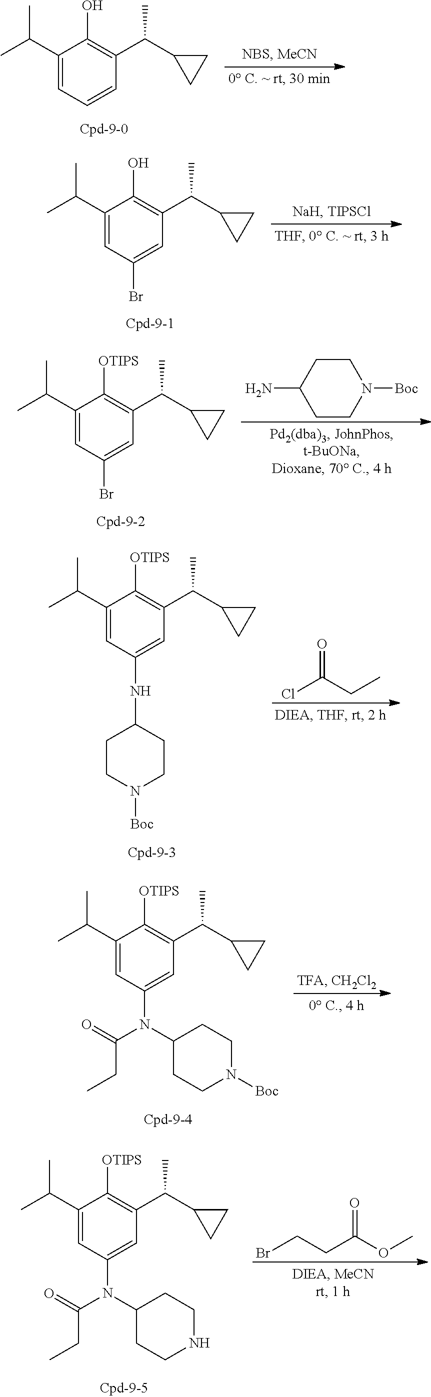

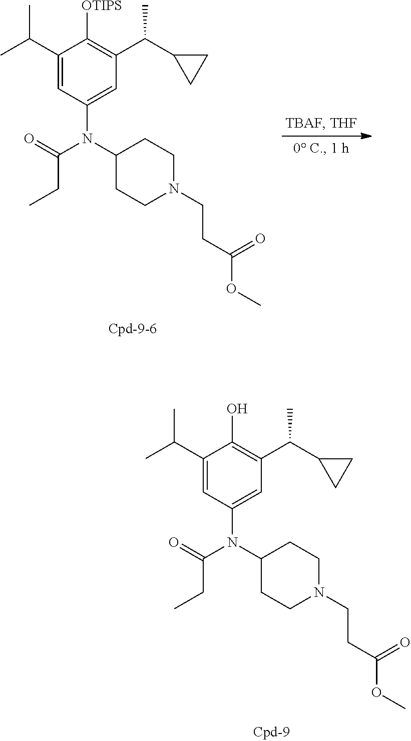

### 1. Preparation of Compound Cpd-9-1

At 0° C., NBS (21.4 g, 120 mmol) was added to a solution of Cpd-9-0 (20.4 g, 100 mmol) in acetonitrile (200 mL), and the mixture was stirred at 0° C. for 30 minutes. The reaction was monitored by TLC until completion, water (100 mL) was added to the reaction system, and extracted with ethyl acetate (3×100 mL), the combined organic layers were dried over anhydrous Na₂SO₄, filtered and concentrated under reduced pressure to obtain a crude produc, the crude product was purified by silica gel column chromatography (ethyl acetate/petroleum ether (v/v)=1/200-1/50), with TLC (ethyl acetate/petroleum ether (v/v)=1/20) monitoring, and the fractions with Rf=0.4-0.5 were collected to yield compound Cpd-9-1 (12.44 g, yield 44.1%) as white solid. ESI[M+H]⁺=283.1.

### 2. Preparation of Compound Cpd-9-2

At 0° C., sodium hydride (2.65 g, 60%, 66.15 mmol) was added portionwise to a solution of Cpd-9-1 (12.44 g, 44.1 mmol) in dry tetrahydrofuran (100 mL), after stirring for 30 minutes, TIPSCl (9.36 g, 48.51 mmol) was added. The reaction system was warmed to room temperature and stirred for 3 hours. The reaction was monitored by TLC until completion, ice-water (100 mL) was added to the reaction system, the organic phase was extracted with ethyl acetate (3×100 mL), the combined organic layers were dried over anhydrous Na₂SO₄, filtered and concentrated under reduced pressure to obtain a crude produc, the crude product was purified by silica gel column chromatography using n-heptane as the mobile phase, with TLC (ethyl acetate/petroleum ether (v/v)=1/50) monitoring, and the fractions with Rf=0.5-0.6 were collected to yield compound Cpd-9-2 (18.12 g, yield 93.6%) as white solid. ESI[M+H]⁺=439.2.

### 3. Preparation of Compound Cpd-9-3

At room temperature, Compound Cpd-9-2 (1.31 g, 3.0 mmol), tert-butyl 4-aminopiperidine-1-carboxylate (1.20 g, 6.0 mmol), Pd₂(dba)₃ (275 mg, 0.30 mmol), 1-BuOK (576 mg, 6.0 mmol) and JohnPhos (179 mg, 0.6 mmol) were dissolved in 1,4-dioxane (20 mL), the reaction system was purged with nitrogen three times, and then stirred at 70° C. for 4 hours under nitrogen protection. The reaction was monitored by TLC until completion, the crude product was obtained by concentration under reduced pressure and purified by silica gel column chromatography (ethyl acetate/petroleum ether (v/v)=1/100-1/5), with TLC (ethyl acetate/petroleum ether (v/v)=1/5) monitoring, and the fractions with Rf=0.3-0.4 were collected to yield compound Cpd-9-3 (1.50 g, yield 89.5%) as white solid. ESI[M+H]⁺=559.4.

### 4. Preparation of Compound Cpd-9-4

At room temperature, DIEA (1.04 g, 8.09 mmol) and propionyl chloride (495 mg, 5.38 mmol) were added to a solution of Cpd-9-3 (1.50 g, 2.69 mmol) in dry tetrahydrofuran (25 mL), and the mixture was stirred at room temperature for 2 hours. The reaction was monitored by TLC until completion, water (50 mL) was added to the reaction system, and extracted with ethyl acetate (3×50 mL), the combined organic layers were dried over anhydrous Na₂SO₄, filtered and concentrated under reduced pressure to obtain a crude product. The crude product was purified by silica gel column chromatography (ethyl acetate/petroleum ether (v/v)=1/100-1/3), with TLC (ethyl acetate/petroleum ether (v/v)=1/3) monitoring, and the fractions with Rf=0.3-0.4 were collected to yield compound Cpd-9-4 (1.39 g, yield 84.3%). ESI[M+H]⁺=615.1.

### 5. Preparation of Compound Cpd-9-5

At room temperature, compound Cpd-9-4 (1.39 g, 2.26 mmol) was dissolved in dichloromethane (15 mL), and trifluoroacetic acid (1.29 g, 11.3 mmol) was added to the above reaction system. The mixture was stirred at 0° C. for 4 hours. After monitoring the completion of the reaction by TLC, saturated aqueous sodium bicarbonate was added to adjust the pH to 7-8. The mixture was concentrated under reduced pressure to obtain the crude product. The crude product was purified by silica gel column chromatography (dichloromethane/methanol (v/v)=1/100-1/10), with TLC (dichloromethane/methanol (v/v)=1/10) monitoring, and the fractions with Rf=0.3-0.4 were collected to yield compound Cpd-9-5 (737 mg, yield 63.5%) as white solid. ESI[M+H]⁺=515.2.

### 6. Preparation of Compound Cpd-9-6

At room temperature, DIEA (557 mg, 4.32 mmol) and methyl 3-bromopropanoate (475 mg, 2.88 mmol) were added to a solution of Cpd-9-5 (737 g, 1.44 mmol) in dry dichloromethane (15 mL), and the mixture was stirred at room temperature for 1 hour. The reaction was monitored by TLC until completion, ice-water (20 mL) was added to the reaction system, the organic phase was extracted with ethyl acetate (3× 20 mL), the combined organic layers were dried over anhydrous Na₂SO₄, filtered and concentrated under reduced pressure to obtain a crude product, the crude product was purified by silica gel column chromatography (ethyl acetate/petroleum ether (v/v)=1/100-1/3), with TLC (ethyl acetate/petroleum ether (v/v)=1/3) monitoring, and the fractions with Rf=0.3-0.4 were collected to yield compound Cpd-9-6 (383 mg, yield 44.3%) as white solid. ESI[M+H]⁺=601.2.

### 7. Preparation of Compound Cpd-9

At 0° C., TBAF (1.28 mL, 1 mol/L in THF, 1.28 mmol) was added to a solution of Cpd-9-6 (383 mg, 0.64 mmol) in dry tetrahydrofuran (10 mL), and the mixture was stirred at 0° C. for 1 hour. The reaction was monitored by TLC until completion, water (10 mL) was added to the reaction system, and extracted with ethyl acetate (3×10 mL), the combined organic layers were dried over anhydrous Na₂SO₄, filtered and concentrated under reduced pressure to obtain a crude product. The crude product was purified by silica gel column chromatography (ethyl acetate/petroleum ether (v/v)=1/100-1/2), with TLC (ethyl acetate/petroleum ether (v/v)=1/2) monitoring, and the fractions with Rf=0.3-0.4 were collected to yield compound Cpd-9 (117.2 mg, yield 69.4%) as white solid. ESI[M+H]⁺=445.3.

¹H NMR (400 MHz, d₆-DMSO) δ 8.31 (s, 1H), 6.89 (d, J=4.8 Hz, 1H), 6.76 (s, 1H), 4.70 (s, 1H), 3.62 (s, 3H), 3.48 (d, J=12.9 Hz, 2H), 3.46 (s, 2H), 3.31-3.24 (m, 1H), 3.26-3.18 (m, 2H), 3.18-3.06 (m, 2H), 2.79 (t, J=7.4 Hz, 2H), 2.54 (s, 1H), 2.02-1.88 (m, 2H), 1.83 (s, 2H), 1.64-1.37 (m, 2H), 1.20 (d, J=5.7 Hz, 3H), 1.15 (d, J=6.6 Hz, 6H), 1.09-0.96 (m, 1H), 0.88 (t, J=7.4 Hz, 3H), 0.56-0.43 (m, 1H), 0.40-0.27 (m, 1H), 0.23-0.12 (m, 1H), 0.11-0.02 (m, 1H).

### Example 8 Preparation of Compound Cpd-10

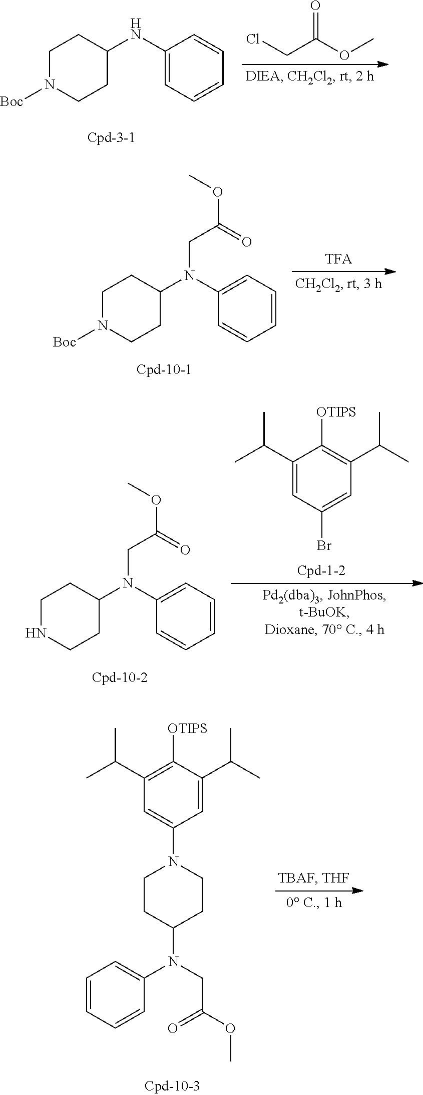

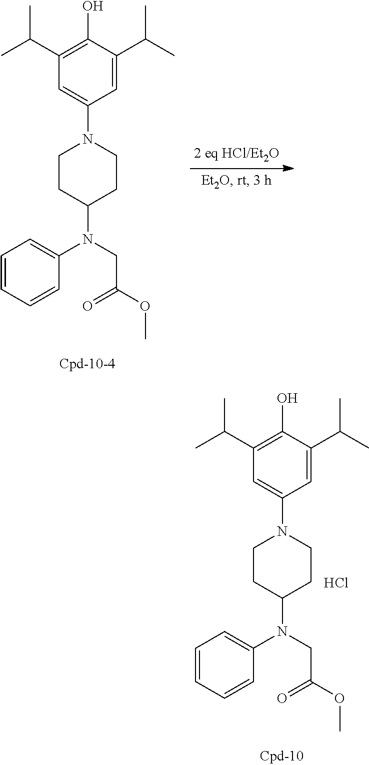 [SMILES: COC(=O)CN(c1ccccc1)C1CCN(c2cc(C(C)C)c(O)c(C(C)C)c2)CC1 ; COC(=O)CN(c1ccccc1)C1CCN(c2cc(C(C)C)c(O)c(C(C)C)c2)CC1 ; Cl]

### 1. Preparation of Compound Cpd-10-1

At room temperature, DIEA (1.94 g, 15 mmol) and methyl 2-chloroacetate (1.08 g, 10.0 mmol) were added into a solution of Cpd-3-1 (1.38 g, 5.0 mmol) in dry dichloromethane (25 mL), then the mixture was stirred at room temperature for 2 hours. The reaction was monitored by TLC until completion, ice-water (50 mL) was added to the reaction system, and extracted with ethyl acetate (3×50 mL), the combined organic layers were dried over anhydrous Na₂SO₄, filtered and concentrated under reduced pressure to obtain a crude product, the crude product was purified by silica gel column chromatography (ethyl acetate/petroleum ether (v/v)=1/100-1/3), with TLC (ethyl acetate/petroleum ether (v/v)=1/3) monitoring, and the fractions with Rf=0.3-0.4 were collected to yield compound Cpd-10-1 (1.27 g, yield 73.1%) as white solid. ESI[M+H]⁺=349.2.

### 2. Preparation of Compound Cpd-10-2

At room temperature, compound Cpd-10-1 (1.27 g, 3.66 mmol) was dissolved in dichloromethane (10 mL), and trifluoroacetic acid (2.08 g, 18.3 mmol) was added to the above reaction system. The mixture was stirred at room temperature for 3 hours. The reaction was monitored by TLC until completion, saturated aqueous sodium bicarbonate was added to adjust the pHI to 7-8, and concentrated under reduced pressure to obtain a crude product, the crude product was purified by silica gel column chromatography (dichloromethane/methanol (v/v)=1/100-1/10), with TLC (dichloromethane/methanol (v/v)=1/10) monitoring, and the fractions with Rf=0.3-0.4 were collected to yield compound Cpd-10-2 (567 mg, yield 62.5%) as white solid. ESI[M+H]⁺=249.2.

### 3. Preparation of Compound Cpd-10-3

At room temperature, Compound Cpd-10-2 (471 mg, 1.90 mmol), Cpd-1-2 (390 mg, 0.95 mmol), Pd₂(dba)₃ (87 mg, 0.095 mmol), t-BuOK (213.2 mg, 1.90 mmol) and JohnPhos (57 mg, 0.19 mmol) were dissolved in 1,4-dioxane (5 ml), the reaction system was purged with nitrogen three times, and then stirred at 70° C. for 4 hours under nitrogen protection. The reaction was monitored by TLC until completion, the crude product was obtained by concentration under reduced pressure and purified by silica gel column chromatography (ethyl acetate/petroleum ether (v/v)=1/100-1/10), with TLC (ethyl acetate/petroleum ether (v/v)=1/10) monitoring, and the fractions with Rf=0.3-0.4 were collected to yield compound Cpd-10-3 (267 mg, yield 48.4%) as white solid. ESI[M+H]⁺=581.3.

### 4. Preparation of Compound Cpd-10-4

At 0° C., TBAF (0.92 mL, 1 mol/L in THE, 0.92 mmol) was added to a solution of Cpd-10-3 (267 mg, 0.46 mmol) in dry tetrahydrofuran (5 mL), and the mixture was stirred at 0° C. for 1 hour. The reaction was monitored by TLC until completion, water (10 mL) was added to the reaction system, and extracted with ethyl acetate (3×10 mL), the combined organic layers were dried over anhydrous Na₂SO₄, filtered and concentrated under reduced pressure to obtain a crude product. The crude product was purified by silica gel column chromatography (ethyl acetate/petroleum ether (v/v)=1/100-1/4), with TLC (ethyl acetate/petroleum ether (v/v)=1/4) monitoring, and the fractions with Rf=0.3-0.4 were collected to yield compound Cpd-10-4 (175 mg, yield 89.7%) as white solid. ESI[M+H]⁺=425.2.

### 5. Preparation of Compound Cpd-10

At room temperature, a solution of HCl/ELO (0.41 mL, 2 mol/L in Et₂O, 0.82 mmol) was slowly added to a solution of Cpd-10-4 (175 mg, 0.41 mmol) in diethyl ether (5 mL). The mixture was stirred at room temperature for 3 hours, and the crude product was obtained by concentration under reduced pressure at low temperature. washed with diethyl ether and dried to yield compound Cpd-10 (178.6 mg, yield 94.2%). ESI[M+H]⁺=425.2.

¹H NMR (400 MHz, CD₃OD) δ 7.34 (s, 2H), 7.23 (d, J=7.4 Hz, 2H), 6.94-6.74 (m, 3H), 4.34 (d, J=4.4 Hz, 1H), 4.16 (s, 2H), 3.92 (d, J=10.7 Hz, 2H), 3.73 (d, J=11.7 Hz, 5H), 3.41-3.32 (m, 2H), 2.31-2.09 (m, 4H), 1.24 (t, J=7.2 Hz, 12H).

### Example 9 Preparation of Compound Cpd-11 to Cpd-15

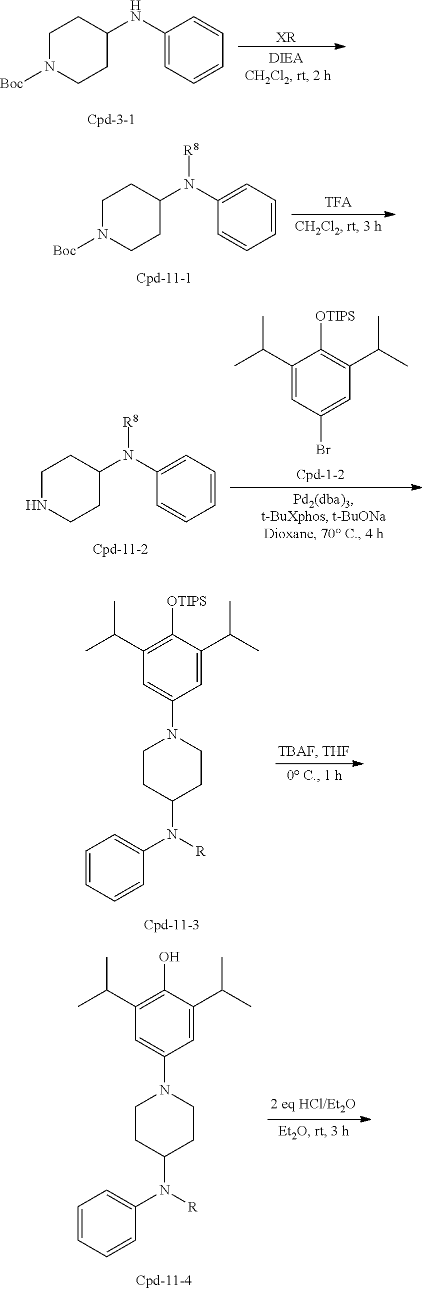

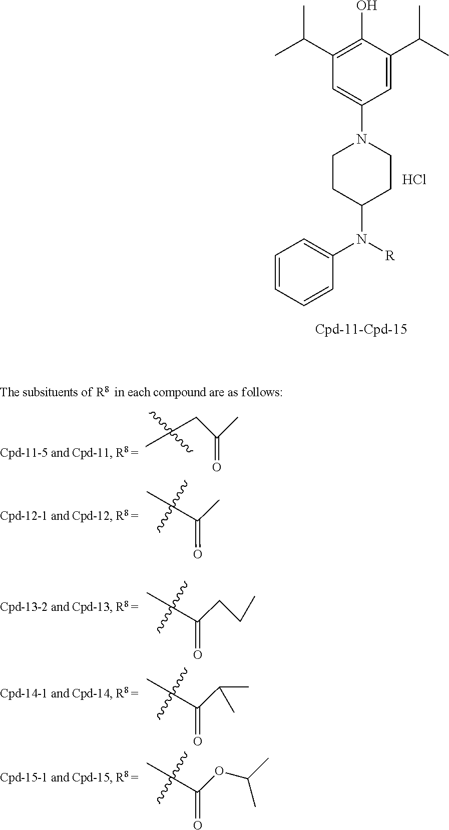

According with the preparation of the Cpd-10, the target compound Cpd-11 to Cpd-15 were obtained starting from Cpd-3-1.

Compound Cpd-11: white solid, 60.2 mg, ESI[M+H]⁺=409.2.

¹H NMR (400 MHz, CD₃OD) δ 7.43-6.83 (m, 7H), 4.38-4.25 (m, 3H), 3.99-3.79 (m, 2H), 3.76-3.61 (m, 2H), 3.41-3.28 (m, 2H), 2.30-2.11 (m, 7H), 1.24 (t, J=5.8 Hz, 12H).

Compound Cpd-12: white solid, 294.1 mg, ESI[M+H]⁺=395.2.

¹H NMR (400 MHz, CD₃OD) δ 7.54 (ddd, J=8.4, 7.6, 6.1 Hz, 3H), 7.36-7.29 (m, 2H), 7.24 (s, 2H), 5.00 (s, 1H), 3.83 (s, 2H), 3.58 (d, J=12.5 Hz, 2H), 3.37-3.31 (m, 2H), 2.21 (d, J=13.7 Hz, 2H), 1.93 (dd, J=12.8, 2.9 Hz, 2H), 1.79 (s, 3H), 1.21 (d, J=6.9 Hz, 12H).

Compound Cpd-13: white solid, 295.7 mg, ESI[M+H]⁺=423.2.

¹H NMR (400 MHz, CD₃OD) δ 7.61-7.47 (m, 3H), 7.36-7.26 (m, 2H), 7.21 (s, 2H), 5.00 (t, J=11.9 Hz, 1H), 3.82 (s, 2H), 3.69-3.48 (m, 2H), 3.32 (d, J=6.9 Hz, 2H), 2.20 (d, J=12.9 Hz, 2H), 1.98 (t, J=7.5 Hz, 2H), 1.88 (dd, J=14.2, 7.6 Hz, 2H), 1.56 (d, J=7.4 Hz, 2H), 1.21 (d, J=6.9 Hz, 12H), 0.81 (t, J=7.4 Hz, 3H).

Compound Cpd-14: white solid, 261.4 mg, ESI[M+H]⁺=423.2.

¹H NMR (400 MHz, CD₃OD) δ 7.60-7.48 (m, 3H), 7.31 (dd, J=8.0, 1.4 Hz, 2H), 7.21 (s, 2H), 4.97 (t, J=12.1 Hz, 1H), 3.88-3.76 (m, 2H), 3.57 (d, J=12.7 Hz, 2H), 3.38-3.31 (m, 2H), 2.40-2.26 (m, 1H), 2.19 (d, J=12.8 Hz, 2H), 2.00-1.77 (m, 2H), 1.21 (d, J=6.9 Hz, 12H), 1.01 (d, J=6.7 Hz, 6H).

Compound Cpd-15: white solid, 254 mg, ESI[M+H]⁺=439.3.

¹H NMR (400 MHz, CD₃OD) δ 7.53-7.34 (m, 3H), 7.22 (dd, J=8.8, 7.5 Hz, 4H), 4.91 (dt, J=10.1, 5.1 Hz, 1H), 4.64 (ddd, J=16.0, 8.0, 3.9 Hz, 1H), 3.84 (t, J=11.9 Hz, 2H), 3.59 (s, 2H), 3.33 (d, J=6.9 Hz, 2H), 2.23 (s, 2H), 2.12-1.92 (m, 2H), 1.21 (d, J=6.9 Hz, 12H), 1.15 (s, 6H).

### Example 10 Preparation of Compound Cpd-17

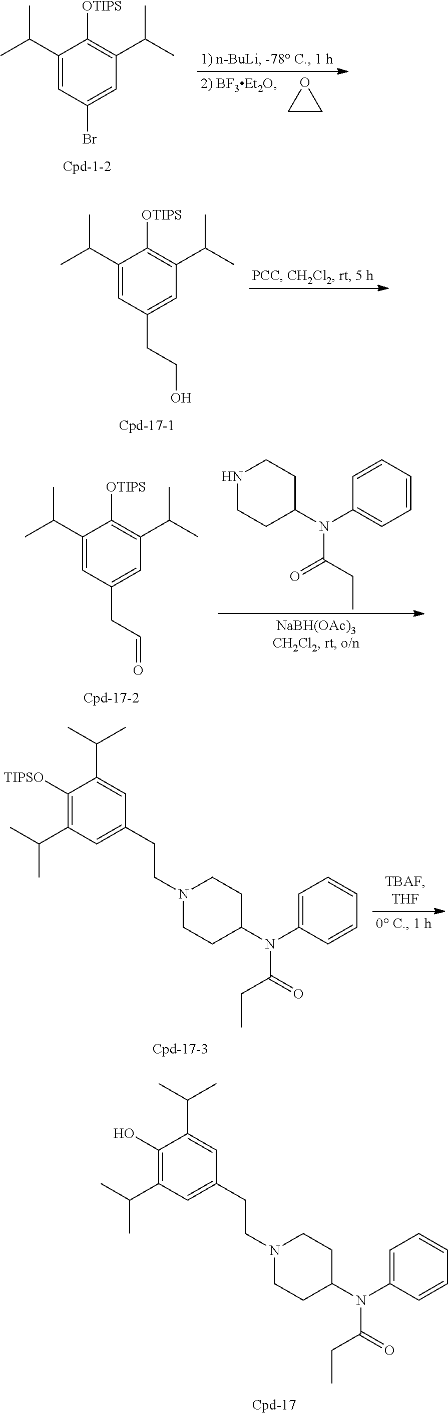

### 1. Preparation of Compound Cpd-17-1

At −78° C., n-BuLi (12 mL, 2.5 mol/L in THE, 30 mmol) was added to a solution of Cpd-1-2 (6.18 g, 15 mmol) in dry tetrahydrofuran (100 mL), and the mixture was stirred at −78° C. for 1 hour. Then boron trifluoride diethyl etherate (21.3 g, 150 mmol) and ethylene oxide (30 mL, 1.0 mol/L in THE, 30 mmol) were added, and the mixture was stirred for 3 hours. The reaction was monitored by TLC until completion, water (100 mL) was added to the reaction system, and the mixture was extracted with ethyl acetate (3×100 mL), the combined organic phases were washed with saturated brine, dried over anhydrous Na₂SO₄, filtered, and concentrated under reduced pressure to obtain the crude product, the crude product was purified by silica gel column chromatography (dichloromethane/petroleum ether (v/v)=1/200-1/50), monitored by TLC (dichloromethane/petroleum ether (v/v)=1/20), and the fractions with Rf=0.4-0.5 were collected to yield compound Cpd-17-1 (1.99 g, yield 35.1%) as yellow oil. ESI[M+H]⁺=379.3.

### 2. Preparation of Compound Cpd-17-2

At room temperature, PCC (5.67 g, 26.3 mmol) was added to a solution of compound Cpd-17-1 (1.99 g, 5.26 mmol) in dichloromethane (30 mL), and the mixture was stirred at room temperature for 5 hours. The reaction was monitored by TLC until completion, the mixture was concentrated under reduced pressure, basified with saturated aqueous sodium bicarbonate (30 mL), and extracted with ethyl acetate (3×30 mL), the combined organic phases were dried over anhydrous Na₂SO₄, filtered, and concentrated under reduced pressure to obtain the crude product, the crude product was purified by silica gel column chromatography (ethyl acetate/petroleum ether (v/v)=1/100-1/50), monitored by TLC (ethyl acetate/petroleum ether (v/v)=1/50), and the fractions with Rf=0.3-0.4 were collected to yield compound Cpd-17-2 (1.23 g, yield 62.3%) as white solid. ESI[M+H]⁺=377.2.

### 3. Preparation of Compound Cpd-17-3

At room temperature, compound Cpd-17-2 (200 mg, 0.53 mmol) and N-phenyl-N-(piperidin-4-yl) propionamide (246 mg, 1.06 mmol) were dissolved in dichloromethane (20 mL), and the mixture was stirred at room temperature for 2 hours. NaBH(OAc)₃ (225 mg, 1.06 mmol) was added to the above reaction system, and the mixture was stirred at room temperature overnight. The reaction was monitored by TLC until completion, saturated aqueous sodium bicarbonate (10 mL) was added to the reaction system, and the mixture was extracted with ethyl acetate (3×10 mL), the combined organic phases were washed with saturated brine, dried over anhydrous Na₂SO₄, filtered, and concentrated under reduced pressure to obtain the crude product, the crude product was purified by silica gel column chromatography (ethyl acetate/petroleum ether (v/v)=1/100-1/5), monitored by TLC (ethyl acetate/petroleum ether (v/v)=1/5), and the fractions with Rf=0.3-0.4 were collected to yield compound Cpd-17-3 (85.0 mg, yield 27.1%) as white solid. ESI[M+H]⁺=593.4.

### 4. Preparation of Compound Cpd-17

At 0° C., TBAF (0.28 mL, 1 mol/L in THF, 0.28 mmol) was added to a solution of Cpd-17-3 (85 mg, 0.14 mmol) in dry tetrahydrofuran (5 mL), and the mixture was stirred at 0° C. for 1 hour. The reaction was monitored by TLC until completion, water (10 mL) was added to the reaction system, and extracted with ethyl acetate (3×10 mL), the combined organic layers were dried over anhydrous Na₂SO₄, filtered and concentrated under reduced pressure to obtain a crude produc. The crude product was purified by silica gel column chromatography (ethyl acetate/petroleum ether (v/v)=1/100-1/2), TLC monitoring (ethyl acetate/petroleum ether (v/v)=1/2) was performed, and the fractions with Rf=0.3-0.4 were collected to compound Cpd-17 (28.4 mg, yield 46.5%) as white solid. ESI[M+H]⁺=437.3.

¹H NMR (400 MHz, CD₃OD) δ 7.47 (dd, J=9.4, 6.4 Hz, 3H), 7.28-7.13 (m, 2H), 6.84 (d, J=44.7 Hz, 2H), 4.59 (t, J=12.3 Hz, 1H), 3.28-3.19 (m, 2H), 2.98 (dd, J=55.7, 11.7 Hz, 2H), 2.65-2.41 (m, 2H), 2.34-2.07 (m, 2H), 2.01-1.90 (m, 2H), 1.89-1.74 (m, 2H), 1.57-1.24 (m, 4H), 1.16 (dd, J=6.9, 3.8 Hz, 12H), 0.98 (dd, J=10.6, 4.3 Hz, 3H).

### Example 11 Preparation of Compound Cpd-18

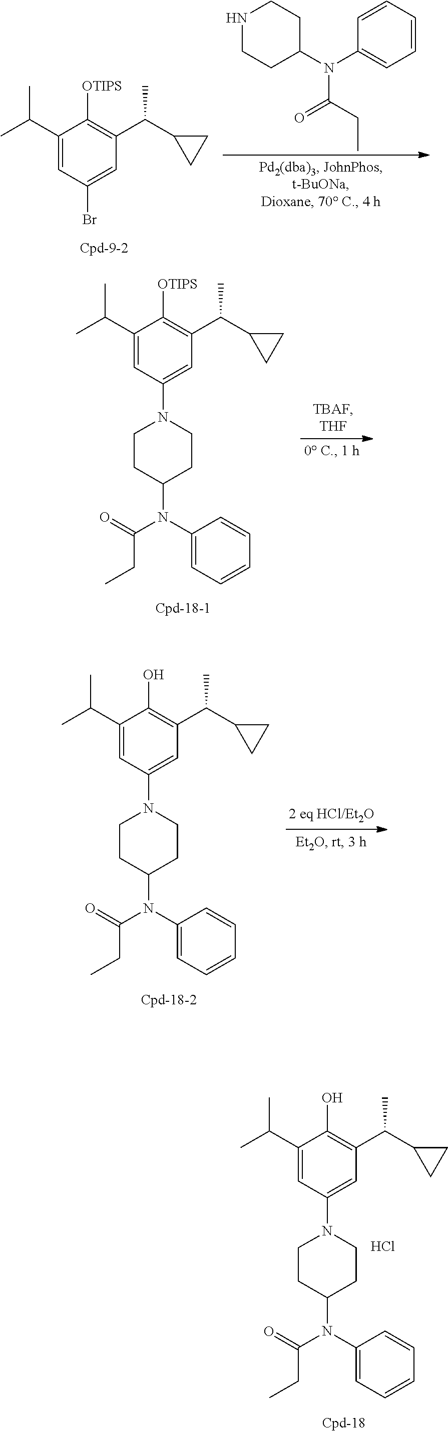

According with the preparation of the Cpd-1, the target compound Cpd-18 was obtained starting from Cpd-9-2.

Compound Cpd-18: white solid, 155.4 mg, ESI[M+H]⁺=435.3.

¹H NMR (400 MHz, CD₃OD) δ 7.57 (dd, J=10.7, 7.2 Hz, 3H), 7.40 (s, 1H), 7.34 (d, J=6.9 Hz, 2H), 7.27 (s, 1H), 5.05 (t, J=12.2 Hz, 1H), 3.89 (s, 2H), 3.64 (d, J=11.9 Hz, 2H), 3.33 (s, 1H), 2.52 (d, J=1.9 Hz, 1H), 2.26 (d, J=13.5 Hz, 2H), 2.06 (q, J=7.4 Hz, 2H), 1.96 (d, J=10.3 Hz, 2H), 1.30 (d, J=6.9 Hz, 3H), 1.26 (d, J=6.8 Hz, 6H), 1.06-1.04 (m, 4H), 0.70-0.57 (m, 1H), 0.47-0.36 (m, 1H), 0.27-0.22 (m, 1H), 0.10-0.04 (m, 1H).

### Example 12 Preparation of Compound Cpd-21

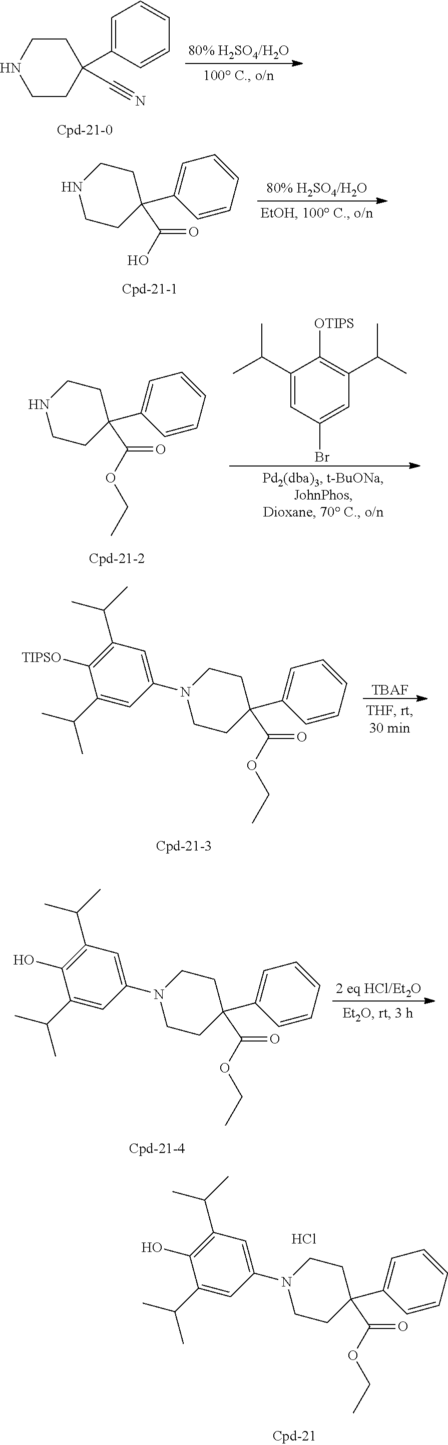

### 1. Preparation of Compound Cpd-21-1

At room temperature, Cpd-21-0 (3.72 g, 20 mmol) and sulfuric acid/water (1.68 mL/1 mL) were sequentially added to a sealed tube, and the mixture was stirred at 100° C. overnight. The reaction was monitored by TLC (methanol/dichloromethane (v/v)=1/5) until completion, the reaction mixture was directly used in the next step.

### 2. Preparation of Compound Cpd-21-2

At room temperature, ethanol (15 mL) was added to the crude Cpd-21-1, and the mixture was stirred at 100° C. overnight. The reaction was monitored by TLC until completion, the mixture was concentrated under reduced pressure to obtain the crude product. The crude product was purified by silica gel column chromatography (methanol/dichloromethane (v/v)=1/100-1/10), monitored by TLC (methanol/dichloromethane (v/v)=1/10), and the fraction with Rf=0.4 was collected to yield crude compound Cpd-21-2 (2.44 g, two-step yield 52.1%) as colorless oil. ESI[M+H]⁺=234.1.

### 3. Preparation of Compound Cpd-21-3

At room temperature, Compound Cpd-21-2 (438 mg, 1.88 mmol), Cpd-1-2 (388 mg, 0.94 mmol), Pd₂(dba)₃ (86 mg, 0.094 mmol), t-BuOK (180 mg, 1.88 mmol), and JohnPhos (56 mg, 0.188 mmol) were dissolved in 1,4-dioxane (5 mL), the reaction system was purged with nitrogen three times, and then stirred at 70° C. for overnight under nitrogen protection. The reaction was monitored by TLC until completion, the crude product was obtained by concentration under reduced pressure and purified by silica gel column chromatography (ethyl acetate/petroleum ether (v/v)=1/100-1/10), with TLC (ethyl acetate/petroleum ether (v/v)=1/10) monitoring, and the fractions with Rf=0.3-0.4 were collected to yield compound Cpd-21-3 (361 mg, yield 67.9%) as white solid. ESI[M+H]⁺=566.4.

### 4. Preparation of Compound Cpd-21-4

At room temperature, TBAF (0.38 mL, 1 mol/L in THF, 0.38 mmol) was added to a solution of Cpd-21-3 (361 mg, 0.64 mmol) in dry tetrahydrofuran (5 mL), and the mixture was stirred at room temperature for 30 mintures. The reaction was monitored by TLC until completion, water (10 mL) was added to the reaction system, and extracted with ethyl acetate (3×10 mL), the combined organic layers were dried over anhydrous Na₂SO₄, filtered and concentrated under reduced pressure to obtain a crude product. The crude product was purified by silica gel column chromatography (ethyl acetate/petroleum ether (v/v)=1/100-1/4), with TLC (ethyl acetate/petroleum ether (v/v)=1/4) monitoring, and the fractions with Rf=0.3-0.4 were collected to yield compound Cpd-21-4 (255 mg, yield 97.6%) as white solid. ESI[M+H]⁺=409.2.

### 5. Preparation of Compound Cpd-21

At room temperature, a solution of HCl/EtO (0.16 mL, 2 mol/L in Et₂O, 0.32 mmol) was slowly added to a solution of Cpd-21-4 (255 mg, 0.62 mmol) in diethyl ether (5 mL). The mixture was stirred at room temperature for 3 hours, and the crude product was obtained by concentration under reduced pressure at low temperature. washed with diethyl ether and dried to yield compound Cpd-21 (253.3 mg, yield 91.7%). ESI[M+H]⁺=410.2.

¹H NMR (400 MHz, CD₃OD) δ 7.64-7.00 (m, 7H), 4.25 (s, 2H), 3.85-3.64 (m, 4H), 3.41-3.33 (m, 2H), 3.07 (d, J=46.8 Hz, 2H), 2.41-2.30 (m, 2H), 1.43-1.20 (m, 15H).

### Example 13 Preparation of Compound Cpd-23

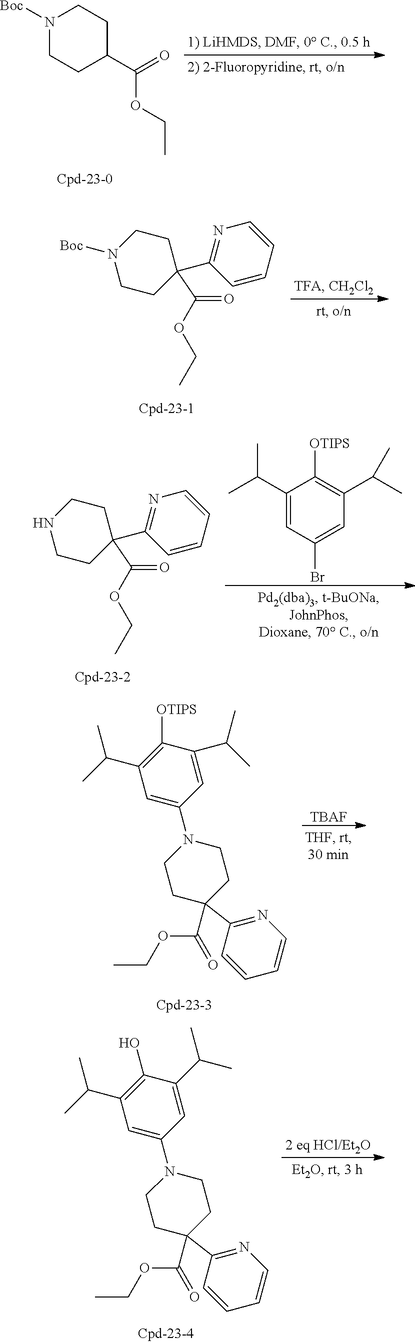

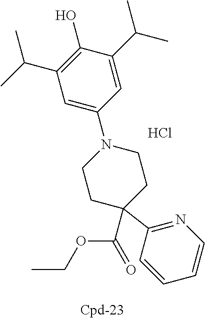 [SMILES: CCOC(=O)C1(c2ccccn2)CCN(c2cc(C(C)C)c(O)c(C(C)C)c2)CC1 ; Cl]

### 1. Preparation of Compound Cpd-23-1

At 0° C., LIHMDS (20 mL, 1 mol/L in THF, 20 mmol) was added to a solution of Cpd-23-0 (2.57 g, 10.0 mmol) in dry DMF (30 mL), and the mixture was stirred at 0° C. for 1 hour. 2-Fluoropyridine (3.14 g, 20.0 mmol) was added to the above reaction system, and the mixture was stirred at room temperature overnight. The reaction was monitored by TLC until completion, water (100 mL) was added to the reaction system, and the mixture was extracted with ethyl acetate (3×100 mL). The combined organic phases were dried over anhydrous Na₂SO₄, filtered, and concentrated under reduced pressure to obtain the crude product. The crude product was purified by silica gel column chromatography (ethyl acetate/petroleum ether (v/v)=1/100 to 1/2), monitored by TLC (ethyl acetate/petroleum ether (v/v)=1/2), and the fraction with Rf=0.5 was collected to yield crude compound Cpd-23-1 (2.22 g, yield 66.4%) as colorless oil. ESI[M+H]⁺=335.1.

### 2. Preparation of Compound Cpd-23-2

At room temperature, Cpd-23-1 (2.22 g, 6.65 mmol) was dissolved in dry dichloromethane (10 mL), trifluoroacetic acid (3.79 g, 33.2 mmol) was added to the above reaction system. The mixture was stirred at room temperature overnight, the reaction was monitored by TLC until completion, saturated aqueous sodium bicarbonate was added to adjust the pH to 7-8. The mixture was concentrated under reduced pressure to obtain the crude product, which was purified by silica gel column chromatography (methanol/dichloromethane (v/v)=1/100-1/10), monitored by TLC (methanol/dichloromethane (v/v)=1/10), the fractions with Rf=0.3-0.4 were collected to yield compound Cpd-23-2 (1.28 g, yield 82.5%) as white solid. ESI[M+H]⁺=235.1.

### 3. Preparation of Compound Cpd-23-3

At room temperature, Compound Cpd-23-2 (440 mg, 1.88 mmol), Cpd-1-2 (388 mg, 0.94 mmol), Pd₂(dba)₃ (86 mg, 0.094 mmol), t-BuOK (180 mg, 1.88 mmol) and JohnPhos (56 mg, 0.188 mmol) were dissolved in 1,4-dioxane (5 mL), the reaction system was purged with nitrogen three times, and then stirred at 70° C. for overnight under nitrogen protection. The reaction was monitored by TLC until completion, the crude product was obtained by concentration under reduced pressure and purified by silica gel column chromatography (ethyl acetate/petroleum ether (v/v)=1/100-1/10), with TLC (ethyl acetate/petroleum ether (v/v)=1/10) monitoring, and the fractions with Rf=0.3-0.4 were collected to yield compound Cpd-23-3 (510 mg, yield 95.8%) as white solid. ESI[M+H]⁺=567.4.

### 4. Preparation of Compound Cpd-23-4

At 0° C., TBAF (1.80 mL, 1 mol/L in THE, 1.80 mmol) was added to a solution of Cpd-23-3 (510 mg, 0.9 mmol) in dry tetrahydrofuran (10 mL), and the mixture was stirred at room temperature for 30 mintures. The reaction was monitored by TLC until completion, water (30 mL) was added to the reaction system, and extracted with ethyl acetate (3×30 mL), the combined organic layers were dried over anhydrous Na₂SO₄, filtered and concentrated under reduced pressure to obtain a crude product. The crude product was purified by silica gel column chromatography (ethyl acetate/petroleum ether (v/v)=1/100-1/4), with TLC (ethyl acetate/petroleum ether (v/v)=1/4) monitoring, and the fractions with Rf=0.3-0.4 were collected to yield compound Cpd-23-4 (360 mg, yield 97.5%) as white solid. ESI[M+H]⁺=411.2.

### 5. Preparation of Compound Cpd-23

At room temperature, a solution of HCl/ELO (0.88 mL, 2 mol/L in EtO, 1.76 mmol) was slowly added to a solution of Cpd-23-4 (360 mg, 0.88 mmol) in diethyl ether (5 mL). The mixture was stirred at room temperature for 3 hours, and the crude product was obtained by concentration under reduced pressure at low temperature. washed with diethyl ether and dried to give compound Cpd-23 (407.3 mg, yield 95.6%). ESI[M+H]⁺=411.2.

¹H NMR (400 MHz, CD₃OD) δ 8.82 (s, 1H), 8.47 (dd, J=7.9, 1.7 Hz, 1H), 8.09 (d, J=8.0 Hz, 1H), 7.91 (ddd, J=7.7, 5.5, 1.0 Hz, 1H), 7.33 (s, 2H), 4.30 (s, 2H), 3.83 (d, J=9.4 Hz, 4H), 3.41-3.32 (m, 2H), 3.16-2.98 (m, 2H), 2.92-2.80 (m, 2H), 1.33-1.18 (m, 15H).

### Example 14 Preparation of Compound Cpd-24

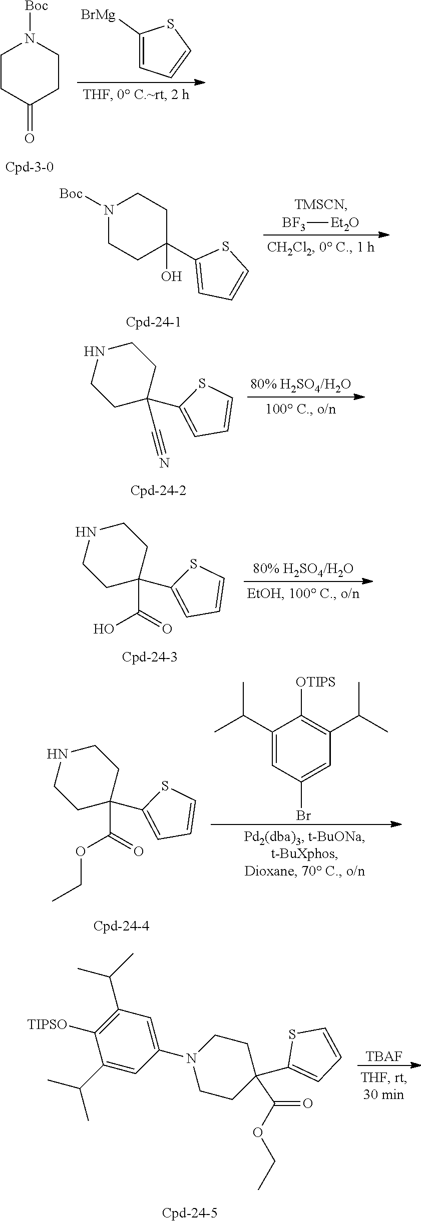

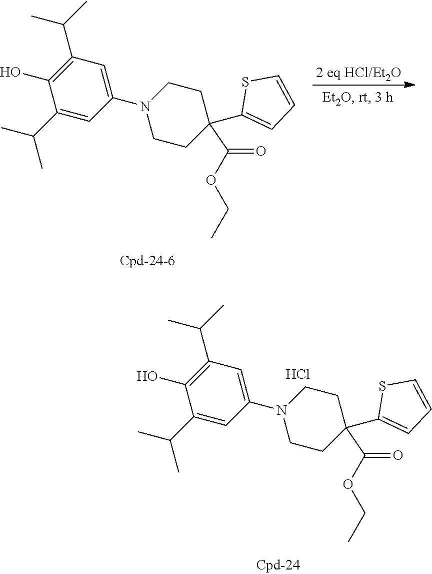 [SMILES: CCOC(=O)C1(c2cccs2)CCN(c2cc(C(C)C)c(O)c(C(C)C)c2)CC1 ; "2 eq HCl/Et2O" ; CCOC(=O)C1(c2cccs2)CCN(c2cc(C(C)C)c(O)c(C(C)C)c2)CC1 ; Cl]

### 1. Preparation of Compound Cpd-24-1

At 0° C., thiophen-2-ylmagnesium bromide (60.0 mL, 1 mol/L in THF, 60.0 mmol) was added to a solution of Cpd-3-0 (5.97 g, 30.0 mmol) in dry tetrahydrofuran (20 mL), and the mixture was stirred at 0° C. for 2 hours. After monitoring the completion of the reaction by TLC, saturated aqueous ammonium chloride (100 mL) was added to the reaction system, and the mixture was extracted with ethyl acetate (3×100 mL). The combined organic phases were dried over anhydrous Na₂SO₄, filtered, and concentrated under reduced pressure to obtain the crude product. The crude product was purified by silica gel column chromatography (ethyl acetate/petroleum ether (v/v)=1/100 to 1/4), monitored by TLC (ethyl acetate/petroleum ether (v/v)=1/4), and the fractions with Rf=0.3-0.4 were collected to give compound Cpd-24-1 (2.3 g, yield 27.1%) as yellow solid. ESI[M+H]⁺=284.1.

### 2. Preparation of Compound Cpd-24-2

At 0° C., Cpd-24-1 (2.3 g, 8.12 mmol), TMSCN (2.41 g, 24.3 mmol) and BF₃-Et₂O (11.6 g, 81.2 mmol) were dissolved in dichloromethane (30 mL), the mixture was stirred at 0° C. for 1 hour. The reaction was monitored by TLC until completion, and concentrated under reduced pressure to obtain a crude product, the crude product was purified by flash column chromatography (methanol/dichloromethane (v/v)=1/100 to 1/10), TLC (methanol/dichloromethane (v/v)=1/10) monitoring was performed, and the fractions with Rf=0.3-0.4 were collected to yield Compound Cpd-24-2 (602 mg, yield 38.6%) as light yellow solid. ESI[M+H]⁺=193.1.

### 3. Preparation of Compound Cpd-24-3

At room temperature, Cpd-24-2 (602 mg, 3.13 mmol) and sulfuric acid/water (0.84 mL/0.5 mL) were sequentially added to a sealed tube, and the mixture was stirred at 100° C. overnight. After monitoring the completion of the reaction by TLC (methanol/dichloromethane (v/v)=1/10), the reaction mixture was directly used in the next step.

### 4. Preparation of Compound Cpd-24-4

At room temperature, ethanol (10 mL) was added to the crude Cpd-24-3, and the mixture was stirred at 100° C. overnight. The reaction was monitored by TLC until completion, the mixture was concentrated under reduced pressure to obtain the crude product, the crude product was purified by silica gel column chromatography (methanol/dichloromethane (v/v)=0-10%), monitored by TLC (methanol/dichloromethane (v/v)=1/10), the fraction with Rf=0.4 was collected to give crude compound Cpd-24-4 (160 mg, two-step yield 21.4%) as colorless oil. ESI[M+H]⁺=240.1.

### 5. Preparation of Compound Cpd-24-5

At room temperature, compound Cpd-24-4 (143 mg, 0.60 mmol), Cpd-1-2 (165 mg, 0.40 mmol), Pd₂(dba)₃ (55 mg, 0.06 mmol), t-BuONa (77 mg, 0.8 mmol) and t-BuXphos (34 mg, 0.08 mmol) were dissolved in 1,4-dioxane (10 mL), the reaction system was purged with nitrogen three times, then stirred at 70° C. overnight under nitrogen protection. The reaction was monitored by TLC until completion, the mixture was concentrated under reduced pressure to obtain the crude product, the crude product was purified by silica gel column chromatography (ethyl acetate/petroleum ether (v/v)=1/100-1/10), monitored by TLC (ethyl acetate/petroleum ether (v/v)=1/10), the fractions with Rf=0.3-0.4 were collected to give compound Cpd-24-5 (48 mg, yield 21.1%) as white solid. ESI[M+H]⁺=572.3.

### 6. Preparation of Compound Cpd-24-6

At room temperature, TBAF (0.16 mL, 1 mol/L in THF, 0.16 mmol) was added to a solution of Cpd-24-5 (48 mg, 0.08 mmol) in dry tetrahydrofuran (5 mL), and the mixture was stirred at room temperature for 30 minutes. The reaction was monitored by TLC until completion, water (10 mL) was added to the reaction system, and the mixture was extracted with ethyl acetate (3×10 mL). The combined organic phases were dried over anhydrous Na₂SO₄, filtered, and concentrated under reduced pressure to obtain the crude product. The crude product was purified by silica gel column chromatography (ethyl acetate/petroleum ether (v/v)=1/100-1/4), monitored by TLC (ethyl acetate/petroleum ether (v/v)=1/4), and the fractions with Rf=0.3-0.4 were collected to give compound Cpd-24-6 (23.4 mg, yield 70.6%) as white solid. ESI[M+H]⁺=416.2.

### 7. Preparation of Compound Cpd-24

At room temperature, HCl/EtO (0.06 mL, 2 mol/L in Et₂O, 0.12 mmol) was slowly added to a solution of Cpd-24-6 (23.4 mg, 0.06 mmol) in diethyl ether (5 mL). The mixture was stirred at room temperature for 3 hours, and the crude product was obtained by low-temperature vacuum concentration. The crude product was washed by diethyl ether and dried to yield compound Cpd-24 (14.6 mg, yield 54.1%). ESI[M+H]⁺=416.2.

¹H NMR (400 MHz, CD₃OD) δ 7.46 (s, 1H), 7.34-7.10 (m, 4H), 4.30 (d, J=6.4 Hz, 2H), 3.84-3.51 (m, 4H), 3.38 (dd, J=13.7, 6.8 Hz, 2H), 3.01 (d, J=34.3 Hz, 2H), 2.55 (s, 2H), 1.32 (s, 3H), 1.27 (d, J=6.8 Hz, 12H).

### Example 15 Preparation of Cpd-25

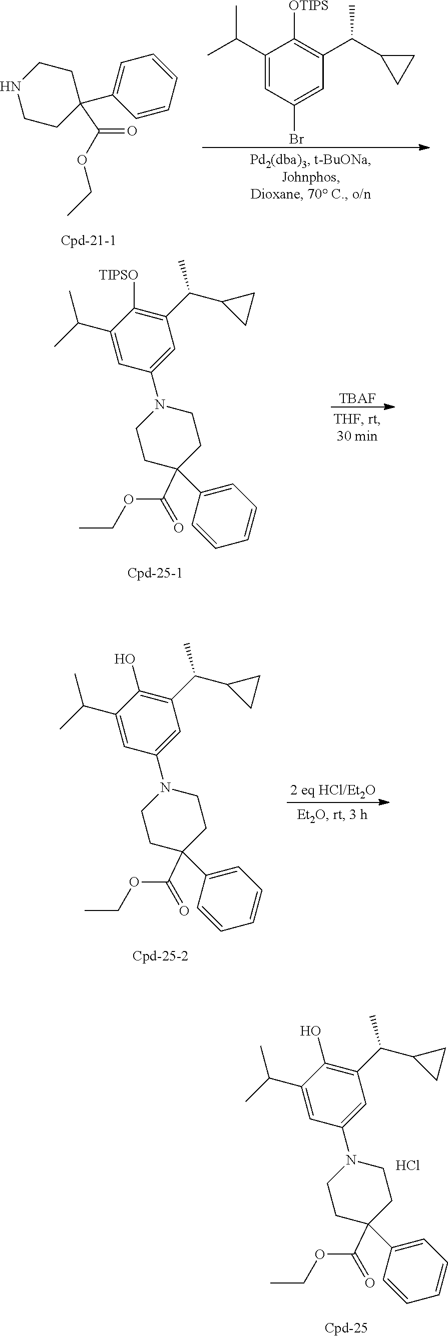

According with the preparation of the Cpd-21, the target compound Cpd-25 was obtained starting from Cpd-21-2.

Compound Cpd-25: white solid, 62.4 mg, ESI[M+H]⁺=436.2.

¹H NMR (400 MHz, CD₃OD) δ 7.68-7.09 (m, 7H), 4.24 (d, J=6.9 Hz, 2H), 3.96-3.54 (m, 4H), 3.33 (d, J=6.9 Hz, 1H), 2.93 (s, 2H), 2.65-2.34 (m, 3H), 1.32-1.16 (m, 12H), 1.07 (d, J=7.8 Hz, 1H), 0.69-0.53 (m, 1H), 0.45-0.39 (m, 1H), 0.28-0.21 (m, 1H), 0.10-0.04 (m, 1H).

### Example 16 Preparation of Compound Cpd-26

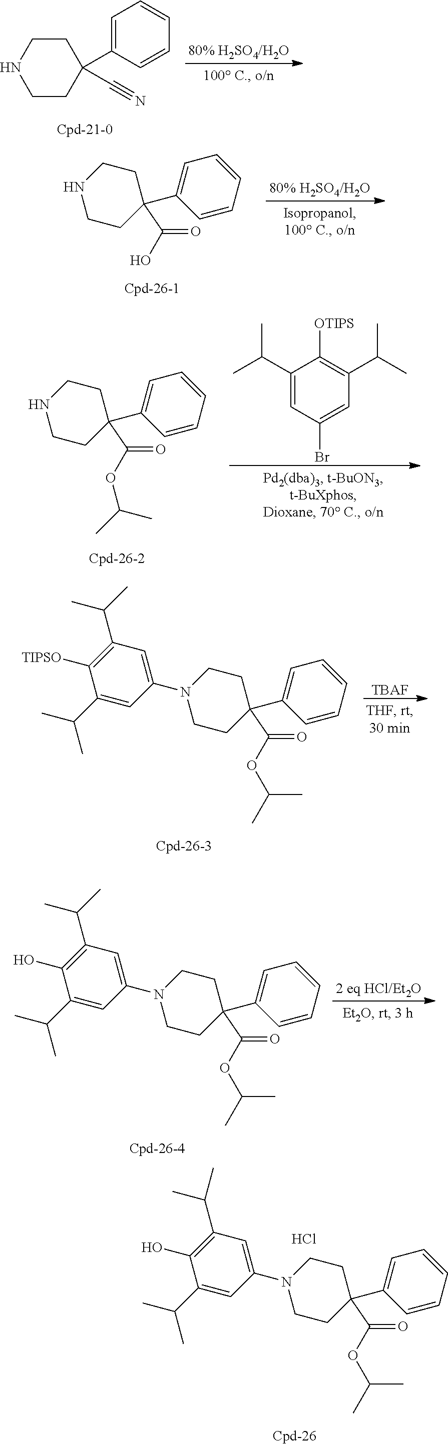

According with the preparation of the Cpd-21, the target compound Cpd-26 was obtained starting from Cpd-21-0.

Compound Cpd-26: white solid, 54.9 mg, ESI[M+H]⁺=424.3.

¹H NMR (400 MHz, CD₃OD) δ 7.59-7.38 (m, 4H), 7.35 (d, J=6.9 Hz, 1H), 7.28-7.21 (m, 2H), 5.11-5.02 (m, 1H), 3.72 (t, J=6.7 Hz, 2H), 3.68-3.60 (m, 2H), 3.31-3.29 (m, 2H), 3.05 (d, J=56.7 Hz, 2H), 2.39-2.31 (m, 2H), 1.23 (d, J=6.7 Hz, 12H), 1.19 (d, J=5.5 Hz, 6H).

### Example 17 Preparation of Compound Cpd-27

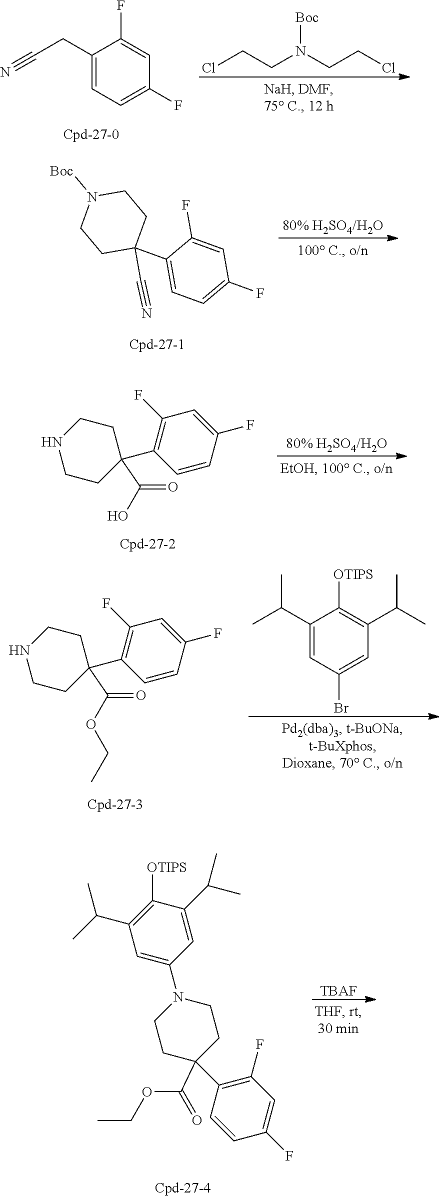

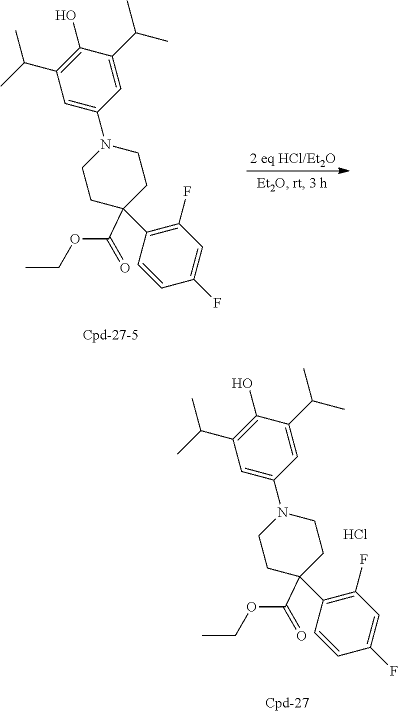 [SMILES: CCOC(=O)C1(c2ccc(F)cc2F)CCN(c2cc(C(C)C)c(O)c(C(C)C)c2)CC1 ; "2 eq HCl/Et2O" ; CCOC(=O)C1(c2ccc(F)cc2F)CCN(c2cc(C(C)C)c(O)c(C(C)C)c2)CC1 ; Cl]

### 1. Preparation of Compound Cpd-27-1

At 0° C., sodium hydride (1.2 g, 60%, 30.0 mmol) was added to a solution of Cpd-27-1 (3.06 g, 20.0 mmol) in DMF (20 mL), and the mixture was stirred at 0° C. for 1 hour. At 0° C., tort-butyl bis(2-chloroethyl)carbamate (932.7 mg, 7.2 mmol) was added to the above reaction system, and the mixture was stirred at 75° C. for 12 hours. The reaction was monitored by TLC until completion, water (100 mL) was added to the reaction system, and the mixture was extracted with ethyl acetate (3×100 mL). The combined organic phases were dried over anhydrous Na₂SO₄, filtered, and concentrated under reduced pressure to obtain the crude product. The crude product was purified by flash column chromatography (ethyl acetate/petroleum ether (v/v)=1/100-1/10), monitored by TLC (ethyl acetate/petroleum ether (v/v)=1/10), and the fraction with Rf=0.5 was collected to give crude compound Cpd-27-1 (3.39 g, yield 52.6%) as colorless oil. ESI[M+H]⁺=323.1.

### 2. Preparation of Compound Cpd-27-2

At room temperature, Cpd-27-1 (2.0 g, 6.21 mmol) and sulfuric acid/water (1.68 mL/1 mL) were sequentially added to a sealed tube, and the mixture was stirred at 100° C. overnight. The reaction was monitored by TLC (methanol/dichloromethane (v/v)=1/5) until completion, the reaction mixture was directly used in the next step.

### 3. Preparation of Compound Cpd-27-3

At room temperature, ethanol (15 mL) was added to the crude Cpd-27-2, and the mixture was stirred at 100° C. overnight. After monitoring the completion of the reaction by TLC, the mixture was concentrated under reduced pressure to obtain the crude product. The crude product was purified by silica gel column chromatography (methanol/dichloromethane (v/v)=1/100 to 1/10), monitored by TLC (methanol/dichloromethane (v/v)=1/10), and the fraction with Rf=0.4 was collected to give compound Cpd-27-3 (790 mg, yield 47.3%) as colorless oil. ESI[M+H]⁺=270.1.

### 4. Preparation of Compound Cpd-27-4

At room temperature, compound Cpd-27-3 (264 mg, 0.98 mmol), Cpd-1-2 (200 mg, 0.49 mmol), Pd₂(dba)₃ (45 mg, 0.049 mmol), t-BuONa (94 mg, 0.98 mmol) and t-BuXphos (42 mg, 0.098 mmol) were dissolved in 1,4-dioxane (10 mL). The reaction system was purged with nitrogen three times, then stirred at 70° C. overnight under nitrogen protection. The reaction was monitored by TLC until completion, the mixture was concentrated under reduced pressure to obtain the crude product. The crude product was purified by silica gel column chromatography (ethyl acetate/petroleum ether (v/v)=1/100-1/10), monitored by TLC (ethyl acetate/petroleum ether (v/v)=1/10). The fractions with Rf=0.3-0.4 were collected to give compound Cpd-27-4 (127 mg, yield 43.2%) as white solid. ESI[M+H]⁺=602.4.

### 5. Preparation of Compound Cpd-27-5

At room temperature, TBAF (0.42 mL, 1 mol/L in THF, 0.42 mmol) was added to a solution of Cpd-10-3 (127 mg, 0.21 mmol) in dry tetrahydrofuran (5 mL), and the mixture was stirred at 0° C. for 1 hour. The reaction was monitored by TLC until completion, water (30 mL) was added to the reaction system, and the mixture was extracted with ethyl acetate (3×20 mL). The combined organic phases were dried over anhydrous Na₂SO₄, filtered, and concentrated under reduced pressure to obtain the crude product. The crude product was purified by silica gel column chromatography (ethyl acetate/petroleum ether (v/v)=1/100-1/4), monitored by TLC (ethyl acetate/petroleum ether (v/v)=1/4). The fractions with Rf=0.3-0.4 were collected to give compound Cpd-27-5 (74 mg, yield 79.6%) as white solid. ESI[M+H]⁺=446.2.

### 6. Preparation of Target Compound Cpd-27

At room temperature, HCl/Et₂O (0.17 mL, 2 mol/L in Et₂O, 0.34 mmol) was slowly added to a solution of Cpd-27-5 (74 mg, 0.17 mmol) in diethyl ether (5 mL). The mixture was stirred at room temperature for 3 hours, and the crude product was obtained by low-temperature vacuum concentration. The crude product was washed with diethyl ether and dried to give compound Cpd-27 (61.9 mg, yield 75.5%). ESI[M+H]⁺=446.2.

¹H NMR (400 MHz, CD₃OD) δ 7.59 (s, 1H), 7.27 (s, 2H), 7.21-7.10 (m, 2H), 4.30 (d, J=6.6 Hz, 2H), 4.02-3.95 (m, 2H), 3.86-3.79 (m, 2H), 3.46-3.36 (m, 2H), 2.85 (d, J=14.2 Hz, 2H), 2.51 (s, 2H), 1.29 (d, J=6.8 Hz, 12H), 1.24 (t, J=7.1 Hz, 3H).

### Example 18 Preparation of Compound Cpd-28

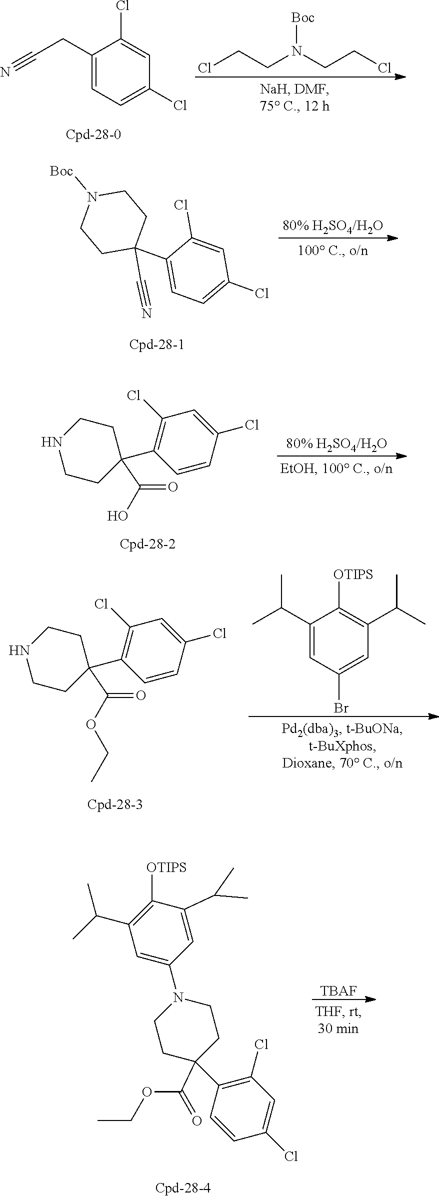

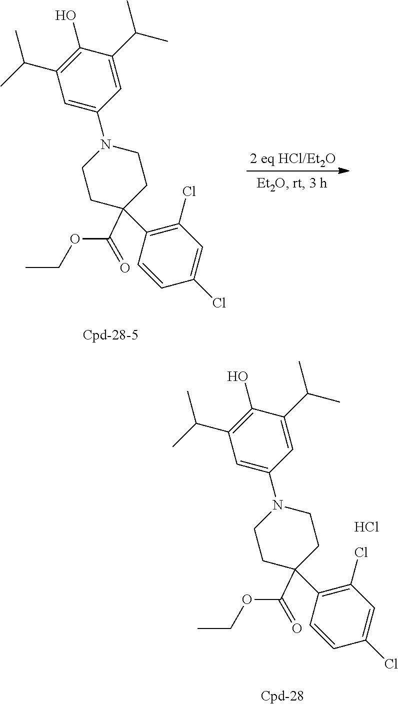 [SMILES: CCOC(=O)C1(c2ccc(Cl)cc2Cl)CCN(c2cc(C(C)C)c(O)c(C(C)C)c2)CC1 ; "2 eq HCl/Et2O" ; CCOC(=O)C1(c2ccc(Cl)cc2Cl)CCN(c2cc(C(C)C)c(O)c(C(C)C)c2)CC1 ; Cl]

According with the preparation of the Cpd-27, the target compound Cpd-28 was obtained starting from Cpd-28-0.
Compound Cpd-28: White solid, 57.6 mg, ESI[M+H]⁺=478.2.
¹H NMR (400 MHz, CD₃OD) δ 7.59 (d, J=2.0 Hz, 2H), 7.52 (s, 1H), 7.28 (s, 2H), 4.29 (d, J=6.8 Hz, 2H), 4.18-2.11 (m, 2H), 3.73 (d, J=11.6 Hz, 2H), 3.48-3.36 (m, 2H), 2.98-2.88 (m, 2H), 2.55-2.43 (m, 2H), 1.30 (d, J=6.8 Hz, 12H), 1.24 (t, J=7.1 Hz, 3H).

### Example 19 Preparation of Compound Cpd-31

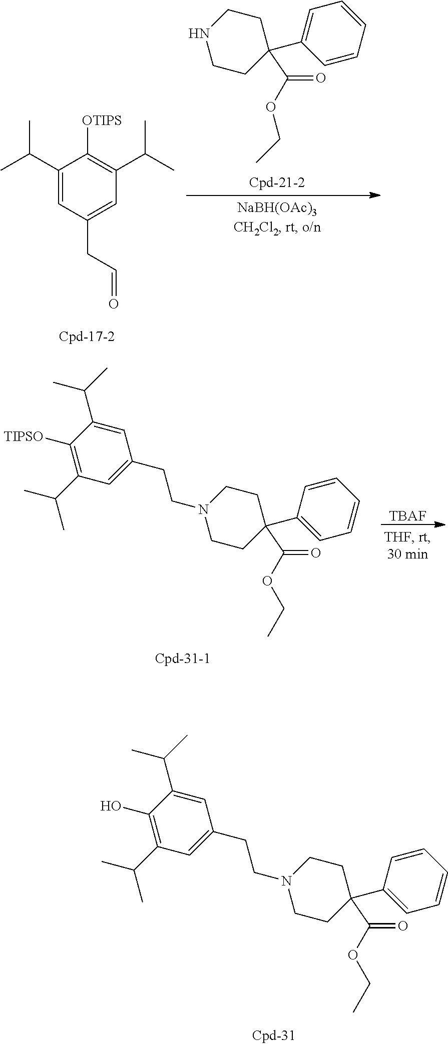

According with the preparation of the Cpd-17, the target compound Cpd-31 was obtained starting from Cpd-17-2.
Compound Cpd-31: White solid, 22.7 mg, ESI[M+H]⁺=438.3.
¹H NMR (400 MHz, CD₃OD) δ 7.55-7.30 (m, 17.9 Hz, 5H), 6.84 (s, 2H), 4.11 (t, J=7.1 Hz, 2H), 3.26 (d, J=6.9 Hz, 2H), 3.10-3.02 (m, 2H), 2.78-2.70 (m, 2H), 2.68-2.63 (m, 2H), 2.42-36 (m, 2H), 2.08-1.97 (m, 2H), 1.32-1.28 (m, 2H), 1.23-1.10 (m, 15H).

### Example 20 Preparation of Compound Cpd-32

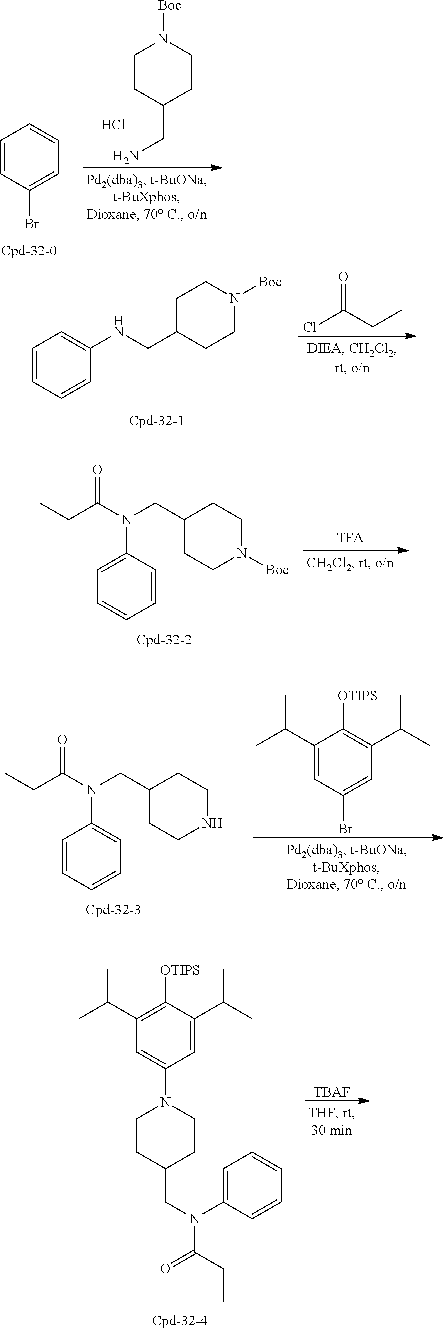

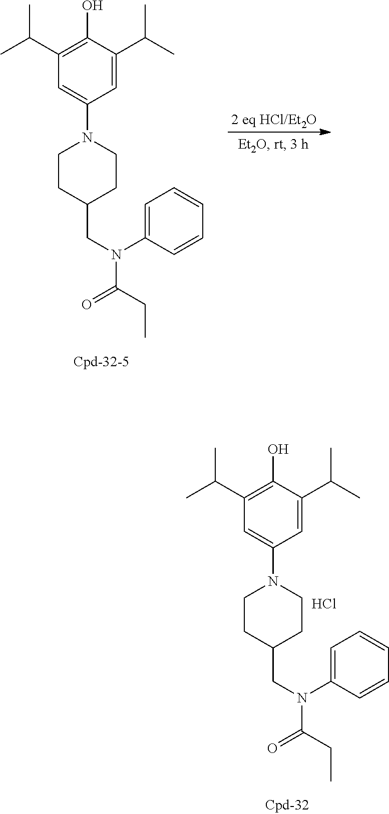 [SMILES: CCC(=O)N(CC1CCN(c2cc(C(C)C)c(O)c(C(C)C)c2)CC1)c1ccccc1 ; "2 eq HCl/Et2O" ; CCC(=O)N(CC1CCN(c2cc(C(C)C)c(O)c(C(C)C)c2)CC1)c1ccccc1 ; Cl]

### 1. Preparation of Compound Cpd-32-1

At room temperature, Cpd-32-0 (800 mg, 5.0 mmol), tert-butyl 4-(aminomethyl) piperidine-1-carboxylate hydrochloride (2.51 g, 10 mmol), Pd₂(dba)₃ (500 mg, 0.5 mmol), t-BuONa (960 mg, 10 mmol) and t-BuXphos (425 mg, 1.0 mmol) were dissolved in 1,4-dioxane (5 mL), the reaction system was purged with nitrogen three times, then stirred at 70° C. overnight under nitrogen protection. The reaction was monitored by TLC until completion, the mixture was concentrated under reduced pressure to obtain the crude product. The crude product was purified by silica gel column chromatography (ethyl acetate/petroleum ether (v/v)=1/100-1/1), monitored by TLC (ethyl acetate/petroleum ether (v/v)=1/1). The fractions with Rf=0.3-0.4 were collected to give compound Cpd-32-1 (870 mg, yield 60.0%) as white solid. ESI[M+H]⁺=291.2.

### 2. Preparation of Compound Cpd-32-2

At room temperature, DIEA (1.16 g, 9.0 mmol) and methyl 2-chloroacetate (552 mg, 6.0 mmol) were added into a solution of Cpd-32-1 (870 mg, 3.0 mmol) in dry dichloromethane (25 mL), then the mixture was stirred at room temperature for 2 hours. The reaction was monitored by TLC until completion, ice-water (50 mL) was added to the reaction system, and extracted with ethyl acetate (3×50 mL), the combined organic layers were dried over anhydrous Na₂SO₄, filtered and concentrated under reduced pressure to obtain a crude produc, the crude product was purified by silica gel column chromatography (ethyl acetate/petroleum ether (v/v)=1/100-1/3), with TLC (ethyl acetate/petroleum ether (v/v)=1/3) monitoring, and the fractions with Rf=0.3-0.4 were collected to yield compound Cpd-32-2 (960 mg, yield 92.3%) as white solid. ESI[M+H]⁺=347.2.

### 3. Preparation of Compound Cpd-32-3

At room temperature, compound Cpd-32-2 (960 mg, 2.77 mmol) was dissolved in dichloromethane (10 mL), and trifluoroacetic acid (1.58 g, 13.85 mmol) was added to the above reaction system. The mixture was stirred at room temperature overnight. The reaction was monitored by TLC until completion, saturated aqueous sodium bicarbonate was added to adjust the pH to 7-8. The mixture was concentrated under reduced pressure to obtain the crude product, which was purified by silica gel column chromatography (methanol/dichloromethane (v/v)=1/100-1/10). Monitored by TLC (methanol/dichloromethane (v/v)=1/10), the fractions with Rf=0.3-0.4 were collected to give compound Cpd-32-3 (470 mg, yield 69.0%) as white solid. ESI[M+H]⁺=247.1.

### 4. Preparation of Compound Cpd-32-4

At room temperature, Cpd-32-3 (246 mg, 1.0 mmol), Cpd-1-2 (206 mg, 0.50 mmol), Pd₂(dba)₃ (46 mg, 0.05 mmol), 1-BuONa (96 mg, 1.0 mmol) and t-BuXphos (43 mg, 0.1 mmol) were dissolved in 1,4-dioxane (15 mL), the reaction system was purged with nitrogen three times, then stirred at 70° C. overnight under nitrogen protection. The reaction was monitored by TLC until completion, the mixture was concentrated under reduced pressure to obtain the crude product. The crude product was purified by silica gel column chromatography (ethyl acetate/petroleum ether (v/v)=1/100-1/8), monitored by TLC (ethyl acetate/petroleum ether (v/v)=1/8). The fractions with R^{f}=0.3-0.4 were collected to give compound Cpd-32-4 (253 mg, yield 87.5%) as white solid. ESI[M+H]⁺=579.4.

### 5. Preparation of Compound Cpd-32-5

At room temperature, TBAF (0.88 mL, 1 mol/L in THE, 0.88 mmol) was added to a solution of Cpd-32-4 (253 mg, 0.44 mmol) in dry tetrahydrofuran (10 mL), and the mixture was stirred at room temperature for 30 minutes. The reaction was monitored by TLC until completion, water (20 mL) was added to the reaction system, and the mixture was extracted with ethyl acetate (3×20 mL). The combined organic phases were dried over anhydrous Na₂SO₄, filtered, and concentrated under reduced pressure to obtain the crude product. The crude product was purified by silica gel column chromatography (ethyl acetate/petroleum ether (v/v)=1/100-1/4), monitored by TLC (ethyl acetate/petroleum ether (v/v)=1/4), and the fractions with Rf=0.3-0.4 were collected to give compound Cpd-32-5 (147.8 mg, yield 79.6%) as white solid. ESI[M+H]⁺=423.3.

### 6. Preparation of Compound Cpd-32

At room temperature, HCl/Et₂O (0.35 mL, 2 mol/L in Et₂O, 0.70 mmol) was slowly added to a solution of Cpd-32-5 (147.8 mg, 0.35 mmol) in diethyl ether (5 mL). The mixture was stirred at room temperature for 3 h, and the crude product was obtained by low-temperature vacuum concentration. The crude product was washed with diethyl ether and dried to afford compound Cpd-32 (118.5 mg, yield 74.1%). ESI[M+H]⁺=423.3.

¹H NMR (400 MHz, CD₃OD) δ 7.50 (d, J=7.8 Hz, 2H), 7.43 (t, J=7.4 Hz, 1H), 7.35 (d, J=7.3 Hz, 2H), 7.25 (s, 2H), 3.76 (s, 2H), 3.58 (s, 4H), 3.39-3.31 (m, 2H), 2.10 (d, J=7.5 Hz, 5H), 1.76 (s, 2H), 1.23 (d, J=6.9 Hz, 12H), 1.01 (t, J=7.5 Hz, 3H).

### Example 21 Preparation of Compound Cpd-33

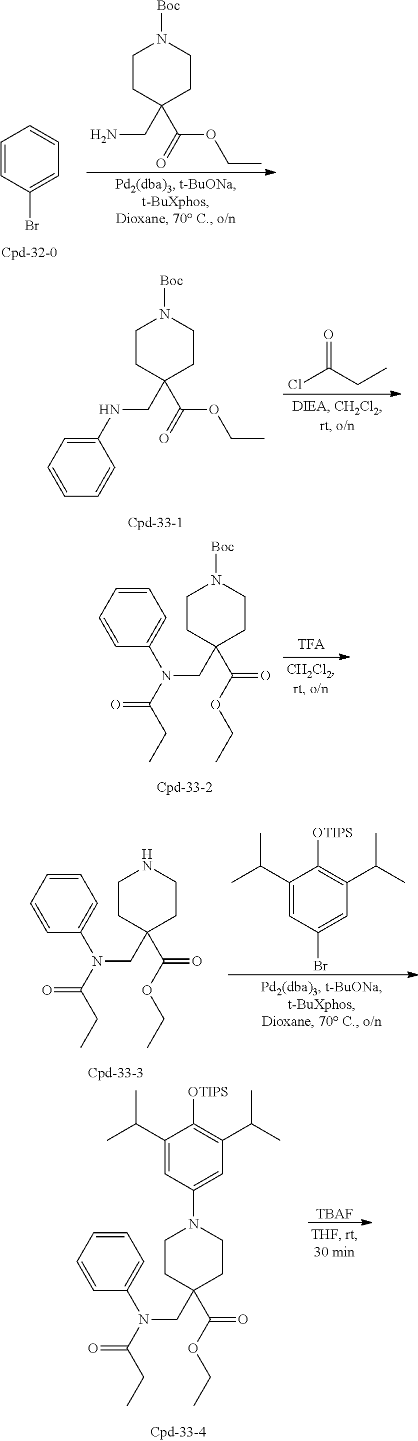

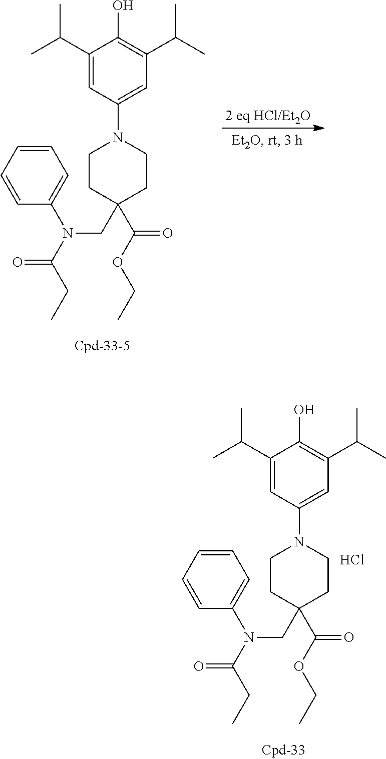 [SMILES: CCOC(=O)C1(CN(C(=O)CC)c2ccccc2)CCN(c2cc(C(C)C)c(O)c(C(C)C)c2)CC1 ; "2 eq HCl/Et2O" ; CCOC(=O)C1(CN(C(=O)CC)c2ccccc2)CCN(c2cc(C(C)C)c(O)c(C(C)C)c2)CC1 ; Cl]

### 1. Preparation of Compound Cpd-33-1

At room temperature, Cpd-32-0 (750 mg, 4.7 mmol), 1-(tert-butyl) 4-ethyl 4-(aminomethyl) piperidine-1,4-dicarboxylate (1.6 g, 5.6 mmol), Pd₂(dba)₃ (430 mg, 0.47 mmol), 1-BuONa (903 mg, 9.4 mmol) and t-BuXphos (400 mg, 0.94 mmol) were dissolved in 1,4-dioxane (5 mL), the reaction system was purged with nitrogen three times, then stirred at 70° C. overnight under nitrogen protection. The reaction was monitored by TLC until completion, the mixture was concentrated under reduced pressure to obtain the crude product. The crude product was purified by silica gel column chromatography (ethyl acetate/petroleum ether (v/v)=1/100-1/1), monitored by TLC (ethyl acetate/petroleum ether (v/v)=1/1). The fractions with Rf=0.3-0.4 were collected to give compound Cpd-33-1 (783 mg, yield 46.1%) as white solid. ESI[M+H]⁺=363.2.

### 2. Preparation of Compound Cpd-33-2

At room temperature, DIEA (836 mg, 6.48 mmol) and methyl 2-chloroacetate (401 mg, 4.32 mmol) were added into a solution of Cpd-33-1 (783 mg, 2.16 mmol) in dry dichloromethane (25 mL), then the mixture was stirred at room temperature for 2 hours. The reaction was monitored by TLC until completion, ice-water (50 mL) was added to the reaction system, and extracted with ethyl acetate (3×50 mL), the combined organic layers were dried over anhydrous Na₂SO₄, filtered and concentrated under reduced pressure to obtain a crude produc, the crude product was purified by silica gel column chromatography (ethyl acetate/petroleum ether (v/v)=1/100-1/3), TLC monitoring (ethyl acetate/petroleum ether (v/v)=1/3) was performed, and the fractions with Rf=0.3-0.4 were collected to yield compound Cpd-33-2 (840 mg, yield 93.1%) as white solid. ESI[M+H]⁺=419.2.

### 3. Preparation of Compound Cpd-33-3

At room temperature, compound Cpd-33-2 (840 mg, 2.0 mmol) was dissolved in dichloromethane (10 mL), and trifluoroacetic acid (1.14 g, 10.0 mmol) was added to the above reaction system. The mixture was stirred at room temperature overnight. The reaction was monitored by TLC until completion, saturated aqueous sodium bicarbonate was added to adjust the pH to 7-8. The mixture was concentrated under reduced pressure to obtain the crude product, which was purified by silica gel column chromatography (methanol/dichloromethane (v/v)=1/100-1/10). Monitored by TLC (methanol/dichloromethane (v/v)=1/10), the fractions with Rf=0.3-0.4 were collected to give compound Cpd-33-3 (509 mg, yield 80.0%) as white solid. ESI[M+H]⁺=319.2.

### 4. Preparation of Compound Cpd-33-4

At room temperature, Cpd-33-3 (318 mg, 1.0 mmol), Cpd-1-2 (100 mg, 0.50 mmol), Pd₂(dba)₃ (46 mg, 0.05 mmol), f-BuONa (96 mg, 1.0 mmol) and t-BuXphos (43 mg, 0.1 mmol) were dissolved in 1,4-dioxane (15 mL), the reaction system was purged with nitrogen three times, then stirred at 70° C. overnight under nitrogen protection. The reaction was monitored by TLC until completion, the mixture was concentrated under reduced pressure to obtain the crude product. The crude product was purified by silica gel column chromatography (ethyl acetate/petroleum ether (v/v)=1/100-1/8), monitored by TLC (ethyl acetate/petroleum ether (v/v)=1/8). The fractions with R^{f}=0.3-0.4 were collected to give compound Cpd-33-4 (244 mg, yield 75.0%) as white solid. ESI[M+H]⁺=651.4.

### 5. Preparation of Compound Cpd-33-5

At room temperature, TBAF (0.768 mL, 1 mol/L in THE, 0.76 mmol) was added to a solution of Cpd-33-4 (244 mg, 0.38 mmol) in dry tetrahydrofuran (10 mL), and the mixture was stirred at room temperature for 30 minutes. The reaction was monitored by TLC until completion, water (30 mL) was added to the reaction system, and the mixture was extracted with ethyl acetate (3×20 mL). The combined organic phases were dried over anhydrous Na₂SO₄, filtered, and concentrated under reduced pressure to obtain the crude product. The crude product was purified by silica gel column chromatography (ethyl acetate/petroleum ether (v/v)=1/100-1/4), monitored by TLC (ethyl acetate/petroleum ether (v/v)=1/4), and the fractions with Rf=0.3-0.4 were collected to give compound Cpd-33-5 (183.2 mg, yield 97.6%) as white solid. ESI[M+H]⁺=495.3.

### 6. Preparation of Compound Cpd-33

At room temperature, HCl/Et₂O (0.37 mL, 2 mol/L in Et₂O, 0.74 mmol) was slowly added to a solution of Cpd-33-5 (183.2 mg, 0.37 mmol) in diethyl ether (5 mL). The mixture was stirred at room temperature for 3 hours, and the crude product was obtained by low-temperature vacuum concentration. The crude product was washed with diethyl ether and dried to give compound Cpd-33 (169.2 mg, yield 86.2%). ESI[M+H]⁺=495.3.

¹H NMR (400 MHz, CD₃OD) δ 7.51-7.20 (m, 7H), 4.15 (s, 2H), 3.85-3.43 (m, 6H), 3.41-3.32 (m, 2H), 2.51-2.39 (m, 2H), 2.09 (d, J=7.4 Hz, 4H), 1.23 (d, J=6.7 Hz, 12H), 0.99 (t, J=7.4 Hz, 6H).

### Example 22 Preparation of Compound Cpd-34 to Cpd-39, Cpd-42

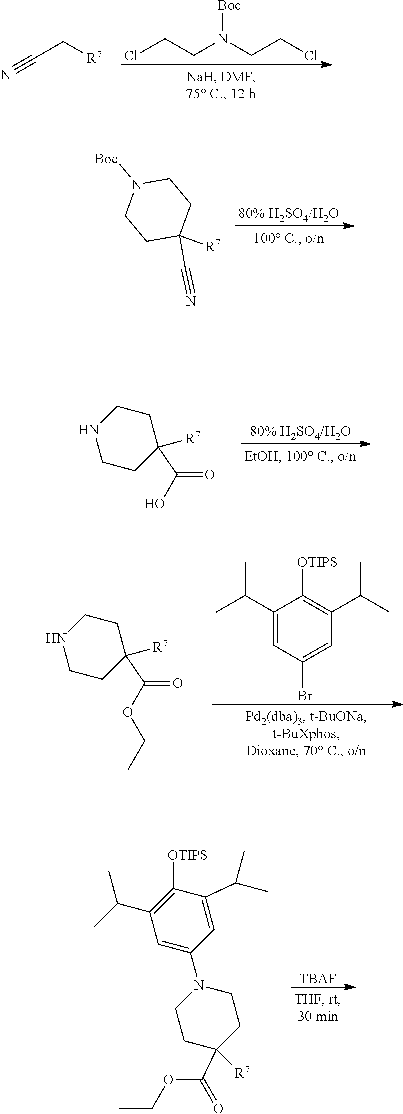

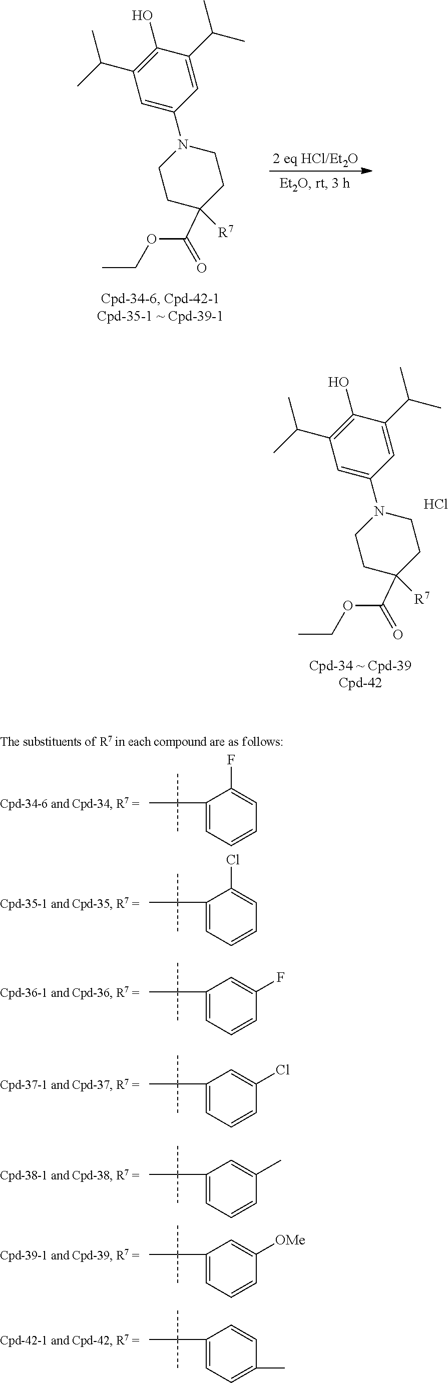

According with the preparation of the Cpd-27, the target compound Cpd-34 to Cpd-39 and Cpd-42 were obtained starting from Cpd-34-0.
Compound Cpd-34: White solid, 111.8 mg, ESI[M+H]⁺=428.3.
¹H NMR (400 MHz, CD₃OD) δ 7.66-7.20 (m, 6H), 4.32-4.28 (m, 2H), 3.99-3.85 (m, 2H), 3.75 (d, J=6.6 Hz, 2H), 3.46-3.36 (m, 2H), 2.95-2.85 (m, 2H), 2.59-2.50 (m, 2H), 1.38-1.17 (m, 15H).
Compound Cpd-35: White solid, 97.4 mg, ESI[M+H]⁺=444.2.
¹H NMR (400 MHz, CD₃OD) δ 7.63 (s, 1H), 7.57-7.36 (m, 3H), 7.29 (s, 2H), 4.28 (d, J=7.0 Hz, 2H), 4.20-4.08 (m, 2H), 3.84-3.65 (m, 2H), 3.46-3.36 (m, 2H), 2.94 (d, J=14.2 Hz, 2H), 2.59-2.50 (m, 2H), 1.30 (d, J=6.8 Hz, 12H), 1.23 (t, J=7.1 Hz, 3H).
Compound Cpd-36: White solid, 104.7 mg, ESI[M+H]⁺=428.3.
¹H NMR (400 MHz, CD₃OD) δ 7.49 (d, J=6.7 Hz, 1H), 7.32 (s, 4H), 7.15 (d, J=7.6 Hz, 1H), 4.32 (s, 2H), 3.75 (dt, J=23.6, 9.5 Hz, 4H), 3.46-3.36 (m, 2H), 3.03 (s, 2H), 2.45 (s, 2H), 1.26 (dd, J=17.3, 6.7 Hz, 15H).
Compound Cpd-37: White solid, 119.1 mg, ESI[M+H]⁺=444.2.
¹H NMR (400 MHz, CD₃OD) δ 7.52-7.32 (m, 6H), 4.39-4.30 (m, 2H), 3.75-3.55 (m, 4H), 3.46-3.36 (m, 2H), 3.13-3.03 (m, 2H), 2.48-2.40 (m, 2H), 1.46-1.18 (m, 15H).
Compound Cpd-38: White solid, 127 mg, ESI[M+H]⁺=424.3.
¹H NMR (400 MHz, CD₃OD) δ 7.57-6.95 (m, 6H), 4.38-4.28 (m, 2H), 3.72-3.53 (m, 4H), 3.46-3.36 (m, 2H), 3.10-3.00 (m, 2H), 2.41 (s, 5H), 1.41-1.15 (m, 15H).
Compound Cpd-39: White solid, 101.2 mg, ESI[M+H]⁺=440.3.
¹H NMR (400 MHz, CD₃OD) δ 7.57-6.79 (m, 6H), 4.38-4.30 (m, 2H), 3.85-3.79 (m, 4H), 3.70 (t, J=11.3 Hz, 2H), 3.46-3.42 (m, 2H), 2.44-2.42 (m, 2H), 1.29-1.20 (m, 15H).
Compound Cpd-42: White solid, 108.6 mg, ESI[M+H]⁺=424.3.
¹H NMR (400 MHz, CD₃OD) δ 7.37-7.29 (m, 6H), 4.29 (s, 2H), 3.75-3.66 (m, 4H), 3.44-3.36 (m, 2H), 3.10-3.00 (m, 1H), 2.38 (s, 5H), 1.96-1.86 (m, 1H), 1.29-1.22 (m, 15H).

### Example 23 Preparation of Compound Cpd-45

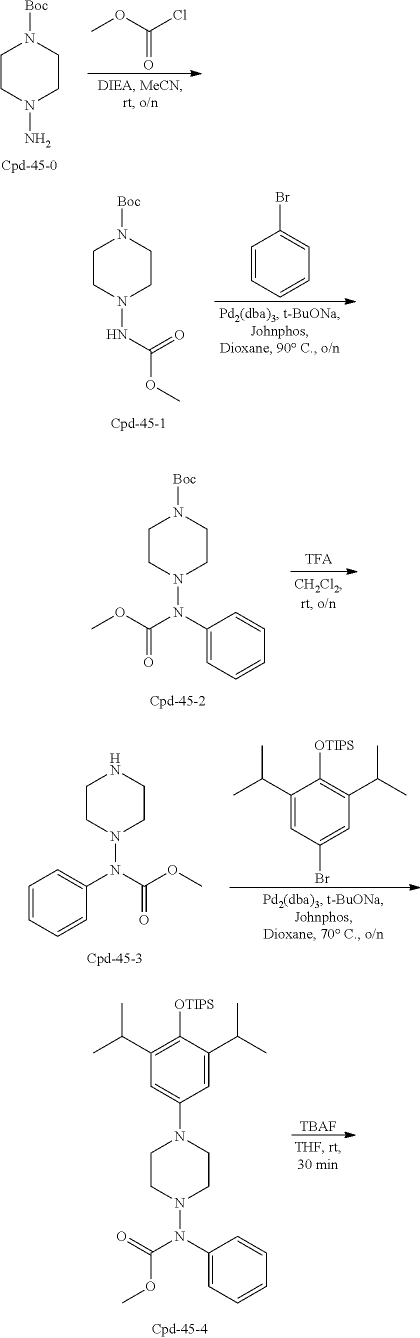

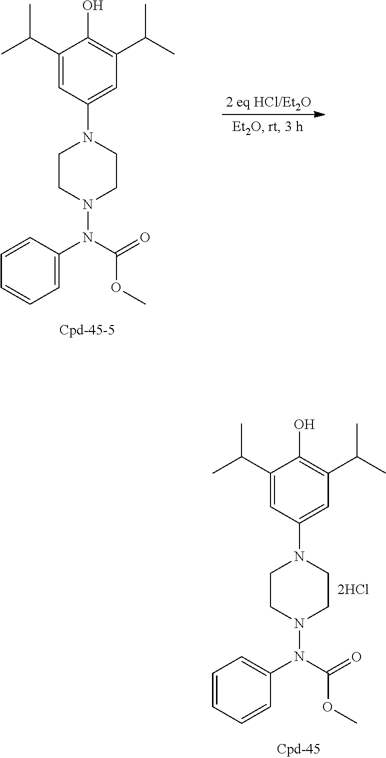 [SMILES: COC(=O)N(c1ccccc1)N1CCN(c2cc(C(C)C)c(O)c(C(C)C)c2)CC1 ; "2 eq HCl/Et2O" ; COC(=O)N(c1ccccc1)N1CCN(c2cc(C(C)C)c(O)c(C(C)C)c2)CC1]

### 1. Preparation of Compound Cpd-45-1

At room temperature, DIEA (3.87 g, 30 mmol) and methyl 2-chloroacetate (1.04 g, 11.0 mmol) were added into a solution of Cpd-45-0 (2.01 g, 10.0 mmol) in dry dichloromethane (25 mL), then the mixture was stirred at room temperature for overnight. The reaction was monitored by TLC until completion, ice-water (50 mL) was added to the reaction system, and extracted with ethyl acetate (3×50 mL), the combined organic layers were dried over anhydrous Na₂SO₄, filtered and concentrated under reduced pressure to obtain a crude produc, the crude product was purified by silica gel column chromatography (ethyl acetate/petroleum ether (v/v)=1/100-1/1), TLC monitoring (ethyl acetate/petroleum ether (v/v)=1/1) was performed, and the fractions with Rf=0.4-0.5 were collected to yield compound Cpd-45-1 (2.33 g, yield 89.9%) as white solid. ESI[M+H]⁺=260.1.

### 2. Preparation of Compound Cpd-45-2

At room temperature, Cpd-32-0 (1.08 g, 6.76 mmol), Cpd-45-1 (2.1 g, 8.11 mmol), Pd₂(dba)₃ (619 mg, 0.68 mmol), t-BuONa (779 mg, 8.11 mmol) and t-BuXphos (402 mg, 1.35 mmol) were dissolved in 1,4-dioxane (30 mL), the reaction system was purged with nitrogen three times, then stirred at 90° C. overnight under nitrogen protection. The reaction was monitored by TLC until completion, the mixture was concentrated under reduced pressure to obtain the crude product. The crude product was purified by silica gel column chromatography (ethyl acetate/petroleum ether (v/v)=1/100-1/3), monitored by TLC (ethyl acetate/petroleum ether (v/v)=1/3). The fractions with Rf=0.3-0.4 were collected to give compound Cpd-45-2 (760 mg, yield 27.9%) as white solid. ESI[M+H]⁺=336.2.

### 3. Preparation of Compound Cpd-45-3

At room temperature, compound Cpd-45-2 (760 mg, 2.27 mmol) was dissolved in dichloromethane (10 mL) and trifluoroacetic acid (1.29 g, 11.4 mmol) was added to the above reaction system. The mixture was stirred at room temperature overnight. The reaction was monitored by TLC until completion, saturated aqueous sodium bicarbonate was added to adjust the pH to 7-8. The mixture was concentrated under reduced pressure to obtain the crude product, which was purified by silica gel column chromatography (methanol/dichloromethane (v/v)=1/100-1/10). Monitored by TLC (methanol/dichloromethane (v/v)=1/10), the fractions with Rf=0.3-0.4 were collected to give compound Cpd-45-3 (470 mg, yield 88.2%) as white solid. ESI[M+H]⁺=236.1.

### 4. Preparation of Compound Cpd-45-4

At room temperature, Cpd-45-3 (236 mg, 1.0 mmol), Cpd-1-2 (206 mg, 0.5 mmol), Pd₂(dba)₃ (46 mg, 0.05 mmol), ¿-BuONa (96 mg, 1.0 mmol) and t-BuXphos (43 mg, 0.1 mmol) were dissolved in 1,4-dioxane (10 mL), the reaction system was purged with nitrogen three times, then stirred at 70° C. overnight under nitrogen protection. The reaction was monitored by TLC until completion, the mixture was concentrated under reduced pressure to obtain the crude product. The crude product was purified by silica gel column chromatography (ethyl acetate/petroleum ether (v/v)=1/100-1/8), monitored by TLC (ethyl acetate/petroleum ether (v/v)=1/8). The fractions with Rf=0.3-0.4 were collected to give compound Cpd-45-4 (142 mg, yield 50.1%) as white solid. ESI[M+H]⁺=568.4.

### 5. Preparation of Compound Cpd-45-5

At room temperature, TBAF (0.50 mL, 1 mol/L in THE, 0.50 mmol) was added to a solution of Cpd-45-4 (142 mg, 0.25 mmol) in dry tetrahydrofuran (10 mL), and the mixture was stirred at room temperature for 30 minutes. The reaction was monitored by TLC until completion, water (30 mL) was added to the reaction system, and the mixture was extracted with ethyl acetate (3×30 mL). The combined organic phases were dried over anhydrous Na₂SO₄, filtered, and concentrated under reduced pressure to obtain the crude product. The crude product was purified by silica gel column chromatography (ethyl acetate/petroleum ether (v/v)=1/100-1/4), monitored by TLC (ethyl acetate/petroleum ether (v/v)=1/4), and the fractions with Rf=0.3-0.4 were collected to give compound Cpd-45-5 (87.1 mg, yield 84.6%) as white solid. ESI[M+H]⁺=412.2.

### 6. Preparation of Compound Cpd-45

At room temperature, HCl/Et₂O (0.21 mL, 2 mol/L in Et₂O, 0.42 mmol) was slowly added to a solution of Cpd-45-5 (87.1 mg, 0.21 mmol) in diethyl ether (5 mL). The mixture was stirred at room temperature for 3 hours, and the crude product was obtained by low-temperature vacuum concentration. The crude product was washed with diethyl ether and dried to give compound Cpd-45 (81.3 mg, yield 79.7%). ESI[M+H]⁺=412.3.

¹H NMR (400 MHz, CD₃OD) δ 7.42 (d, J=7.5 Hz, 2H), 7.39-7.28 (m, 3H), 7.21 (s, 2H), 3.73 (s, 3H), 3.76-3.62 (m, 4H), 3.60-3.38 (m, 4H), 3.33 (d, J=6.9 Hz, 2H), 1.22 (d, J=6.9 Hz, 12H).

### Example 24 Preparation of Compound Cpd-46 and Cpd-47

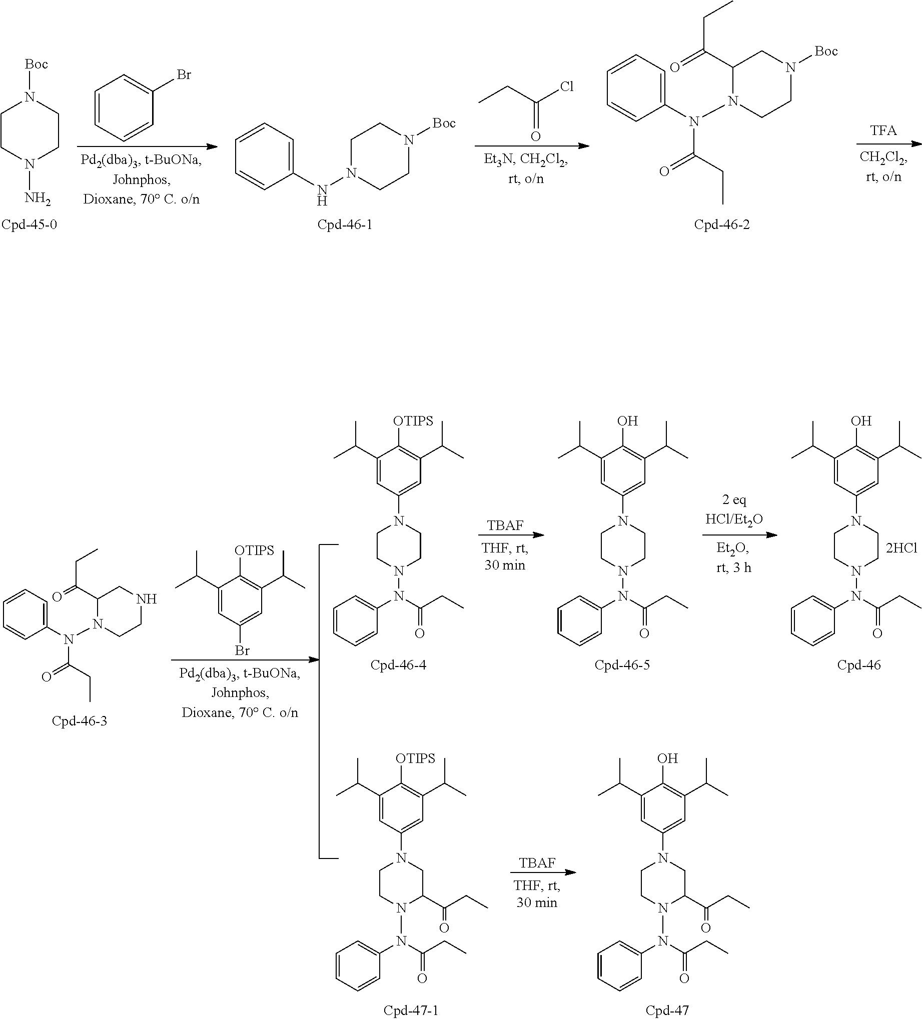

### 1. Preparation of Compound Cpd-46-1

At room temperature, Cpd-32-0 (1.41 g, 9.0 mmol), Cpd-45-0 (2.01 g, 10 mmol), Pd₂(dba)₃ (825 mg, 0.9 mmol), t-BuONa (1.73 g, 18 mmol) and Johnphos (536 mg, 1.8 mmol) were dissolved in 1,4-dioxane (5 mL), the reaction system was purged with nitrogen three times, then stirred at 90° C. overnight under nitrogen protection. The reaction was monitored by TLC until completion, the mixture was concentrated under reduced pressure to obtain the crude product. The crude product was purified by silica gel column chromatography (ethyl acetate/petroleum ether (v/v)=1/100-1/2), monitored by TLC (ethyl acetate/petroleum ether (v/v)=1/2). The fractions with Rf=0.3-0.4 were collected to give compound Cpd-46-1 (960 mg, yield 38.5%) as white solid. ESI[M+H]⁺=278.2.

### 2. Preparation of Compound Cpd-46-2

At room temperature, triethylamine (3.96 g, 39.2 mmol) and propionyl chloride (2.43 g, 26.2 mmol) were added to a solution of Cpd-46-1 (724 mg, 2.62 mmol) in dry dichloromethane (25 mL), and the mixture was stirred at room temperature for 2 hours. The reaction was monitored by TLC until completion, ice-water (50 mL) was added to the reaction system, and the mixture was extracted with ethyl acetate (3×50 mL). The combined organic phases were washed with saturated brine, dried over anhydrous Na₂SO₄, filtered, and concentrated under reduced pressure to obtain the crude product. The crude product was purified by silica gel column chromatography (ethyl acetate/petroleum ether (v/v)=1/100-1/3), monitored by TLC (ethyl acetate/petroleum ether (v/v)=1/3). The fractions with Rf=0.3-0.4 were collected to give compound Cpd-46-2 (541 mg, yield 53.0%) as white solid. ESI[M+H]⁺=390.2.

### 3. Preparation of Compound Cpd-46-3

At room temperature, compound Cpd-46-2 (500 mg, 1.29 mmol) was dissolved in dichloromethane (10 mL), and trifluoroacetic acid (735 mg, 6.45 mmol) was added to the above reaction system. The mixture was stirred at room temperature overnight. The reaction was monitored by TLC until completion, saturated aqueous sodium bicarbonate was added to adjust the pH to 7-8. The mixture was concentrated under reduced pressure to obtain the crude product, which was purified by silica gel column chromatography (methanol/dichloromethane (v/v)=1/100-1/10). Monitored by TLC (methanol/dichloromethane (v/v)=1/10), the fractions with Rf=0.3-0.4 were collected to give compound Cpd-46-3 (364 mg, yield 97.5%) as white solid. ESI[M+H]⁺=290.2.

### 4. Preparation of Compound Cpd-46-4 and Compound Cpd-47-1

At room temperature, Cpd-46-3 (173 mg, 0.6 mmol), Cpd-1-2 (206 mg, 0.5 mmol), Pd₂(dba)₃ (46 mg, 0.05 mmol), t-BuONa (96 mg, 1.0 mmol) and Johnphos (60 mg, 0.2 mmol) were dissolved in 1,4-dioxane (20 mL), the reaction system was purged with nitrogen three times, then stirred at 70° C. overnight under nitrogen protection. The reaction was monitored by TLC until completion, the mixture was concentrated under reduced pressure to obtain the crude product. The crude product was purified by silica gel column chromatography (ethyl acetate/petroleum ether (v/v)=1/100-1/8), monitored by TLC (ethyl acetate/petroleum ether (v/v)=1/8). The fractions with Rf=0.3-0.4 were collected to give compound Cpd-46-4 (103 mg, yield 18.2%) as white solid. ESI[M+H]⁺=566.4 and Cpd-47-1 (37 mg, yield 6.0%), ESI[M+H]⁺=622.4.

### 5. Preparation of Compound Cpd-46-5

At room temperature, TBAF (0.36 mL, 1 mol/L in THF, 0.36 mmol) was added to a solution of Cpd-46-4 (103 mg, 0.18 mmol) in dry tetrahydrofuran (5 mL), and the mixture was stirred at room temperature for 30 minutes. The reaction was monitored by TLC until completion, water (10 mL) was added to the reaction system, and the mixture was extracted with ethyl acetate (3×10 mL). The combined organic phases were dried over anhydrous Na₂SO₄, filtered, and concentrated under reduced pressure to obtain the crude product. The crude product was purified by silica gel column chromatography (ethyl acetate/petroleum ether (v/v)=1/100-1/4), monitored by TLC (ethyl acetate/petroleum ether (v/v)=1/4), and the fractions with Rf=0.3-0.4 were collected to give compound Cpd-46-5 (59 mg, yield 79.7%) as white solid. ESI[M+H]⁺=410.3.

### 6. Preparation of Compound Cpd-46

At room temperature, HCl/Et₂O (0.14 mL, 2 mol/L in Et₂O, 0.28 mmol) was slowly added to a solution of Cpd-46-5 (59 mg, 0.14 mmol) in diethyl ether (5 mL). The mixture was stirred at room temperature for 3 hours, and the crude product was obtained by low-temperature vacuum concentration. The crude product was washed with diethyl ether and dried to give compound Cpd-46 (58.4 mg, yield 86.5%). ESI[M+H]⁺=410.2.

¹H NMR (400 MHz, CD₃OD) δ 7.60-7.26 (m, 7H), 4.04 (s, 2H), 3.63 (d, J=9.2 Hz, 2H), 3.46 (d, J=10.0 Hz, 2H), 3.34 (dd, J=13.7, 6.9 Hz, 2H), 2.98 (dd, J=41.6, 8.9 Hz, 2H), 2.05 (s, 2H), 1.41-1.20 (m, 15H).

### 7. Preparation of Compound Cpd-47

At room temperature, TBAF (0.12 mL, 1 mol/L in THE, 0.12 mmol) was added to a solution of Cpd-47-1 (37 mg, 0.06 mmol) in dry tetrahydrofuran (5 mL), and the mixture was stirred at room temperature for 30 minutes. The reaction was monitored by TLC until completion, water (10 mL) was added to the reaction system, and the mixture was extracted with ethyl acetate (3×10 mL). The combined organic phases were dried over anhydrous Na₂SO₄, filtered, and concentrated under reduced pressure to give the crude product. The crude product was purified by silica gel column chromatography (ethyl acetate/petroleum ether (v/v)=1/100-1/4), monitored by TLC (ethyl acetate/petroleum ether (v/v)=1/4), and the fractions with Rf=0.3-0.4 were collected to give compound Cpd-47 (4.4 mg, yield 15.7%) as white solid. ESI[M+H]⁺=466.3.

¹H NMR (400 MHz, CD₃OD) δ 7.56-7.42 (m, 3H), 7.22 (d, J=7.0 Hz, 2H), 6.61 (s, 2H), 4.32 (d, J=7.2 Hz, 1H), 3.26 (d, J=6.8 Hz, 2H), 3.03-2.38 (m, 6H), 1.32 (d, J=7.2 Hz, 4H), 1.29 (s, 3H), 1.17 (d, J=6.9 Hz, 12H), 1.11-1.00 (m, 3H).

### Example 25 Preparation of Compound Cpd-49

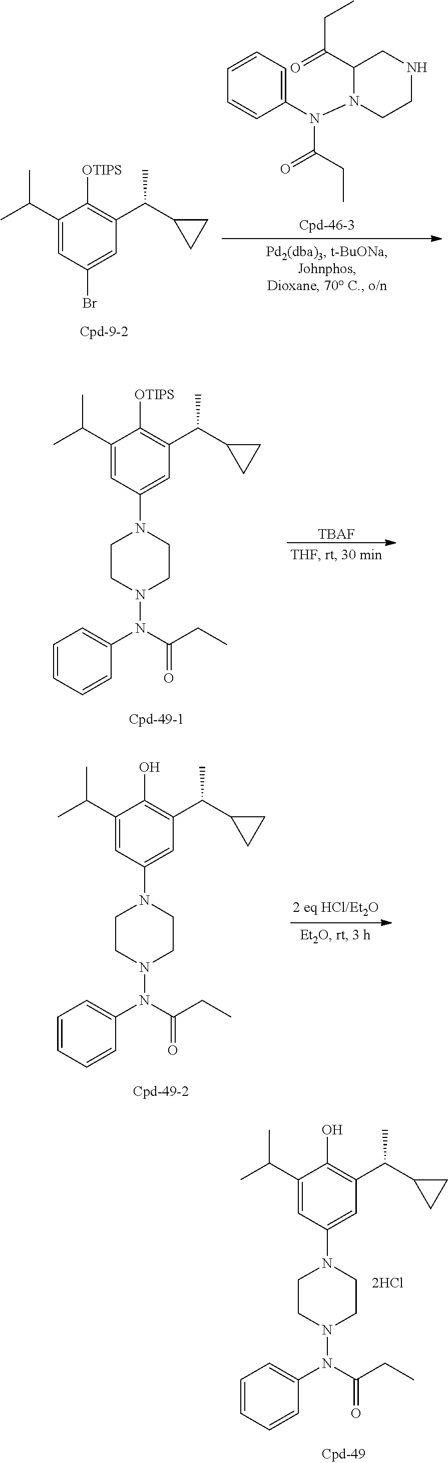

According with the preparation of the Cpd-46, the target compound Cpd-49 was obtained starting from Cpd-46-3 and Cpd-9-2.
Compound Cpd-49: White solid, 28.3 mg, ESI[M+H]⁺=436.3.
¹H NMR (400 MHz, CD₃OD) δ 7.54 (d, J=7.5 Hz, 3H), 7.38 (s, 2H), 7.24 (s, 2H), 3.96 (s, 2H), 3.64 (s, 2H), 3.50-3.38 (m, 2H), 3.33 (t, J=5.4 Hz, 1H), 3.08-2.85 (m, 2H), 2.54-2.41 (m, 1H), 2.05 (s, 1H), 1.86 (s, 1H), 1.25 (dd, J=19.6, 6.9 Hz, 12H), 1.04 (s, 1H), 0.67-0.53 (m, 1H), 0.42-0.30 (m, 1H), 0.28-0.21 (m, 1H), 0.11-0.04 (m, 1H).

### Example 26 Preparation of Compound Cpd-50

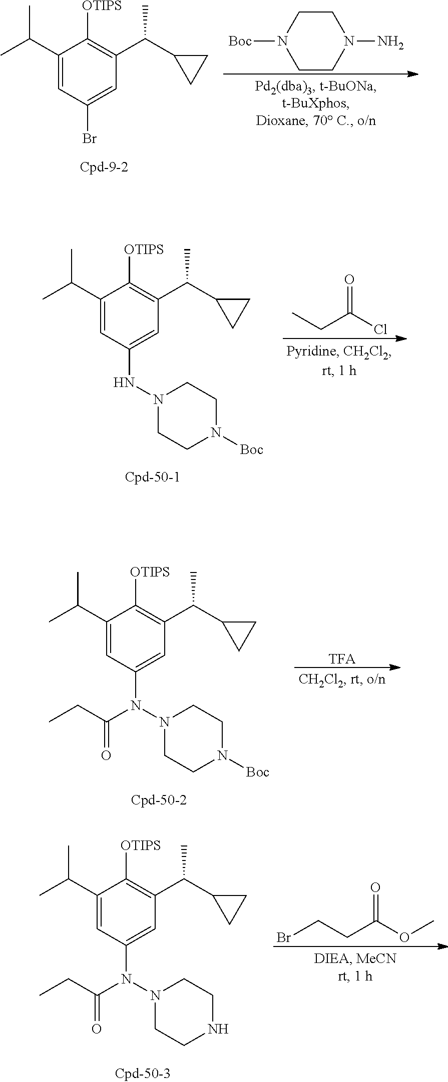

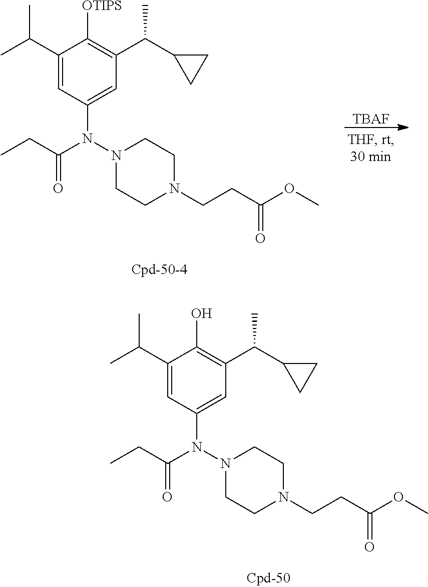

### 1. Preparation of Compound Cpd-50-1

At room temperature, Cpd-9-2 (1.32 g, 3.0 mmol), Cpd-45-0 (1.04 g, 4.5 mmol), Pd₂(dba)₃ (314 mg, 0.3 mmol), t-BuONa (493 mg, 4.5 mmol) and t-BuXphos (291 mg, 0.6 mmol) were dissolved in 1,4-dioxane (5 mL), the reaction system was purged with nitrogen three times, then stirred at 70° C. overnight under nitrogen protection. The reaction was monitored by TLC until completion, the mixture was concentrated under reduced pressure to obtain the crude product. The crude product was purified by silica gel column chromatography (ethyl acetate/petroleum ether (v/v)=1/100-1/1), monitored by TLC (ethyl acetate/petroleum ether (v/v)=1/1). The fractions with Rf=0.3-0.4 were collected to give compound Cpd-50-1 (1.1 g, yield 65.6%) as white solid. ESI[M+H]⁺=560.4.

### 2. Preparation of Compound Cpd-50-2

At room temperature, pyridine (474 mg, 6.0 mmol) and propionyl chloride (495 mg, 5.0 mmol) were added to a solution of Cpd-50-1 (559 mg, 1.0 mmol) in dry dichloromethane (20 mL), and the mixture was stirred at room temperature for 1 hour. The reaction was monitored by TLC until completion, ice (50 mL) was added to the reaction system, and the mixture was extracted with ethyl acetate (3×50 mL). The combined organic phases were washed with saturated brine, dried over anhydrous Na₂SO₄, filtered, and concentrated under reduced pressure to obtain the crude product. The crude product was purified by silica gel column chromatography (ethyl acetate/petroleum ether (v/v)=1/100-1/10), monitored by TLC (ethyl acetate/petroleum ether (v/v)=1/10). The fractions with Rf=0.3-0.4 were collected to give compound Cpd-50-2 (601 mg, yield 97.7%) as white solid. ESI[M+H]⁺=616.4.

### 3. Preparation of Compound Cpd-50-3

At room temperature, compound Cpd-50-2 (601 mg, 0.98 mmol) was dissolved in dichloromethane (15 mL), and trifluoroacetic acid (559 mg, 4.9 mmol) was added to the above reaction system. The mixture was stirred at room temperature overnight. The reaction was monitored by TLC until completion, saturated aqueous sodium bicarbonate was added to adjust the pH to 7-8. The mixture was concentrated under reduced pressure to obtain the crude product, which was purified by silica gel column chromatography (methanol/dichloromethane (v/v)=1/100-1/10). Monitored by TLC (methanol/dichloromethane (v/v)=1/10), the fractions with Rf=0.3-0.4 were collected to give compound Cpd-50-3 (345 mg, yield 68.3%) as white solid. ESI[M+H]⁺=516.4.

### 4. Preparation of Compound Cpd-50-4

At room temperature, DIEA (259 mg, 2.01 mmol) and methyl 2-chloroacetate (223 mg, 1.34 mmol) were added into a solution of Cpd-50-3 (345 mg, 0.67 mmol) in dry acetonitrile (15 mL), then the mixture was stirred at room temperature for 1 hour. The reaction was monitored by TLC until completion, ice-water (20 mL) was added to the reaction system, and extracted with ethyl acetate (3×20 mL), the combined organic layers were dried over anhydrous Na₂SO₄, filtered and concentrated under reduced pressure to obtain a crude produc, the crude product was purified by silica gel column chromatography (ethyl acetate/petroleum ether (v/v)=1/100-1/10), TLC monitoring (ethyl acetate/petroleum ether (v/v)=1/10) was performed, and the fractions with Rf=0.3-0.4 were collected to yield compound Cpd-50-4 (313 mg, yield 77.6%) as white solid. ESI[M+H]⁺=602.4.

### 5. Preparation of Compound Cpd-50

At room temperature, TBAF (1.04 mL, 1 mol/L in THE, 1.04 mmol) was added to a solution of Cpd-50-4 (313 mg, 0.52 mmol) in dry tetrahydrofuran (10 mL), and the mixture was stirred at room temperature for 30 minutes. The reaction was monitored by TLC until completion, water (30 mL) was added to the reaction system, and the mixture was extracted with ethyl acetate (3×30 mL). The combined organic phases were dried over anhydrous Na₂SO₄, filtered, and concentrated under reduced pressure to obtain the crude product. The crude product was purified by silica gel column chromatography (ethyl acetate/petroleum ether (v/v)=1/100-1/4), monitored by TLC (ethyl acetate/petroleum ether (v/v)=1/4), and the fractions with Rf=0.3-0.4 were collected to give compound Cpd-50 (176.4 mg, yield 76.2%) as white solid. ESI[M+H]⁺=446.3.

¹H NMR (400 MHz, CD₃OD) δ 6.79 (dd, J=54.5, 2.4 Hz, 2H), 3.62 (s, 3H), 3.40-3.34 (m, 1H), 2.97 (d, J=10.2 Hz, 2H), 2.87 (d, J=10.7 Hz, 2H), 2.75 (d, J=7.5 Hz, 2H), 2.65 (t, J=7.3 Hz, 2H), 2.57 (s, 2H), 2.48 (d, J=7.2 Hz, 3H), 2.36 (dd, J=11.5, 9.0 Hz, 2H), 1.39-1.07 (m, 12H), 1.05-0.98 (m, 2H), 0.64-0.46 (m, 1H), 0.38-032 (m, 1H), 0.19-0.16 (m, 1H), 0.10-0.04 (m, 1H).

### Example 27 Preparation of Compound Cpd-51

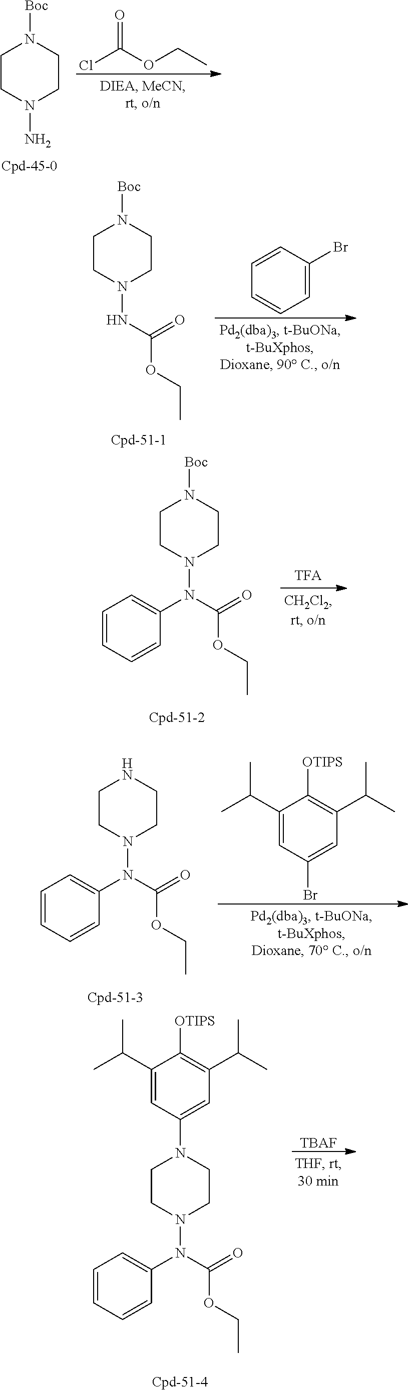

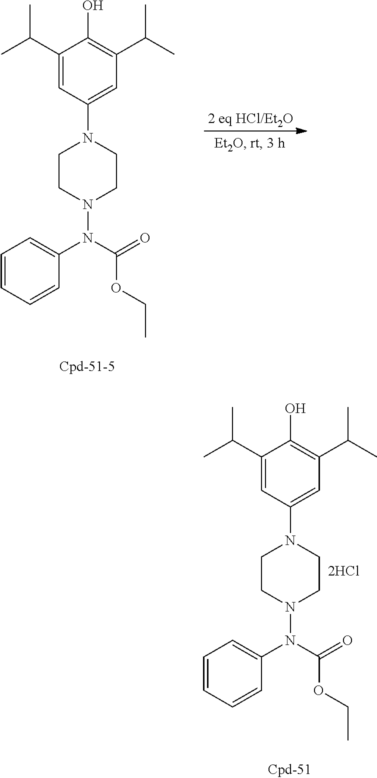 [SMILES: CCOC(=O)N(c1ccccc1)N1CCN(c2cc(C(C)C)c(O)c(C(C)C)c2)CC1 ; "2 eq HCl/Et2O" ; CCOC(=O)N(c1ccccc1)N1CCN(c2cc(C(C)C)c(O)c(C(C)C)c2)CC1]

### 1. Preparation of Compound Cpd-51-1

At room temperature, DIEA (9.62 g, 74.6 mmol) and ethyl carbonochloridate (4.02 g, 37.3 mmol) were added into a solution of Cpd-45-0 (5.0 g, 24.8 mmol) in dry acetonitrile (25 mL), then the mixture was stirred at room temperature for overnight. The reaction was monitored by TLC until completion, ice-water (50 mL) was added to the reaction system, and extracted with ethyl acetate (3×50 mL), the combined organic layers were dried over anhydrous Na₂SO₄, filtered and concentrated under reduced pressure to obtain a crude produc, the crude product was purified by silica gel column chromatography (ethyl acetate/petroleum ether (v/v)=1/100-1/1), TLC monitoring (ethyl acetate/petroleum ether (v/v)=1/1) was performed, and the fractions with Rf=0.4-0.5 were collected to yield compound Cpd-51-1 (4.88 g, yield 72.1%) as white solid. ESI[M+H]⁺=274.2.

### 2. Preparation of Compound Cpd-51-2

At room temperature, Cpd-32-0 (1.72 g, 10.84 mmol), Cpd-51-1 (1.48 g, 5.42 mmol), Pd₂(dba)₃ (496 mg, 0.542 mmol), t-BuONa (1.05 g, 10.84 mmol) and t-BuXphos (461 mg, 1.084 mmol) were dissolved in 1,4-dioxane (30 mL), the reaction system was purged with nitrogen three times, then stirred at 90° C. overnight under nitrogen protection. The reaction was monitored by TLC until completion, the mixture was concentrated under reduced pressure to obtain the crude product. The crude product was purified by silica gel column chromatography (ethyl acetate/petroleum ether (v/v)=1/100-1/1), monitored by TLC (ethyl acetate/petroleum ether (v/v)=1/1). The fractions with Rf=0.3-0.4 were collected to give compound Cpd-51-2 (1.69 g, yield 89.4%) as white solid. ESI[M+H]⁺=350.2.

### 3. Preparation of Compound Cpd-51-3

At room temperature, compound Cpd-51-2 (1.69 g, 4.84 mmol) was dissolved in dichloromethane (10 mL), and trifluoroacetic acid (2.76 g, 24.2 mmol) was added to the above reaction system. The mixture was stirred at room temperature overnight. The reaction was monitored by TLC until completion, saturated aqueous sodium bicarbonate was added to adjust the pH to 7-8. The mixture was concentrated under reduced pressure to obtain the crude product, which was purified by silica gel column chromatography (methanol/dichloromethane (v/v)=1/100-1/10) Monitored by TLC (methanol/dichloromethane (v/v)=1/10), the fractions with Rf=0.3-0.4 were collected to give compound Cpd-51-3 (1.01 g, yield 83.4%) as white solid. ESI[M+H]⁺=250.1.

### 4. Preparation of Compound Cpd-51-4

At room temperature, Cpd-51-3 (349 mg, 1.4 mmol), Cpd-1-2 (100 mg, 0.7 mmol), Pd₂(dba)₃ (64 mg, 0.07 mmol), t-BuONa (135 mg, 1.4 mmol) and t-BuXphos (60 mg, 0.14 mmol) were dissolved in 1,4-dioxane (15 mL), the reaction system was purged with nitrogen three times, then stirred at 70° C. overnight under nitrogen protection. The reaction was monitored by TLC until completion, the mixture was concentrated under reduced pressure to obtain the crude product. The crude product was purified by silica gel column chromatography (ethyl acetate/petroleum ether (v/v)=1/100-1/8), monitored by TLC (ethyl acetate/petroleum ether (v/v)=1/8). The fractions with Rf=0.3-0.4 were collected to give compound Cpd-51-4 (394 mg, yield 96.8%) as white solid. ESI[M+H]⁺=582.4.

### 5. Preparation of Compound Cpd-51-5

At room temperature, TBAF (1.36 mL, 1 mol/L in THE, 1.36 mmol) was added to a solution of Cpd-51-4 (394 mg, 0.68 mmol) in dry tetrahydrofuran (20 mL), and the mixture was stirred at room temperature for 30 minutes. The reaction was monitored by TLC until completion, water (30 mL) was added to the reaction system, and the mixture was extracted with ethyl acetate (3×30 mL). The combined organic phases were dried over anhydrous Na₂SO₄, filtered, and concentrated under reduced pressure to obtain the crude product. The crude product was purified by silica gel column chromatography (ethyl acetate/petroleum ether (v/v)=1/100-1/4), monitored by TLC (ethyl acetate/petroleum ether (v/v)=1/4), and the fractions with Rf=0.3-0.4 were collected to give compound Cpd-51-5 (210 mg, yield 72.7%) as white solid. ESI[M+H]⁺=426.3.

### 6. Preparation of Compound Cpd-51

At room temperature, HCl/Et₂O (0.49 mL, 2 mol/L in Et₂O, 0.92 mmol) was slowly added to a solution of Cpd-51-5 (210 mg, 0.49 mmol) in diethyl ether (5 mL). The mixture was stirred at room temperature for 3 hours, and the crude product was obtained by low-temperature vacuum concentration. The crude product was washed with diethyl ether and dried to give compound Cpd-51 (205.7 mg, yield 84.3%). ESI[M+H]⁺=426.3.

¹H NMR (400 MHz, CD₃OD) δ 7.42 (d, J=7.2 Hz, 2H), 7.34 (d, J=7.7 Hz, 3H), 7.18 (s, 2H), 4.20 (d, J=7.1 Hz, 2H), 3.77-3.41 (m, 8H), 3.33 (d, J=6.9 Hz, 2H), 1.22 (d, J=6.9 Hz, 15H).

### Example 28 Preparation of Compound Cpd-52

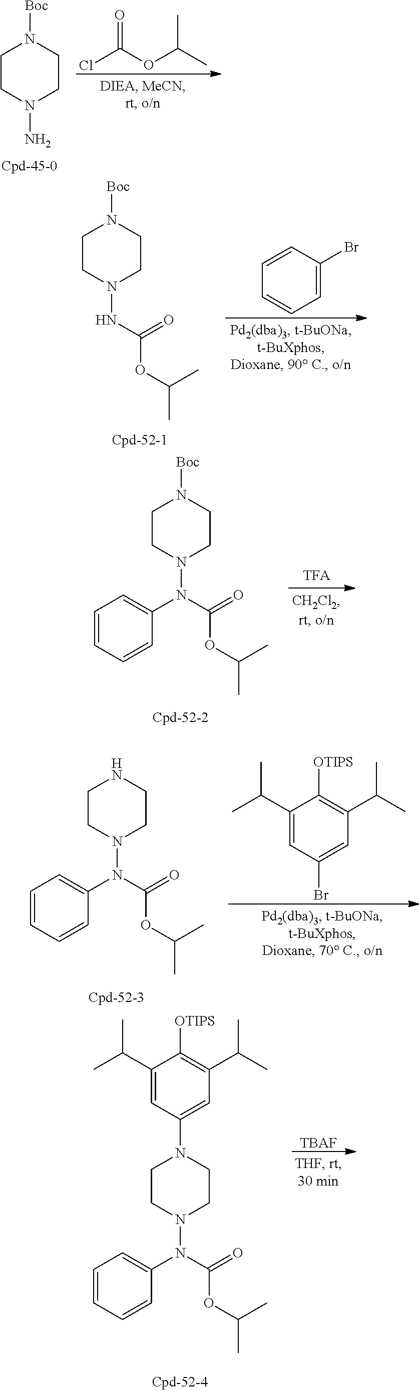

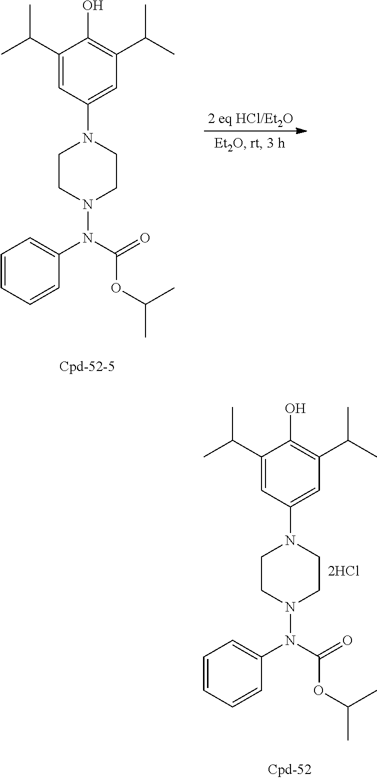 [SMILES: CC(C)OC(=O)N(c1ccccc1)N1CCN(c2cc(C(C)C)c(O)c(C(C)C)c2)CC1 ; "2 eq HCl/Et2O" ; CC(C)OC(=O)N(c1ccccc1)N1CCN(c2cc(C(C)C)c(O)c(C(C)C)c2)CC1]

According with the preparation of the Cpd-51, the target compound Cpd-52 was obtained starting from Cpd-45-0.

Compound Cpd-52: White solid, 182.8 mg, ESI[M+H]⁺=440.3.
¹H NMR (400 MHz, CD₃OD) δ 7.41 (d, J=7.4 Hz, 2H), 7.34 (d, J=7.5 Hz, 3H), 7.26 (s, 2H), 5.00 (dt, J=12.5, 6.3 Hz, 1H), 3.60 (d, J=98.5 Hz, 7H), 3.38-3.31 (m, 2H), 1.23 (dd, J=6.5, 3.0 Hz, 18H).

### Example 29 Preparation of Compound Cpd-53 and Cpd-54

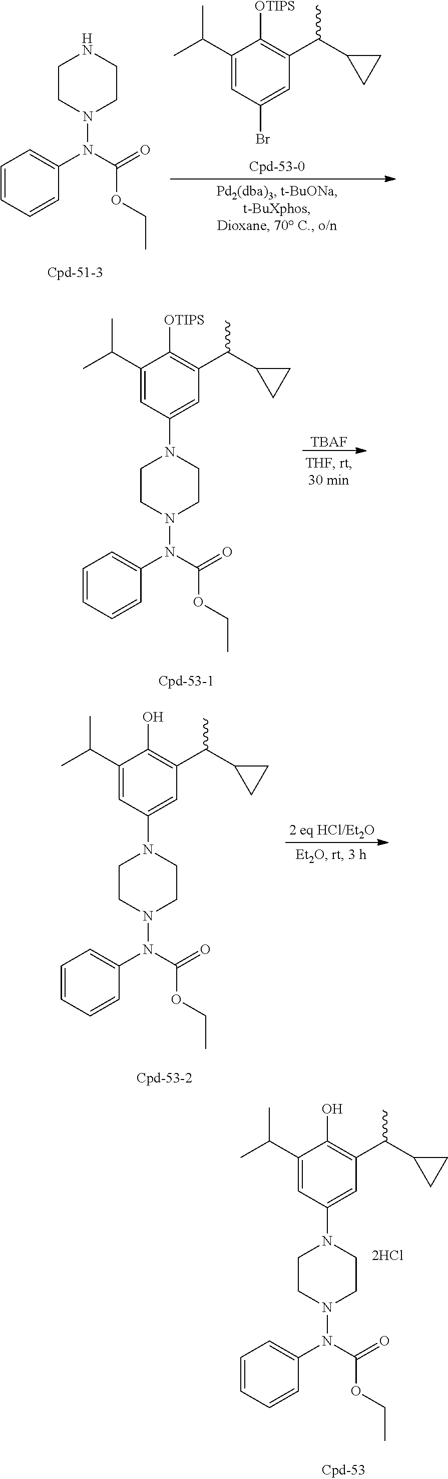

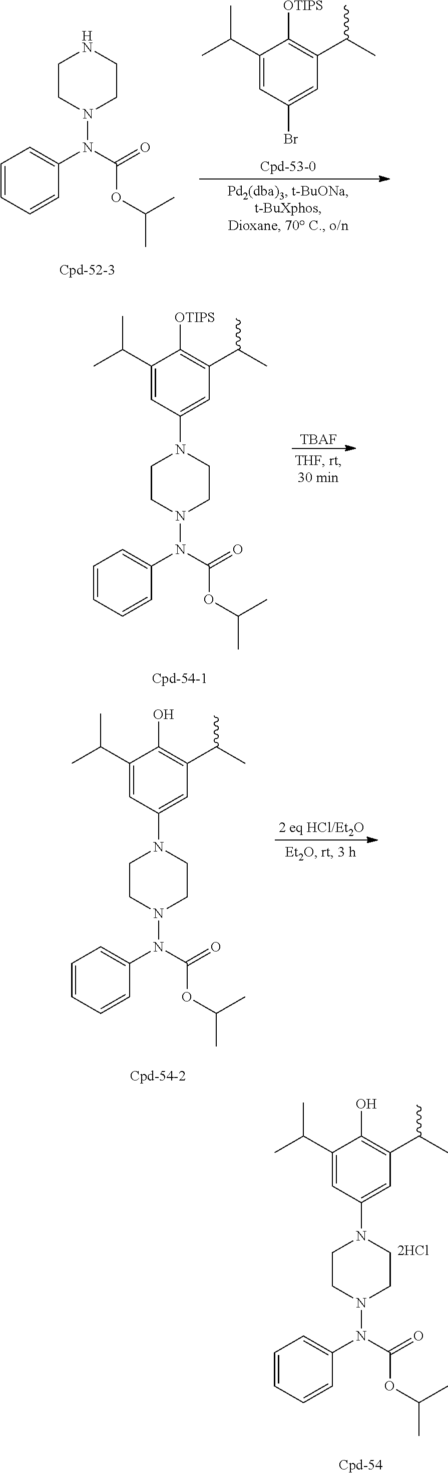

According with the preparation of the Cpd-51, the target compound Cpd-53 and Cpd-54 were obtained starting from Cpd-51-3 and Cpd-53-0.
Compound Cpd-53: white solid, 65.3 mg, ESI[M+H]⁺=452.3.
¹H NMR (400 MHz, CD₃OD) δ 7.45-7.21 (m, 7H), 4.20 (d, J=7.1 Hz, 2H), 3.97-3.41 (m, 8H), 3.37-3.31 (m, 1H), 2.51-2.40 (m, 1H), 1.27 (d, J=6.9 Hz, 3H), 1.24-1.20 (m, 9H), 1.11-1.03 (m, 1H), 0.70-0.60 (m, 1H), 0.44-0.34 (m, 1H), 0.28-0.22 (m, 1H), 0.08-0.02 (m, 1H). compound Cpd-54: white solid, 65.9 mg, ESI[M+H]⁺=466.3.
¹H NMR (400 MHz, CD₃OD) δ 7.45-7.21 (m, 7H), 4.20 (d, J=7.1 Hz, 2H), 3.97-3.41 (m, 8H), 3.37-3.31 (m, 1H), 2.51-2.40 (m, 1H), 1.27 (d, J=6.9 Hz, 3H), 1.24-1.20 (m, 9H), 1.11-1.03 (m, 1H), 0.70-0.60 (m, 1H), 0.44-0.34 (m, 1H), 0.28-0.22 (m, 1H), 0.08-0.02 (m, 1H).

### Example 30 Preparation of Compound Cpd-59

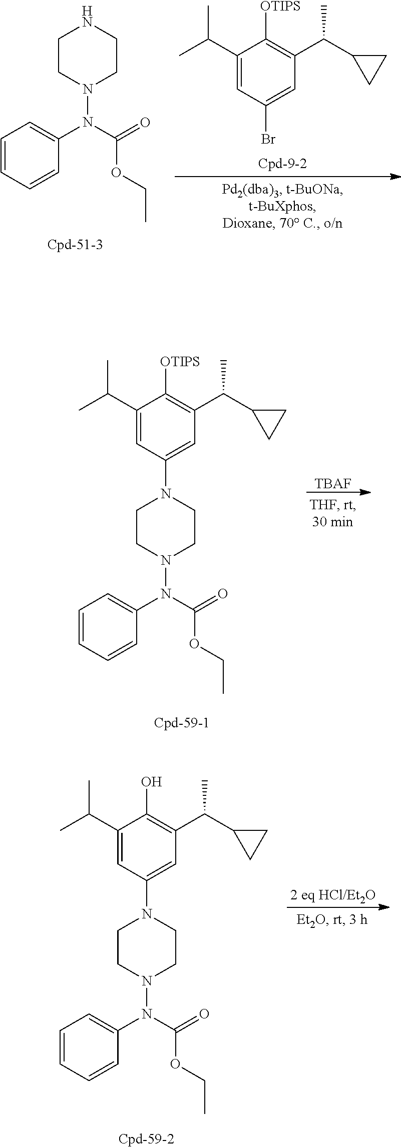

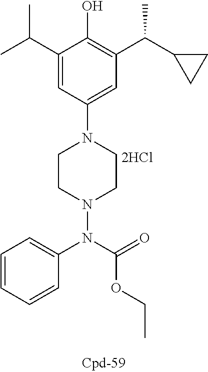 [SMILES: CCOC(=O)N(c1ccccc1)N1CCN(c2cc(C(C)C)c(O)c([C@H](C)C3CC3)c2)CC1]

According with the preparation of the Cpd-51, the target compound Cpd-59 was obtained starting from Cpd-51-3 and Cpd-9-2.
Compound Cpd-59: white solid, 178.1 mg, ESI[M+H]⁺=452.3.
¹H NMR (400 MHz, CD₃OD) δ 7.53-7.34 (m, 6H), 7.31 (d, J=2.7 Hz, 1H), 4.25 (q, J=7.1 Hz, 2H), 3.52 (d, J=7.0 Hz, 8H), 3.38 (s, 1H), 2.53 (dd, J=9.0, 7.0 Hz, 1H), 1.32 (d, J=6.9 Hz, 3H), 1.28 (t, J=6.0 Hz, 9H), 1.16-1.02 (m, 1H), 0.64 (s, 1H), 0.44 (s, 1H), 0.35-0.21 (m, 1H), 0.18-0.07 (m, 1H).

### Example 31 Preparation of Compound Cpd-60 to Cpd-67

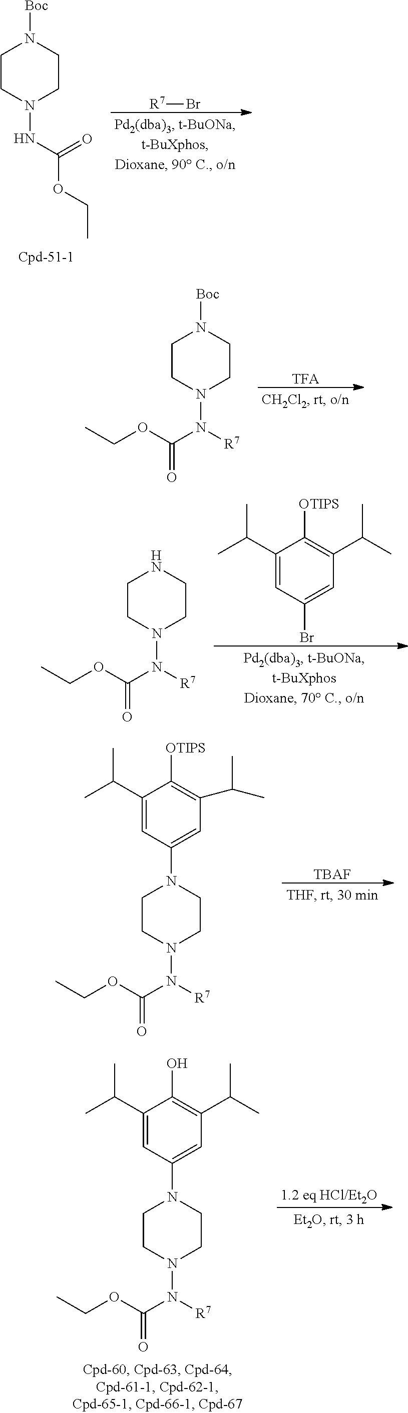

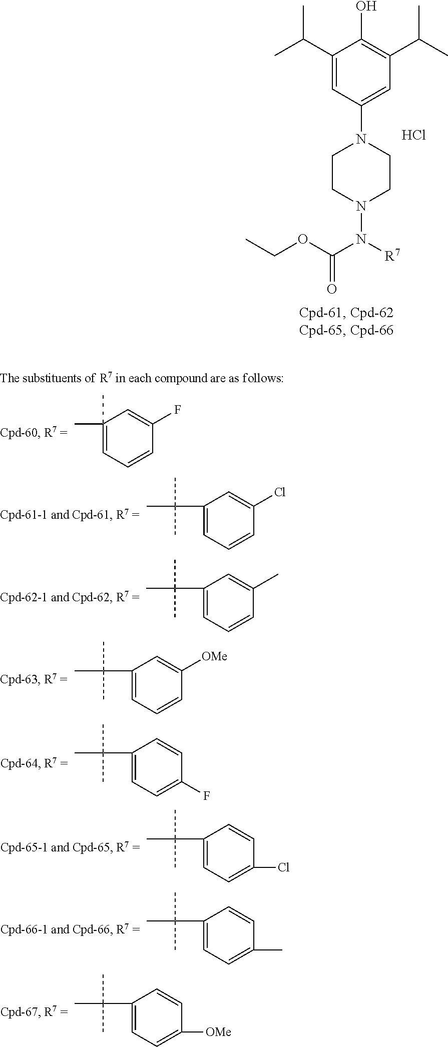

According with the preparation of the Cpd-51, the target compound Cpd-60 to Cpd-67 were obtained starting from Cpd-51-1.
Compound Cpd-60: white solid, 137.4 mg, ESI[M+H]⁺=444.3.
¹H NMR (400 MHz, CD₃OD) δ 7.45 (d, J=6.6 Hz, 1H), 7.35 (s, 2H), 7.32-7.25 (m, 2H), 7.09 (s, 1H), 4.30 (q, J=7.1 Hz, 2H), 4.23-3.45 (m, 8H), 3.39 (dd, J=13.7, 6.8 Hz, 2H), 1.32 (t, J=7.1 Hz, 3H), 1.28 (d, J=6.9 Hz, 13H).
Compound Cpd-61: white solid, 117.6 mg, ESI[M+H]⁺=460.2.
¹H NMR (400 MHz, CD₃OD) δ 7.52 (s, 1H), 7.46-7.30 (m, 5H), 4.29 (q, J=7.1 Hz, 2H), 3.52 (d, J=7.0 Hz, 8H), 3.44-3.36 (m, 2H), 1.32 (t, J=7.1 Hz, 3H), 1.27 (d, J=6.9 Hz, 13H).
Compound Cpd-62: white solid, 119.1 mg, ESI[M+H]⁺=440.3.
¹H NMR (400 MHz, CD₃OD) δ 7.34 (d, J=7.8 Hz, 1H), 7.32 (s, 2H), 7.19 (dd, J=21.8, 8.5 Hz, 3H), 4.24 (q, J=7.1 Hz, 2H), 3.82-3.48 (m, 8H), 3.39 (dd, J=13.7, 6.9 Hz, 2H), 2.42 (s, 3H), 1.32-1.22 (m, 15H).
Compound Cpd-63: white solid, 97.3 mg, ESI[M+H]⁺=456.4.
¹H NMR (400 MHz, CD₃OD) δ 7.37 (d, J=7.7 Hz, 1H), 7.32 (s, 2H), 6.97 (d, J=7.8 Hz, 3H), 4.25 (q, J=7.1 Hz, 2H), 4.20-3.43 (m, 11H), 3.39 (dd, J=13.7, 6.9 Hz, 2H), 1.27 (d, J=6.9 Hz, 15H).
Compound Cpd-64: white solid, 75.6 mg, ESI[M+H]⁺=444.3.
¹H NMR (400 MHz, CD₃OD) δ 7.35 (d, J=5.0 Hz, 2H), 7.18 (d, J=8.6 Hz, 2H), 6.70 (s, 2H), 4.21 (d, J=7.1 Hz, 2H), 3.31 (d, J=7.0 Hz, 2H), 3.26-3.08 (m, 8H), 1.27 (t, J=7.1 Hz, 3H), 1.22 (d, J=6.9 Hz, 12H).
Compound Cpd-65: white solid, 90.3 mg, ESI[M+H]⁺=460.3.
¹H NMR (400 MHz, CD₃OD) δ 7.43 (t, J=5.8 Hz, 4H), 7.33 (s, 2H), 4.27 (q, J=7.0 Hz, 2H), 3.52 (d, J=6.8 Hz, 8H), 3.39 (dd, J=13.7, 6.9 Hz, 2H), 1.29 (dd, J=14.8, 7.0 Hz, 15H).
Compound Cpd-66: white solid, 133.2 mg, ESI[M+H]⁺=440.3.
¹H NMR (400 MHz, CD₃OD) δ 7.27 (dd, J=19.0, 7.3 Hz, 6H), 4.22 (d, J=7.1 Hz, OH), 3.52 (s, 1H), 3.38 (d, J=6.8 Hz, OH), 2.41 (s, OH), 1.26 (t, J=8.2 Hz, 3H).
Compound Cpd-67: white solid, 84.9 mg, ESI[M+H]⁺=456.4.
¹H NMR (400 MHz, MeOD) δ 7.94 (s, 1H), 7.21 (d, J=8.8 Hz, 2H), 6.99 (d, J=8.9 Hz, 2H), 6.69 (s, 2H), 4.17 (d, J=7.2 Hz, 2H), 3.86 (s, 3H), 3.31 (d, J=6.9 Hz, 2H), 3.16 (d, J=4.4 Hz, 4H), 3.13-3.01 (m, 4H), 1.27 (t, J=7.1 Hz, 3H), 1.23 (d, J=6.9 Hz, 12H).

### Example 32 Preparation of Compound Cpd-68 to Cpd-71 and Cpd-73 to Cpd-75

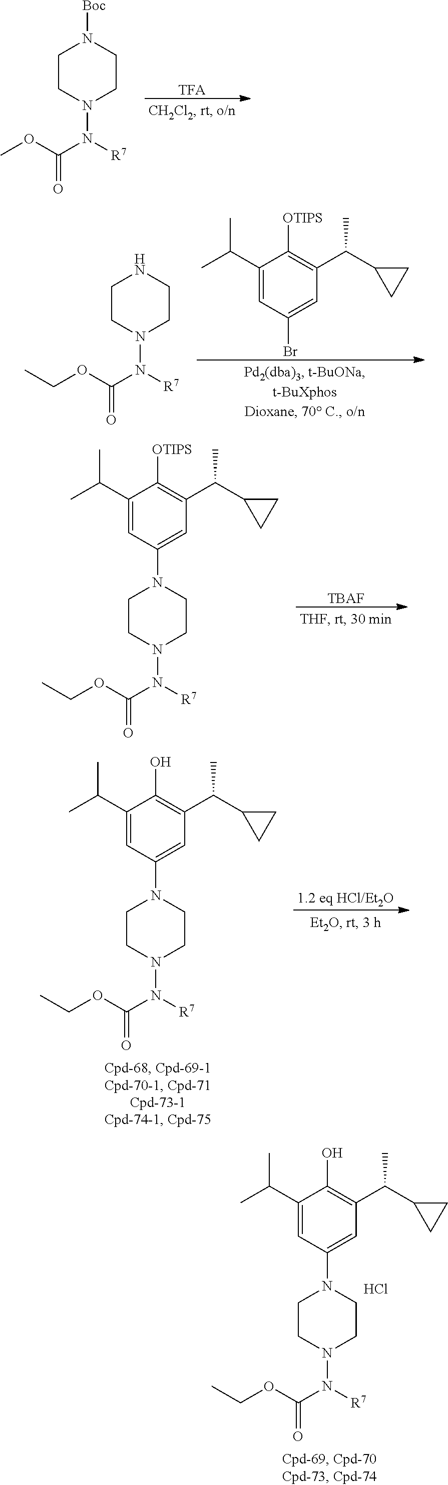

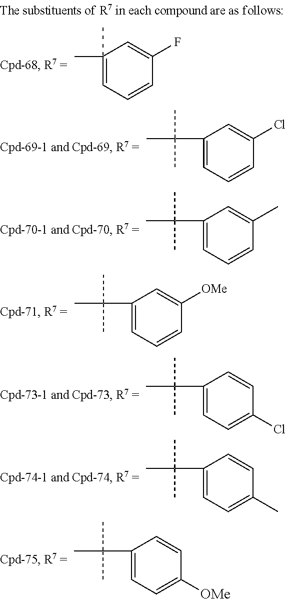

According with the preparation of the Cpd-51, the target compound Cpd-68 to Cpd-71, Cpd-73 to Cpd-75 were obtained starting from Cpd-68-1.
Compound Cpd-68: white solid, 64.6 mg, ESI[M+H]⁺=470.4.
¹H NMR (400 MHz, CD₃OD) δ 7.52-7.40 (m, 2H), 7.39-7.23 (m, 3H), 7.08 (s, 1H), 4.30 (q, J=7.1 Hz, 2H), 4.23-3.41 (m, 8H), 3.37 (d, J=6.9 Hz, 1H), 2.60-2.46 (m, 1H), 1.32 (d, J=7.0, 3.6 Hz, 6H), 1.28 (d, J=6.8 Hz, 6H), 1.10-0.90 (m, 1H), 0.64-0.60 (m, 1H), 0.44-0.39 (m, 1H), 0.33-0.22 (m, 1H), 0.12-0.04 (m, 1H).
Compound Cpd-69: white solid, 121.8 mg, ESI[M+H]⁺=486.3.
¹H NMR (400 MHz, CD₃OD) δ 7.52 (s, 1H), 7.48-7.29 (m, 5H), 4.29 (d, J=7.1 Hz, 2H), 4.22-3.41 (m, 8H), 3.36 (s, 1H), 2.53-2.40 (m, 1H), 1.32 (d, J=6.6 Hz, 5H), 1.27 (d, J=6.8 Hz, 7H), 1.12-1.08 (m, 1H), 0.71-0.58 (m, 1H), 0.49-0.38 (m, 1H), 0.28-0.20 (m, 1H), 0.12-0.09 (m, 1H).
Compound Cpd-70: white solid, 156.4 mg, ESI[M+H]⁺=466.4.
¹H NMR (400 MHz, CD₃OD) δ 7.45 (d, J=2.8 Hz, 1H), 7.33 (d, J=13.1, 5.2 Hz, 2H), 7.19 (d, J=22.0, 8.7 Hz, 3H), 4.24 (d, J=7.1 Hz, 2H), 3.52-3.40 (m, 8H), 3.37 (s, 1H), 2.60-2.47 (m, 1H), 2.42-2.36 (m, 3H), 1.32 (d, J=6.9 Hz, 3H), 1.26 (d, J=5.7 Hz, 9H), 1.10-1.02 (m, 1H), 0.71-0.57 (m, 1H), 0.50-0.36 (m, 1H), 0.28-0.22 (m, 1H), 0.12-0.04 (m, 1H).
Compound Cpd-71: white solid, 135.1 mg, ESI[M+H]⁺=482.4.
¹H NMR (400 MHz, CD₃OD) δ 7.47 (d, J=2.7 Hz, 1H), 7.42-7.28 (m, 2H), 6.97 (d, J=7.8 Hz, 3H), 4.25 (q, J=7.1 Hz, 2H), 4.16-3.42 (m, 12H), 3.38-3.34 (m, 1H), 2.53-2.51 (m, 1H), 1.35-1.29 (m, 4H), 1.27 (d, J=7.0 Hz, 8H), 1.16-1.02 (m, 1H), 0.71-0.57 (m, 1H), 0.50-0.38 (m, 1H), 0.28-0.26 (m, 1H), 0.12-0.04 (m, 1H).
Compound Cpd-73: white solid, 152.9 mg, ESI[M+H]⁺=486.3.
¹H NMR (400 MHz, CD₃OD) δ 7.44 (d, J=3.1 Hz, 5H), 7.31 (d, J=2.6 Hz, 1H), 4.27 (d, J=7.1 Hz, 2H), 3.59-3.48 (m, 8H), 3.36-3.33 (m, 1H), 2.53 (dq, J=14.3, 7.1 Hz, 1H), 1.31 (dd, J=6.9, 4.6 Hz, 5H), 1.27 (d, J=7.0 Hz, 7H), 1.16-1.04 (m, 1H), 0.69-0.58 (m, 1H), 0.44-0.41 (m, 1H), 0.28-0.19 (m, 1H), 0.12-0.08 (m, 1H).
Compound Cpd-74: white solid, 140.3 mg, ESI[M+H]⁺=466.4.
¹H NMR (400 MHz, CD₃OD) δ 7.44 (d, J=2.8 Hz, 1H), 7.33-7.27 (m, 3H), 7.24 (d, J=8.4 Hz, 2H), 4.22 (d, J=7.1 Hz, 2H), 4.15-3.43 (m, 8H), 3.40-3.36 (m, 1H), 2.53 (dq, J=14.4, 7.3 Hz, 1H), 2.41 (s, 3H), 1.31 (d, J=6.9 Hz, 3H), 1.26 (t, J=8.4 Hz, 9H), 1.09 (td, J=8.3, 3.7 Hz, 1H), 0.70-0.58 (m, 1H), 0.48-0.38 (m, 1H), 0.34-0.22 (m, 1H), 0.18-0.06 (m, 1H).
Compound Cpd-75: white solid, 104 mg, ESI[M+H]⁺=481.2.
¹H NMR (400 MHz, MeOD) δ 7.21 (d, J=8.9 Hz, 2H), 6.99 (d, J=8.8 Hz, 2H), 6.81 (d, J=2.8 Hz, 1H), 6.70 (d, J=2.9 Hz, 1H), 4.17 (q, J=7.1 Hz, 2H), 3.85 (s, 3H), 3.33-3.24 (m, 1H), 3.22-3.15 (m, 4H), 3.14-3.03 (m, 4H), 2.56-2.38 (m, 1H), 1.27 (d, J=7.0 Hz, 3H), 1.24 (d, J=7.7 Hz, 3H), 1.22 (dd, J=6.8, 1.0 Hz, 6H), 1.08-0.99 (m, 1H), 0.64-0.50 (m, 1H), 0.44-0.32 (m, 1H), 0.25-0.15 (m, 1H), 0.16-0.07 (m, 1H).

### Example 33 Preparation of Compound Cpd-76 to Cpd-79, Cpd-78-1 to Cpd-79-1

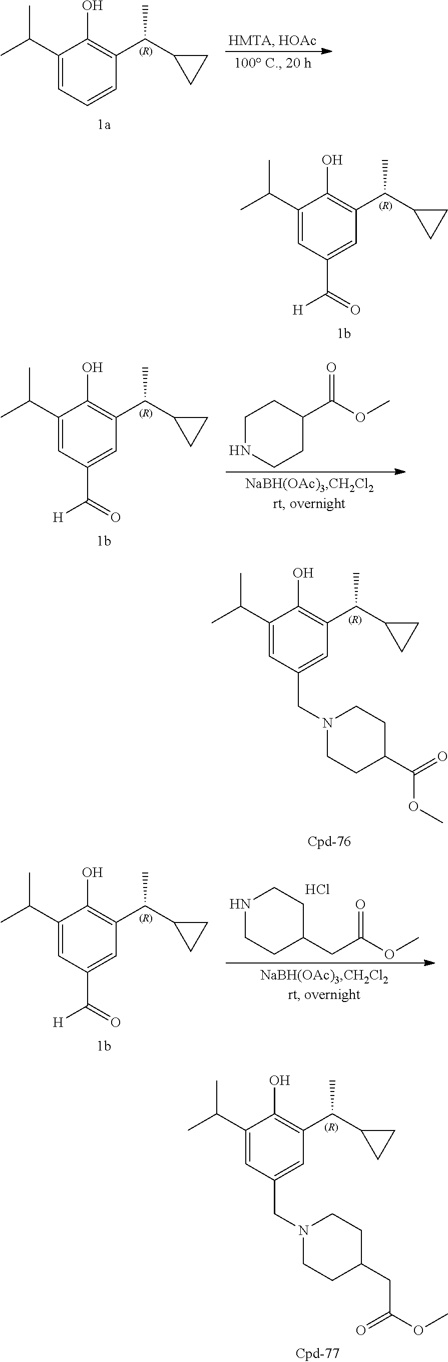

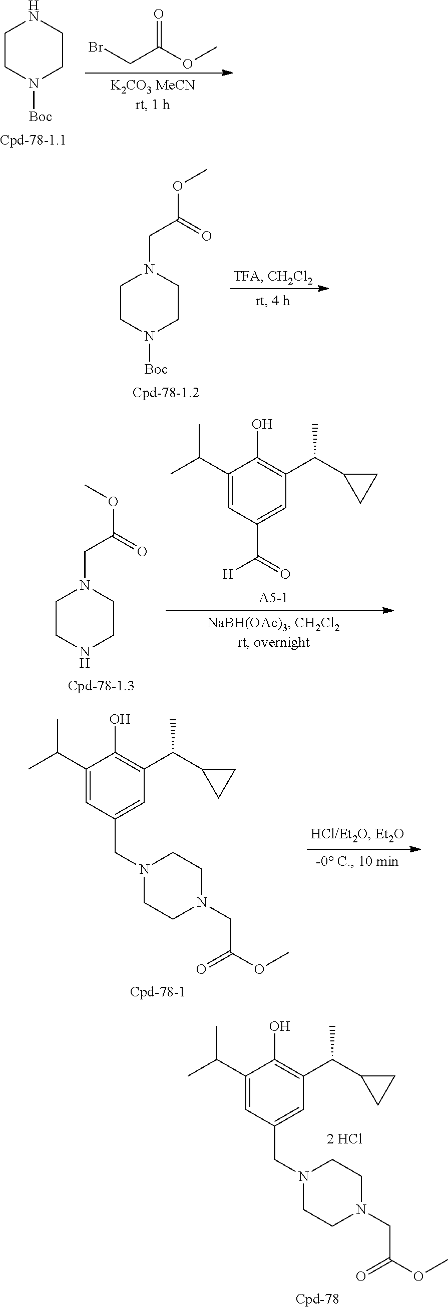

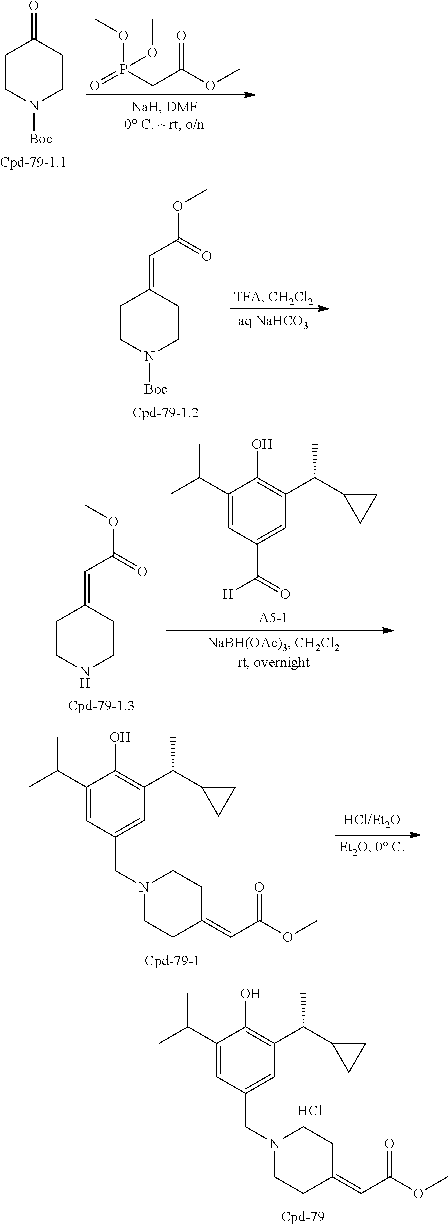

### 1. Preparation of Intermediate Compounds

### Preparation of Intermediate Compound 1b

At room temperature, 1a (8 g, 39.16 mmol) was dissolved in acetic acid (80 mL), and urotropine (3.92 g, 313.29 mmol) was added in batches. After the addition, the mixture was stirred at 100° C. for 20 hours. Water (500 mL) was added to the reaction solution, which was extracted with ethyl acetate (3×80 mL). The combined organic phases were dried over anhydrous sodium sulfate, filtered, and the filtrate was concentrated under reduced pressure to obtain the crude product. The crude product was purified by silica gel column chromatography (petroleum ether/ethyl acetate (V/V)=20/1-10/1), monitored by TLC (petroleum other/ethyl acetate (V/V)=10/1). The fractions with Rf=0.4-0.5 were collected to give the compound 1b (2.1 g, yield 41.0%) as light yellow. ESI[M+H]⁺=233.2.

### 3.5 g of the Starting Material 1a was Recovered.

### Preparation of Intermediate Compound Cpd-78-1.3

At room temperature, Cpd-78-1.1 (3.72 g, 20.0 mmol), methyl bromoacetate (3.37 g, 22.03 mmol) and potassium carbonate (4.15 g, 30.03 mmol) were dissolved in acetonitrile (20 mL), and the mixture was stirred at room temperature for 1 hour. The mixture was filtered and concentrated to obtain the crude product, which was purified by silica gel column chromatography [petroleum ether/ethyl acetate (V/V)=5/1-1/1]. Monitored by TLC (ethyl acetate/petroleum ether (v/v)=1/3), the fractions with Rf=0.5-0.6 were collected to give the colorless oily compound Cpd-78-1.2 (4.1 g, yield 79.4%). Compound Cpd-78-1.2 was deprotected with trifluoroacetic acid to obtain the crude compound Cpd-78-1.3, which was directly used for the preparation of compound Cpd-78 without purification.

### Preparation of Intermediate Compound Cpd-79-1.3

At 0° C., trimethyl phosphonoacetate (1.2 g, 6.59 mmol) was slowly added via syringe to a suspension of NaH (265.0 mg, 60%, 6.62 mmol) in dry DMF (10 mL). After stirring for 30 minutes, a solution of Cpd-79-1.1 (1.0 g, 5.02 mmol) in DMF (1 mL) was added. The reaction mixture was allowed to warm to room temperature and stirred overnight. The reaction was monitored by TLC until completion, the reaction was quenched with H₂O (20 mL) and extracted with ethyl acetate (3×10 mL). The combined organic layers were washed with brine, dried over anhydrous Na₂SO₄, filtered, and concentrated under reduced pressure. The crude product was purified by silica gel column chromatography (petroleum ether/ethyl acetate (V/V)=10/1-1/1), monitored by TLC (ethyl acetate/petroleum ether (v/v)=1/3). The fractions with Rf=0.5-0.6 were collected to afford compound Cpd-79-1.2 (876 mg, yield 68.3%) as white solid. Deprotection of Cpd-79-1.2 with trifluoroacetic acid provided the crude compound Cpd-79-1.3, which was used directly in the preparation of compound Cpd-79 without further purification.

### 2. Preparation of Compound Cpd-78-1 to Cpd-79-1, Cpd-76 to Cpd-79

### General Procedure:

Compound 1b (1.0 eq) and the corresponding amine (prepared in the first step or commercially available, 1.0-3.0 eq) were dissolved in dichloromethane and stirred at room temperature for 1 hour. Sodium triacetoxyborohydride (3.0 eq) was then added, and the mixture was stirred overnight at room temperature. The reaction was monitored by TLC until completion, the reaction was quenched with H₂O (20 mL) and extracted with ethyl acetate (3×10 mL). The combined organic layers were washed with brine, dried over anhydrous Na₂SO₄, filtered, and concentrated under reduced pressure. The crude product was purified by silica gel column chromatography or preparative TLC to give the target compounds Cpd-76, Cpd-77, Cpd-78-1, and Cpd-79-1.

### Preparation of Hydrochloride:

The free base was dissolved in diethyl ether, cooled to 0° C. in an ice-water bath, and a solution of HCl in EtO was slowly added via syringe. The mixture was stirred for 10 minutes, filtered, washed with diethyl ether, and dried to afford the target Cpd-78 hydrochloride and Cpd-79 hydrochloride.

Compound Cpd-76: colorless oil, 174 mg, ESI[M+H]⁺=360.3.
¹H NMR (400 MHz, CDCl₃) δ 7.05 (s, 1H), 7.00 (s, 1H), 4.92 (s, 1H), 3.68 (s, 3H), 3.58-3.33 (m, 2H), 3.21-3.07 (m, 1H), 3.00-2.77 (m, 2H), 2.53-2.42 (m, 1H), 2.37-2.18 (m, 1H), 2.04-1.72 (m, 5H), 1.30 (d, J=7.0 Hz, 3H), 1.28-1.23 (m, 8H), 1.10-1.01 (m, 1H), 0.61-0.52 (m, 1H), 0.50-0.41 (m, 1H), 0.26-0.19 (m, 1H), 0.18-0.11 (m, 1H).
Compound Cpd-77: colorless oil, 68 mg, ESI[M+H]⁺=374.0.
¹H NMR (400 MHz, CDCl₃) δ 7.02 (s, 2H), 4.94 (s, 1H), 3.68 (s, 3H), 3.60-3.33 (m, 2H), 3.21-3.11 (m, 1H), 3.06-2.76 (m, 1H), 2.56-2.45 (m, 1H), 2.38-2.23 (m, 2H), 2.12-1.53 (m, 9H), 1.32 (d, J=7.0 Hz, 3H), 1.29 (d, J=7.0 Hz, 6H), 1.14-1.03 (m, 1H), 0.64-0.55 (m, 1H), 0.53-0.45 (m, 1H), 0.29-0.21 (m, 1H), 0.21-0.13 (m, 1H).
Compound Cpd-78-1: colorless oil, 27.6 mg, ESI[M+H]⁺=375.1.
¹H NMR (400 MHz, CDCl₃) δ 7.09 (s, 2H), 5.03 (s, 1H), 3.87-3.56 (m, 5H), 3.27 (s, 2H), 3.20-3.12 (m, 1H), 3.07-2.42 (m, 9H), 1.32 (d, J=7.0 Hz, 3H), 1.29 (d, J=6.9 Hz, 6H), 1.13-1.02 (m, 1H), 0.65-0.56 (m, 1H), 0.55-0.44 (m, 1H), 0.31-0.22 (m, 1H), 0.21-0.13 (m, 1H).
Compound Cpd-78: white solid, 316.4 mg, ESI[M+H]⁺=375.1.
¹H NMR (400 MHz, d₆-DMSO) δ 10.98 (brs, 1H), 8.34 (s, 1H), 7.32 (s, 1H), 7.21 (s, 1H), 4.21 (s, 2H), 3.92-3.72 (m, 2H), 3.68 (s, 3H), 3.47-2.88 (m, 10H), 1.21 (d, J=6.8 Hz, 3H), 1.17 (d, J=6.5 Hz, 6H), 1.07-0.99 (m, 1H), 0.55-0.46 (m, 1H), 0.36-0.27 (m, 1H), 0.20-0.13 (m, 1H), 0.12-0.04 (m, 1H).
Compound Cpd-79: white solid, 71.2 mg, ESI[M+H]⁺=372.1.
¹H NMR (400 MHz, CDCl₃) δ 12.91 (s, 1H), 7.32 (s, 1H), 7.23 (s, 1H), 5.77 (s, 1H), 5.28 (s, 1H), 4.19-3.96 (m, 3H), 3.70 (s, 3H), 3.64-3.45 (m, 2H), 3.45-3.31 (m, 1H), 3.20-3.08 (m, 1H), 3.07-2.93 (m, 1H), 2.73-2.57 (m, 2H), 2.53-2.43 (m, 1H), 2.44-2.33 (m, 1H), 1.33-1.24 (m, 9H), 1.12-1.01 (m, 1H), 0.66-0.57 (m, 1H), 0.54-0.45 (m, 1H), 0.30-0.22 (m, 1H), 0.20-0.10 (m, 1H).

### Example 34 Preparation of Compound Cpd-80-1 to Cpd-82-1, Cpd-84-1 to Cpd-85-1, Cpd-80 to Cpd-82, Cpd-84 to Cpd-85

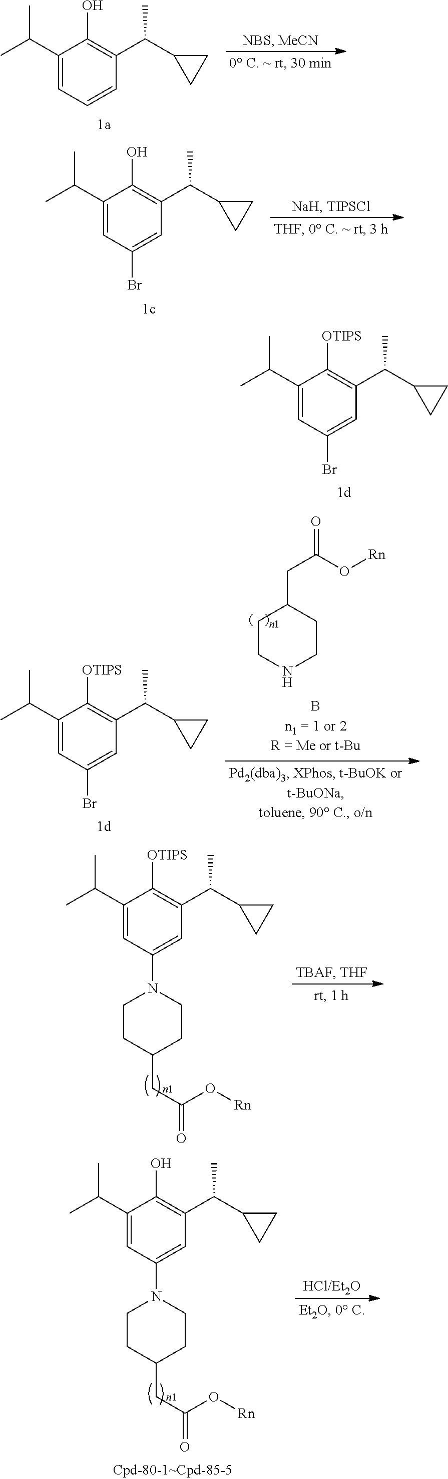

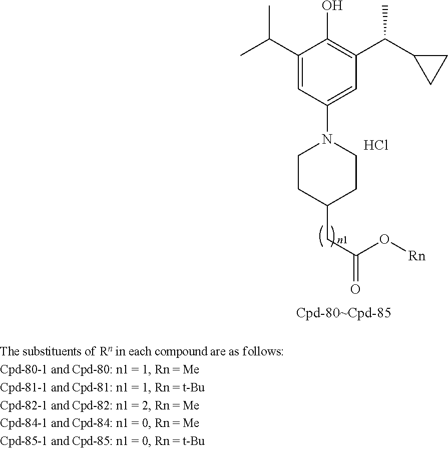

### 1. Preparation of Intermediate Compound 1d

At 0° C., NBS (1.75 g, 9.83 mmol) was added to a solution of 1a (2.00 g, 9.79 mmol) in acetonitrile (20 mL), and the mixture was stirred at 0° C. for 30 minutes. The reaction was monitored by TLC until completion, the reaction was quenched with H₂O (30 mL) and extracted with ethyl acetate (3×10 mL). The combined organic layers were washed with brine, dried over anhydrous Na₂SO₄, filtered, and concentrated under reduced pressure. The crude product was purified by silica gel column chromatography (dichloromethane/petroleum ether (v/v)=1/200-1/50), monitored by TLC (dichloromethane/petroleum ether (v/v)=1/20). The fractions with Rf=0.4-0.5 were collected to afford compound 1c (1.14 g, yield 41.1%) as yellow oil. ESI[M+H]⁺=283.1.

At 0° C., NaH (161.5 mg, 60%, 4.04 mmol) was added portionwise to a solution of 1c (1.04 g, 3.67 mmol) in dry tetrahydrofuran (12 mL). After stirring for 30 minutes, TIPSCl (778.34 mg, 4.04 mmol) was added. The reaction mixture was allowed to warm to room temperature and stirred for 3 hours. The reaction was monitored by TLC until completion, the reaction was quenched with H₂O (20 mL) and extracted with Ethyl acetate (3×10 mL). The combined organic layers were washed with brine, dried over anhydrous Na₂SO₄, filtered, and concentrated under reduced pressure. The crude product was purified by silica gel column chromatography (eluent: n-heptane), monitored by TLC (ethyl acetate/petroleum ether (v/v)=1/50). The fractions with Rf=0.5-0.6 were collected to afford compound 1d (1.35 g, yield 83.7%) as white solid. ESI[M+H]⁺=439.3.

2. Preparation of Compound 10 to 15 and their Corresponding Hydrochloride Salts, Compounds 17-18 and their Corresponding Hydrochloride Salts

### General Procedure:

At room temperature, compound 1d (1.0 eq), the corresponding amine B (1.0-3.0 eq), Pd₂(dba)₃ (0.5 eq), t-BuONa (1.5 eq) or t-BuOK (1.5 eq) and XPhos (0.5 eq) were dissolved in toluene. The reaction mixture was degassed with nitrogen three times and stirred at 90° C. overnight under nitrogen atmosphere. The reaction was monitored by TLC until completion, the solvent was removed under reduced pressure, and the crude product was purified by silica gel column chromatography or preparative TLC to afford the intermediate compound. Deprotection of the intermediate with TBAF followed by purification via silica gel column chromatography or preparative TLC provided the target compounds 10-15 and 17-18.

### 3. Preparation of the Corresponding Hydrochloride Salts of the Target Compounds

The free base was dissolved in diethyl ether and cooled to 0° C. using an ice-water bath. A solution of HCl in Et₂O was slowly added to the reaction mixture via syringe. After stirring for 10 minutes, the resulting precipitate was collected by filtration, washed with diethyl ether, and dried to afford the hydrochloride salt of the target compound.

Cpd-80-1: light brown oil, 140.0 mg, ESI[M+H]⁺=360.1.
¹H NMR (400 MHz, d6-DMSO) δ 7.35 (s, 1H), 6.69-6.64 (m, 1H), 6.57-6.52 (m, 1H), 3.60 (s, 3H), 3.46-3.35 (m, 4H), 3.28-3.20 (m, 1H), 2.45-2.40 (m, 1H), 2.29 (d, J=6.2 Hz, 2H), 1.79-1.67 (m, 3H), 1.38-1.26 (m, 2H), 1.17 (d, J=6.8 Hz, 3H), 1.12 (d, J=5.7 Hz, 6H), 1.03-0.96 (m, 1H), 0.51-0.43 (m, 1H), 0.33-0.25 (m, 1H), 0.16-0.09 (m, 1H), 0.08-0.01 (m, 1H).
Cpd-80: white solid, 155.7 mg, ESI[M+H]⁺=360.3.
¹H NMR (400 MHz, d6-DMSO) δ 11.57 (s, 1H), 8.59 (s, 1H), 7.58 (s, 1H), 7.46 (s, 1H), 3.77-3.57 (m, 5H), 3.51-3.44 (m, 2H), 3.36-3.23 (m, 1H), 2.58-2.50 (m, 1H), 2.37 (d, J=6.9 Hz, 2H), 2.25-2.10 (m, 1H), 1.97-1.85 (m, 2H), 1.85-1.69 (m, 2H), 1.20 (d, J=6.9 Hz, 3H), 1.16 (d, J=6.8 Hz, 6H), 1.06-0.96 (m, 1H), 0.56-0.47 (m, 1H), 0.37-0.30 (m, 1H), 0.22-0.13 (m, 1H), 0.11-0.03 (m, 1H).
Cpd-81-1: colorless oil, 7.6 mg, ESI[M+H]⁺=402.1.
¹H NMR (400 MHz, DMSO) δ 7.34 (s, 1H), 6.66 (s, 1H), 6.55 (s, 1H), 3.45-3.37 (m, 4H), 3.27-3.20 (m, 1H), 2.46-2.39 (m, 1H), 2.19-2.12 (m, 2H), 1.78-1.65 (m, 3H), 1.41 (s, 9H), 1.26-1.21 (m, 2H), 1.16 (d, J=6.9 Hz, 3H), 1.13 (d, J=1.6 Hz, 3H), 1.11 (d, J=1.7 Hz, 3H), 1.05-0.93 (m, 1H), 0.52-0.41 (m, 1H), 0.35-0.23 (m, 1H), 0.17-0.09 (m, 1H), 0.08-0.01 (m, 1H).
Cpd-81: white solid, 227.9 mg, ESI[M+H]⁺=402.4.
¹H NMR (400 MHz, d₆-DMSO) δ 11.39 (s, 1H), 8.60 (s, 1H), 7.55 (s, 1H), 7.43 (s, 1H), 3.73-3.57 (m, 2H), 3.51-3.42 (m, 2H), 3.33-3.24 (m, 1H), 2.56-2.52 (m, 1H), 2.24 (d, J=6.6 Hz, 2H), 2.18-2.04 (m, 1H), 1.96-1.84 (m, 2H), 1.82-1.68 (m, 2H), 1.43 (s, 9H), 1.20 (d, J=6.9 Hz, 3H), 1.16 (d, J=6.6 Hz, 6H), 1.07-0.97 (m, 1H), 0.56-0.48 (m, 1H), 0.38-0.28 (m, 1H), 0.21-0.13 (m, 1H), 0.11-0.03 (m, 1H).
Cpd-82: white solid, 120.0 mg, ESI[M+H]⁺=374.3.
¹H NMR (400 MHz, d₆-DMSO) δ 11.55 (s, 1H), 8.59 (s, 1H), 7.57 (s, 1H), 7.44 (s, 1H), 3.67-3.54 (m, 5H), 3.50-3.42 (m, 2H), 3.35-3.27 (m, 1H), 2.57-2.52 (m, 1H), 2.40 (t, J=7.5 Hz, 2H), 1.97-1.83 (m, 2H), 1.78-1.65 (m, 3H), 1.60-1.51 (m, 2H), 1.20 (d, J=6.9 Hz, 3H), 1.16 (d, J=6.8 Hz, 3H), 1.15 (d, J=6.8 Hz, 3H), 1.07-0.97 (m, 1H), 0.56-0.47 (m, 1H), 0.38-0.28 (m, 1H), 0.21-0.13 (m, 1H), 0.11-0.03 (m, 1H).
Cpd-84-1: white solid, 36.2 mg, ESI[M+H]⁺=346.1.
¹H NMR (400 MHz, d₆-DMSO) δ 7.38 (s, 1H), 6.67 (d, J=2.3 Hz, 1H), 6.56 (d, J=2.3 Hz, 1H), 3.62 (s, 3H), 3.43-3.36 (m, 2H), 3.29-3.21 (m, 1H), 2.68-2.56 (m, 2H), 2.47-2.38 (m, 2H), 1.96-1.86 (m, 2H), 1.75-1.61 (m, 2H), 1.17 (d, J=6.9 Hz, 3H), 1.13 (d, J=2.2 Hz, 3H), 1.11 (d, J=2.2 Hz, 3H), 1.05-0.97 (m, 1H), 0.51-0.42 (m, 1H), 0.33-0.26 (m, 1H), 0.17-0.10 (m, 1H), 0.09-0.02 (m, 1H).
Cpd-84: white solid, 245.0 mg, ESI[M+H]⁺=346.3.
¹H NMR (400 MHz, d₆-DMSO) δ 11.68 (s, 1H), 8.62 (s, 1H), 7.54 (s, 1H), 7.42 (s, 1H), 3.80-3.47 (m, 8H), 3.34-3.26 (m, 1H), 2.88-2.73 (m, 1H), 2.58-2.53 (m, 1H), 2.19-2.05 (m, 3H), 1.20 (d, J=6.9 Hz, 3H), 1.16 (d, J=6.8 Hz, 6H), 1.06-0.96 (m, 1H), 0.57-0.48 (m, 1H), 0.38-0.29 (m, 1H), 0.21-0.13 (m, 1H), 0.11-0.02 (m, 1H).
Cpd-85-1: white solid, 63 mg, ESI[M+H]⁺=388.1.
¹H NMR (400 MHz, d₆-DMSO) δ 7.37 (s, 1H), 6.67 (d, J=2.8 Hz, 1H), 6.55 (d, J=2.9 Hz, 1H), 3.40-3.35 (m, 12H), 3.28-3.20 (m, 1H), 2.63-2.56 (m, 2H), 2.46-2.39 (m, 1H), 2.34-2.22 (m, 1H), 1.89-1.83 (m, 2H), 1.69-1.59 (m, 2H), 1.41 (s, 9H), 1.17 (d, J=6.9 Hz, 3H), 1.13 (d, J=2.3 Hz, 3H), 1.11 (d, J=2.3 Hz, 3H), 1.02-0.94 (m, 4H), 0.50-0.43 (m, 1H), 0.34-0.26 (m, 1H), 0.17-0.10 (m, 1H), 0.08-0.02 (m, 1H).
Cpd-85: white solid, 263.1 mg, ESI[M+H]⁺=388.3.
¹H NMR (400 MHz, d₆-DMSO) δ 11.66 (s, 1H), 8.63 (s, 1H), 7.54 (s, 1H), 7.41 (s, 1H), 3.71-3.47 (m, 5H), 3.35-3.26 (m, 1H), 2.75-2.61 (m, 1H), 2.58-2.52 (m, 1H), 2.13-2.01 (m, 3H), 1.43 (s, 9H), 1.20 (d, J=6.9 Hz, 3H), 1.16 (d, J=6.8 Hz, 6H), 1.05-0.95 (m, 1H), 0.58-0.49 (m, 1H), 0.38-0.28 (m, 1H), 0.21-0.12 (m, 1H), 0.10-0.01 (m, 1H).

### Example 35 Preparation of Compound Cpd-83-1 to Cpd-83, Cpd-86-1 to Cpd-86

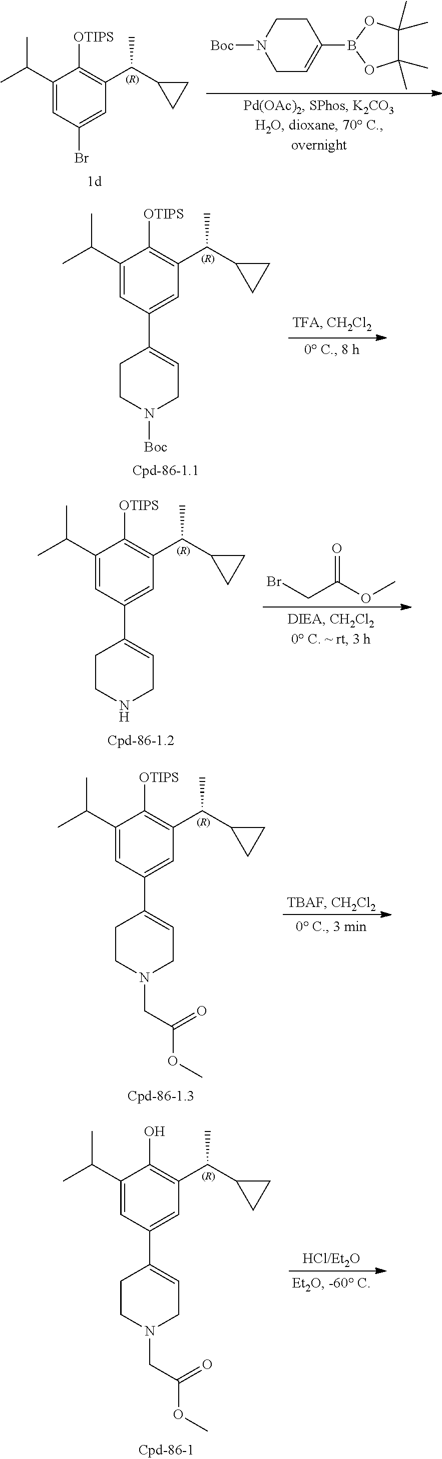

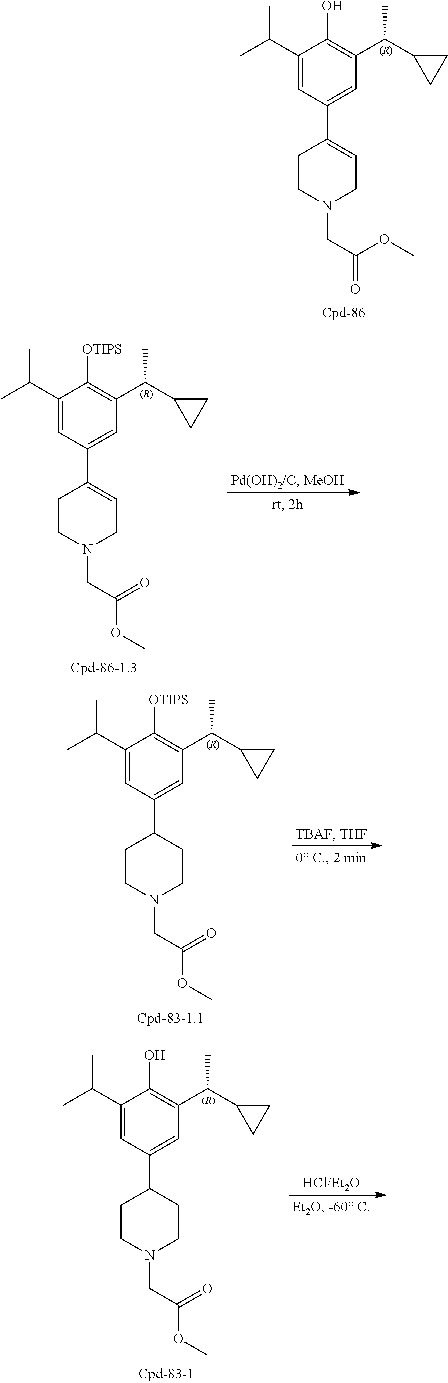

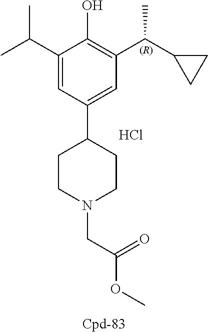

### 1. Preparation of Compound Cpd-86-1 and Cpd-86

At room temperature, 1d (879.1 g, 2.00 mmol), tert-butyl-4-(4,4,5,5-tetramethyl-1,3,2-dioxaborolan-2-yl)-3,6-dihydropyridine-1(2H)-carboxylate (927.6 mg, 3.00 mmol), Pd(OAc)₂ (44.9 mg, 0.20 mmol), K₂CO₃ (829.2 mg, 6.0 mmol) and SPhos (82.11 mg, 0.20 mmol) were dissolved in 1,4-dioxane/H₂O (15 mL), the reaction system was purged with nitrogen three times, then stirred at 70° C. overnight under nitrogen protection. The reaction was monitored by TLC until completion, the mixture was concentrated under reduced pressure to give the crude product. The crude product was purified by silica gel column chromatography (ethyl acetate/petroleum ether (v/v)=1/10), monitored by TLC (ethyl acetate/petroleum ether (v/v)=1/10). The fractions with Rf=0.3-0.4 were collected to give compound Cpd-86-1.1 (1.2 g, crude) as white solid.

At 0° C., TFA (2 mL) was added to a solution of Cpd-86-1.1 (1.2 g) in dichloromethane (10 mL), and the mixture was stirred at 0° C. for 8 hours. The reaction was monitored by TLC until completion, the reaction mixture was concentrated under reduced pressure, basified with saturated aqueous sodium bicarbonate (20 mL), and extracted with Ethyl acetate (3×30 mL). The combined organic layers were dried over anhydrous Na₂SO₄, filtered, and concentrated under reduced pressure. The crude product was purified by silica gel column chromatography (dichloromethane/methanol (v/v)=100/1-20/1), monitored by TLC (dichloromethane/methanol (v/v)=10/1). The fractions with Rf=0.4-0.5 were collected to afford compound Cpd-86-1.2 (1.0 g, crude) as white solid. ESI[M+H]⁺=445.3.

At 0° C., DIEA (349 mg, 2.70 mmol) and methyl bromoacetate (303 mg, 1.98 mmol) were successively added to a solution of the crude compound Cpd-86-1.2 (1.0 g) in dichloromethane (10 mL), and the mixture was stirred at 0° C. for 1 hour. The reaction was monitored by TLC until completion, H₂O (10 mL) was added to the reaction mixture, which was then extracted with ethyl acetate (3×15 mL). The combined organic layers were dried over anhydrous Na₂SO₄, filtered, and concentrated under reduced pressure. The crude product was purified by silica gel column chromatography (ethyl acetate/petroleum ether (v/v)=1/10-1/7), monitored by TLC (ethyl acetate/petroleum ether (v/v)=1/3). The fractions with Rf=0.5-0.6 were collected to afford compound Cpd-86-1.3 (661 mg, yield 64.3% over three steps) as white solid. ESI[M+H]⁺=517.4.

At 0° C., TBAF (0.8 mL, 1 mol/L in THE, 0.8 mmol) was added to a solution of Cpd-86-1.3 (400 mg, 0.78 mmol) in tetrahydrofuran (10 mL), and the mixture was stirred at 0° C. for 3 minutes. The reaction was monitored by TLC until completion, H₂O (10 mL) was added to the reaction mixture, which was then extracted with ethyl acetate (3×15 mL). The combined organic layers were dried over anhydrous Na₂SO₄, filtered, and concentrated under reduced pressure. The crude product was purified by preparative TLC (ethyl acetate/petroleum ether (v/v)=1/3), and the fractions with Rf=0.5-0.6 were collected to afford compound Cpd-86-1 (168 mg, yield 60.2%) as colorless oil. ESI[M+H]⁺=361.3.

At −60° C., a solution of HCl in Et₂O (0.35 mL, 2 mol/L in THE, 0.70 mmol) was slowly added to a solution of compound 19 (168 mg, 0.47 mmol) in diethyl ether (10 mL). The mixture was stirred at −60° C. for 5 minutes, then concentrated under reduced pressure at low temperature to give the crude product. The crude product was washed by diethyl ether to give compound Cpd-86 (169.6 mg, yield 91.6%). ESI[M+H]⁺=358.3.

¹H NMR (400 MHz, CD₃OD) δ 7.23 (d, J=2.2 Hz, 1H), 7.12 (d, J=2.2 Hz, 1H), 5.97 (s, 1H), 4.25 (s, 2H), 4.08-3.94 (m, 2H), 3.87 (s, 3H), 3.72-3.51 (m, 2H), 3.28-3.20 (m, 1H), 2.94-2.84 (m, 2H), 2.52-2.39 (m, 1H), 1.27 (d. J=6.9 Hz, 3H), 1.22 (d, J=6.8 Hz, 6H), 1.11-1.00 (m, 1H), 0.61-0.50 (m, 1H), 0.40-0.29 (m, 1H), 0.23-0.15 (m, 1H), 0.11-0.03 (m, 1H).

### 1. Preparation of Compound Cpd-83-1 and Cpd-83

At room temperature, compound Cpd-86-1.3 (425 mg, 0.83 mmol) and 10% palladium on carbon (43 mg) were dissolved in methanol (15 mL). The reaction system was purged with hydrogen three times, then stirred at room temperature under a hydrogen atmosphere for 2 hours. The reaction was monitored by TLC until completion, the mixture was concentrated under reduced pressure to give the crude product, which was purified by preparative TLC (ethyl acetate/petroleum ether (v/v)=1/4). Fractions with Rf=0.5-0.6 were collected to afford compound Cpd-83-1.1 (325 mg, yield 76.2%) as white solid. ESI[M+H]⁺=545.3.

At 0° C., TBAF (0.7 mL, 1 mol/L in THE, 0.7 mmol) was added to a solution of Cpd-83-1.1 (325 mg, 0.63 mmol) in tetrahydrofuran (5 mL), and the mixture was stirred at 0° C. for 2 minutes. The reaction was monitored by TLC until completion, H₂O (10 mL) was added to the reaction mixture, which was then extracted with Ethyl acetate (3×8 mL). The combined organic layers were dried over anhydrous Na₂SO₄, filtered, and concentrated under reduced pressure. The crude product was purified by preparative TLC (ethyl acetate/petroleum ether (v/v)=1/100-1/2), and the fractions with Rf=0.5-0.6 were collected to afford compound Cpd-83-1 (193.4 mg, yield 85.4%) as colorless oil. ESI[M+H]⁺=361.3.

At −60° C., a solution of HCl in Et₂O (0.4 mL, 2 mol/L in THE, 0.80 mmol) was slowly added to a solution of compound Cpd-83-1 (193.4 mg, 0.54 mmol) in diethyl ether (10 mL). The mixture was stirred at −60° C. for 5 minutes, then concentrated under reduced pressure at low temperature to give the crude product. The crude product was washed with diethyl ether to give compound Cpd-83 (194.6 mg, yield 91.4%). ESI[M+H]⁺=361.3.

### Example 36 Preparation of Compound Cpd-91

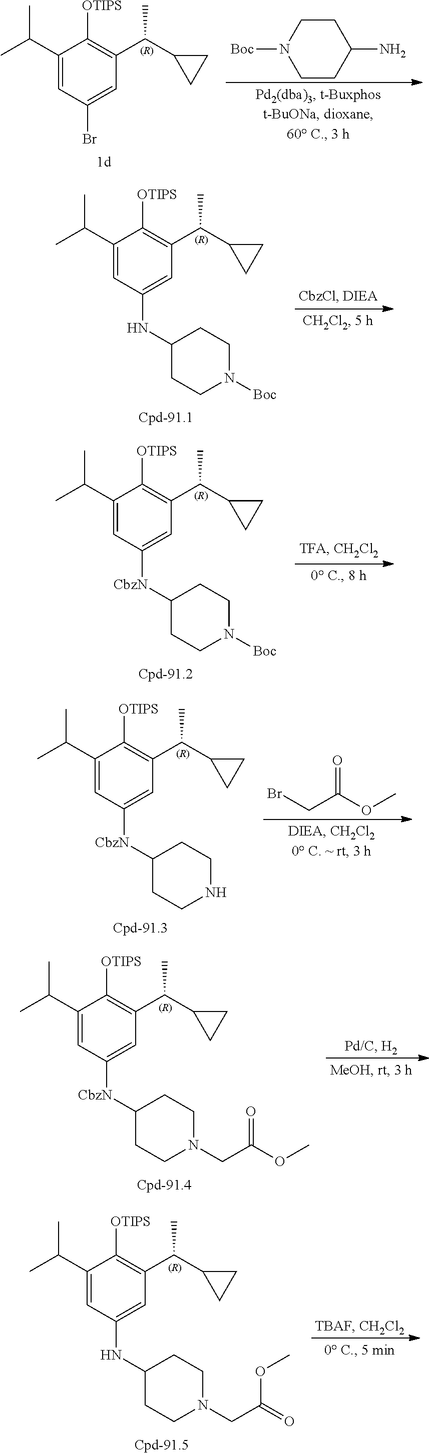

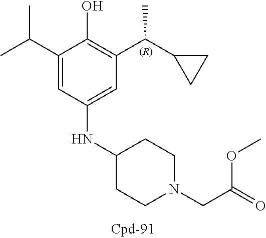

At room temperature, 1d (879.1 g, 2.0 mmol), tert-butyl 4-aminopiperidine-1-carboxylate (801.1 mg, 4.0 mmol), Pd₂(dba)₃ (183.1 mg, 0.2 mmol), t-BuOK (336.6 mg, 3.0 mmol) and t-Buxphos (84.9 mg, 0.2 mmol) were dissolved in 1,4-dioxan (10 mL), the reaction system was purged with nitrogen three times, then stirred at 60° C. for 3 hours under nitrogen protection. The reaction was monitored by TLC until completion, the mixture was concentrated under reduced pressure to obtain the crude product. The crude product was purified by silica gel column chromatography (ethyl acetate/petroleum ether (v/v)=1/10), monitored by TLC (ethyl acetate/petroleum ether (v/v)=1/5). The fractions with Rf=0.5 were collected to give crude compound Cpd-91.1 (1.153 g) as colorless oil. ESI[M+H−56]⁺=504.1.

At 0° C. in an ice-water bath, DIEA (297.3 mg, 2.30 mmol) and CbzCl (235.4 mg, 1.38 mmol) were successively added to a solution of Cpd-91.1 (640 mg, 1.15 mmol) in dichloromethane (10 mL). The mixture was stirred at room temperature for 5 hours. The reaction was monitored by TLC (ethyl acetate/petroleum ether (v/v)=1/5) until completion, H₂O (20 mL) was added to the reaction mixture, which was then extracted with dichloromethane (3% 10 mL). The combined organic layers were dried over anhydrous Na₂SO₄, filtered, and concentrated under reduced pressure. The crude product was purified by flash column chromatography (ethyl acetate/petroleum ether (v/v)=0-15%), monitored by TLC (ethyl acetate/petroleum ether (v/v)=1/8). Fractions with Rf=0.5 were collected to give compound Cpd-91.2 (530 mg, yield 66.5%) as colorless oil. ESI[M+H]⁺=543.4.

At 0° C. in an ice-salt bath, TFA (1 mL) was added to a solution of the crude compound Cpd-91.2 (530 mg, 0.76 mmol) in dichloromethane (4 mL), and the mixture was stirred at 0° C. for 8 hours. The reaction was monitored by TLC (ethyl acetate/petroleum ether (v/v)=1/8) until completion, the reaction mixture was concentrated under reduced pressure, basified with saturated aqueous sodium bicarbonate (10 mL), and extracted with dichloromethane (3× 10 mL). The combined organic layers were dried over anhydrous Na₂SO₄, filtered, and concentrated under reduced pressure. The crude product was purified by flash column chromatography (ethyl acetate/petroleum ether (v/v)=1/1, then methanol/dichloromethane (v/v)=0-1/1), monitored by TLC (methanol/dichloromethane (v/v)=1/10). Fractions with Rf=0.5 were collected to give compound Cpd-91.3 (277.2 mg, yield 61.5%) as colorless oil. ESI[M+H]⁺=593.3.

At 0° C. in an ice-water bath, DIEA (121.5 mg, 0.94 mmol) and methyl bromoacetate (107.2 mg, 0.7 mmol) were successively added to a solution of Cpd-91.3 (277 mg, 0.47 mmol) in dichloromethane (10 mL). The mixture was stirred at room temperature overnight. The reaction was monitored by TLC (methanol/dichloromethane (v/v)=1/10) until completion, H₂O (20 mL) was added to the reaction mixture, which was then extracted with ethyl acetate (3×10 mL). The combined organic layers were dried over anhydrous Na₂SO₄, filtered, and concentrated under reduced pressure. The crude product was purified by flash column chromatography (ethyl acetate/petroleum ether (v/v)=10-50%), monitored by TLC (ethyl acetate/petroleum ether (v/v)=1/1). Fractions with Rf=0.5 were collected to give compound Cpd-91.4 (210.2 mg, yield 67.3%) as colorless oil. ESI[M+H]⁺=665.4.

At room temperature, compound Cpd-91.4 (210 mg, 0.32 mmol) and 10% palladium hydroxide on carbon (21 mg) were dissolved in methanol (5 mL). The reaction system was purged with hydrogen three times, then stirred under a hydrogen atmosphere at room temperature for 3 hours. The reaction was monitored by TLC until completion, the mixture was concentrated under reduced pressure. The crude product was purified by preparative TLC (ethyl acetate/petroleum ether (v/v)=1/1), and fractions with Rf=0.5-0.6 were collected to give compound Cpd-91.5 (132.4 mg, yield 77.9%) as white solid. ESI[M+H]⁺=531.3.

At 0° C. in an ice-salt bath, TBAF (0.25 mL, 1 mol/L in THE, 0.25 mmol) was added to a solution of Cpd-91.5 (132 mg, 0.25 mmol) in dry THF (5 mL). The mixture was stirred at 0° C. for 1 hour. The reaction was monitored by TLC until completion, H₂O (20 mL) was added to the reaction mixture, which was then extracted with ethyl acetate (3×10 mL). The combined organic layers were dried over anhydrous Na₂SO₄, filtered, and concentrated under reduced pressure. The crude product was purified by flash column chromatography (ethyl acetate/petroleum ether (v/v)=0-20%), monitored by TLC (ethyl acetate/petroleum ether (v/v)=1/5). Fractions with Rf=0.5 were collected to give crude compound Cpd-91 (35.1 mg, yield 37.5%) as colorless oil. ESI[M+H]⁺=375.3.

### Example 37 Preparation of Compound Cpd-87-1 and Cpd-87

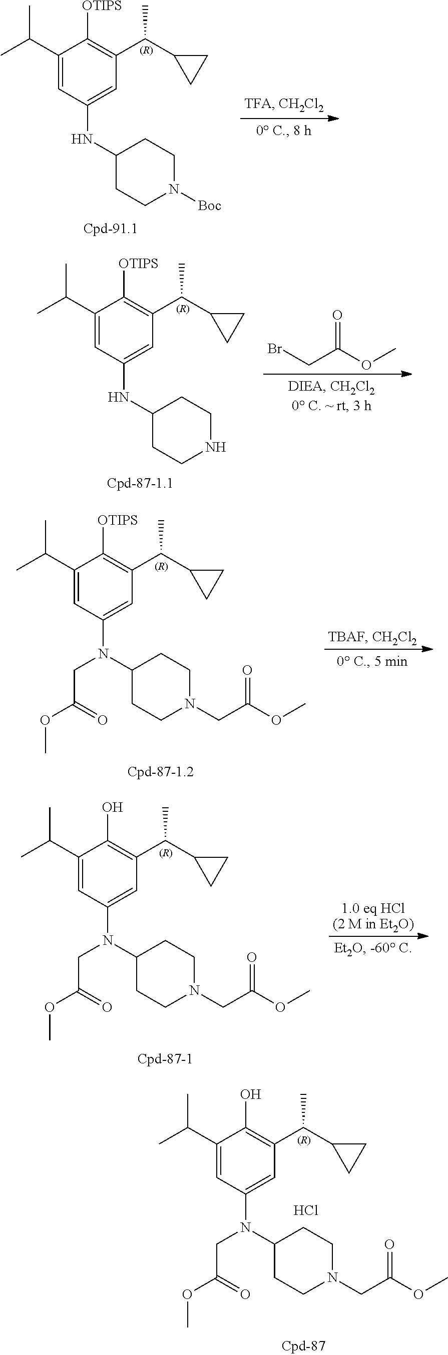

At 0° C. in an ice-salt bath, TFA (1 mL) was added to a solution of the crude compound Cpd-91.1 (668.2 mg, 1.20 mmol) in dichloromethane (4 mL), and the mixture was stirred at 0° C. for 8 hours. The reaction was monitored by TLC (ethyl acetate/petroleum ether (v/v)=1/8) until completion, the reaction mixture was concentrated under reduced pressure, basified with saturated aqueous sodium bicarbonate (10 mL), and extracted with dichloromethane (3×10 mL). The combined organic layers were dried over anhydrous Na₂SO₄, filtered, and concentrated under reduced pressure. The crude product was purified by flash column chromatography (ethyl acetate/petroleum ether (v/v)=1/1, then methanol/dichloromethane (v/v)=0-1/1), monitored by TLC (methanol/dichloromethane (v/v)=1/10). Fractions with Rf=0.5 were collected to give compound Cpd-87-1.1 (337.1 mg, yield 61.5%) as colorless oil. ESI[M+H]⁺=459.3.

At 0° C. in an ice-water bath, DIEA (471.7 mg, 3.65 mmol) and methyl bromoacetate (336.6 mg, 2.20 mmol) were successively added to a solution of Cpd-87-1.1 (337 mg, 0.73 mmol) in dichloromethane (10 mL). The mixture was stirred at room temperature overnight. The reaction was monitored by TLC (methanol/dichloromethane (v/v)=1/10) until completion, H₂O (20 mL) was added to the reaction mixture, which was then extracted with ethyl acetate (3×10 mL). The combined organic layers were dried over anhydrous Na₂SO₄, filtered, and concentrated under reduced pressure. The crude product was purified by flash column chromatography (ethyl acetate/petroleum ether (v/v)=10-50%), monitored by TLC (ethyl acetate/petroleum ether (v/v)=1/1). Fractions with Rf=0.5 were collected to afford compound Cpd-87-1.2 (150.6 mg, yield 34.2%) as colorless oil. ESI[M+H]⁺=603.4.

At 0° C. in an ice-salt bath, TBAF (2.0 mL, 1 mol/L in THF, 2.0 mmol) was added to a solution of Cpd-87-1.2 (150 mg, 0.25 mmol) in dry THF (10 mL), the mixture was stirred at 0° C. for 1 hour. Upon completion monitored by TLC, H₂O (20 mL) was added to the reaction mixture, which was then extracted with ethyl acetate (3×10 mL). The combined organic layers were dried over anhydrous Na₂SO₄, filtered, and concentrated under reduced pressure. The crude product was purified by flash column chromatography (ethyl acetate/petroleum ether (v/v)=10-50%), monitored by TLC (ethyl acetate/petroleum ether (v/v)=1/1). Fractions with Rf=0.5 were collected to give crude compound Cpd-87-1 (73.3 mg, yield 65.7%) as colorless oil. ESI[M+H]⁺=447.3.

At −60° C., a solution of HCl in Et₂O (0.1 mL, 2 mol/L in THE, 0.2 mmol) was slowly added to a solution of compound Cpd-87-1 (73 mg, 0.16 mmol) in diethyl ether (5 mL). The mixture was stirred at −60° C. for 5 minutes, then concentrated under reduced pressure at low temperature to give the crude product. The crude product was washed with diethyl ether to give compound Cpd-87 (49.5 mg, yield 64.1%). ESI[M+H]⁺=447.3.

¹H NMR (400 MHz, CD₃OD) δ 6.80 (s, 1H), 6.68 (s, 1H), 4.28-4.07 (m, 2H), 4.06-3.86 (m, 2H), 3.84 (s, 3H), 3.80-3.59 (m, 6H), 3.28-3.09 (m, 3H), 2.51-2.38 (m, 1H), 2.24-1.74 (m, 4H), 1.24 (d, J=6.9 Hz, 3H), 1.19 (d, J=6.8 Hz, 3H), 1.18 (d, J=6.8 Hz, 3H), 1.06-0.95 (m, 1H), 0.59-0.50 (m, 1H), 0.39-0.29 (m, 1H), 0.23-0.13 (m, 1H), 0.12-0.02 (m, 1H).

### Example 38 Preparation of Compound Cpd-88

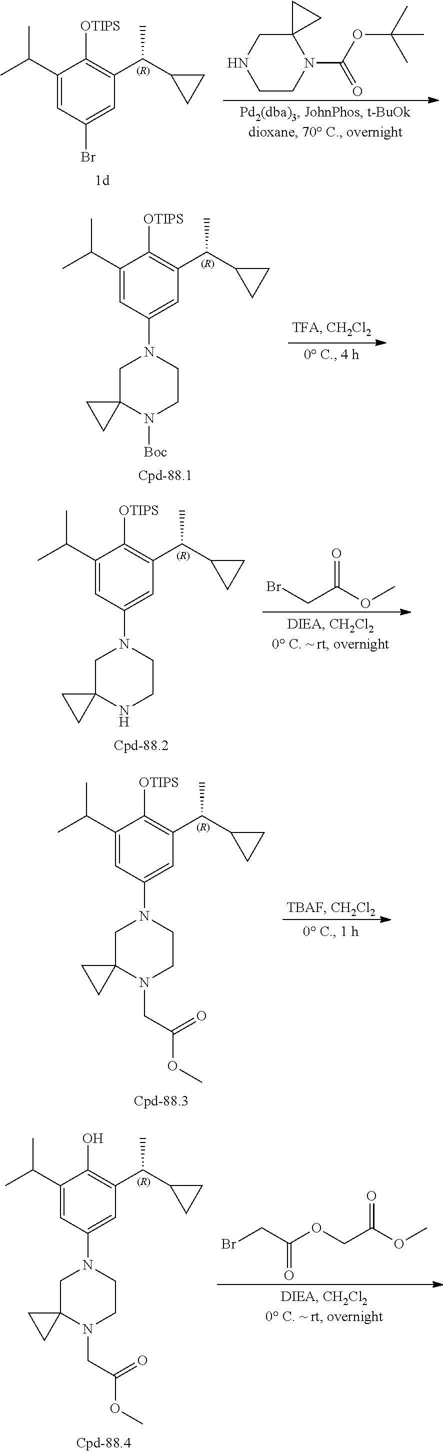

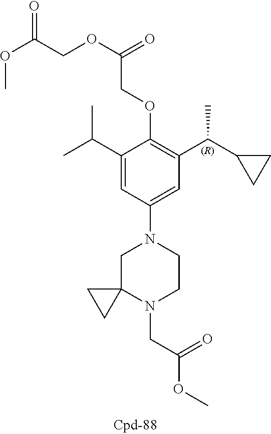

At room temperature, compound 1d (6.0 g, 13.65 mmol), tert-butyl 4,7-diazaspiro[2.5]octane-4-carboxylate (5.0 g, 23.55 mmol), Pd₂(dba)₃ (1.25 g, 1.37 mmol), t-BuOK (3.06 g, 27.30 mmol) and JohnPhos (814.7 mg, 2.73 mmol) were dissolved in 1,4-dioxane (40 mL). The reaction system was purged with nitrogen three times, then stirred at 70° C. overnight under nitrogen atmosphere. The reaction was monitored by TLC (n-heptane) until completion, the mixture was concentrated under reduced pressure. The crude product was purified by flash column chromatography (ethyl acetate/petroleum ether (v/v)=0-15%), monitored by TLC (ethyl acetate/petroleum ether (v/v)=1/5). Fractions with Rf=0.5 were collected to give crude compound Cpd-88.1 (5.7 g) as the colorless oil. ESI[M+H]⁺=571.4.

At 0° C. in an ice-salt bath, TFA (5 mL) was added to a solution of the crude compound Cpd-88.1 (5.7 g, 9.98 mmol) in dichloromethane (15 mL). The mixture was stirred at 0° C. for 4 hours. The reaction was monitored by TLC until completion, the reaction mixture was concentrated under reduced pressure, basified with saturated aqueous sodium bicarbonate (20 mL), and extracted with ethyl acetate (3×10 mL). The combined organic layers were dried over anhydrous Na₂SO₄, filtered, and concentrated under reduced pressure. The crude product was purified by flash column chromatography (ethyl acetate/petroleum ether (v/v)=1/1, then methanol/dichloromethane (v/v)=0-1/1), monitored by TLC (methanol/dichloromethane (v/v)=1/10). Fractions with Rf=0.5 were collected to give compound Cpd-88.2 (3.617 g, yield 56.3% over two steps) as colorless oil. ESI[M+H]⁺=471.4.

At 0° C. in an ice-water bath, DIEA (517.0 mg, 4.0 mmol) and methyl bromoacetate (458.9 mg, 3.0 mmol) were successively added to a solution of Cpd-88.2 (941.6 mg, 2.0 mmol) in dichloromethane (20 mL). The mixture was stirred at room temperature overnight. The reaction was monitored by TLC (methanol/dichloromethane (v/v)=1/10) until completion, H₂O (20 mL) was added to the reaction mixture, which was then extracted with ethyl acetate (3×10 mL). The combined organic layers were dried over anhydrous Na₂SO₄, filtered, and concentrated under reduced pressure. The crude product was purified by flash column chromatography (ethyl acetate/petroleum ether (v/v)=0-15%), monitored by TLC (ethyl acetate/petroleum ether (v/v)=1/8). Fractions with Rf=0.5 were collected to give compound Cpd-88.3 (920 mg, yield 77.0%) as colorless oil. ESI[M+H]⁺=543.4.

At 0° C. in an ice-salt bath, TBAF (2.0 mL, 1 mol/L in THE, 2.0 mmol) was added to a solution of Cpd-88.3 (920 mg, 1.69 mmol) in dry THF (10 mL). The mixture was stirred at 0° C. for 1 hour. The reaction was monitored by TLC until completion, H₂O (20 mL) was added to the reaction mixture, which was then extracted with ethyl acetate (3×10 mL). The combined organic layers were dried over anhydrous Na₂SO₄, filtered, and concentrated under reduced pressure. The crude product was purified by flash column chromatography (ethyl acetate/petroleum ether (v/v)=0-20%), monitored by TLC (ethyl acetate/petroleum ether (v/v)=1/5). Fractions with Rf=0.5 were collected to afford crude compound Cpd-88.4 (660.2 mg, yield 100%) as colorless oil. ESI[M+H]⁺=387.2.

At 0° C. in an ice-water bath, DIEA (53.0 mg, 0.41 mmol) and 2-methoxy-2-oxoethyl 2-bromoacetate (53 mg, 0.25 mmol) were successively added to a solution of Cpd-88.4 (80 mg, 0.21 mmol) in dichloromethane (8 mL). The mixture was stirred at room temperature overnight. The reaction was monitored by TLC (ethyl acetate/petroleum ether (v/v)=1/5) until completion, H₂O (20 mL) was added to the reaction mixture, which was then extracted with ethyl acetate (3×10 mL). The combined organic layers were dried over anhydrous Na₂SO₄, filtered, and concentrated under reduced pressure. The crude product was purified by Prep-TLC (ethyl acetate/petroleum ether (v/v)=1/8) to give compound Cpd-88 (49.5 mg, yield 45.6%) as white solid. ESI[M+H]⁺=375.4.

### Example 39 Preparation of Compound Cpd-89-1 and Cpd-89

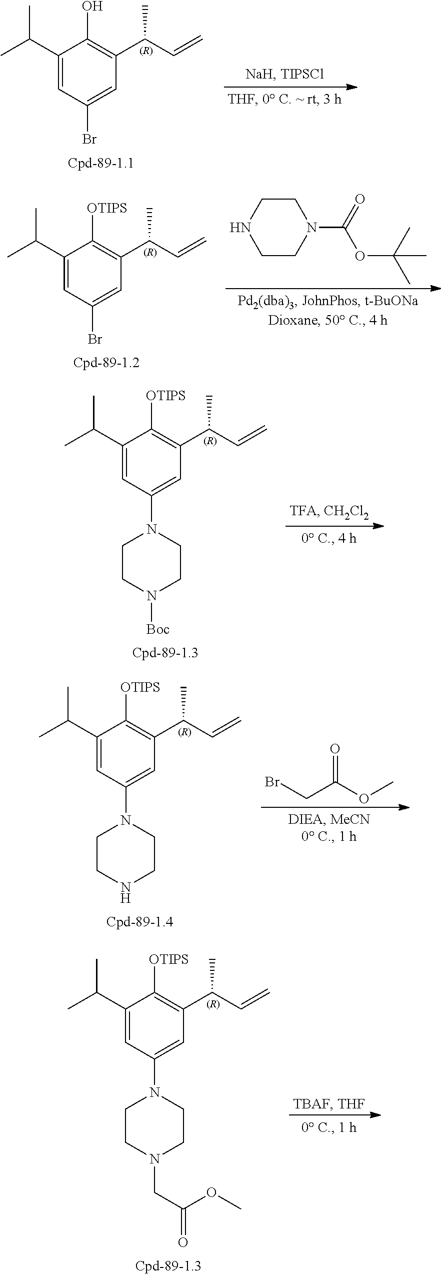

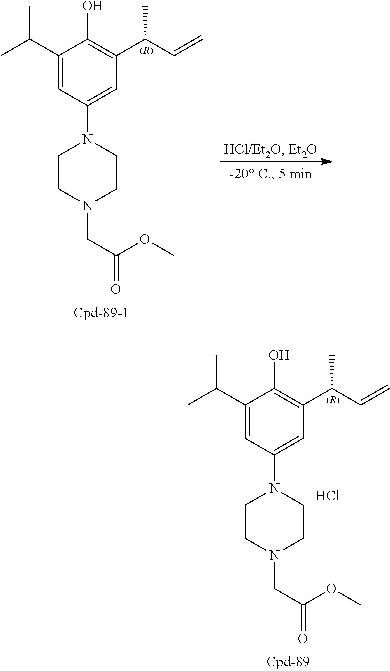

According with the preparation of the Cpd-88, the target compound Cpd-89-1 and Cpd-89 were obtained starting from Cpd-89-1.1.
Cpd-89-1: white solid, 1.96 g, ESI[M+H]⁺=347.3.
¹H NMR (400 MHz, CD₃OD) δ 6.75 (d, J=2.9 Hz, 1H), 6.65 (d, J=2.9 Hz, 1H), 6.16-6.01 (m, 1H), 5.14-5.00 (m, 2H), 3.97-3.83 (m, 1H), 3.76 (s, 3H), 3.39-3.24 (m, 1H), 3.14-3.04 (m, 4H), 2.82-2.70 (m, 4H), 1.32 (d, J=7.0 Hz, 3H), 1.24 (d, J=6.9 Hz, 6H).
Cpd-89: white solid, 216 mg, ESI[M+H]⁺=347.3.
¹H NMR (400 MHz, CD₃OD) δ 7.16 (d, J=2.8 Hz, 1H), 7.05 (d, J=2.8 Hz, 1H), 6.16-6.03 (m, 1H), 5.16-5.06 (m, 2H), 4.28 (s, 2H), 3.97 (p, J=6.9 Hz, 1H), 3.78-3.68 (m, 8H), 3.44-3.35 (m, 1H), 1.36 (d, J=7.0 Hz, 3H), 1.27 (d, J=6.9 Hz, 6H).

### Example 40 Preparation of Compound Cpd-92 to Cpd-104

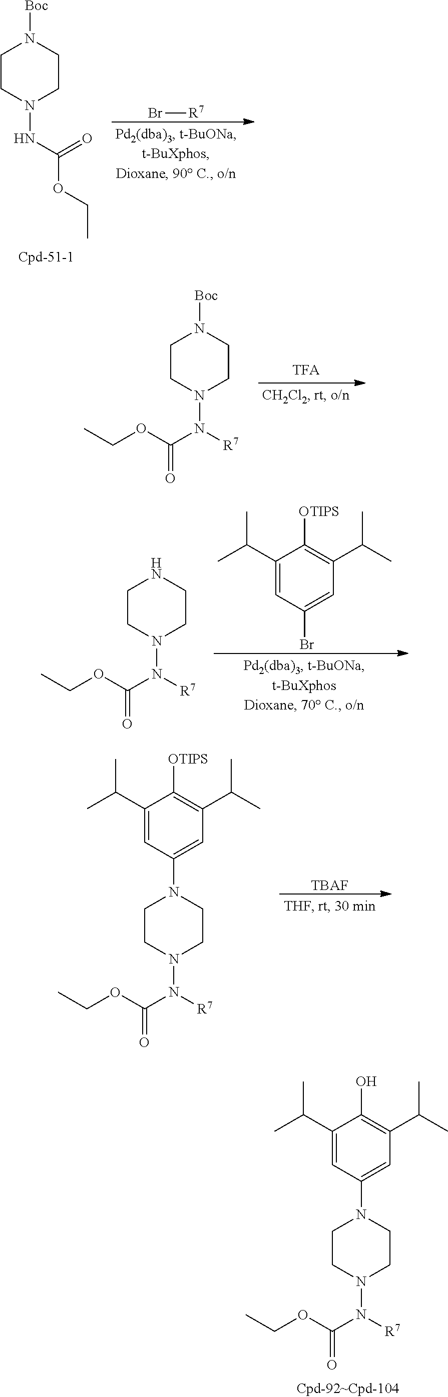

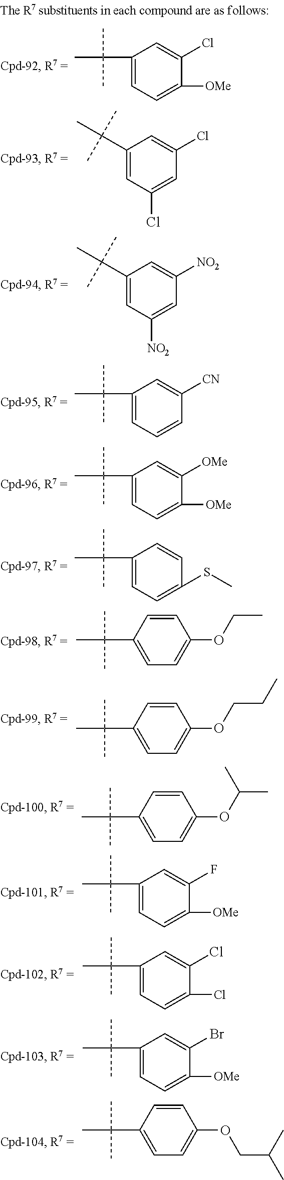

According with the preparation of the Cpd-51, the target compound Cpd-92 to Cpd-104 were obtained starting from Cpd-51-1.
Compound Cpd-92: white solid, 189 mg, ESI[M+H]⁺=489.2.

### ¹H NMR (400 MHz, MeOD) δ 7.38 (d, J=2.4 Hz, 1H), 7.26 (dd, J=8.8, 2.4 Hz, 1H), 7.13 (d, J=8.8 Hz, 1H), 6.70 (s, 2H), 4.21 (q, J=7.0 Hz, 2H), 3.95 (s, 3H), 3.30 (dd, J=12.5, 5.5 Hz, 2H), 3.17 (s, 8H), 1.27 (t, J=7.1 Hz, 3H), 1.22 (d, J=6.9 Hz, 12H).

Compound Cpd-93: white solid, 171 mg, ESI[M+H]⁺=493.1.

### ¹H NMR (400 MHz, MeOD) δ 7.53 (d, J=1.7 Hz, 2H), 7.30 (t, J=1.7 Hz, 1H), 6.74 (s, 2H), 4.32 (q, J=7.1 Hz, 2H), 3.43-3.36 (m, 4H), 3.33-3.27 (m, 2H), 3.24-3.11 (m, 4H), 1.37 (t, J=7.1 Hz, 3H), 1.24 (d, J=6.9 Hz, 12H).

Compound Cpd-94: white solid, 21 mg, ESI[M+H]⁺=515.2.

### ¹H NMR (400 MHz, MeOD) δ 8.93 (d, J=1.9 Hz, 2H), 8.77-8.75 (m, 1H), 6.77 (s, 2H), 4.44 (q, J=7.1 Hz, 2H), 3.76-3.41 (m, 4H), 3.41-3.36 (m, 2H), 3.33-3.15 (m, 4H), 1.45 (t, J=7.1 Hz, 3H), 1.25 (d, J=6.9 Hz, 12H).

Compound Cpd-95: white solid, 72.6 mg, ESI[M+H]⁺=450.2.

### ¹H NMR (400 MHz, MeOD) δ 7.90-7.84 (m, 1H), 7.80 (dt, J=7.1, 2.1 Hz, 1H), 7.62-7.53 (m, 2H), 6.73 (s, 2H), 4.31 (q, J=7.1 Hz, 2H), 3.50-3.36 (m, 4H), 3.32-3.27 (m, 2H), 3.24-3.12 (m, 4H), 1.35 (t, J=7.1 Hz, 3H), 1.23 (d, J=6.9 Hz, 12H).

Compound Cpd-96: white solid, 152.4 mg, ESI[M+H]⁺=485.2.
¹H NMR (400 MHz, DMSO) δ 8.08 (s, 1H), 7.54 (s, 2H), 6.95 (s, 1H), 6.63 (s, 2H), 4.14 (q, J=7.1 Hz, 2H), 3.77 (s, 3H), 3.74 (s, 3H), 3.34-3.26 (m, 2H), 3.20-3.10 (m, 4H), 3.00-2.87 (m, 4H), 1.25 (t, J=7.1 Hz, 3H), 1.17 (d, J=6.9 Hz, 12H).
Compound Cpd-97: white solid, 185 mg, ESI[M+H]⁺=471.2.

### ¹H NMR (400 MHz, MeOD) δ 7.33 (d, J=8.7 Hz, 2H), 7.27 (d, J=8.7 Hz, 2H), 6.70 (s, 2H), 4.21 (q, J=7.1 Hz, 2H), 3.33-3.27 (m, 2H), 3.24-3.09 (m, 8H), 2.53 (s, 3H), 1.27 (t, J=7.1 Hz, 3H), 1.22 (d, J=6.9 Hz, 12H).

Compound Cpd-98: white solid, 178 mg, ESI[M+H]⁺=469.2.
¹H NMR (400 MHz, MeOD) δ 7.19 (d, J=8.9 Hz, 2H), 6.97 (d, J=8.9 Hz, 2H), 6.69 (s, 2H), 4.17 (q, J=7.1 Hz, 2H), 4.10 (q, J=7.0 Hz, 2H), 3.32-3.24 (m, 2H), 3.22-3.13 (m, 4H), 3.13-3.05 (m, 4H), 1.43 (t, J=7.0 Hz, 3H), 1.24 (d, J=7.2 Hz, 3H), 1.22 (d, J=6.9 Hz, 12H).
Compound Cpd-99: white solid, 181 mg, ESI[M+H]⁺=483.3.
¹H NMR (400 MHz, MeOD) δ 7.19 (d, J=8.9 Hz, 2H), 6.98 (d, J=8.9 Hz, 2H), 6.69 (s, 2H), 4.17 (q, J=7.1 Hz, 2H), 3.99 (t, J=6.5 Hz, 2H), 3.33-3.25 (m, 2H), 3.24-3.13 (m, 4H), 3.13-3.02 (m, 4H), 1.92-1.76 (m, 2H), 1.29-1.23 (m, 3H), 1.22 (d, J=6.9 Hz, 12H), 1.09 (t, J=7.4 Hz, 3H).
Compound Cpd-100: white solid, 180 mg, ESI[M+H]⁺=483.3.
¹H NMR (400 MHz, MeOD) δ 7.19 (d, J=8.8 Hz, 2H), 6.96 (d, J=8.8 Hz, 2H), 6.71 (s, 2H), 4.65 (dt, J=12.0, 6.0 Hz, 1H), 4.18 (q, J=7.1 Hz, 2H), 3.34-3.27 (m, 2H), 3.24-3.14 (m, 4H), 3.15-3.07 (m, 4H), 1.36 (d, J=6.0 Hz, 6H), 1.26 (dd, J=9.8, 4.8 Hz, 3H), 1.22 (d, J=6.9 Hz, 12H).
Compound Cpd-101: white solid, 218 mg, ESI[M+H]⁺=473.2.

### ¹H NMR (400 MHz, MeOD) δ 7.18-7.08 (m, 3H), 6.70 (s, 2H), 4.21 (q, J=7.0 Hz, 2H), 3.93 (s, 3H), 3.33-3.26 (m, 2H), 3.24-3.10 (m, 8H), 1.27 (t, J=7.0 Hz, 3H), 1.22 (d, J=6.9 Hz, 12H).

Compound Cpd-102: white solid, 91 mg, ESI[M+H]⁺=493.1.

### ¹H NMR (400 MHz, MeOD) δ 7.67 (d, J=2.5 Hz, OH), 7.53 (d, J=8.7 Hz, OH), 7.42 (dd, J=8.7, 2.5 Hz, OH), 6.73 (s, 1H), 4.29 (q, J=7.1 Hz, 2H), 3.41-3.36 (m, 2H), 3.33-3.26 (m, 4H), 3.24-3.12 (m, 4H), 1.34 (t, J=7.1 Hz, 3H), 1.23 (d, J=6.9 Hz, 12H).

Compound Cpd-103: white solid, 107 mg, ESI[M+H]⁺=533.1.

### ¹H NMR (400 MHz, MeOD) δ 7.53 (d, J=2.5 Hz, 1H), 7.30 (dd, J=8.7, 2.5 Hz, 1H), 7.10 (d, J=8.8 Hz, 1H), 6.70 (s, 2H), 4.21 (q, J=7.1 Hz, 2H), 3.94 (s, 3H), 3.33-3.28 (m, 2H), 3.23-3.09 (m, 8H), 1.27 (t, J=7.1 Hz, 3H), 1.22 (d, J=6.9 Hz, 12H).

Compound Cpd-104: white solid, 145 mg, ESI[M+H]⁺=497.3.
¹H NMR (400 MHz, MeOD) δ 7.19 (d, J=8.8 Hz, 2H), 6.98 (d, J=8.9 Hz, 2H), 6.69 (s, 2H), 4.17 (q, J=7.0 Hz, 2H), 3.80 (d, J=6.5 Hz, 2H), 3.32-3.28 (m, 2H), 3.22-3.14 (m, 4H), 3.14-3.04 (m, 4H), 2.10 (td, J=13.2, 6.6 Hz, 1H), 1.28-1.23 (m, 3H), 1.22 (d, J=6.9 Hz, 12H), 1.08 (d, J=6.7 Hz, 6H).

### Example 41 Preparation of Compound Cpd-105 to Cpd-107

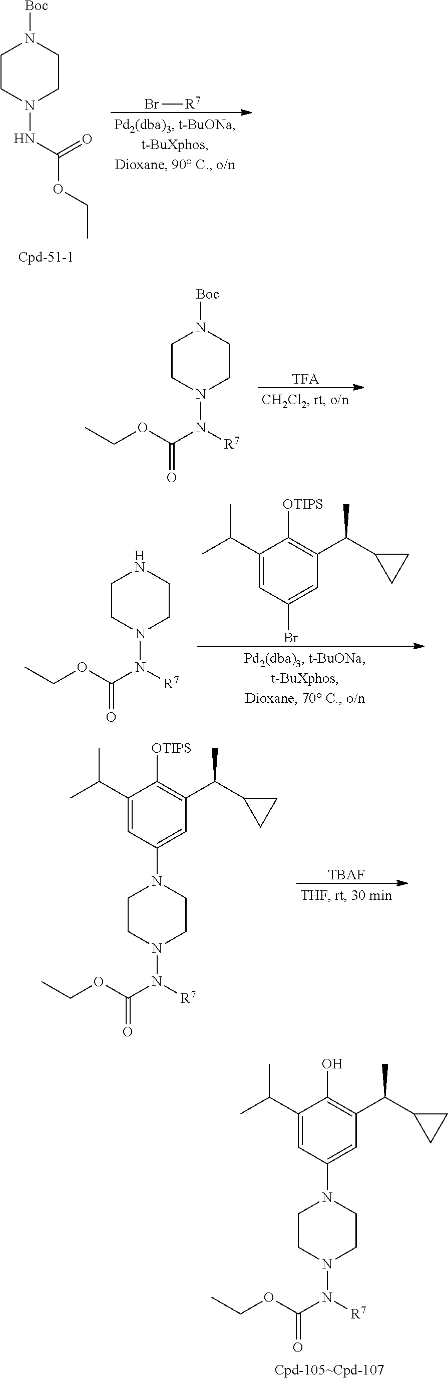

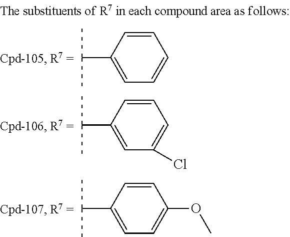

According with the preparation of the Cpd-51, the target compound Cpd-105 to Cpd-107 were obtained starting from Cpd-51-1.
Compound Cpd-105: white solid, 55 mg, ESI[M+H]⁺=451.2.
¹H NMR (400 MHz, MeOD) δ 7.42 (d, J=6.9 Hz, 2H), 7.34 (d, J=7.5 Hz, 3H), 6.82 (d, J=2.9 Hz, 1H), 6.71 (d, J=2.9 Hz, 1H), 4.21 (q, J=7.1 Hz, 2H), 3.33-3.26 (m, 1H), 3.26-3.21 (m, 4H), 3.20-3.13 (m, 4H), 2.48 (dq, J=13.9, 6.8 Hz, 1H), 1.27 (dd, J=6.9, 6.1 Hz, 6H), 1.22 (dd, J=6.9, 1.3 Hz, 6H), 1.14-1.01 (m, 1H), 0.65-0.52 (m, 1H), 0.45-0.32 (m, 1H), 0.29-0.16 (m, 1H), 0.14-0.08 (m, 1H).
Compound Cpd-106: white solid, 82 mg, ESI[M+H]⁺=485.2.
¹H NMR (400 MHz, MeOD) δ 7.49-7.45 (m, 1H), 7.42-7.36 (m, 2H), 7.29 (dt, J=7.0, 2.1 Hz, 1H), 6.84 (d, J=2.9 Hz, 1H), 6.73 (d, J=2.9 Hz, 1H), 4.27 (q, J=7.1 Hz, 2H), 3.41-3.35 (m, 1H), 3.33-3.26 (m, 4H), 3.23-3.13 (m, 4H), 2.58-2.39 (m, 1H), 1.32 (t, J=7.1 Hz, 3H), 1.29 (d, J=7.0 Hz, 3H), 1.23 (dd, J=6.9, 1.3 Hz, 6H), 1.11-1.01 (m, 1H), 0.64-0.53 (m, 1H), 0.44-0.34 (m, 1H), 0.26-0.17 (m, 1H), 0.16-0.09 (m, 1H).
Compound Cpd-107: white solid, 74 mg, ESI[M+H]⁺=481.2.
¹H NMR (400 MHz, MeOD) δ 7.21 (d, J=8.9 Hz, 2H), 6.99 (d, J=8.9 Hz, 2H), 6.81 (d, J=2.9 Hz, 1H), 6.70 (d, J=2.9 Hz, 1H), 4.18 (q, J=7.1 Hz, 2H), 3.86 (s, 3H), 3.32-3.25 (m, 1H), 3.22-3.15 (m, 4H), 3.14-3.04 (m, 4H), 2.55-2.42 (m, 1H), 1.27 (d, J=7.0 Hz, 3H), 1.25 (d, J=7.9 Hz, 3H), 1.21 (d, J=7.4 Hz, 6H), 1.11-0.97 (m, 1H), 0.63-0.52 (m, 1H), 0.42-0.35 (m, 1H), 0.24-0.16 (m, 1H), 0.15-0.06 (m, 1H).

### Example 42 Preparation of Compound Cpd-122 to Cpd-123

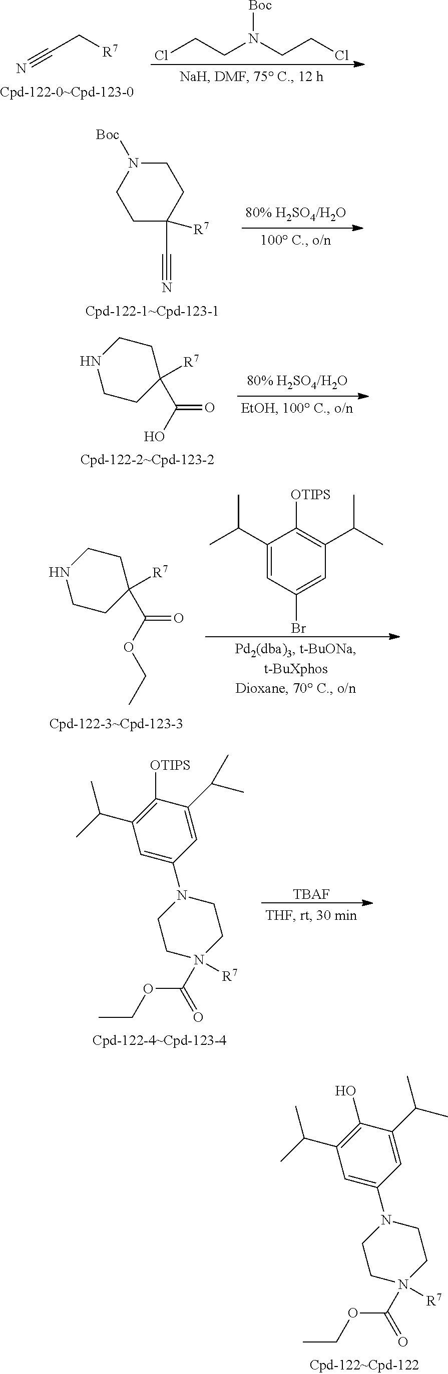

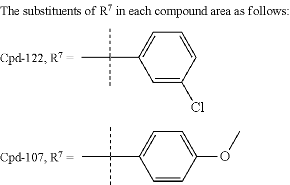

According with the preparation of the Cpd-27, the target compound Cpd-122 to Cpd-123 were obtained starting from Cpd-122-0 to Cpd-123-0.
Compound Cpd-122: white solid, 147 mg, ESI[M+H]⁺=457.2.

### 1H NMR (400 MHz, MeOD) δ 7.50-7.46 (m, 1H), 7.45-7.36 (m, 2H), 7.35-7.30 (m, 1H), 6.75 (s, 2H), 5.13-4.99 (m, 1H), 3.47-3.37 (m, 2H), 3.33-3.28 (m, 2H), 2.90-2.66 (m, 4H), 2.18-2.04 (m, 2H), 1.24 (d, J=6.9 Hz, 12H), 1.21 (d, J=6.2 Hz, 6H).

Compound Cpd-123: white solid, 91 mg, ESI[M+H]⁺=453.2.
1H NMR (400 MHz, MeOD) δ 7.39 (d, J=8.9 Hz, 2H), 6.94 (d, J=8.9 Hz, 2H), 6.75 (s, 2H), 5.09-4.97 (m, 1H), 3.82 (s, 3H), 3.45-3.36 (m, 2H), 3.31 (d, J=6.9 Hz, 2H), 2.91-2.77 (m, 2H), 2.76-2.65 (m, 2H), 2.17-2.00 (m, 2H), 1.24 (d, J=6.9 Hz, 12H), 1.19 (d, J=6.2 Hz, 6H),

### Example 43 Preparation of Compound Cpd-124

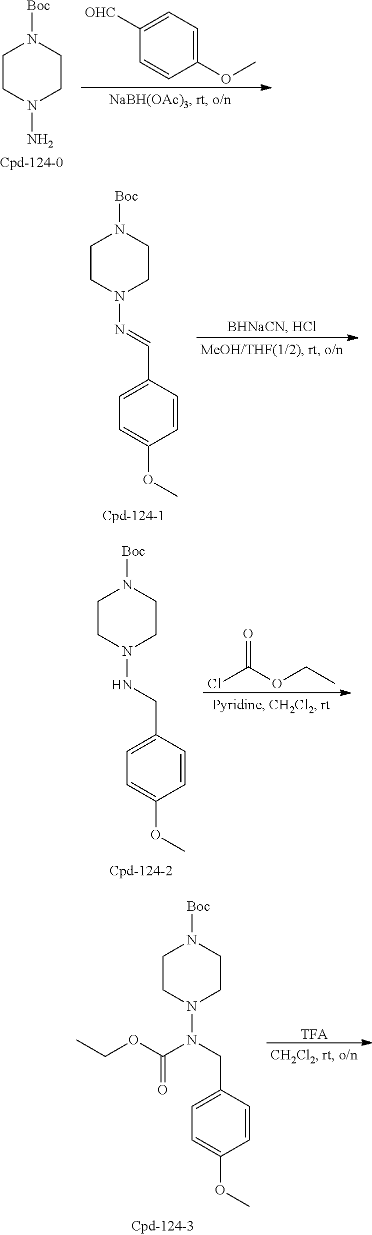

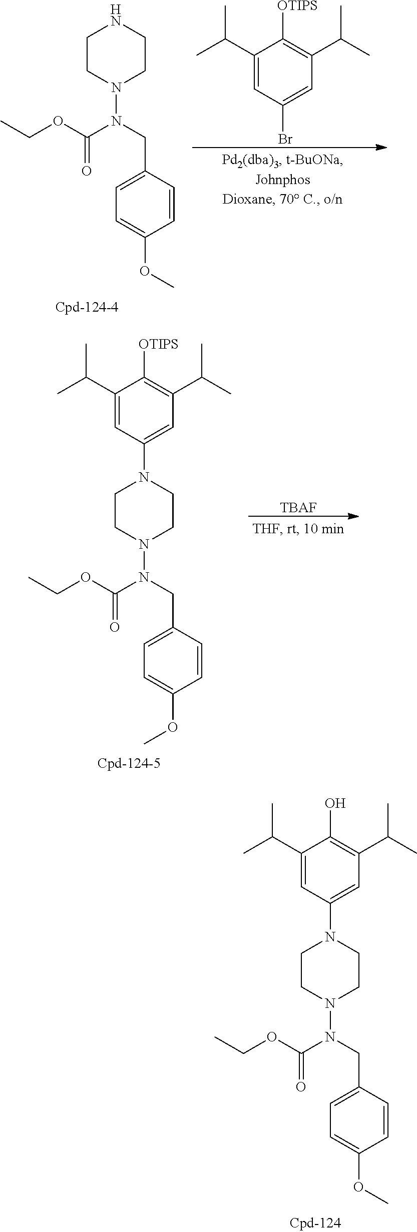

### 1. Preparation of Compound Cpd-124-1

At room temperature, compound Cpd-124-0 (2 g, 10.0 mmol) and p-anisaldehyde (2.76 g, 20.0 mmol) were dissolved in dichloromethane (150 mL) and stirred at room temperature for 2 hours. NaBH(OAc)₃ (6.36 g, 30.0 mmol) was added to the reaction mixture, which was then stirred at room temperature overnight. The reaction was monitored by TLC until completion, saturated aqueous sodium bicarbonate (50 mL) was added to the reaction mixture, which was extracted with ethyl acetate (3×50 mL). The combined organic layers were washed with brine, dried over anhydrous Na₂SO₄, filtered, and concentrated under reduced pressure. The crude product was purified by silica gel column chromatography (ethyl acetate/petroleum ether (v/v)=1/100-1/2), monitored by TLC (ethyl acetate/petroleum ether (v/v)=1/2). Fractions with Rf=0.3-0.4 were collected to afford compound Cpd-124-1 (2.1 g, yield 65.8%) as white solid. ESI[M+H]⁺=319.1.

### 2. Preparation of Compound Cpd-124-2

At room temperature, sodium cyanoborohydride (773 mg, 12.3 mmol) and 1M hydrochloric acid in ethanol (0.6 mL) were added to a solution of Cpd-124-1 (1.3 g, 4.1 mmol) in methanol/tetrahydrofuran (15 mL/30 mL). The mixture was stirred at room temperature for 5 hours. The reaction was monitored by TLC until completion, ice-water (50 mL) was added to the reaction mixture, which was then extracted with ethyl acetate (3×50 mL). The combined organic layers were washed with brine, dried over anhydrous Na₂SO₄, filtered, and concentrated under reduced pressure. The crude product was purified by silica gel column chromatography (ethyl acetate/petroleum ether (v/v)=1/100-1/1), monitored by TLC (ethyl acetate/petroleum ether (v/v)=1/1). Fractions with Rf=0.4-0.5 were collected to give compound Cpd-124-2 (1.2 g, yield 91.3%) as white solid. ESI[M+H]⁺=321.1.

### 3. Preparation of Compound Cpd-124-3

At room temperature, pyridine (441 mg, 5.58 mmol) and methyl carbonochloridate (242 mg, 2.24 mmol) were added to a solution of Cpd-124-2 (358 mg, 1.12 mmol) in dry dichloromethane (10 mL). The mixture was stirred at room temperature for 2 hours. The reaction was monitored by TLC until completion, ice-water (20 mL) was added to the reaction mixture, which was then extracted with ethyl acetate (3×20 mL). The combined organic layers were washed with brine, dried over anhydrous Na₂SO₄, filtered, and concentrated under reduced pressure. The crude product was purified by silica gel column chromatography (ethyl acetate/petroleum ether (v/v)=1/100-1/3), monitored by TLC (ethyl acetate/petroleum ether (v/v)=1/3). Fractions with Rf=0.3-0.4 were collected to give compound Cpd-124-3 (388 mg, yield 88.1%) as white solid. ESI[M+H]⁺=393.2.

### 4. Preparation of Compound Cpd-124-4

At room temperature, compound Cpd-124-3 (348 mg, 0.89 mmol) was dissolved in dichloromethane (5 mL), and trifluoroacetic acid (1.03 g, 8.9 mmol) was added to the reaction mixture. The solution was stirred at room temperature for 8 hours. The reaction was monitored by TLC until completion, saturated aqueous sodium bicarbonate was added to adjust the pH to 7-8. The mixture was concentrated under reduced pressure, and the crude product was purified by silica gel column chromatography (methanol/dichloromethane (v/v)=1/100-1/10), monitored by TLC (methanol/dichloromethane (v/v)=1/10). Fractions with Rf=0.3-0.4 were collected to afford compound Cpd-124-4 (216 mg, yield 82.8%) as white solid. ESI[M+H]⁺=293.1.

### 5. Preparation of Compound Cpd-124-5

At room temperature, Compound Cpd-124-4 (187 mg, 0.64 mmol), Cpd-1-2 (262 mg, 0.64 mmol), Pd₂(dba)₃ (58.6 mg, 0.064 mmol), t-BuONa (122.9 mg, 1.28 mmol) and JohnPhos (20.3 mg, 0.068 mmol) were dissolved in 1,4-dioxan (5 mL), the reaction system was purged with nitrogen three times, then stirred at 70° C. for 8 hours under nitrogen protection. The reaction was monitored by TLC until completion, the crude product was obtained by concentration under reduced pressure and purified by silica gel column chromatography (ethyl acetate/petroleum ether (v/v)=1/100-1/10). TLC monitoring (ethyl acetate/petroleum ether (v/v)=1/10) was performed, and the fractions with Rf=0.3-0.4 were collected to yield compound Cpd-124-5 (287 mg, yield 71.7%) as white solid. ESI[M+H]⁺=625.4.

### 6. Preparation of Compound Cpd-124

At 0° C. in an ice-salt bath, TBAF (0.23 mL, 1 mol/L in THF, 0.23 mmol) was added to a solution of Cpd-124-5 (287 mg, 0.46 mmol) in dry tetrahydrofuran (5 mL), and the mixture was stirred at 0° C. for 1 hour. The reaction was monitored by TLC until completion, water (10 mL) was added to the reaction system, and the mixture was extracted with ethyl acetate (3×10 mL). The combined organic phases were dried over anhydrous Na₂SO₄, filtered, and concentrated under reduced pressure to obtain the crude product. The crude product was purified by silica gel column chromatography (ethyl acetate/petroleum ether (v/v)=1/100-1/3), monitored by TLC (ethyl acetate/petroleum ether (v/v)=1/4), and the fractions with Rf=0.3-0.4 were collected to give compound Cpd-124 (169 mg, yield 78.3%) as white solid. ESI[M+H]⁺=469.2.

### ¹H NMR (400 MHz, MeOD) δ 7.34 (d, J=8.6 Hz, 2H), 6.92 (d, J=8.7 Hz, 2H), 6.68 (s, 2H), 4.49 (s, 2H), 4.25 (q, J=6.9 Hz, 2H), 3.82 (s, 3H), 3.33-3.28 (m, 2H), 3.24-2.46 (m, 8H), 1.36 (t, J=7.1 Hz, 3H), 1.22 (d, J=6.9 Hz, 12H).

### Example 44 Preparation of Compound Cpd-125 to Cpd-129

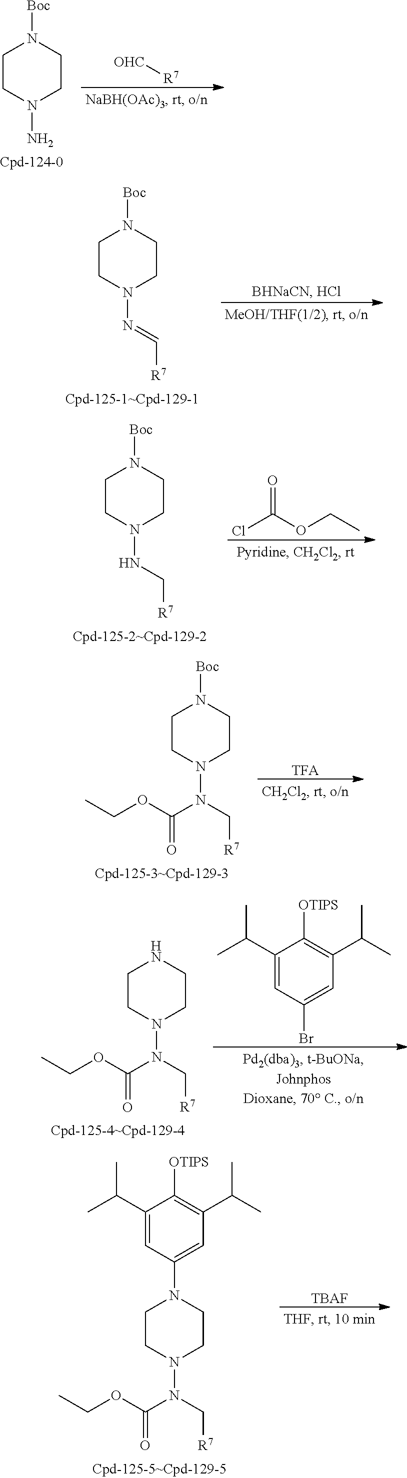

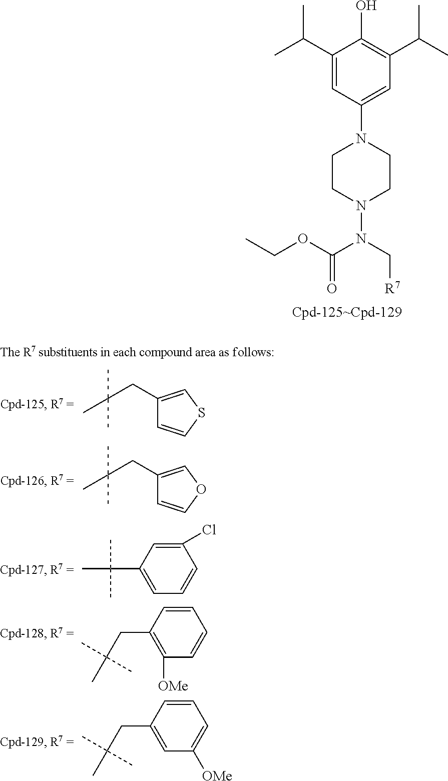

According with the preparation of the Cpd-124, the target compound Cpd-125 to Cpd-129 were obtained starting from Cpd-124-0.
Compound Cpd-125: white solid, 175 mg, ESI[M+H]⁺=445.2.

### ¹H NMR (400 MHz, MeOD) δ 7.39 (dd, J=4.9, 3.0 Hz, 1H), 7.35 (s, 1H), 7.16 (dd, J=4.9, 1.2 Hz, 1H), 6.69 (s, 2H), 4.57 (s, 2H), 4.25 (q, J=7.0 Hz, 2H), 3.33-3.25 (m, 2H), 3.23-2.60 (m, 8H), 1.36 (t, J=7.1 Hz, 3H), 1.23 (d, J=6.9 Hz, 12H).

Compound Cpd-126: white solid, 125 mg, ESI[M+H]⁺=429.2.

### ¹H NMR (400 MHz, MeOD) δ 7.53 (s, 1H), 7.48 (t, J=1.7 Hz, 1H), 6.70 (s, 2H), 6.50 (d, J=1.1 Hz, 1H), 4.42 (s, 2H), 4.25 (q, J=7.1 Hz, 2H), 3.32-3.26 (m, 2H), 3.26-2.53 (m, 8H), 1.36 (t, J=7.1 Hz, 3H), 1.23 (d, J=6.9 Hz, 12H).

Compound Cpd-127: white solid, 172 mg, ESI[M+H]⁺=473.2.

### ¹H NMR (400 MHz, MeOD) δ 7.42 (s, 1H), 7.39-7.28 (m, 3H), 6.68 (s, 2H), 4.54 (s, 2H), 4.32-4.21 (m, 2H), 3.31-3.24 (m, 2H), 3.19-2.55 (m, 8H), 1.35 (t, J=7.1 Hz, 3H), 1.21 (d, J=6.9 Hz, 12H).

Compound Cpd-128: white solid, 221 mg, ESI[M+H]⁺=469.2.

### ¹H NMR (400 MHz, MeOD) δ 7.35-7.24 (m, 2H), 7.03-6.90 (m, 2H), 6.66 (s, 2H), 4.62 (s, 2H), 4.24 (dd, J=12.7, 5.9 Hz, 2H), 3.88 (s, 3H), 3.33-3.24 (m, 2H), 3.20-2.51 (m, 8H), 1.34 (t, J=7.0 Hz, 3H), 1.22 (d, J=6.9 Hz, 12H).

Compound Cpd-129: white solid, 210 mg, ESI[M+H]⁺=469.2.

### ¹H NMR (400 MHz, MeOD) δ 7.27 (t, J=7.9 Hz, 1H), 6.98 (s, 2H), 6.91-6.82 (m, 1H), 6.68 (s, 2H), 4.54 (s, 2H), 4.26 (dd, J=13.9, 7.0 Hz, 2H), 3.83 (s, 3H), 3.33-3.25 (m, 2H), 3.23-2.69 (m, 8H), 1.36 (s, 3H), 1.22 (d, J=6.9 Hz, 12H).

The following examples illustrate the beneficial effects of the present invention:

### Example 1 Determination of Pharmacological Data for Compounds of the Present Invention

### 1. Experiment Method

Determination of the anesthetic effect (determination of the minimum effective anesthetic dose) of the representative compound of the present invention in rats via tail vein injection:

SD rats were used, with a single intravenous injection via the tail vein (administration rate of 0.02 ml/s and administration volume of 0.6 ml per rat) in the experiment. The initial dose of the test compound started at 1 mg/kg, and the actual administration dose was calculated based on the body weight measured before the experiment for each animal. The increase or decrease in subsequent doses was determined based on the results of the experimental animals (whether the LORR (Loss of righting reflex) occurred). The dose at which the LORR first occurred was determined as the minimum effective anesthetic dose.

When testing the anesthetic effect (minimum anesthetic effective dose) of the compounds via tail vein injection in rats, once the compound was determined to have nesthetic effect (loss of righting reflex for ≥30 s), the compound's response to noxious stimuli was assessed. A 30-second alligator clip pinch was applied to the middle-outer third of the rat's tail. If the rat showed no response within 30 s, the compound was deemed to have analgesic activity. If the compound failed to induce anesthesia (loss of righting reflex <30 s), the noxious stimulus (same clip method) was applied 1 minute after testing. The absence of response within 30 s indicated analgesic efficacy, while any reaction classified the compound as lacking analgesia.

The minimal effective anesthetic dose (MEAD) and minimal effective analgesic dose (MEAD) were further classified as: A≤5 mg/kg; 5 mg/kg<B≤10 mg/kg; 10 mg/kg<C≤20 mg/kg; 20 mg/kg<D≤40 mg/k; E>40 mg/kg.

### Pharmacological Characteristics at Equivalent Doses:

In the above experiments, in addition to recording the dose at which loss of the righting reflex occurs, the onset time and recovery time of the anesthetic effect from the start of administration, the duration of righting reflex loss, and the duration of sedative effect can also be recorded. While measuring the dose of the compounds of the present invention causing loss of the righting reflex, the effect of the compounds on the respiration of experimental animals can also be observed.

### 2. The Results of Experimental

TABLE 1
Pharmacological Data of a Single Intravenous Injection
of the Compounds of the Present Invention

| | Minimal effective | Onset time | Righting Reflex | Sedation Duration |
|---|---|---|---|---|
| Compound | anesthetic dose(mg/kg) | (min) | Duration (min) | (min) |
| Cpd-1 | A | 0.38-0.62 | 6.27-10.78 | 19.02-22.97 |
| Cpd-3 | A | 0.20-0.67 | 5.83-22.20 | 24.65-38.02 |
| Cpd-4 | B | 0.62 | 4.17 | 28.03 |
| Cpd-5 | B | 0.45 | 6.40 | 17.12 |
| Cpd-8 | B | 0.40 | 7.73 | 17.93 |
| Cpd-9 | C | 0.90-1.43 | 2.00-6.50 | 9.87-89.25 |
| Cpd-10 | B | 0.42-0.45 | 6.47-27.50 | 47.22-66.70 |
| Cpd-12 | C | 0.45 | 6.85 | 55.12 |
| Cpd-13 | B | 0.35 | 20.27 | 41.57 |
| Cpd-14 | B | 0.48 | 17.60 | 42.83 |
| Cpd-15 | B | 0.67 | 16.28 | 29.90 |
| Cpd-17 | A | 0.62 | 8.50 | 21.53 |
| Cpd-18 | B | 0.20-0.57 | 5.55-20.30 | 26.77-47.53 |
| Cpd-21 | A | 1.35 | 1.32 | 20.10 |
| Cpd-23 | C | 0.53 | 13.95 | 41.02 |
| Cpd-24 | B | 0.85 | 11.60 | 21.70 |
| Cpd-25 | B | 1.28-2.95 | 3.45-6.43 | 17.48-23.03 |
| Cpd-26 | B | 0.98-1.77 | 12.33-28.05 | 26.52-182.97 |
| Cpd-27 | D | 1.47 | 36.80 | 67.40 |
| Cpd-28 | D | 0.73-1.02 | 2.97-12.00 | 31.98-218.72 |
| Cpd-32 | B | 0.52 | 2.67 | 19.68 |
| Cpd-34 | C | 0.88 | 10.28 | 29.75 |
| Cpd-37 | B | 2.63 | 0.65 | 21.73 |
| Cpd-39 | C | 0.73-1.07 | 7.45-24.00 | 34.33-39.48 |
| Cpd-45 | B | 0.38-0.58 | 9.45-21.03 | 21.22-30.92 |
| Cpd-46 | B | 1.58 | 2.17 | 12.95 |
| Cpd-50 | D | 1.03 | 1.47 | 66.48 |
| Cpd-51 | B | 0.65-0.97 | 2.52-14.47 | 22.27-35.75 |
| Cpd-52 | B | 0.52-0.98 | 13.92-16.42 | 28.53-94.30 |
| Cpd-53 | A | 0.47-1.27 | 2.00-37.20 | 27.75-167.40 |
| Cpd-59 | C | 0.37-0.85 | 21.18-24.15 | 37.27-47.68 |
| Cpd-61 | C | 0.55-1.25 | 4.15-41.88 | 89.98-162.23 |
| Cpd-62 | C | 1.15-2.15 | 1.22-17.28 | 55.48-85.02 |
| Cpd-63 | C | 0.18-0.50 | 6.92-33.82 | 50.33-193.58 |
| Cpd-64 | C | 0.43-0.47 | 3.70-7.22 | 26.92-42.58 |
| Cpd-65 | C | 0.92-1.17 | 11.30-16.67 | 101.42-123.97 |
| Cpd-66 | D | 0.22 | 40.40 | 115.20 |
| Cpd-67 | A | 0.23-0.95 | 1.57-3.98 | 53.10-71.03 |
| Cpd-68 | C | 0.50-1.17 | 10.83-45.48 | 38.12-126.35 |
| Cpd-69 | C | 0.68-2.12 | 1.28-74.05 | 19.78-160.07 |
| Cpd-70 | C | 0.38-1.07 | 5.02-39.35 | 48.32-106.60 |
| Cpd-71 | C | 0.55 | 17.52 | 37.65 |
| Cpd-73 | C | 0.83-1.65 | 8.78-25.28 | 44.83-119.62 |
| Cpd-74 | D | 0.50 | 26.28 | 69.23 |
| Cpd-76 | A | 0.37-0.78 | 0.97-3.00 | 6.97-12.63 |
| Cpd-77 | A | 1.00-1.80 | 0.67-1.95 | 5.00-22.33 |
| Cpd-78-1 | B | 0.62 | 2.73 | 22.58 |
| Cpd-80-1 | A | 0.22-1.20 | 2.07-20.22 | 7.73-40.32 |
| Cpd-80 | A | 0.17-0.97 | 1.50-12.97 | 10.17-28.53 |
| Cpd-81 | A | 0.18-0.75 | 9.28-40.37 | 27.22-57.86 |
| Cpd-82 | A | 0.17-0.52 | 0.87-3.42 | 3.67-13.02 |
| Cpd-83 | A | 0.17-0.50 | 1.78-10.62 | 11.48-26.15 |
| Cpd-84-1 | A | 0.43-0.77 | 0.77-4.62 | 5.72-10.28 |
| Cpd-84 | A | 0.30-0.75 | 1.10-17.23 | 18.08-35.02 |
| Cpd-85-1 | A | 0.40-1.33 | 1.38-29.00 | 20.97-44.48 |
| Cpd-85 | A | 0.18-0.55 | 1.28-4.92 | 5.05-15.13 |
| Cpd-86 | A | 0.17-0.20 | 1.15-10.33 | 16.40-19.62 |
| Cpd-87 | C | 0.42 | 1.85 | 19.32 |
| Cpd-89 | C | 0.18 | 0.88 | 22.78 |
| Cpd-92 | D | 0.97-1.05 | 18.85-23.75 | 167.85-172.63 |
| Cpd-95 | D | 0.72 | 13.75 | 54.33 |
| Cpd-96 | D | 0.13-0.75 | 1.32-43.20 | 86.20-295.93 |
| Cpd-97 | B | 0.20-0.60 | 5.25-32.28 | 31.60-83.28 |
| Cpd-98 | C | 0.18-0.73 | 9.92-32.65 | 28.52-93.83 |
| Cpd-99 | C | 1.03-1.38 | 25.58-25.87 | 67.73-88.57 |
| Cpd-100 | D | 0.37-0.95 | 6.65-26.68 | 62.20-114.60 |
| Cpd-101 | C | 0.70-0.73 | 2.05-2.58 | 38.73-57.17 |
| Cpd-102 | B | 0.72-1.95 | 2.40-10.52 | 22.78-315.75 |
| Cpd-103 | D | 0.15 | 14.85 | 107.67 |
| Cpd-104 | D | 0.42 | 59.33 | 377.87 |
| Cpd-105 | B | 0.68-1.80 | 0.24-22.82 | 50.17-52.20 |
| Cpd-106 | C | 1.55-1.60 | 3.35-24.10 | 98.82-103.67 |
| Cpd-107 | B | 0.42-1.52 | 0.72-34.8 | 34.37-73.58 |
| Cpd-123 | D | 0.77 | 24.08 | 75.50 |
| Cpd-124 | D | 0.15-0.68 | 2.83-65.08 | 95.00->220.0 |
| Cpd-125 | C | 1.22 | 6.87 | 53.93 |
| Cpd-126 | C | 0.42 | 14.90 | 21.85 |
| Cpd-127 | B | 0.55 | 21.20 | 71.18 |
| Cpd-128 | C | 1.00 | 20.68 | 111.25 |
| Cpd-129 | C | 0.37-1.05 | 11.98-27.68 | 40.60-70.88 |

TABLE 2
Minimum Effective Analgesic Dose of Single Intravenous
Injection for Compounds of the Present Invention

| | Minimal effective analgesic |
|---|---|
| Compound | dose(mg/kg) |
| Cpd-1 | A |
| Cpd-9 | C |
| Cpd-13 | B |
| Cpd-21 | A |
| Cpd-24 | B |
| Cpd-25 | B |
| Cpd-26 | B |
| Cpd-28 | D |
| Cpd-39 | C |
| Cpd-45 | B |
| Cpd-46 | B |
| Cpd-53 | B |
| Cpd-61 | C |
| Cpd-63 | D |
| Cpd-65 | C |
| Cpd-66 | D |
| Cpd-67 | C |
| Cpd-68 | C |
| Cpd-69 | C |
| Cpd-70 | C |
| Cpd-73 | D |
| Cpd-93 | C |
| Cpd-96 | D |
| Cpd-97 | C |
| Cpd-98 | C |
| Cpd-99 | C |
| Cpd-100 | D |
| Cpd-102 | C |
| Cpd-104 | D |
| Cpd-105 | C |
| Cpd-107 | C |
| Cpd-123 | D |
| Cpd-124 | D |
| Cpd-125 | C |
| Cpd-128 | C |
| Cpd-129 | D |

As shown in Table 1, the representative compounds of the present invention exhibit general anesthetic effects in tests, characterized by rapid onset and short recovery time. Significantly, these compounds simultaneously demonstrate analgesic effects during their general anesthetic action.

In summary, the present invention provides novel substituted phenol derivative represented by formula I. The derivative not only has sedative, hypnotic and/or anesthetic effects, but also has analgesic effects simultaneously. Therefore, the compounds provided by the present invention have broad application prospects in the preparation of drugs with sedative, hypnotic and/or anesthetic effects, as well as drugs capable of controlling status epilepticus, providing a new choice for clinically preparing drugs with both analgesic effects and sedative, hypnotic and/or anesthetic effects, and for controlling status epilepticus, etc.

1. A compound of formula IA, or a stereoisomer thereof, a pharmaceutically acceptable salt thereof, a solvate thereof, a prodrug thereof, a metabolite thereof, or a deuterated derivative thereof: 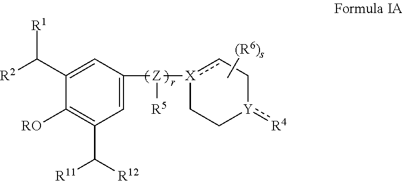
wherein, R is selected from hydrogen, COR^{a}, COCH(NH₂)R^{a1}, COOR^{h1}, (CH₂COO)ᵥR^{h1}, PO(OR^{h1})(OR^{h2}) or CH₂OPO(OR^{h1})(OR^{h2}); R^{a} and R^{a1} are each independently selected from hydrogen, C₁₋₄ alkyl, C₂₋₄ alkenyl, C₂₋₄ alkynyl, 3-6 membered cycloalkyl, 3-6 membered heterocyclyl, aryl or heteroaryl; R^{h1}, R^{h2} are each independently selected from hydrogen, C₁₋₄ alkyl, 3-6 membered cycloalkyl, 3-6 membered heterocyclyl, aryl or heteroaryl; v is an integer from 0-2;
The bond [symbol] connecting X can be a double bond or a single bond; when the bond [symbol] connecting X is a double bond, X is selected from C; when the bond [symbol] connecting X is a single bond, X is selected from CR³ and N;
The bond [symbol] connecting Y can be a double bond or a single bond; when the bond [symbol] connecting Y is a double bond, Y is selected from C; when the bond [symbol] connecting Y is a single bond, Y is selected from CR³ and N;
Z is selected from none, CR^{5′}, N, O;
R¹, R², R¹¹ and R¹² are each independently selected from hydrogen, halogen, C₁₋₄ alkyl, C₁₋₄ alkenyl or 3-6 membered cycloalkyl;
s is selected from an integer from 0 to 8, and each R⁶ is independently selected from hydrogen, C₁₋₄ alkyl, halogenated C₁₋₄ alkyl, C₂₋₄ alkenyl, C₂₋₄ alkynyl, 3-6 membered cycloalkyl, 3-6 membered heterocyclyl, halogen, C₁₋₄ alkoxy, CN, NO₂, OR^{f}, SR^{f}, NR^{d1}R^{d2}, CORⁱ, (CR^{a1}R^{a2})ₘCOR^{b}; or, s is selected from an integer from 2 to 8, wherein two R⁶ are bounded form a ring, and the remaining R⁶ is each independently selected from hydrogen, C₁₋₄ alkyl, halogenated C₁₋₄ alkyl, halogen, C₁₋₄ alkoxy, CN, NO₂, OR^{f}, SR^{f}, NR^{d1}R^{d2}, CORⁱ, (CR^{a1}R^{a2})ₘCOR^{b};
R⁵ and R^{5′} are each independently selected from none, hydrogen, C₁₋₈ alkyl, (CR^{a1}R^{a2})ₘCOR^{b};
R³ is hydrogen, (CR^{a1}R^{a2})ₘCOOR^{b1}, or groups including C₁₋₄ alkyl, C₂₋₄ alkonyl, C₂₋₄ alkynyl, 3-6 membered cycloalkyl, 3-6 membered heterocyclyl, aryl or heteroaryl unsubstituted or optionally substituted by one or more R^{x}; wherein R^{x} is each independently selected from cyano, nitro, hydroxyl, carboxyl, halogen, C₁₋₄ alkyl, C₁₋₄ alkoxy, (CR^{a1}R^{a2})ₘNR^{d1}R^{d2}, 3-8 membered cycloalkyl or its halogenated derivatives, 3-8 membered heterocyclyl or its halogenated derivatives, aryl or its halogenated or deuterated derivatives, heteroaryl or its halogenated or deuterated derivatives;
R⁴ is hydrogen, 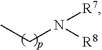
(CR^{a1}R^{a2})ₘCOOR^{b1}, (CR^{a1}R^{a2})ₘOCOR^{b}, (CR^{a1}R^{a2})ₙCOR^{c}, CONR^{d1}R^{d2}, (CR^{a1}R^{a2})ₙOR^{f};
when Z is none, X is N, Y is N, R⁴ is 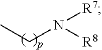
R⁷ is hydrogen or groups consisting of C₁₋₄ alkyl, 3-6 membered cycloalkyl, 3-6 membered heterocyclyl, aryl or heteroaryl unsubstituted or optionally substituted by one or more R^{y}; wherein R^{y} is each independently selected from cyano, nitro, hydroxyl, carboxyl, halo, C₁₋₄ alkyl, C₁₋₄ alkoxy, C₁₋₄ alkylthio, O(CR^{a1}R^{a2})ₘNR^{d1}R^{d2}, (CR^{a1}R^{a2})ₘNR^{d1}R^{d2}, 3-8 membered cycloalkyl or its halogenated derivatives, 3-8 membered heterocyclyl or its halogenated derivatives, aryl or its halogenated or deuterated derivatives, heteroaryl or its halogenated or deuterated derivatives;
R⁸ is hydrogen, C₁₋₄ alkyl, (CR^{a1}R^{a2})ₘCOOR^{b1}, (CR^{a1}R^{a2})ₘOCOR^{b}, (CR^{a1}R^{a2})ₙCOR^{c}, CONR^{d1}R^{d2}, (CR^{a1}R^{a2})ₙOR^{f}, (CR^{a1}R^{a2})ₘSO₂OR^{b1}, (CR^{a1}R^{a2})ₘSOOR^{b1};
wherein, R^{a1}, R^{a2} are each independently selected from hydrogen, halogen, C₁₋₄ alkyl, halogenated C₁₋₄ alkyl, C₂₋₄ alkenyl, C₂₋₄ alkynyl, 3-6 membered cycloalkyl, 3-6 membered heterocyclyl, aryl, heteroaryl, CN, OR^{f}, SR^{f}, or NR^{d1}R^{d2}; or, R^{a1} and R^{a2}, together with the carbon atom to which they are attached, form a 3-6 membered cyclic structure containing 0 or 1 heteroatom selected from N, O, or S;
wherein, R^{b1} is hydrogen, C₁₋₄ alkyl, C₁₋₄ alkoxy, halogenated C₁₋₄ alkyl, 3-6 membered cycloalkyl, 3-6 membered heterocyclyl, aryl, or heteroaryl;
R^{b} is hydrogen, C₁₋₄ alkyl, C₁₋₄ alkoxy, halogenated C₁₋₄ alkyl, C₂₋₄ alkenyl, C₂₋₄ alkynyl, 3-6 membered cycloalkyl, 3-6 membered heterocyclyl, aryl, or heteroaryl;
R^{c} is hydrogen, C₁₋₄ alkyl, C₁₋₄ alkoxy, C₂₋₄ alkenyl, C₂₋₄ alkynyl, 3-6 membered cycloalkyl, 3-6 membered heterocyclyl, aryl, or heteroaryl;
R^{d1} and R^{d2} are each independently selected from hydrogen, C₁₋₄ alkyl, C₁₋₄ alkoxy, 3-6 membered cycloalkyl, 3-6 membered heterocyclyl; or, R^{d1} and R^{d2} taken together with the nitrogen atom to which they are attached form a 3-6 membered ring containinng 1 or 2 heteroatom selected from N, O, or S;
wherein the alkyl, alkenyl, alkynyl, cycloalkyl, heterocyclyl, aryl, or heteroaryl in R^{a1}, R^{a2}, R^{d1}, R^{d2}, R^{b}, R^{b1}, R^{c}, R⁵ and R^{5′} are unsubstituted or optionally substituted by one or more groups selected from halogen, C₁₋₄ alkyl, C₁₋₄ alkoxy, halogenated C₁₋₄ alkyl, C₂₋₄ alkenyl, C₂₋₄ alkynyl, 3-6 membered cycloalkyl, 3-6 membered heterocyclyl, CN, NO₂, OR^{f}, SR^{f}, NR^{d1}R^{d2}, CORⁱ, COOR^{f} OCORⁱ, CONR^{d1}R^{d2}, NR^{d1}CORⁱ, NR^{d1}SO₂Rⁱ, or SO₂Rⁱ;
R^{f} is selected from hydrogen, C₁₋₄ alkyl, or halogenated C₁₋₄ alkyl;
Rⁱ is selected from hydrogen, C₁₋₄ alkyl, C₂₋₄ alkenyl, C₂₋₄ alkynyl, 3-6 membered cycloalkyl, or 3-6 membered heterocyclyl;
m is 0, 1, 2;
n is 0, 1, 2;
r is 0, 1, 2, 3;
p is 0, 1, 2, 3.

2. The compound according to claim 1, or a stereoisomer thereof, or a pharmaceutically acceptable salt thereof, or a solvate thereof, or a prodrug thereof, or a metabolite thereof, or a deuterated derivative thereof, characterized in that said compound has a structure of formula I: 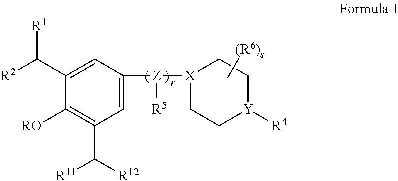
wherein, R is selected from hydrogen, COR^{a}, COCH(NH₂)R^{a1}, COOR^{h1}, (CH₂COO) (R^{h1} PO(OR^{h1})(OR^{h2}) or CH₂OPO(OR^{h1})(OR^{h2}); R^{a} and R^{a1} are each independently selected from hydrogen, C₁₋₄ alkyl, C₂₋₄ alkenyl, C₂₋₄ alkynyl, 3-6 membered cycloalkyl, 3-6 membered heterocyclyl, aryl or heteroaryl; R^{h1}, R^{h2} are each independently selected from hydrogen, C₁₋₄ alkyl, 3-6 membered cycloalkyl, 3-6 membered heterocyclyl, aryl or heteroaryl; v is an integer from 1 to 2;
X is selected from CR³, N;
Y is selected from CR³, N;
Z is selected from none, CR⁵′, N, O;
R¹, R², R¹¹ and R¹² are each independently selected from hydrogen, halogen, C₁₋₄ alkyl, C₂₋₄ alkenyl, or 3-6 membered cycloalkyl;
s is selected from an integer from 0 to 8, and each R^{e} is independently selected from hydrogen, C₁₋₄ alkyl, halogenated C₁₋₄ alkyl, C₂₋₄ alkenyl, C₂₋₄ alkynyl, 3-6 membered cycloalkyl, 3-6 membered heterocyclyl, halogen, C₁₋₄ alkoxy, CN, NO₂, OR^{f}, SR^{f}, NR^{d1}R^{d2}, CORⁱ, (CR^{a1}R^{a2})ₘCOR^{b}; or s is selected from an integer from 2 to 8, wherein two R⁶ groups are bounded form a ring, and the remaining Reis each independently selected from hydrogen, C₁₋₄ alkyl, halogenated C₁₋₄ alkyl, halo, C₁₋₄ alkoxy, CN, NO₂, OR^{f}, SR^{f}, NR^{d1}R^{d2}, CORⁱ, (CR^{a1}R^{a2})ₘCOR^{b};
R⁵ and R^{5′} are each independently selected from none, hydrogen, C₁₋₈ alkyl, (CR^{a1}R^{a2})ₘCOR^{b};
R³ is hydrogen, (CR^{a1}R^{a2})ₘCOOR^{b1}, or unsubstituted or optionally substituted by one or more R^{y} groups consisting of C₁₋₄ alkyl, C₂₋₄ alkenyl, C₂₋₄ alkynyl, 3-6 membered cycloalkyl, 3-6 membered heterocyclyl, aryl or heteroaryl; wherein R^{x} is each independently selected from cyano, nitro, hydroxyl, carboxy, halo, C₁₋₄ alkyl, C₁₋₄ alkoxy, (CR^{a1}R^{a2})ₘNR^{d1}R^{d2}, 3-8 membered cycloalkyl or its halogenated derivatives, 3-8 membered heterocyclyl or its halogenated derivatives, aryl or its halogenated or deuterated derivatives, heteroaryl or its halogenated or deuterated derivatives;
R⁴ is hydrogen, 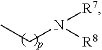
(CR^{a1}R^{a2})ₘCOOR^{b1}, (CR^{a1}R^{a2})ₘOCOR^{b}, (CR^{a1}R^{a2})ₙCOR^{c}, CONR^{d1}R^{d2}, (CR^{a1}R^{a2})ₙOR^{f};
when Z is none, X is N, Y is N, R⁴ is 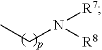
R⁷ is hydrogen, or groups consisting of C₁₋₄ alkyl, 3-6 membered cycloalkyl, 3-6 membered heterocyclyl, aryl or heteroaryl unsubstituted or optionally substituted with one or more R^{y}; wherein R^{y} is each independently selected from cyano, nitro, hydroxyl, carboxy, halo, C₁₋₄ alkyl, C₁₋₄ alkoxy, C₁₋₄ alkylthio, O(CR^{a1}R^{a2})ₘNR^{d1}R^{d2}, (CR^{a1}R^{a2})ₘNR^{d1}R^{d2}, 3-8 membered cycloalkyl or its halogenated derivatives, 3-8 membered heterocyclyl or its halogenated derivatives, aryl or its halogenated or deuterated derivatives, heteroaryl or its halogenated or deuterated derivatives;
R⁸ is hydrogen, C₁₋₄ alkyl, (CR^{a1}R^{a2})ₘCOOR^{b1}, (CR^{a1}R^{a2})ₘOCOR^{b}, (CR^{a1}R^{a2})ₙCOR^{c}, CONR^{d1}R^{d2}, (CR^{a1}R^{a2})ₙOR^{f}, (CR^{a1}R^{a2})ₘSO₂OR^{b1}, (CR^{a1}R^{a2})ₘSOOR^{b1};
wherein, R^{a1}, R^{a2} are each independently selected from hydrogen, halo, C₁₋₄ alkyl, halogenated C₁₋₄ alkyl, C₂₋₄ alkenyl, C₂₋₄ alkynyl, 3-6 membered cycloalkyl, 3-6 membered heterocyclyl, aryl, heteroaryl, CN, OR^{f}, SR^{f}, or NR^{d1}R^{d2}; or R^{a1} and R^{a2} taken together with the carbon atom to which they are attached, form a 3-6 membered ring containing 0 or 1 heteroatom selected from N, O, or S;
R^{b1} is hydrogen, C₁₋₄ alkyl, C₁₋₄ alkoxy, halogenated C₁₋₄ alkyl, 3-6 membered cycloalkyl, 3-6 membered heterocyclyl, aryl, or heteroaryl;
R^{b} is hydrogen, C₁₋₄ alkyl, C₁₋₄ alkoxy, halogenated C₁₋₄ alkyl, C₂₋₄ alkenyl, C₂₋₄ alkynyl, 3-6 membered cycloalkyl, 3-6 membered heterocyclyl, aryl, or heteroaryl;
R^{c} is hydrogen, C₁₋₄ alkyl, C₁₋₄ alkoxy, C₂₋₄ alkenyl, C₂₋₄ alkynyl, 3-6 membered cycloalkyl, 3-6 membered heterocyclyl, aryl, or heteroaryl;
R^{d1}, R^{d2} are each independently selected from hydrogen, C₁₋₄ alkyl, C₁₋₄ alkoxy, 3-6 membered cycloalkyl, 3-6 membered heterocyclyl; or, R^{d1} and R^{d2} taken together with the nitrogen atom to which they are attached, form a 3-6 membered ring containing 1 or 2 heteroatom selected from N, O, or S;
Wherein the alkyl, alkenyl, alkynyl, cycloalkyl, heterocyclyl, aryl, or heteroaryl in R^{a1}, R^{a2}, R^{d1}, R^{d2}, R^{b}, R^{b1}, R^{c}, R⁵ and R^{5′} are unsubstituted or optionally substituted by one or more groups selected from halogen, C₁₋₄ alkyl, C₁₋₄ alkoxy, halogenated C₁₋₄ alkyl, C₂₋₄ alkenyl, C₂₋₄ alkynyl, 3-6 membered cycloalkyl, 3-6 membered heterocyclyl, CN, NO₂, OR^{f}, SR^{f}, NR^{d1}R^{d2}, CORⁱ, COOR^{f}, OCORⁱ, CONR^{d1}R^{d2}, NR^{d1}CORⁱ, NR^{d1}SO₂Rⁱ, or SO₂Rⁱ;
R^{f} is selected from hydrogen, C₁₋₄ alkyl, or halogenated C₁₋₄ alkyl;
Rⁱ is selected from hydrogen, C₁₋₄ alkyl, C₂₋₄ alkenyl, C₂₋₄ alkynyl, 3-6 membered cycloalkyl, or 3-6 membered heterocyclyl;
m is 0, 1, 2;
n is 0, 1, 2;
r is 0, 1, 2, 3;
p is 0, 1, 2, 3.

3. The compound according to claim 2, or a stereoisomer thereof, or a pharmaceutically acceptable salt thereof, or a solvate thereof, or a prodrug thereof, or a metabolite thereof, or a deuterated derivative thereof, characterized in that said compound has a structure of formula II: 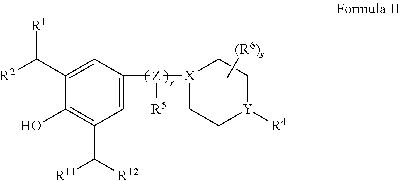
wherein, X is selected from CR³, N;
Y is selected from CR³, N;
Z is selected from none, CR⁵, N, O;
R¹, R², R¹, R¹² are each independently selected from hydrogen, halo, C₁₋₄ alkyl, C₂₋₄ alkenyl, or 3-6 membered cycloalkyl;
s is selected from an integer from 0 to 8, and each R⁶ is independently selected from hydrogen, C₁₋₄ alkyl, halogenated C₁₋₄ alkyl, C₂₋₄ alkenyl, C₂₋₄ alkynyl, 3-6 membered cycloalkyl, 3-6 membered heterocyclyl, halogen, C₁₋₄ alkoxy, CN, NO₂, OR^{f}, SR^{f}, NR^{d1}R^{d2}, CORⁱ, (CR^{a1}R^{a2})ₘCOR^{b}; or, s is selected from an integer from 2 to 8, wherein two R⁶ groups are bounded form a ring, and the remaining R⁶ each independently selected from hydrogen, C₁₋₄ alkyl, halogenated C₁₋₄ alkyl, halogen, C₁₋₄ alkoxy, CN, NO₂, OR^{f}, SR^{f}, NR^{d1}R^{d2}, CORⁱ, (CR^{a1}R^{a2})ₘCOR^{b};
R⁵ and R^{5′} are each independently selected from none, hydrogen, C₁₋₈ alkyl, (CR^{a1}R^{a2})ₘCOR^{b};
R³ is hydrogen, (CR^{a1}R^{a2})ₘCOOR^{b1}, unsubstituted or optionally substituted by one or more R^{a} groups consisting of C₁₋₄ alkyl, C₂₋₄ alkenyl, C₂₋₄ alkynyl, 3-6 membered cycloalkyl, 3-6 membered heterocyclyl, aryl or heteroaryl; wherein R^{y} is each independently selected from cyano, nitro, hydroxyl, carboxy, halo, C₁₋₄ alkyl, C₁₋₄ alkoxy, (CR^{a1}R^{a2})ₘNR^{d1}R^{d2}, 3-8 membered cycloalkyl or its halogenated derivatives, 3-8 membered heterocyclyl or its halogenated derivatives, aryl or its halogenated or deuterated derivatives, heteroaryl or its halogenated or deuterated derivatives;
R⁴ is hydrogen, 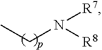
(CR^{a1}R^{a2})ₘCOOR^{b1}, (CR^{a1}R^{a2})ₘOCOR^{b1}, (CR^{a1}R^{a2})ₙCOR^{c}, CONR^{d1}R^{d2}, (CR^{a1}R^{a2})ₙOR^{f};
when Z is none, X is N, Y is N, R⁴ is 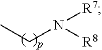
R⁷ is hydrogen, groups consisting of C₁₋₄ alkyl, 3-6 membered cycloalkyl, 3-6 membered heterocyclyl, aryl or heteroaryl unsubstituted or optionally substituted by one or more R^{y}; R^{y} is each independently selected from cyano, nitro, hydroxyl, carboxy, halogen, C₁₋₄ alkyl, alkoxy, C₁₋₄ Alkylthio, O(CR^{a1}R^{a2})ₘNR^{d1}R^{d2}, (CR^{a1}R^{a2})ₘNR^{d1}R^{d2}, 3-8 membered cycloalkyl or its halogen-substituted derivatives, 3-8 membered heterocyclyl or its halogen-substituted derivatives, aryl or its halogen-substituted or deuterated derivatives, heteroaryl or its halogen-substituted or deuterated derivatives;
R⁸ is hydrogen, C₁₋₄ alkyl, (CR^{a1}R^{a2})ₘCOOR^{b1}, (CR^{a1}R^{a2})ₘOCOR^{b}, (CR^{a1}R^{a2})ₙCOR^{c}, CONR^{d1}R^{d2}, (CR^{a1}R^{a2})ₙOR^{f}, (CR^{a1}R^{a2})ₘSO₂OR^{b1}, (CR^{a1}R^{a2})ₘSOOR^{b1};
wherein, R^{a1}, R^{a2} are each independently selected from hydrogen, halogen, C₁₋₄ alkyl, halogenated C₁₋₄ alkyl, C₂₋₄ alkenyl, C₂₋₄ alkynyl, 3-6 membered cycloalkyl, 3-6 membered heterocyclyl, aryl, heteroaryl, CN, OR^{f}, SR^{f}, or NR^{d1}R^{d2}; or, R^{a1} and R^{a2} taken together with the carbon atom to which they are attached form a 3-6 membered ring containing 0 or 1 heteroatom selected from N, O, or S;
R^{b1} is hydrogen, C₁₋₄ alkyl, C₁₋₄ alkoxy, halogenated C₁₋₄ alkyl, 3-6 membered cycloalkyl, 3-6 membered heterocyclyl, aryl, or heteroaryl;
R^{b} is hydrogen, C₁₋₄ alkyl, C₁₋₄ alkoxy, halogenated C₁₋₄ alkyl, C₂₋₄ alkenyl, C₂₋₄ alkynyl, 3-6 membered cycloalkyl, 3-6 membered heterocyclyl, aryl, or heteroaryl;
R^{c} is hydrogen, C₁₋₄ alkyl, C₁₋₄ alkoxy, C₂₋₄ alkenyl, C₂₋₄ alkynyl, 3-6 membered cycloalkyl, 3-6 membered heterocyclyl, aryl, or heteroaryl;
R^{d1}, R^{d2} are each independently selected from hydrogen, C₁₋₄ alkyl, C₁₋₄ alkoxy, 3-6 membered cycloalkyl, 3-6 membered heterocyclyl; or, R^{d1} and R^{d2} taken together with the nitrogen atom to which they are attached form a 3-6 membered ring containing 1 or 2 heteroatom selected from N, O, or S;
Wherein the alkyl, alkenyl, alkynyl, cycloalkyl, heterocyclyl, aryl, or heteroaryl in R^{a1}, R^{a2}, R^{d1}, R^{d2}, R^{b}, R^{b1}, R^{c}, R⁵ and R^{5′} are unsubstituted or optionally substituted by one or more groups consisting of halogen, C₁₋₄ alkyl, C₁₋₄ alkoxy, halogenated C₁₋₄ alkyl, C₂₋₄ alkenyl, C₂₋₄ alkynyl, 3-6 membered cycloalkyl, 3-6 membered heterocyclyl, CN, NO₂, OR^{f}, SR^{f}, NR^{d1}R^{d2}, CORⁱ, COOR^{f}, OCORⁱ, CONR^{d1}R^{d2}, NR^{d1}CORⁱ, NR^{d1}SO₂Rⁱ, or SO₂Rⁱ;
R^{f} is selected from hydrogen, C₁₋₄ alkyl, or halogenated C₁₋₄ alkyl;
Rⁱ is selected from hydrogen, C₁₋₄ alkyl, C₂₋₄ alkenyl, C₂₋₄ alkynyl, 3-6 membered cycloalkyl, or 3-6 membered heterocyclyl;
m is selected from 0, 1, 2;
n is selected from 0, 1, 2;
r is selected from 0, 1, 2, 3;
p is selected from 0, 1, 2, 3.

4. The compound according to claim 3, or a stereoisomer thereof, or a pharmaceutically acceptable salt thereof, or a solvate thereof, or a prodrug thereof, or a metabolite thereof, or a deuterated derivative thereof, characterized in that said compound has a structure of formula IIa: 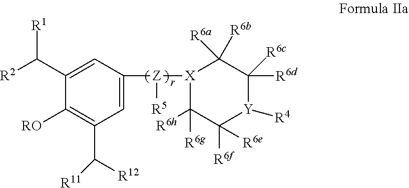
wherein, X is selected from CR³, N;
Y is selected from CR³, N;
Z is selected from none, CR⁵, N, O;
R¹, R², R¹¹, R¹² are each independently selected from hydrogen, halogen, C₁₋₄ alkyl, C₂₋₄ alkenyl, or 3-6 membered cycloalkyl;
R^{6a}, R^{6b}, R^{6c}, R^{6d}, R^{6e}, R^{6f}, R^{6g}, R^{6h} are each independently selected from hydrogen, C₁₋₄ alkyl, halogenated C₁₋₄ alkyl, C₂₋₄ alkenyl, C₂₋₄ alkynyl, 3-6 membered cycloalkyl, 3-6 membered heterocyclyl, halogen, C₁₋₄ alkoxy, CN, NO₂, OR^{f}, SR^{f}, NR^{d1}R^{d2}, CORⁱ, (CR^{a1}R^{a2})ₘCOR^{b}; or, any two groups among R^{6a}, R^{6b}, R^{6c}, R^{6d}, R^{6e}, R^{6f}, R^{6g}, R^{6h} cyclize to form a spiro ring, bridged ring, or fused ring, when the remaining groups are each independently selected from hydrogen, C₁₋₄ alkyl, halogenated C₁₋₄ alkyl, halogen, C₁₋₄ alkoxy, CN, NO₂, OR^{f}, SR^{f}, NR^{d1}R^{d2}, CORⁱ, (CR^{a1}R^{a2})mCOR^{b},
R⁵ and R^{5′} are each independently selected from none, hydrogen, C₁₋₈ alkyl, (CR^{a1}R^{a2})ₘCOR^{b};
R³ is hydrogen, (CR^{a1}R^{a2})mCOOR^{b1}, or groups consisting of C₁₋₄ alkyl, C₂₋₄ alkenyl, C₂₋₄ alkynyl, 3-6 membered cycloalkyl, 3-6 membered heterocyclyl, aryl or heteroaryl unsubstituted or optionally substituted by one or more R^{x}, wherein R^{x} is each independently selected from cyano, nitro, hydroxyl, carboxy, halogen, C₁₋₄ alkyl, C₁₋₄ alkoxy, (CR^{a1}R^{a2})ₘNR^{d1}R^{d2}, 3-8 membered cycloalkyl or its halogenated derivatives, 3-8 membered heterocyclyl or its halogenated derivatives, aryl or its halogenated or deuterated derivatives, heteroaryl or its halogenated or deuterated derivatives;
R⁴ is hydrogen, 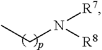
(CR^{a1}R^{a2})ₘCOOR^{b1}, (CR^{a1}R^{a2})ₘOCOR^{b}, (CR^{a1}R^{a2})ₙCOR^{c}, CONR^{d1}R^{d2}, (CR^{a1}R^{a2})ₙOR^{f};
when Z is none, X is N, Y is N, R⁴ is 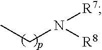
R⁷ is hydrogen, or groups consisting of C₁₋₄ alkyl, 3-6 membered cycloalkyl, 3-6 membered heterocyclyl, aryl or heteroaryl unsubstituted or optionally substituted with one or more R^{y}; wherein R^{y} is each independently selected from cyano, nitro, hydroxyl, carboxy, halogen, C₁₋₄ alkyl, C₁₋₄ alkoxy, C₁₋₄ alkylthio, O(CR^{a1}R^{a2})ₘNR^{d1}R^{d2}, (CR^{a1}R^{a2})ₘNR^{d1}R^{d2}, 3-8 membered cycloalkyl or its halogenated derivatives, 3-8 membered heterocyclyl or its halogenated derivatives, aryl or its halogenated or deuterated derivatives, heteroaryl or its halogenated or deuterated derivatives;
R⁸ is hydrogen, C₁₋₄ alkyl, (CR^{a1}R^{a2})ₘCOOR^{b1}, (CR^{a1}R^{a2})ₘOCOR^{b}, (CR^{a1}R^{a2})ₙCOR^{c}, CONR^{d1}R^{d2}, (CR^{a1}R^{a2})ₙOR^{f}, (CR^{a1}R^{a2})ₘSO₂OR^{b1}, (CR^{a1}R^{a2})ₘSOOR^{b1};
wherein, R^{a1}, R^{a2} are each independently selected from hydrogen, halogen, C₁₋₄ alkyl, halogenated C₁₋₄ alkyl, C₂₋₄ alkenyl, C₂₋₄ alkynyl, 3-6 membered cycloalkyl, 3-6 membered heterocyclyl, aryl, heteroaryl, CN, OR^{f}, SR^{f}, or NR^{d1}R^{d2}; or, R^{a1} and R^{a2} taken together with the carbon atom to which they are attached form a 3-6 membered ring containing 0 or 1 heteroatom selected from N, O, or S;
R^{b1} is hydrogen, C₁₋₄ alkyl, C₁₋₄ alkoxy, halogenated C₁₋₄ alkyl, 3-6 membered cycloalkyl, 3-6 membered heterocyclyl, aryl, or heteroaryl;
R^{b} is hydrogen, C₁₋₄ alkyl, C₁₋₄ alkoxy, halogenated C₁₋₄ alkyl, C₂₋₄ alkenyl, C₂₋₄ alkynyl, 3-6 membered cycloalkyl, 3-6 membered heterocyclyl, aryl, or heteroaryl;
R^{c} is hydrogen, C₁₋₄ alkyl, C₁₋₄ alkoxy, C₂₋₄ alkenyl, C₂₋₄ alkynyl, 3-6 membered cycloalkyl, 3-6 membered heterocyclyl, aryl, or heteroaryl;
R^{d1}, R^{d2} are each independently selected from hydrogen, C₁₋₄ alkyl, C₁₋₄ alkoxy, 3-6 membered cycloalkyl, 3-6 membered heterocyclyl; or, R^{d1} and R^{d2} taken together with the nitrogen atom to which they are attached, form a 3-6 membered ring containing 1 or 2 heteroatom selected from N, O, or S;
Wherein the alkyl, alkenyl, alkynyl, cycloalkyl, heterocyclyl, aryl, or heteroaryl in R^{a1}, R^{a2}, R^{d1}, R^{d2}, R^{b}, R^{b1}, R^{c}, R⁵ and R^{5′} are unsubstituted or optionally substituted by one or more groups selected from halogen, C₁₋₄ alkyl, C₁₋₄ alkoxy, halogenated C₁₋₄ alkyl, C₂₋₄ alkenyl, C₂₋₄ alkynyl, 3-6 membered cycloalkyl, 3-6 membered heterocyclyl, CN, NO₂, OR^{f}, SR^{f}, NR^{d1}R^{d2}, CORⁱ, COOR^{f}, OCORⁱ, CONR^{d1}R^{d2}, NR^{d1}CORⁱ, NR^{d1}SO₂Rⁱ, or SO₂Rⁱ;
R^{f} is selected from hydrogen, C₁₋₄ alkyl, or halogenated C₁₋₄ alkyl;
Rⁱ is selected from hydrogen, C₁₋₄ alkyl, C₂₋₄ alkenyl, C₂₋₄ alkynyl, 3-6 membered cycloalkyl, or 3-6 membered heterocyclyl;
m is selected from 0, 1, 2;
n is selected from 0, 1, 2;
r is selected from 0, 1, 2, 3;
p is selected from 0, 1, 2, 3.

5. The compound according to claim 4, or a stereoisomer thereof, or a pharmaceutically acceptable salt thereof, or a solvate thereof, or a prodrug thereof, or a metabolite thereof, or a deuterated derivative thereof, characterized in that said 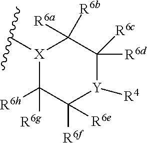
structural fragment is one of the following structures: 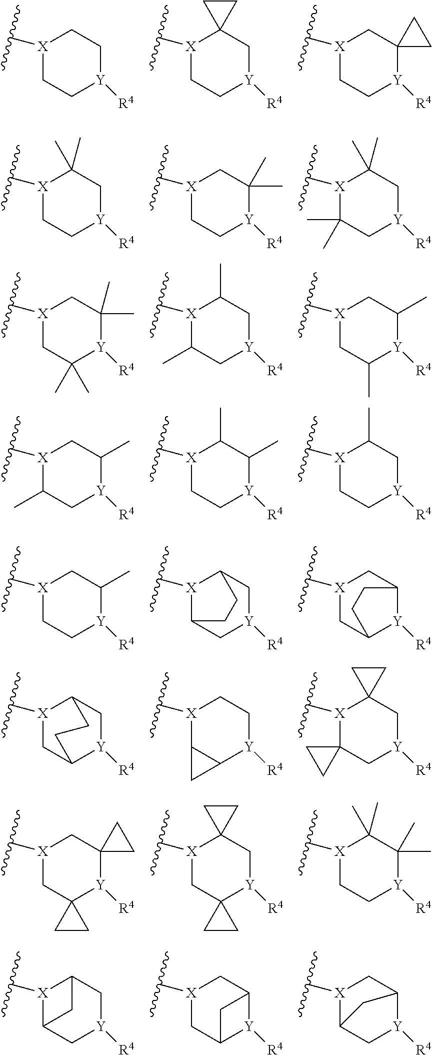
X, Y, R⁴ are as described in claim 4.

6. The compound according to claim 3, or a stereoisomer thereof, or a pharmaceutically acceptable salt thereof, or a solvate thereof, or a prodrug thereof, or a metabolite thereof, or a deuterated derivative thereof, characterized in that said compound has a structure of formula III: 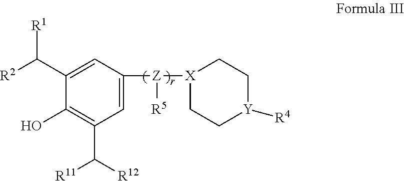
wherein,
X is selected from CR³, N;
Y is selected from CR³, N;
Z is selected from none, CR⁵, N, O;
R¹, R², R¹¹, R¹² are each independently selected from hydrogen, halogen, C₁₋₄ alkyl, C₂₋₄ alkenyl, or 3-6 membered cycloalkyl;
R⁵ and R^{5′} are each independently selected from none, hydrogen, C₁₋₈ alkyl, (CR^{a1}R^{a2})ₘCOR^{b},
R³ is hydrogen, (CR^{a1}R^{a2})mCOOR^{b1}, or groups consisiting of C₁₋₄ alkyl, C₂₋₄ alkenyl, C₂₋₄ alkynyl, 3-6 membered cycloalkyl, 3-6 membered heterocyclyl, aryl or heteroaryl unsubstituted or optionally substituted by one or more R^{x}; wherein R^{x} is each independently selected from cyano, nitro, hydroxyl, carboxy, halogen, C₁₋₄ alkyl, C₁₋₄ alkoxy, (CR^{a1}R^{a2})ₘNR^{d1}R^{d2}, 3-8 membered cycloalkyl or its halogenated derivatives, 3-8 membered heterocyclyl or its halogenated derivatives, aryl or its halogenated or deuterated derivatives, heteroaryl or its halogenated or deuterated derivatives;
R⁴ is hydrogen, 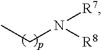
(CR^{a1}R^{a2})ₘCOOR^{b1}, (CR^{a1}R^{a2})ₘOCOR^{b}, (CR^{a1}R^{a2})ₙCOR^{c}, CONR^{d1}R^{d2}, (CR^{a1}R^{a2})ₙOR^{f};
when Z is none, X is N, Y is N, R⁴ is 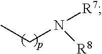
R⁷ is hydrogen, or groups consisting of Ct-4 alkyl, 3-6 membered cycloalkyl, 3-6 membered heterocyclyl, aryl or heteroaryl unsubstituted or optionally substituted by one or more R^{y}, wherein R^{y} is each independently selected from cyano, nitro, hydroxyl, carboxy, halogen, C₁₋₄ alkyl, C₁₋₄ alkoxy, C₁₋₄ alkylthio, O(CR^{a1}R^{a2})ₘNR^{d1}R^{d2}, (CR^{a1}R^{a2})ₘNR^{d1}R^{d2}, 3-8 membered cycloalkyl or its halogenated derivatives, 3-8 membered heterocyclyl or its halogenated derivatives, aryl or its halogenated or deuterated derivatives, heteroaryl or its halogenated or deuterated derivatives;
R⁸ is hydrogen, C₁₋₄ alkyl, (CR^{a1}R^{a2})ₘCOOR^{b1}, (CR^{a1}R^{a2})ₘOCOR^{b}, (CR^{a1}R^{a2})ₙCOR^{c}, CONR^{d1}R^{d2}, (CR^{a1}R^{a2})ₙOR^{f}, (CR^{a1}R^{a2})ₘSO₂OR^{b1}, (CR^{a1}R^{a2})ₘSOOR^{b1};
wherein, R^{a1}, R^{a2} are each independently selected from hydrogen, halogen, C₁₋₄ alkyl, halogenated C₁₋₄ alkyl, C₂₋₄ alkenyl, C₂₋₄ alkynyl, 3-6 membered cycloalkyl, 3-6 membered heterocyclyl, aryl, heteroaryl, CN, OR, SR^{f}, or NR^{d1}R^{d2}; or, R^{a1} and R^{a2} taken together with the carbon atom to which they are attached, form a 3-6 membered ring containing 0 or 1 heteroatom selected from N, O, or S;
R^{b1} is hydrogen, C₁₋₄ alkyl, C₁₋₄ alkoxy, halogenated C₁₋₄ alkyl, 3-6 membered cycloalkyl, 3-6 membered heterocyclyl, aryl, or heteroaryl;
R^{b} is hydrogen, C₁₋₄ alkyl, C₁₋₄ alkoxy, halogenated C₁₋₄ alkyl, C₂₋₄ alkenyl, C₂₋₄ alkynyl, 3-6 membered cycloalkyl, 3-6 membered heterocyclyl, aryl, or heteroaryl;
R^{c} is hydrogen, C₁₋₄ alkyl, C₁₋₄ alkoxy, C₂₋₄ alkenyl, C₂₋₄ alkynyl, 3-6 membered cycloalkyl, 3-6 membered heterocyclyl, aryl, or heteroaryl;
R^{d1}, R^{d2} are each independently selected from hydrogen, C₁₋₄ alkyl, C₁₋₄ alkoxy, 3-6 membered cycloalkyl, 3-6 membered heterocyclyl; or, R^{d1} and R^{d2} taken together with the nitrogen atom to which they are attached, form a 3-6 membered ring containing 1 or 2 heteroatom selected from N, O, or S;
Wherein the alkyl, alkenyl, alkynyl, cycloalkyl, heterocyclyl, aryl, or heteroaryl in R^{a1}, R^{a2}, R^{d1}, R^{d2}, R^{b}, R^{b1}, R^{c}, R⁵ and R^{5′} are unsubstituted or optionally substituted by one or more groups selected from halogen, C₁₋₄ alkyl, C₁₋₄ alkoxy, halogenated C₁₋₄ alkyl, C₂₋₄ alkenyl, C₂₋₄ alkynyl, 3-6 membered cycloalkyl, 3-6 membered heterocyclyl, CN, NO₂, OR^{f}, SR^{f}, NR^{d1}R^{d2}, CORⁱ, COOR^{f}, OCORⁱ, CONR^{d1}R^{d2}, NR^{d1}CORⁱ, NR^{d1}SO₂Rⁱ, or SO₂Rⁱ;
R^{f} is selected from hydrogen, C₁₋₄ alkyl, or halogenated C₁₋₄ alkyl;
Rⁱ is selected from hydrogen, C₁₋₄ alkyl, C₂₋₄ alkenyl, C₂₋₄ alkynyl, 3-6 membered cycloalkyl, or 3-6 membered heterocyclyl;
m is selected from 0, 1, 2;
n is selected from 0, 1, 2;
r is selected from 0, 1, 2, 3;
p is selected from 0, 1, 2, 3.

7. The compound according to claim 6, or a stereoisomer thereof, or a pharmaceutically acceptable salt thereof, or a solvate thereof, or a prodrug thereof, or a metabolite thereof, or a deuterated derivative thereof, characterized in that said compound has a structure of formula IV: 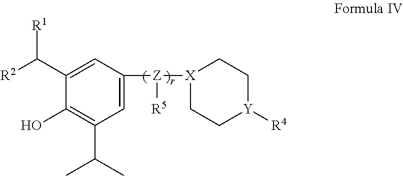
wherein, X is selected from CR³, N;
Y is selected from CR³, N;
Z is selected from none, CR^{5′}, N, O;
R¹, R² are each independently selected from hydrogen, halogen, C₁₋₄ alkyl, C₂₋₄ alkenyl, or 3-6 membered cycloalkyl;
R⁵ and R^{5′} are each independently selected from none, hydrogen, C₁₋₈ alkyl, (CR^{a1}R^{a2})ₘCOR^{b};
R³ is hydrogen, (CR^{a1}R^{a2})ₘCOOR^{b1}, or groups consisting of C₁₋₄ alkyl, C₂₋₄ alkenyl, C₂₋₄ alkynyl, 3-6 membered cycloalkyl, 3-6 membered heterocyclyl, aryl or heteroaryl unsubstituted or optionally substituted by one or more R^{x}, wherein R^{x} is each independently selected from cyano, nitro, hydroxyl, carboxy, halogen, C₁₋₄ alkyl, C₁₋₄ alkoxy, (CR^{a1}R^{a2})ₘNR^{d1}R^{d2}, 3-8 membered cycloalkyl or its halogenated derivatives, 3-8 membered heterocyclyl or its halogenated derivatives, aryl or its halogenated or deuterated derivatives, heteroaryl or its halogenated or deuterated derivatives;
R⁴ is hydrogen, 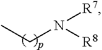
(CR^{a1}R^{a2})ₘCOOR^{b1}, (CR^{a1}R^{a2})ₘOCOR^{b}, (CR^{a1}R^{a2})ₙCOR^{c}, CONR^{d1}R^{d2}, (CR^{a1}R^{a2})ₙOR^{f};
when Z is none, X is N, Y is N, R⁴ is 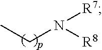
R⁷ is hydrogen, or groups consisting of C₁₋₄ alkyl, 3-6 membered cycloalkyl, 3-6 membered heterocyclyl, aryl or heteroaryl unsubstituted or optionally substituted by one or more R^{y}; wherein R^{y} is each independently selected from cyano, nitro, hydroxyl, carboxy, halogen, C₁₋₄ alkyl, C₁₋₄ alkoxy, C₁₋₄ alkylthio, O(CR^{a1}R^{a2})ₘNR^{d1}R^{d2}, (CR^{a1}R^{a2})ₘNR^{d1}R^{d2}, 3-8 membered cycloalkyl or its halogenated derivatives, 3-8 membered heterocyclyl or its halogenated derivatives, aryl or its halogenated or deuterated derivatives, heteroaryl or its halogenated or deuterated derivatives;
R⁸ is hydrogen, C₁₋₄ alkyl, (CR^{a1}R^{a2})ₘCOOR^{b1}, (CR^{a1}R^{a2})ₘOCOR^{b}, (CR^{a1}R^{a2})ₙCOR^{c}, CONR^{d1}R^{d2}, (CR^{a1}R^{a2})ₙOR^{f}, (CR^{a1}R^{a2})ₘSO₂OR^{b1}, (CR^{a1}R^{a2})ₘSOOR^{b1};
wherein, R^{a1}, R^{a2} are each independently selected from hydrogen, halogen, C₁₋₄ alkyl, halogenated C₁₋₄ alkyl, C₂₋₄ alkenyl, C₂₋₄ alkynyl, 3-6 membered cycloalkyl, 3-6 membered heterocyclyl, aryl, heteroaryl, CN, OR^{f}, SR^{f}, or NR^{d1}R^{d2}; or, R^{a1} and R^{a2} taken together with the carbon atom to which they are attached, form a 3-6 membered ring containing 0 or 1 heteroatom selected from N, O, or S;
R^{b1} is hydrogen, C₁₋₄ alkyl, C₁₋₄ alkoxy, halogenated C₁₋₄ alkyl, 3-6 membered cycloalkyl, 3-6 membered heterocyclyl, aryl, or heteroaryl;
R^{b} is hydrogen, C₁₋₄ alkyl, C₁₋₄ alkoxy, halogenated C₁₋₄ alkyl, C₂₋₄ alkenyl, C₂₋₄ alkynyl, 3-6 membered cycloalkyl, 3-6 membered heterocyclyl, aryl, or heteroaryl;
R^{c} is hydrogen, C₁₋₄ alkyl, C₁₋₄ alkoxy, C₂₋₄ alkenyl, C₂₋₄ alkynyl, 3-6 membered cycloalkyl, 3-6 membered heterocyclyl, aryl, or heteroaryl;
R^{d1}, R^{d2} are each independently selected from hydrogen, C₁₋₄ alkyl, C₁₋₄ alkoxy, 3-6 membered cycloalkyl, 3-6 membered heterocyclyl; or R^{d1} and R^{d2} taken together with the nitrogen atom to which they are attached, form a 3-6 membered ring containing 1 or 2 heteroatom selected from N, O, or S;
Wherein the alkyl, alkenyl, alkynyl, cycloalkyl, heterocyclyl, aryl, or heteroaryl in R^{a1}, R^{a2}, R^{d1}, R^{d2}, R^{b}, R^{b1}, R^{c}, R⁵ and R^{5′} are unsubstituted or optionally substituted by one or more groups selected from halogen, C₁₋₄ alkyl, C₁₋₄ alkoxy, halogenated C₁₋₄ alkyl, C₂₋₄ alkenyl, C₂₋₄ alkynyl, 3-6 membered cycloalkyl, 3-6 membered heterocyclyl, CN, NO₂, OR^{f}, SR^{f}, NR^{d1}R^{d2}, CORⁱ, COOR^{f}, OCORⁱ, CONR^{d1}R^{d2}, NR^{d1}CORⁱ, NR^{d1}SO₂Rⁱ, or SO₂Rⁱ;
R^{f} is selected from hydrogen, C₁₋₄ alkyl, or halogenated C₁₋₄ alkyl;
Rⁱ is selected from hydrogen, C₁₋₄ alkyl, C₂₋₄ alkenyl, C₂₋₄ alkynyl, 3-6 membered cycloalkyl, or 3-6 membered heterocyclyl;
m is selected from 0, 1, 2;
n is selected from 0, 1, 2;
r is selected from 0, 1, 2, 3;
p is selected from 0, 1, 2, 3.

8. The compound according to claim 7, or a stereoisomer thereof, or a pharmaceutically acceptable salt thereof, or a solvate thereof, or a prodrug thereof, or a metabolite thereof, or a deuterated derivative thereof, characterized in that said compound has a structure of formula V-1 or formula V-2: 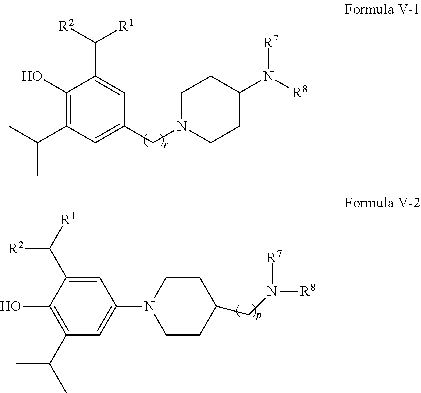
wherein,
R¹, R² are each independently selected from hydrogen, halogen, C₁₋₄ alkyl, C₂₋₄ alkenyl, or 3-6 membered cycloalkyl;
R⁷ is hydrogen, or groups consisting of C₁₋₄ alkyl, 3-6 membered cycloalkyl, 3-6 membered heterocyclyl, aryl or heteroaryl unsubstituted or optionally substituted by one or more R^{y}; wherein R^{y} is each independently selected from cyano, nitro, hydroxyl, carboxy, halogen, C₁₋₄ alkyl, C₁₋₄ alkoxy, C₁₋₄ alkylthio, O(CR^{a1}R^{a2})ₘNR^{d1}R^{d2}, (CR^{a1}R^{a2})ₘNR^{d1}R^{d2}, 3-8 membered cycloalkyl or its halogenated derivatives, 3-8 membered heterocyclyl or its halogenated derivatives, aryl or its halogenated or deuterated derivatives, heteroaryl or its halogenated or deuterated derivatives;
R⁸ is hydrogen, C₁₋₄ alkyl, (CR^{a1}R^{a2})ₘCOOR^{b1}, (CR^{a1}R^{a2})ₘOCOR^{b}, (CR^{a1}R^{a2})ₙCOR^{c}, CONR^{d1}R^{d2}, (CR^{a1}R^{a2})ₙOR^{f}, (CR^{a1}R^{a2})ₘSO₂OR^{b1}, (CR^{a1}R^{a2})ₘSOOR^{b1};
wherein, R^{a1}, R^{a2} are each independently selected from hydrogen, halogen, C₁₋₄ alkyl, halogenated C₁₋₄ alkyl, C₂₋₄ alkenyl, C₂₋₄ alkynyl, 3-6 membered cycloalkyl, 3-6 membered heterocyclyl, aryl, heteroaryl, CN, OR^{f}, SR^{f}, or NR^{d1}R^{d2}; or, R^{a1} and R^{a2} taken together with the carbon atom to which they are attached, form a 3-6 membered ring containing 0 or 1 heteroatom selected from N, O, or S;
R^{b1} is hydrogen, C₁₋₄ alkyl, C₁₋₄ alkoxy, halogenated C₁₋₄ alkyl, 3-6 membered cycloalkyl, 3-6 membered heterocyclyl, aryl, or heteroaryl;
R^{b} is hydrogen, C₁₋₄ alkyl, C₁₋₄ alkoxy, halogenated C₁₋₄ alkyl, C₂₋₄ alkenyl, C₂₋₄ alkynyl, 3-6 membered cycloalkyl, 3-6 membered heterocyclyl, aryl, or heteroaryl;
R^{c} is hydrogen, C₁₋₄ alkyl, C₁₋₄ alkoxy, C₂₋₄ alkenyl, C₂₋₄ alkynyl, 3-6 membered cycloalkyl, 3-6 membered heterocyclyl, aryl, or heteroaryl;
R^{d1}, R^{d2} are each independently selected from hydrogen, C₁₋₄ alkyl, C₁₋₄ alkoxy, 3-6 membered cycloalkyl, 3-6 membered heterocyclyl; or, R^{d1} and R^{d2} taken together with the nitrogen atom to which they are attached, form a 3-6 membered ring containing 1 or 2 heteroatom selected from N, O, or S;
Wherein the alkyl, alkenyl, alkynyl, cycloalkyl, heterocyclyl, aryl, or heteroaryl in R^{a1}, R^{a2}, R^{d1}, R^{d2}, R^{b}, R^{b1} and R^{c} are unsubstituted or optionally substituted by one or more groups selected from halogen, C₁₋₄ alkyl, C₁₋₄ alkoxy, halogenated C₁₋₄ alkyl, C₂₋₄ alkenyl, C₂₋₄ alkynyl, 3-6 membered cycloalkyl, 3-6 membered heterocyclyl, CN, NO₂, OR^{f}, SR^{f}, NR^{d1}R^{d2}, CORⁱ, COOR^{f}, OCORⁱ, CONR^{d1}R^{d2}, NR^{d1}CORⁱ, NR^{d1}SO₂Rⁱ, or SO₂Rⁱ,
R^{f} is selected from hydrogen, C₁₋₄ alkyl or halogenated C₁₋₄ alkyl;
Rⁱ is selected from hydrogen, C₁₋₄ alkyl, C₂₋₄ alkenyl, C₂₋₄ alkynyl, 3-6 membered cycloalkyl, or 3-6 membered heterocyclyl;
m is selected from 0, 1, 2;
n is selected from 0, 1, 2;
r is selected from 0, 1, 2, 3;
p is selected from 0, 1, 2, 3.

9. The compound according to claim 7, or a stereoisomer thereof, or a pharmaceutically acceptable salt thereof, or a solvate thereof, or a prodrug thereof, or a metabolite thereof, or a deuterated derivative thereof, characterized in that said compound has a structure of formula V-3: 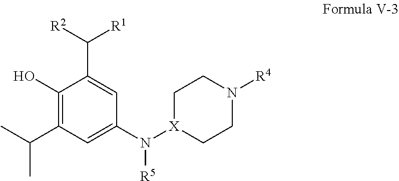
wherein,
X is selected from CR³, N;
R¹, R² are each independently selected from hydrogen, halogen, C₁₋₄ alkyl, C₂₋₄ alkenyl, or 3-6 membered cycloalkyl;
R⁵ is selected from none, hydrogen, C₁₋₈ alkyl, (CR^{a1}R^{a2})ₘCOR^{b};
R³ is hydrogen, (CR^{a1}R^{a2})ₘCOOR^{b1}, or groups consisting of C₁₋₄ alkyl, C₂₋₄ alkenyl, C₂₋₄ alkynyl, 3-6 membered cycloalkyl, 3-6 membered heterocyclyl, aryl or heteroaryl unsubstituted or optionally substituted by one or more R^{x}; wherein R^{x} is each independently selected from cyano, nitro, hydroxyl, carboxy, halogen, C₁₋₄ alkyl, C₁₋₄ alkoxy, (CR^{a1}R^{a2})ₘNR^{d1}R^{d2}, 3-8 membered cycloalkyl or its halogenated derivatives, 3-8 membered heterocyclyl or its halogenated derivatives, aryl or its halogenated or deuterated derivatives, heteroaryl or its halogenated or deuterated derivatives;
R⁴ is hydrogen, (CR^{a1}R^{a2})ₘCOOR^{b1}, (CR^{a1}R^{a2})ₘOCOR^{b}, (CR^{a1}R^{a2})ₙCOR^{c}, CONR^{d1}R^{d2}, (CR^{a1}R^{a2})ₙOR^{f};
wherein, R^{a1}, R^{a2} are each independently selected from hydrogen, halogen, C₁₋₄ alkyl, halogenated C₁₋₄ alkyl, C₂₋₄ alkenyl, C₂₋₄ alkynyl, 3-6 membered cycloalkyl, 3-6 membered heterocyclyl, aryl, heteroaryl, CN, OR^{f}, SR^{f} or NR^{d1}R^{d2}; or, R^{a1} and R^{a2} taken together with the carbon atom to which they are attached, form a 3-6 membered ring containing 0 or 1 heteroatom selected from N, O, or S;
R^{b1} is hydrogen, C₁₋₄ alkyl, C₁₋₄ alkoxy, halogenated C₁₋₄ alkyl, 3-6 membered cycloalkyl, 3-6 membered heterocyclyl, aryl, or heteroaryl;
R^{b} is hydrogen, C₁₋₄ alkyl, C₁₋₄ alkoxy, halogenated C₁₋₄ alkyl, C₂₋₄ alkenyl, C₂₋₄ alkynyl, 3-6 membered cycloalkyl, 3-6 membered heterocyclyl, aryl, or heteroaryl;
R^{c} is hydrogen, C₁₋₄ alkyl, C₁₋₄ alkoxy, C₂₋₄ alkenyl, C₂₋₄ alkynyl, 3-6 membered cycloalkyl, 3-6 membered heterocyclyl, aryl, or heteroaryl;
R^{d1}, R^{d2} are each independently selected from hydrogen, C₁₋₄ alkyl, C₁₋₄ alkoxy, 3-6 membered cycloalkyl, 3-6 membered heterocyclyl; or, R^{d1} and R^{d2} taken together with the nitrogen atom to which they are attached, form a 3-6 membered ring containing 1 or 2 heteroatom selected from N, O, or S;
Wherein, the alkyl, alkenyl, alkynyl, cycloalkyl, heterocyclyl, aryl, or heteroaryl in R^{a1}, R^{a2}, R^{d1}, R^{d2}, R^{b}, R^{b1}, R^{c} and R⁵ are unsubstituted or optionally substituted by one or more groups selected from halogen, C₁₋₄ alkyl, C₁₋₄ alkoxy, halogenated C₁₋₄ alkyl, C₂₋₄ alkenyl, C₂₋₄ alkynyl, 3-6 membered cycloalkyl, 3-6 membered heterocyclyl, CN, NO₂, OR^{f}, SR^{f}, NR^{d1}R^{d2}, CORⁱ, COOR^{f}, OCORⁱ, CONR^{d1}R^{d2}, NR^{d1}CORⁱ, NR^{d1}SO₂Rⁱ, or SO₂Rⁱ,
R^{f} is selected from hydrogen, C₁₋₄ alkyl or halogenated C₁₋₄ alkyl;
Rⁱ is selected from hydrogen, C₁₋₄ alkyl, C₂₋₄ alkenyl, C₂₋₄ alkynyl, 3-6 membered cycloalkyl, or 3-6 membered heterocyclyl;
m is selected from 0, 1, 2;
n is selected from 0, 1, 2.

10. The compound according to claim 7, or a stereoisomer thereof, or a pharmaceutically acceptable salt thereof, or a solvate thereof, or a prodrug thereof, or a metabolite thereof, or a deuterated derivative thereof, characterized in that said compound has a structure of formula V-4: 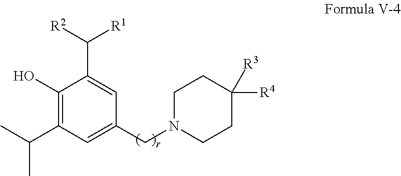
wherein,
R¹, R² are each independently selected from hydrogen, halogen, C₁₋₄ alkyl, C₂₋₄ alkenyl or 3-6 membered cycloalkyl;
R³ is hydrogen, (CR^{a1}R^{a2})ₘCOOR^{b1}, or groups consisiting of C₁₋₄ alkyl, C₂₋₄ alkenyl, C₂₋₄ alkynyl, 3-6 membered cycloalkyl, 3-6 membered heterocyclyl, aryl or heteroaryl unsubstituted or optionally substituted by one or more R^{x}, wherein R′ is each independently selected from cyano, nitro, hydroxyl, carboxy, halogen, C₁₋₄ alkyl, C₁₋₄ alkoxy, (CR^{a1}R^{a2})ₘNR^{d1}R^{d2}, 3-8 membered cycloalkyl or its halogenated derivatives, 3-8 membered heterocyclyl or its halogenated derivatives, aryl or its halogenated or deuterated derivatives, heteroaryl or its halogenated or deuterated derivatives;
R⁴ is hydrogen, 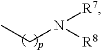
(CR^{a1}R^{a2})ₘCOOR^{b1}, (CR^{a1}R^{a2})ₘOCOR^{b}, (CR^{a1}R^{a2})ₙCOR^{c}, CONR^{d1}R^{d2}, (CR^{a1}R^{a2})ₙOR^{f};
R⁷ is hydrogen, or groups consisiting of C₁₋₄ alkyl, 3-6 membered cycloalkyl, 3-6 membered heterocyclyl, aryl or heteroaryl unsubstituted or optionally substituted by one or more R^{y}; wherein R^{y} is each independently selected from cyano, nitro, hydroxyl, carboxy, halogen, C₁₋₄ alkyl, C₁₋₄ alkoxy, C₁₋₄ alkylthio, O(CR^{a1}R^{a2})ₘNR^{d1}R^{d2}, (CR^{a1}R^{a2})ₘNR^{d1}R^{d2}, 3-8 membered cycloalkyl or its halogenated derivatives, 3-8 membered heterocyclyl or its halogenated derivatives, aryl or its halogenated or deuterated derivatives, heteroaryl or its halogenated or deuterated derivatives;
R⁸ is hydrogen, C₁₋₄ alkyl, (CR^{a1}R^{a2})ₘCOOR^{b1}, (CR^{a1}R^{a2})ₘOCOR^{b}, (CR^{a1}R^{a2})ₙCOR^{c}, CONR^{d1}R^{d2}, (CR^{a1}R^{a2})ₙOR^{f}, (CR^{a1}R^{a2})ₘSO₂OR^{b1}, (CR^{a1}R^{a2})ₘSOOR^{b1};
wherein, R^{a1}, R^{a2} are each independently selected from hydrogen, halogen, C₁₋₄ alkyl, halogenated C₁₋₄ alkyl, C₂₋₄ alkenyl, C₂₋₄ alkynyl, 3-6 membered cycloalkyl, 3-6 membered heterocyclyl, aryl, heteroaryl, CN, OR^{f}, SR^{f}, or NR^{d1}R^{d2}; or, R^{a1} and R^{a2} taken together with the carbon atom to which they are attached, form a 3-6 membered ring containing 0 or 1 heteroatom selected from N, O, or S;
R^{b1} is hydrogen, C₁₋₄ alkyl, C₁₋₄ alkoxy, halogenated C₁₋₄ alkyl, 3-6 membered cycloalkyl, 3-6 membered heterocyclyl, aryl, or heteroaryl;
R^{b} is hydrogen, C₁₋₄ alkyl, C₁₋₄ alkoxy, halogenated C₁₋₄ alkyl, C₂₋₄ alkenyl, C₂₋₄ alkynyl, 3-6 membered cycloalkyl, 3-6 membered heterocyclyl, aryl, or heteroaryl;
R^{c} is hydrogen, C₁₋₄ alkyl, C₁₋₄ alkoxy, C₂₋₄ alkenyl, C₂₋₄ alkynyl, 3-6 membered cycloalkyl, 3-6 membered heterocyclyl, aryl, or heteroaryl;
R^{d1}, R^{d2} are each independently selected from hydrogen, C₁₋₄ alkyl, C₁₋₄ alkoxy, 3-6 membered cycloalkyl, 3-6 membered heterocyclyl; or, R^{d1} and R^{d2} taken together with the nitrogen atom to which they are attached, form a 3-6 membered ring containing 1 or 2 heteroatom selected from N, O, or S;
Wherein, the alkyl, alkenyl, alkynyl, cycloalkyl, heterocyclyl, aryl, or heteroaryl in R^{a1}, R^{a2}, R^{d1}, R^{d2}, R^{b}, R^{b1} and R^{c} are unsubstituted or optionally substituted by one or more groups selected from halogen, C₁₋₄ alkyl, C₁₋₄ alkoxy, halogenated C₁₋₄ alkyl, C₂₋₄ alkenyl, C₂₋₄ alkynyl, 3-6 membered cycloalkyl, 3-6 membered heterocyclyl, CN, NO₂, OR^{f}, SR^{f}, NR^{d1}R^{d2}, CORⁱ, COOR^{f}, OCORⁱ, CONR^{d1}R^{d2}, NR^{d1}CORⁱ, NR^{d1}SO₂Rⁱ, or SO₂Rⁱ;
R^{f} is selected from hydrogen, C₁₋₄ alkyl, or halogenated C₁₋₄ alkyl;
Rⁱ is selected from hydrogen, C₁₋₄ alkyl, C₂₋₄ alkenyl, C₂₋₄ alkynyl, 3-6 membered cycloalkyl, or 3-6 membered heterocyclyl;
m is selected from 0, 1, 2;
n is selected from 0, 1, 2;
r is selected from 0, 1, 2, 3;
p is selected from 0, 1, 2, 3.

11. The compound according to claim 7, or a stereoisomer thereof, or a pharmaceutically acceptable salt thereof, or a solvate thereof, or a prodrug thereof, or a metabolite thereof, or a deuterated derivative thereof, characterized in that said compound has a structure of formula V-5 or V-6: 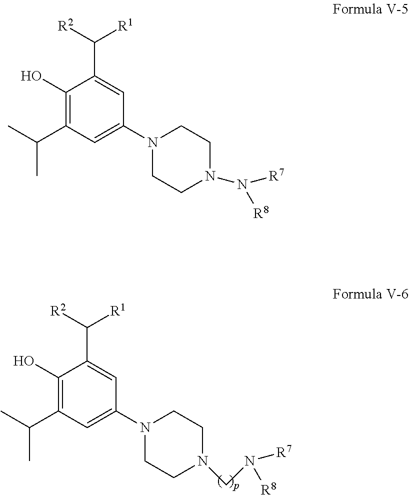
wherein,
R¹, R² are each independently selected from hydrogen, halogen, C₁₋₄ alkyl, C₂₋₄ alkenyl or 3-6 membered cycloalkyl;
R⁷ is hydrogen, or groups consisiting of C₁₋₄ alkyl, 3-6 membered cycloalkyl, 3-6 membered heterocyclyl, aryl or heteroaryl unsubstituted or optionally substituted by one or more R^{y}; R^{y} is each independently selected from cyano, nitro, hydroxyl, carboxy, halogen, C₁₋₄ alkyl, C₁₋₄ alkoxy, C₁₋₄ alkylthio, O(CR^{a1}R^{a2})ₘNR^{d1}R^{d2}, (CR^{a1}R^{a2})ₘNR^{d1}R^{d2}, 3-8 membered cycloalkyl or its halogenated derivatives, 3-8 membered heterocyclyl or its halogenated derivatives, aryl or its halogenated or deuterated derivatives, heteroaryl or its halogenated or deuterated derivatives;
R⁸ is hydrogen, C₁₋₄ alkyl, (CR^{a1}R^{a2})ₘCOOR^{b1}, (CR^{a1}R^{a2})ₘOCOR^{b}, (CR^{a1}R^{a2})ₙCOR^{c}, CONR^{d1}R^{d2}, (CR^{a1}R^{a2})ₙOR^{f}, (CR^{a1}R^{a2})ₘSO₂OR^{b1}, (CR^{a1}R^{a2})ₘSOOR^{b1};
wherein, R^{a1}, R^{a2} are each independently selected from hydrogen, halogen, C₁₋₄ alkyl, halogenated C₁₋₄ alkyl, C₂₋₄ alkenyl, C₂₋₄ alkynyl, 3-6 membered cycloalkyl, 3-6 membered heterocyclyl, aryl, heteroaryl, CN, OR^{f}, SR^{f}, or NR^{d1}R^{d2}; or, R^{a1} and R^{a2} taken together with the carbon atom to which they are attached, form a 3-6 membered ring containing 0 or 1 heteroatom selected from N, O, or S;
R^{b1} is hydrogen, C₁₋₄ alkyl, C₁₋₄ alkoxy, halogenated C₁₋₄ alkyl, 3-6 membered cycloalkyl, 3-6 membered heterocyclyl, aryl, or heteroaryl;
R^{b} is hydrogen, C₁₋₄ alkyl, C₁₋₄ alkoxy, halogenated C₁₋₄ alkyl, C₂₋₄ alkenyl, C₂₋₄ alkynyl, 3-6 membered cycloalkyl, 3-6 membered heterocyclyl, aryl, or heteroaryl;
R^{c} is hydrogen, C₁₋₄ alkyl, C₁₋₄ alkoxy, C₂₋₄ alkenyl, C₂₋₄ alkynyl, 3-6 membered cycloalkyl, 3-6 membered heterocyclyl, aryl, or heteroaryl;
R^{d1}, R^{d2} are each independently selected from hydrogen, C₁₋₄ alkyl, C₁₋₄ alkoxy, 3-6 membered cycloalkyl, 3-6 membered heterocyclyl; or, R^{d1} and R^{d2} taken together with the nitrogen atom to which they are attached, form a 3-6 membered ring containing 1 or 2 heteroatom selected from N, O, or S;
Wherein, the alkyl, alkenyl, alkynyl, cycloalkyl, heterocyclyl, aryl, or heteroaryl in R^{a1}, R^{a2}, R^{d1}, R^{d2}, R^{b}, R^{b1} and R^{c} are unsubstituted or optionally substituted by one or more groups selected from halogen, C₁₋₄ alkyl, C₁₋₄ alkoxy, halogenated C₁₋₄ alkyl, C₂₋₄ alkenyl, C₂₋₄ alkynyl, 3-6 membered cycloalkyl, 3-6 membered heterocyclyl, CN, NO₂, OR^{f}, SR^{f}, NR^{d1}R^{d2}, CORⁱ, COOR^{f}, OCORⁱ, CONR^{d1}R^{d2}, NR^{d1}CORⁱ, NR^{d1}SO₂Rⁱ, or SO₂Rⁱ;
R^{f} is selected from hydrogen, C₁₋₄ alkyl, or halogenated C₁₋₄ alkyl;
Rⁱ is selected from hydrogen, C₁₋₄ alkyl, C₂₋₄ alkenyl, C₂₋₄ alkynyl, 3-6 membered cycloalkyl, or 3-6 membered heterocyclyl;
m is selected from 0, 1, 2;
n is selected from 0, 1, 2;
p is selected from 0, 1, 2, 3.

12. The compound according to claim 4, or a stereoisomer thereof, or a pharmaceutically acceptable salt thereof, or a solvate thereof, or a prodrug thereof, or a metabolite thereof, or a deuterated derivative thereof, characterized in that said compound has a structure of formula VI-1 or VI-2: 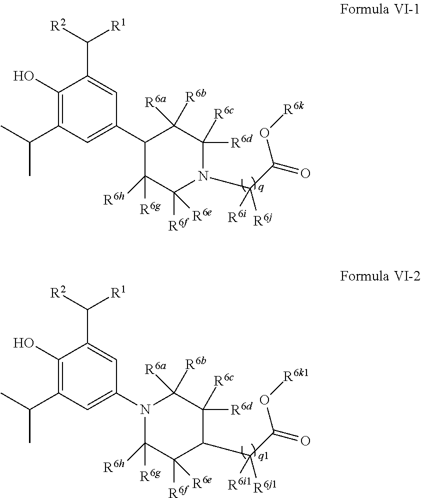
R¹, R², R¹¹, R¹², R^{6a}, R^{6b}, R^{6c}, R^{6d}, R^{6e}, R^{6f}, R^{6g} and R^{6h} are as described in claim 4;
R⁶ⁱ, R^{6j}, R⁶ⁱ¹, R^{6j1} are each independently selected from hydrogen, halogen, C₁₋₆ alkyl, halogenated C₁₋₆ alkyl, C₂₋₆ alkenyl, C₂₋₆ alkynyl, 3-6 membered cycloalkyl, 3-6 membered heterocyclyl, aryl, heteroaryl, CN, OR^{f}, SR^{f} or NR^{d1}R^{d2}; or, R⁶ⁱ and R^{6j}, or, R⁶ⁱ¹ and R^{6j1} taken together with the carbon atom to which they are attached, form a 3-6 menbered ring containing 0 or 1 heteroatom selected from N, O, or S;
R^{6k}, R^{6k1} are each independently selected from hydrogen, C₁₋₆ alkyl, halogenated C₁₋₆ alkyl, C₂₋₆ alkenyl, C₂₋₆ alkynyl, 3-6 membered cycloalkyl, 3-6 membered heterocyclyl, aryl, heteroaryl;
R^{f} is selected from hydrogen, C₁₋₄ alkyl or halogenated C₁₋₄ alkyl;
R^{d1}, R^{d2} are each independently selected from hydrogen, C₁₋₄ alkyl, C₁₋₄ alkoxy, 3-6 membered cycloalkyl, 3-6 membered heterocyclyl; or, R^{d1} and R^{d2} taken together with the nitrogen atom to which they are attached, form a 3-6 membered ring containing 1 or 2 heteroatom selected from N, O, or S;
q is selected from 0, 1, 2, 3, 4;
q1 is selected from 0, 1, 2, 3, 4.

13. The compound according to claim 1, or a stereoisomer thereof, or a pharmaceutically acceptable salt thereof, or a solvate thereof, or a prodrug thereof, or a metabolite thereof, or a deuterated derivative thereof, characterized in that the structure of said compound is selected from:

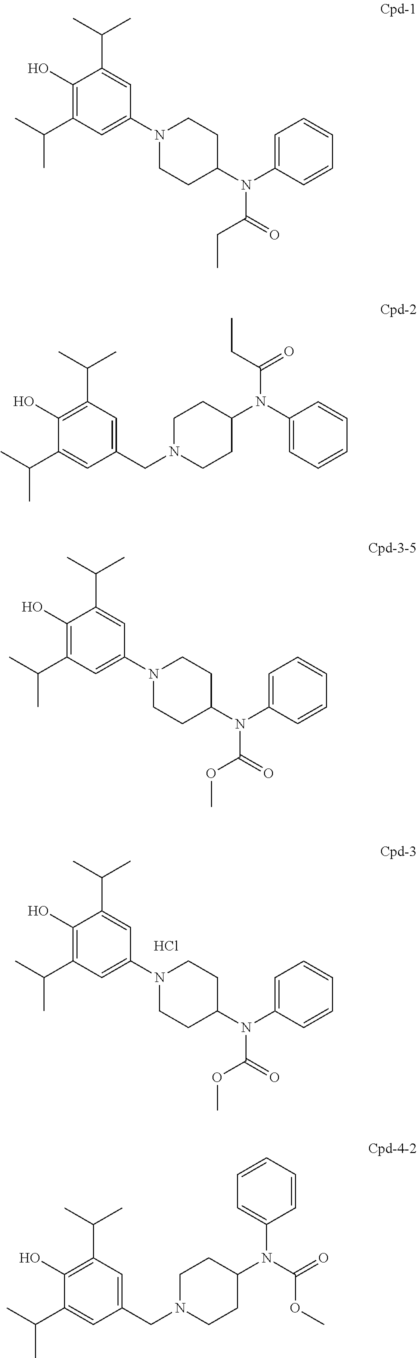 [SMILES: CCC(=O)N(c1ccccc1)C1CCN(c2cc(C(C)C)c(O)c(C(C)C)c2)CC1 ; CCC(=O)N(c1ccccc1)C1CCN(Cc2cc(C(C)C)c(O)c(C(C)C)c2)CC1 ; COC(=O)N(c1ccccc1)C1CCN(c2cc(C(C)C)c(O)c(C(C)C)c2)CC1 ; COC(=O)N(c1ccccc1)C1CCN(c2cc(C(C)C)c(O)c(C(C)C)c2)CC1 ; Cl ; COC(=O)N(c1ccccc1)C1CCN(Cc2cc(C(C)C)c(O)c(C(C)C)c2)CC1]

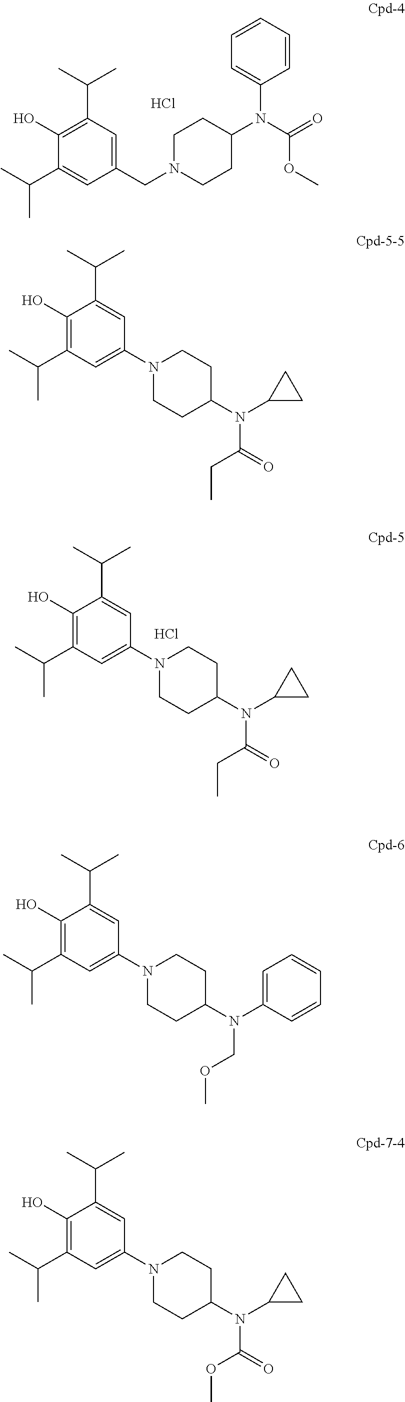

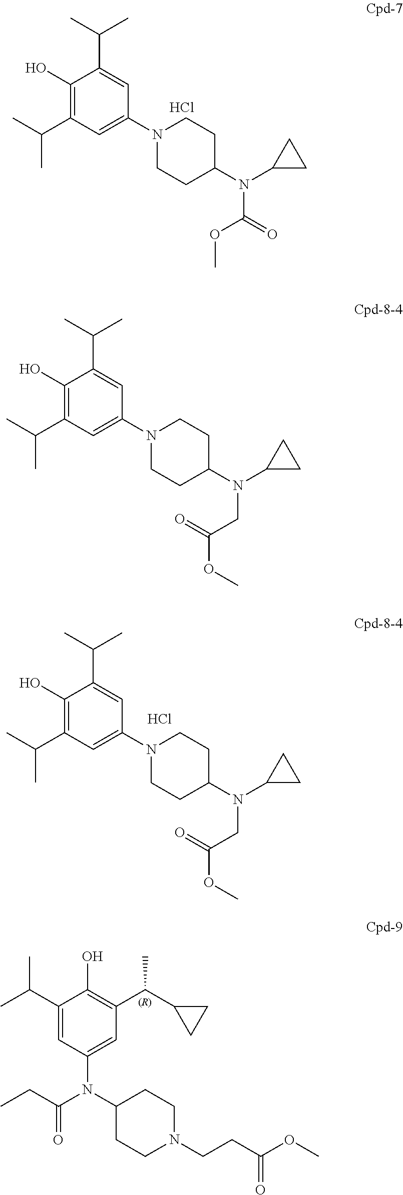

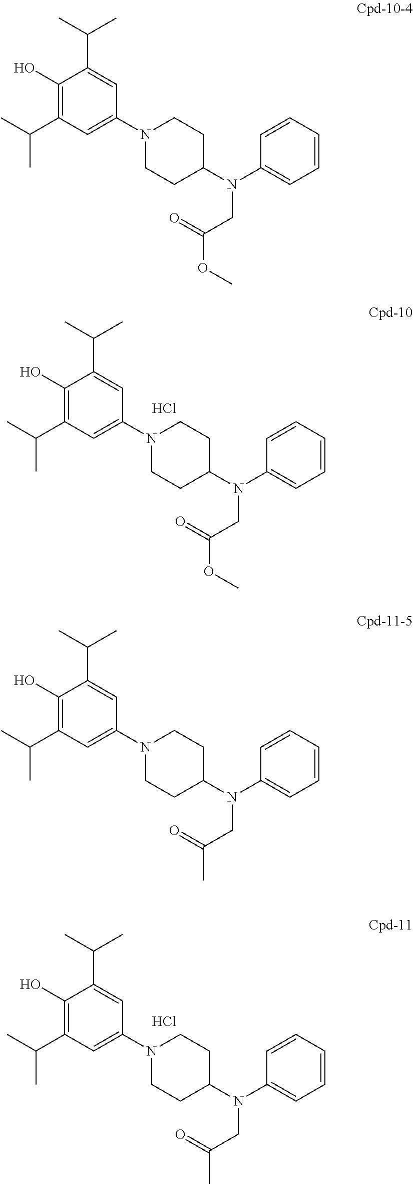 [SMILES: COC(=O)CN(c1ccccc1)C1CCN(c2cc(C(C)C)c(O)c(C(C)C)c2)CC1 ; COC(=O)CN(c1ccccc1)C1CCN(c2cc(C(C)C)c(O)c(C(C)C)c2)CC1 ; Cl ; CC(=O)CN(c1ccccc1)C1CCN(c2cc(C(C)C)c(O)c(C(C)C)c2)CC1 ; CC(=O)CN(c1ccccc1)C1CCN(c2cc(C(C)C)c(O)c(C(C)C)c2)CC1 ; Cl]

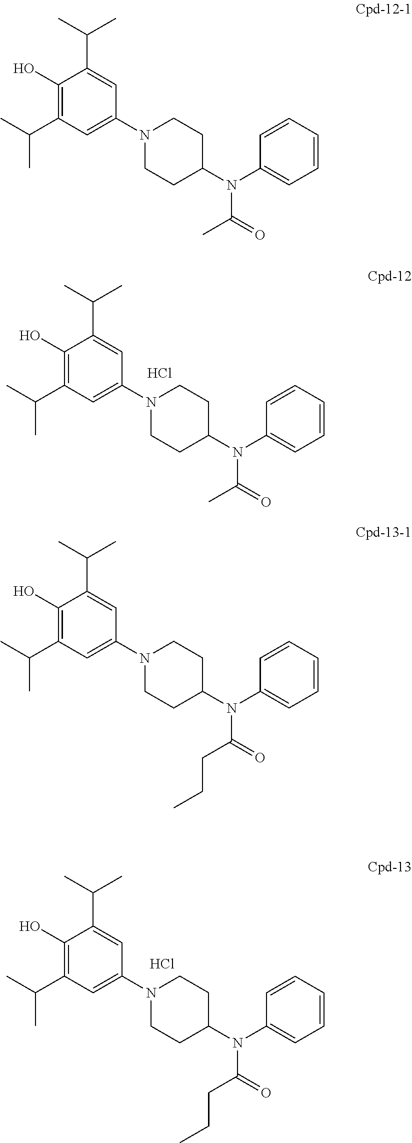 [SMILES: CC(=O)N(c1ccccc1)C1CCN(c2cc(C(C)C)c(O)c(C(C)C)c2)CC1 ; CC(=O)N(c1ccccc1)C1CCN(c2cc(C(C)C)c(O)c(C(C)C)c2)CC1 ; Cl ; CCCC(=O)N(c1ccccc1)C1CCN(c2cc(C(C)C)c(O)c(C(C)C)c2)CC1 ; CCCC(=O)N(c1ccccc1)C1CCN(c2cc(C(C)C)c(O)c(C(C)C)c2)CC1 ; Cl]

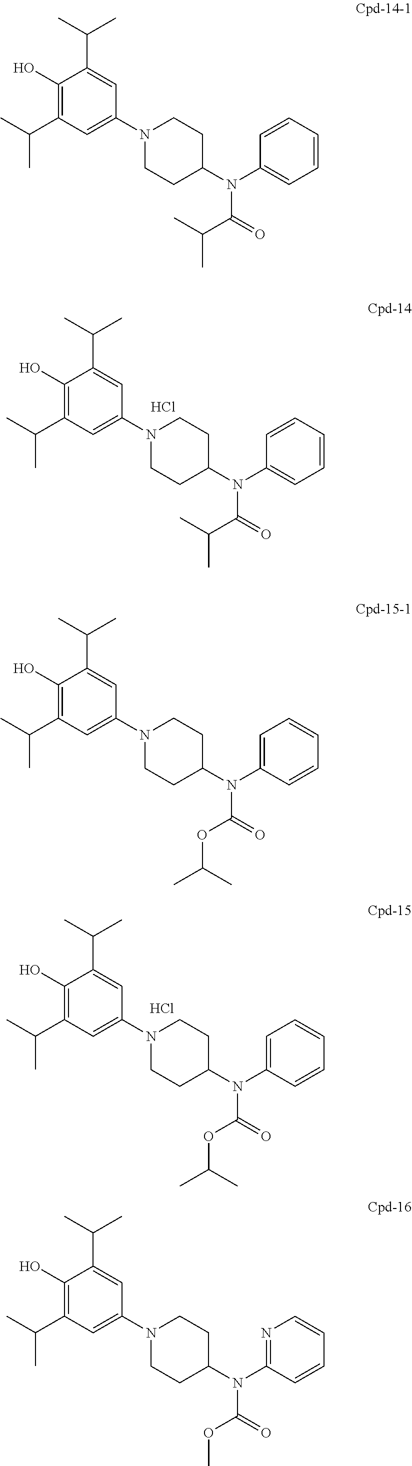

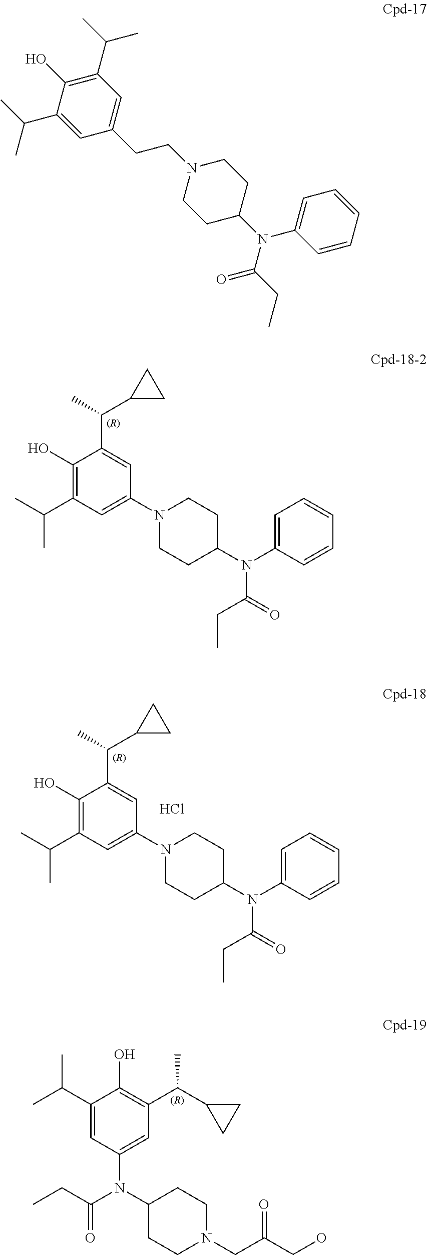

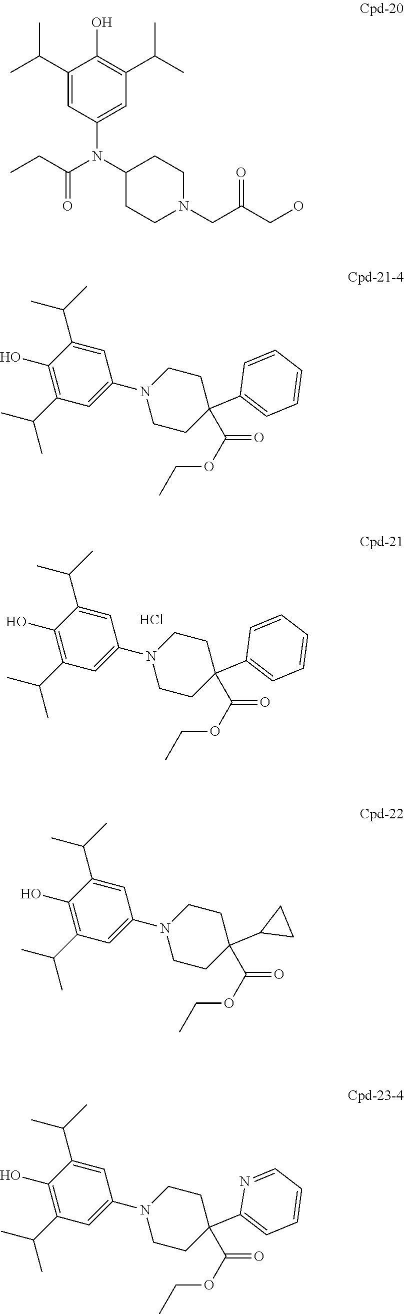 [SMILES: CCC(=O)N(c1cc(C(C)C)c(O)c(C(C)C)c1)C1CCN(CC(=O)CO)CC1 ; CCOC(=O)C1(c2ccccc2)CCN(c2cc(C(C)C)c(O)c(C(C)C)c2)CC1 ; CCOC(=O)C1(c2ccccc2)CCN(c2cc(C(C)C)c(O)c(C(C)C)c2)CC1 ; Cl ; CCOC(=O)C1(C2CC2)CCN(c2cc(C(C)C)c(O)c(C(C)C)c2)CC1 ; CCOC(=O)C1(c2ccccn2)CCN(c2cc(C(C)C)c(O)c(C(C)C)c2)CC1]

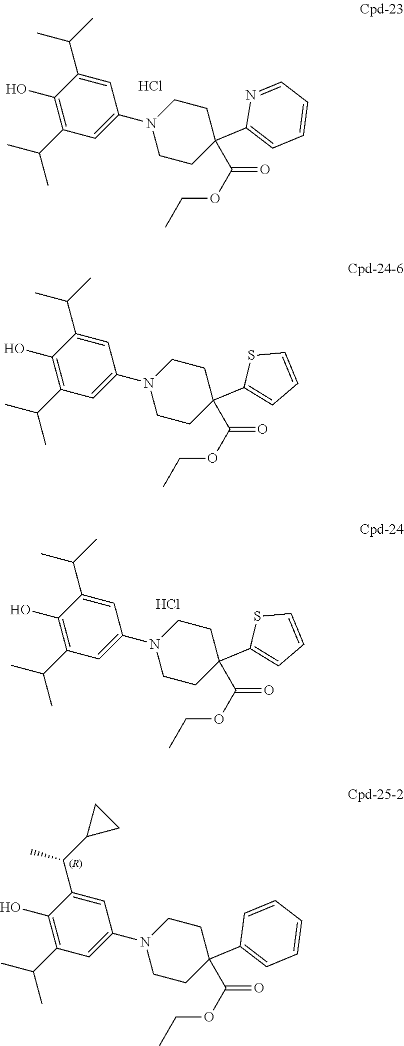

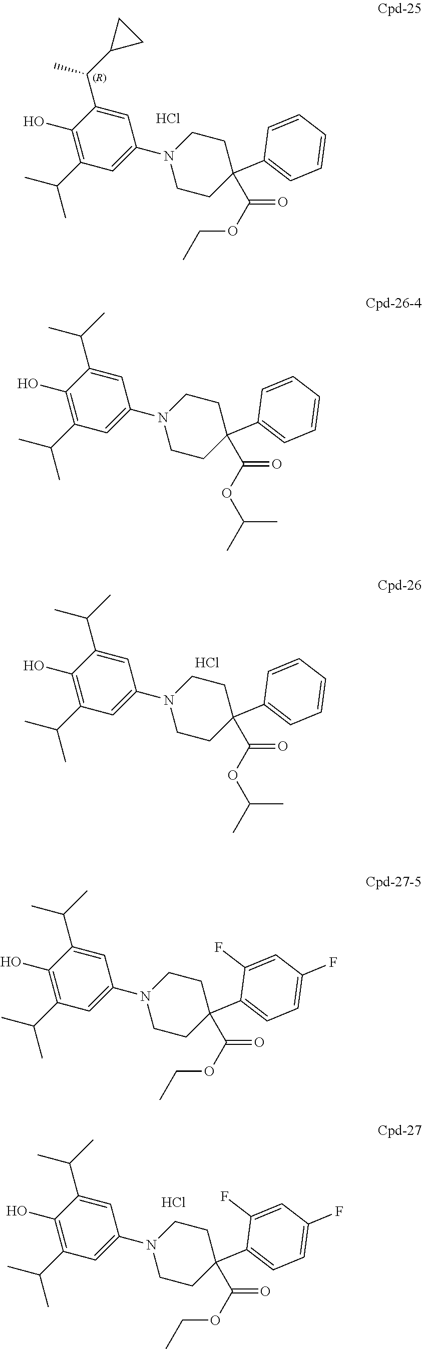

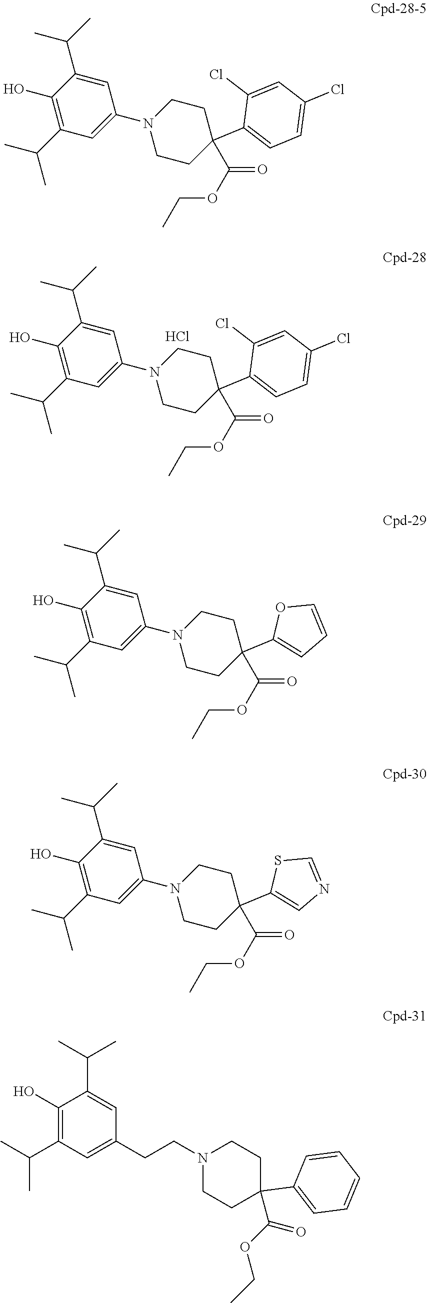 [SMILES: CCOC(=O)C1(c2ccc(Cl)cc2Cl)CCN(c2cc(C(C)C)c(O)c(C(C)C)c2)CC1 ; CCOC(=O)C1(c2ccc(Cl)cc2Cl)CCN(c2cc(C(C)C)c(O)c(C(C)C)c2)CC1 ; Cl ; CCOC(=O)C1(c2ccco2)CCN(c2cc(C(C)C)c(O)c(C(C)C)c2)CC1 ; CCOC(=O)C1(c2cncs2)CCN(c2cc(C(C)C)c(O)c(C(C)C)c2)CC1 ; CCOC(=O)C1(c2ccccc2)CCN(CCc2cc(C(C)C)c(O)c(C(C)C)c2)CC1]

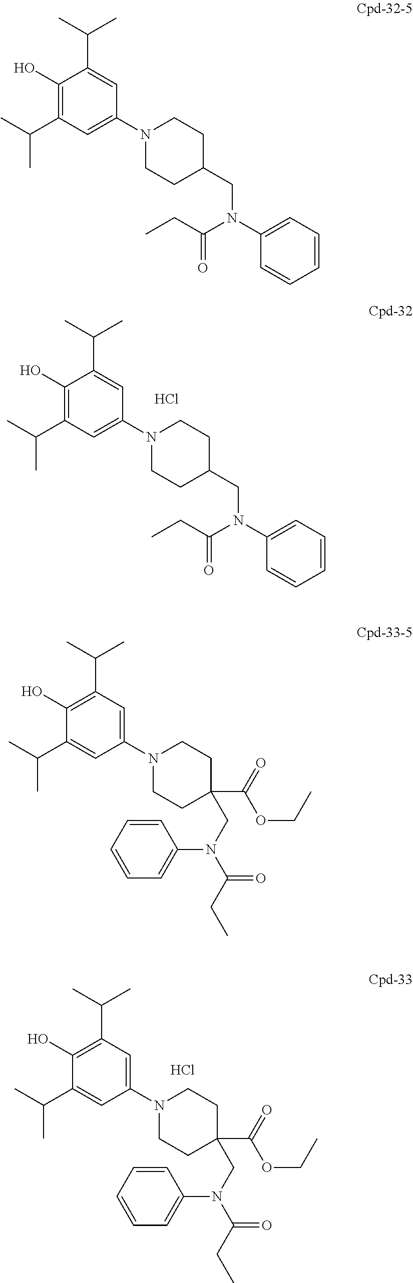

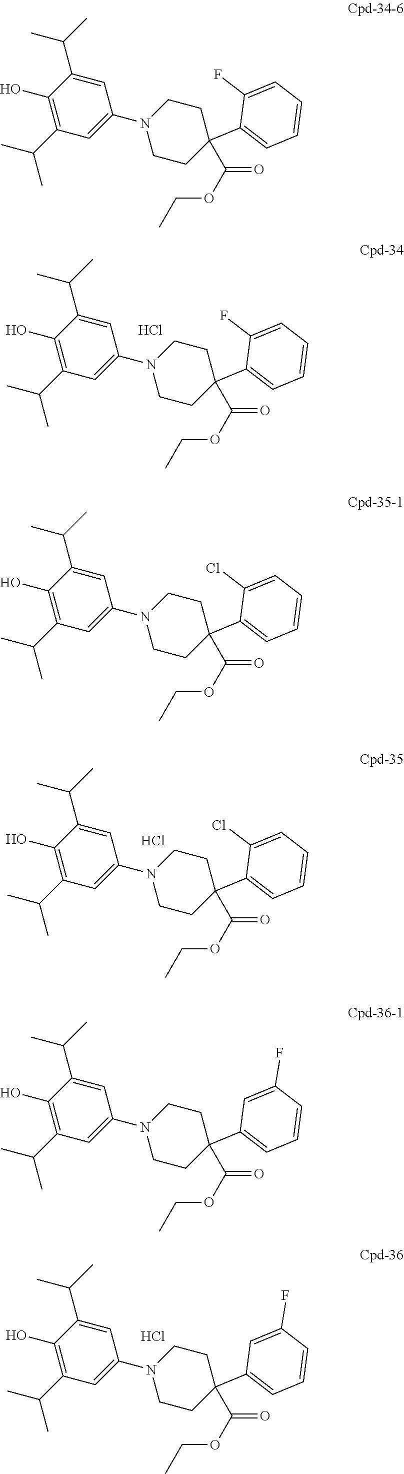

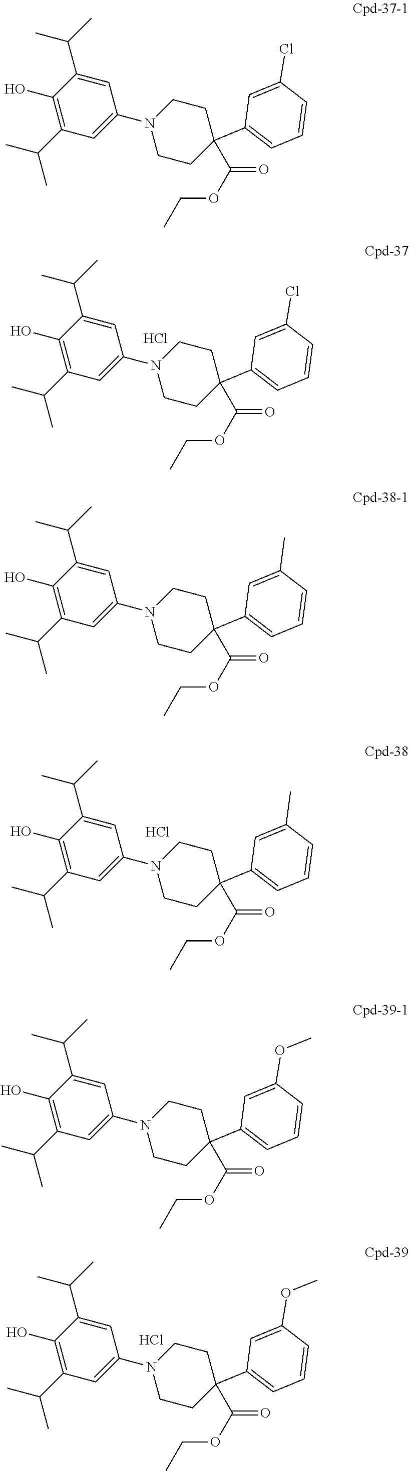

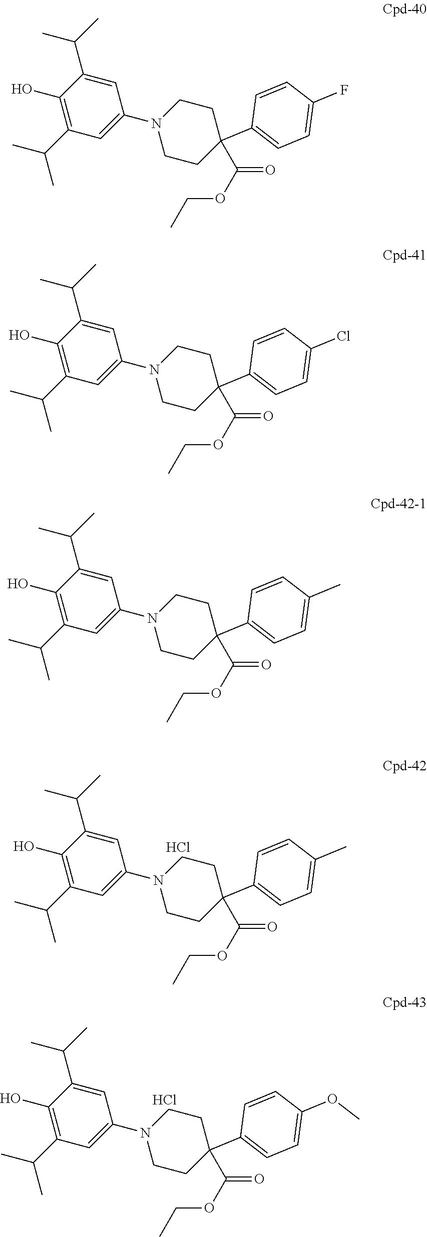

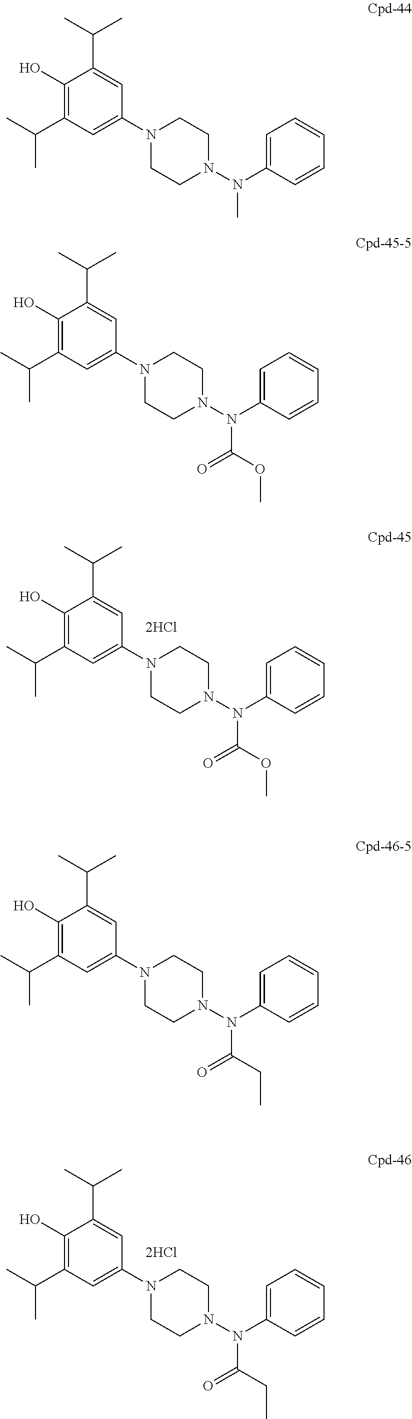 [SMILES: CC(C)c1cc(N2CCN(N(C)c3ccccc3)CC2)cc(C(C)C)c1O ; COC(=O)N(c1ccccc1)N1CCN(c2cc(C(C)C)c(O)c(C(C)C)c2)CC1 ; COC(=O)N(c1ccccc1)N1CCN(c2cc(C(C)C)c(O)c(C(C)C)c2)CC1 ; CCC(=O)N(c1ccccc1)N1CCN(c2cc(C(C)C)c(O)c(C(C)C)c2)CC1 ; CCC(=O)N(c1ccccc1)N1CCN(c2cc(C(C)C)c(O)c(C(C)C)c2)CC1]

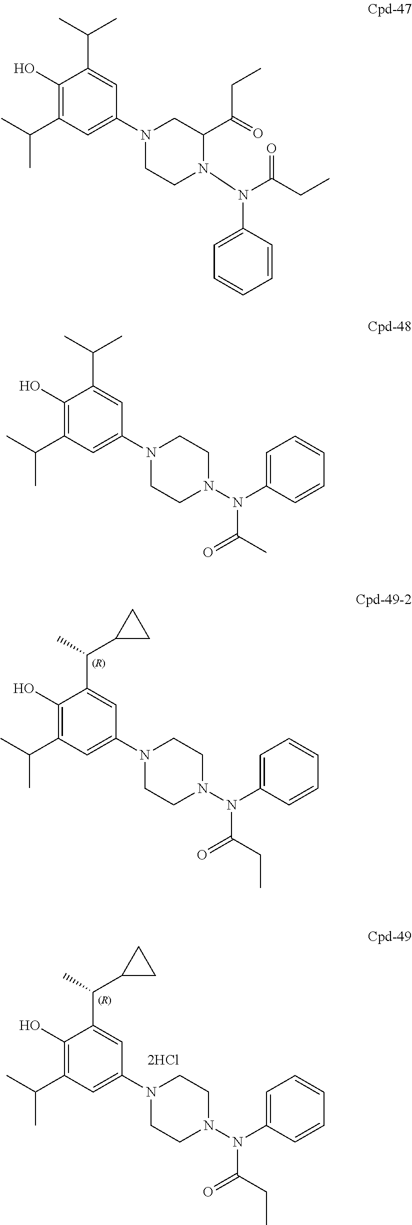

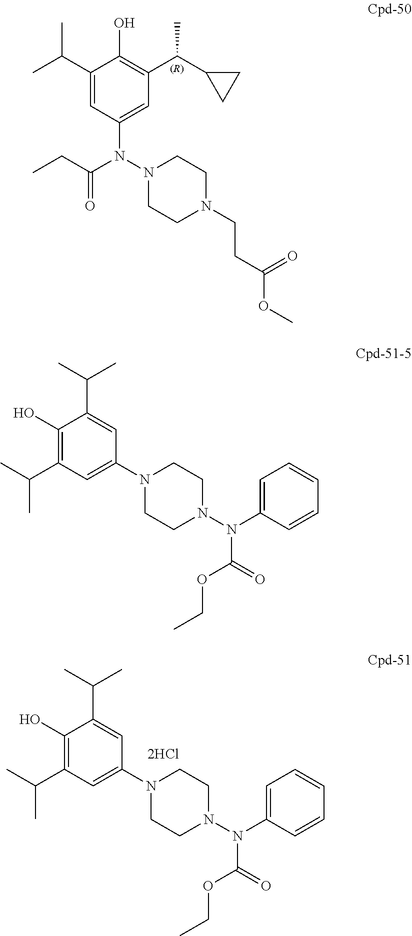

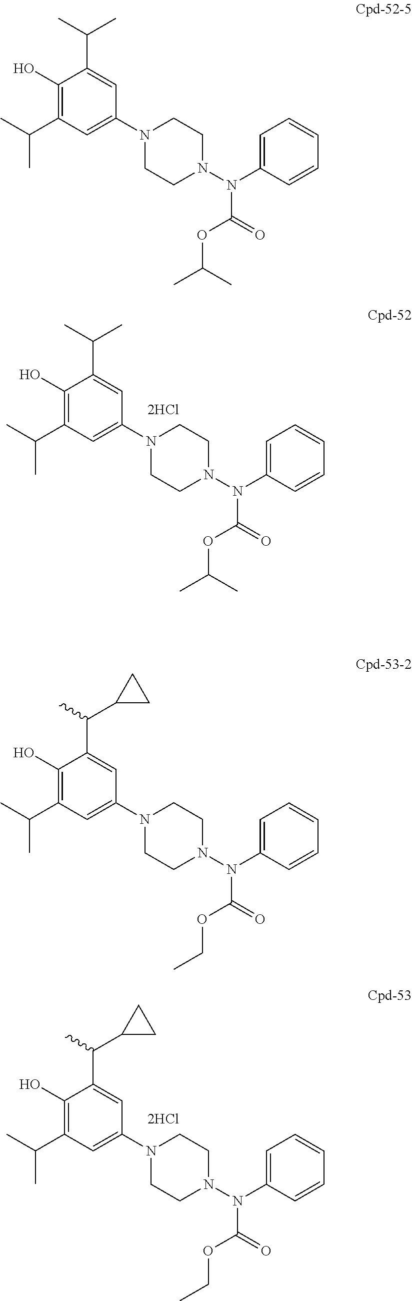

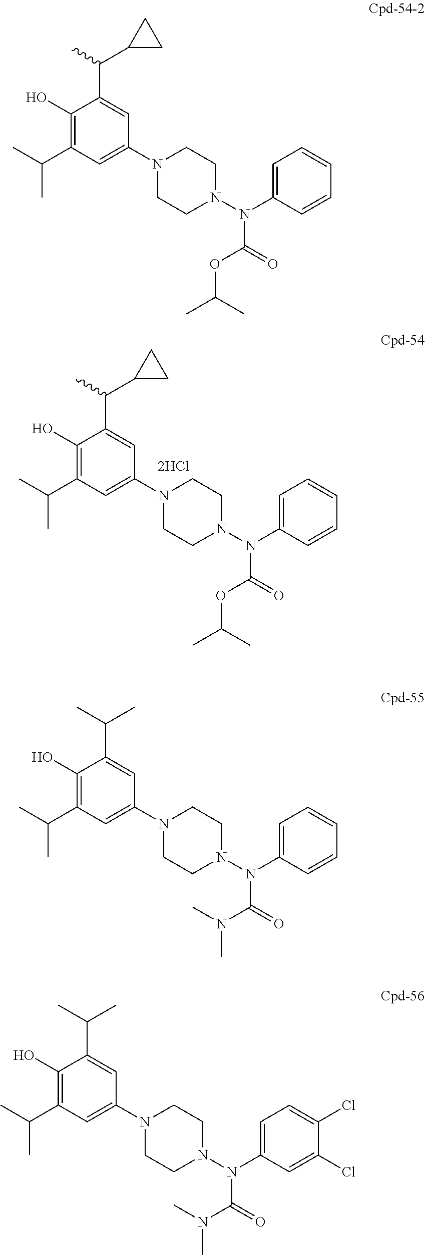

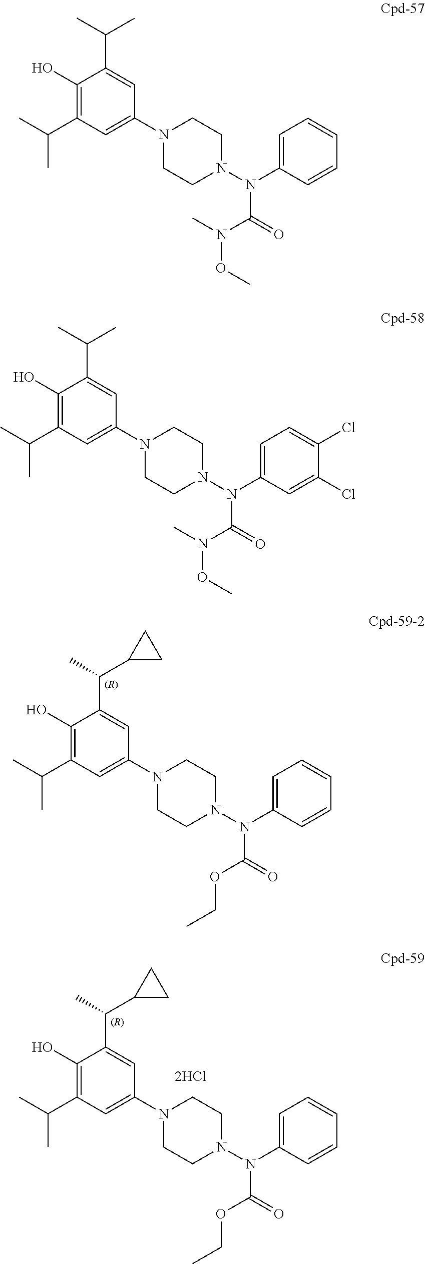

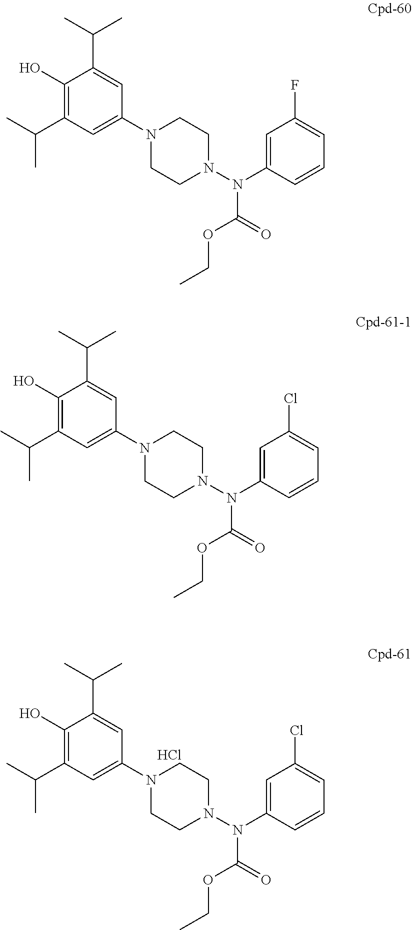 [SMILES: CCOC(=O)N(c1cccc(F)c1)N1CCN(c2cc(C(C)C)c(O)c(C(C)C)c2)CC1 ; CCOC(=O)N(c1cccc(Cl)c1)N1CCN(c2cc(C(C)C)c(O)c(C(C)C)c2)CC1 ; CCOC(=O)N(c1cccc(Cl)c1)N1CCN(c2cc(C(C)C)c(O)c(C(C)C)c2)CC1 ; Cl]

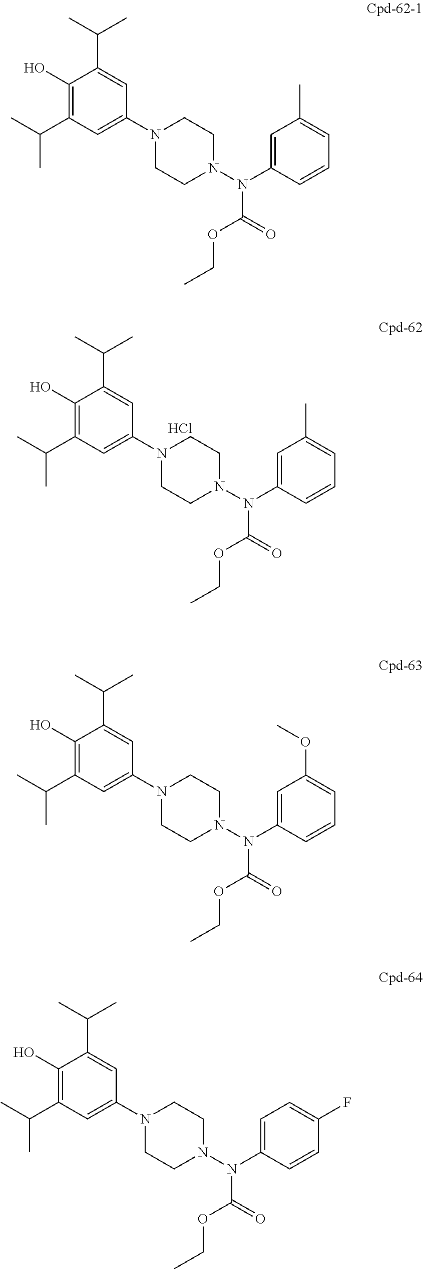 [SMILES: CCOC(=O)N(c1cccc(C)c1)N1CCN(c2cc(C(C)C)c(O)c(C(C)C)c2)CC1 ; CCOC(=O)N(c1cccc(C)c1)N1CCN(c2cc(C(C)C)c(O)c(C(C)C)c2)CC1 ; Cl ; CCOC(=O)N(c1cccc(OC)c1)N1CCN(c2cc(C(C)C)c(O)c(C(C)C)c2)CC1 ; CCOC(=O)N(c1ccc(F)cc1)N1CCN(c2cc(C(C)C)c(O)c(C(C)C)c2)CC1]

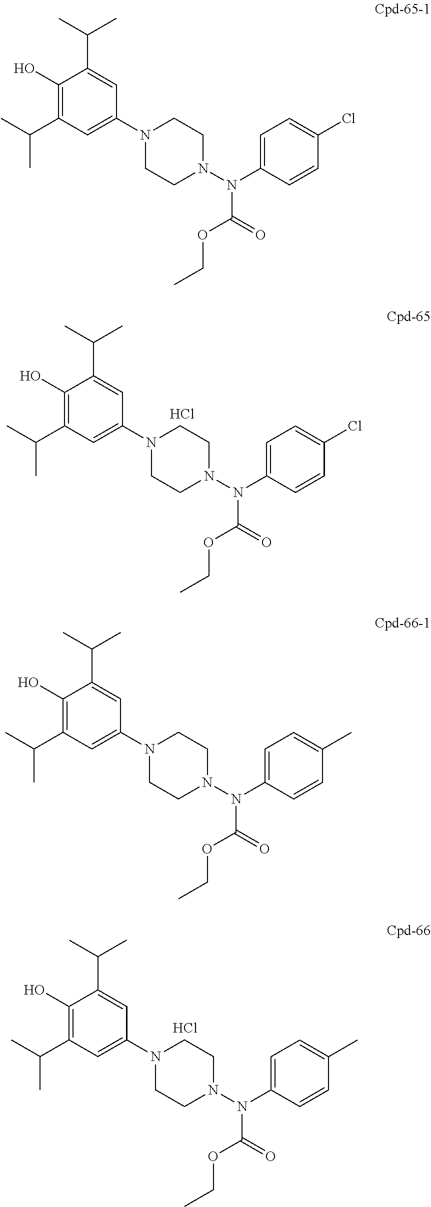

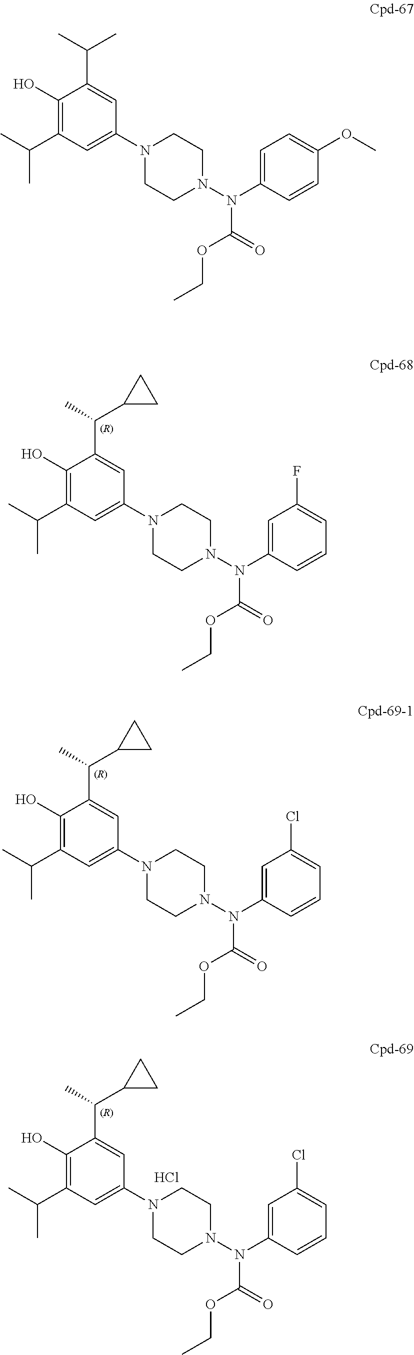

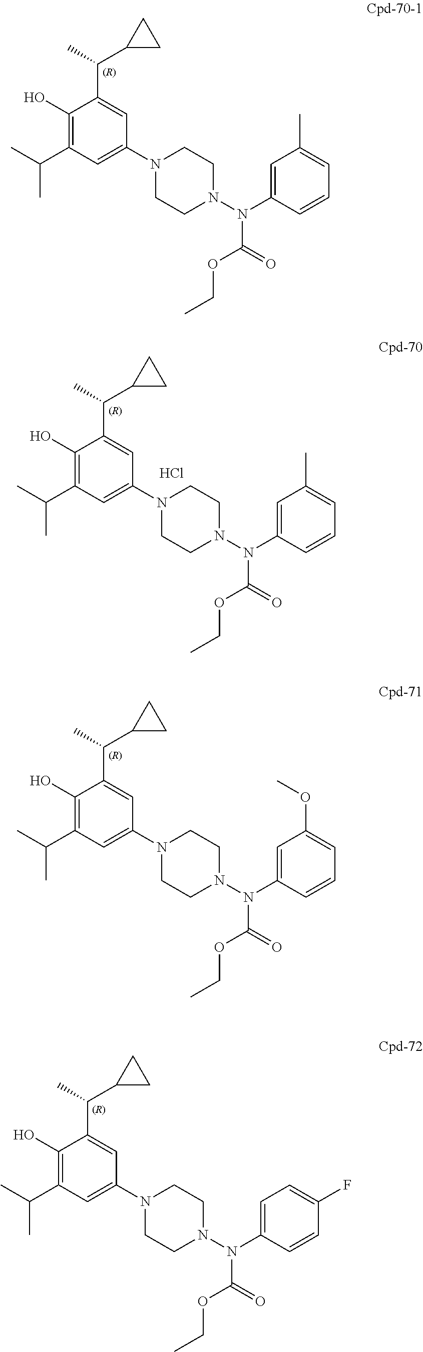

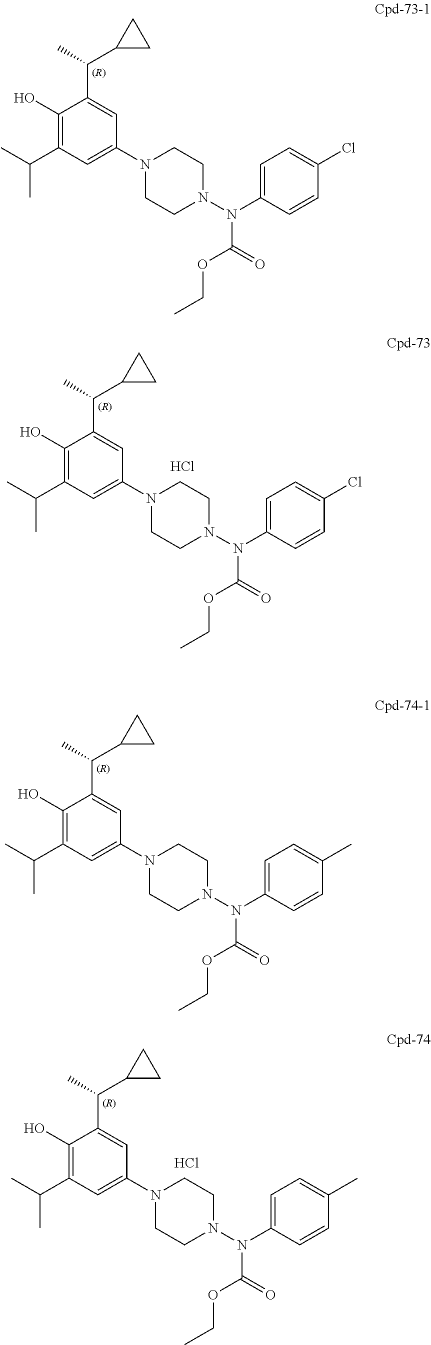

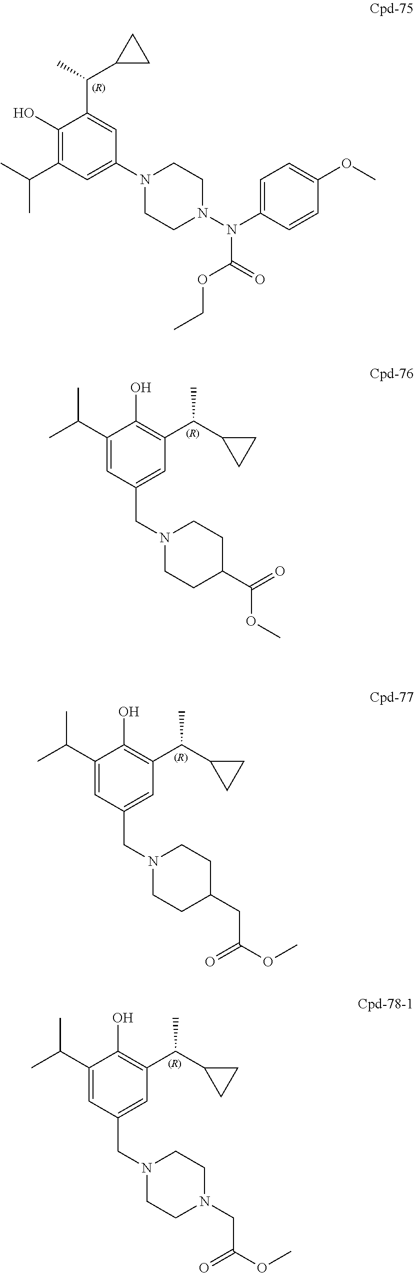

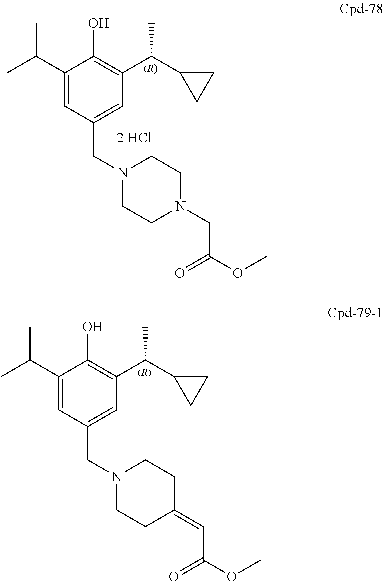

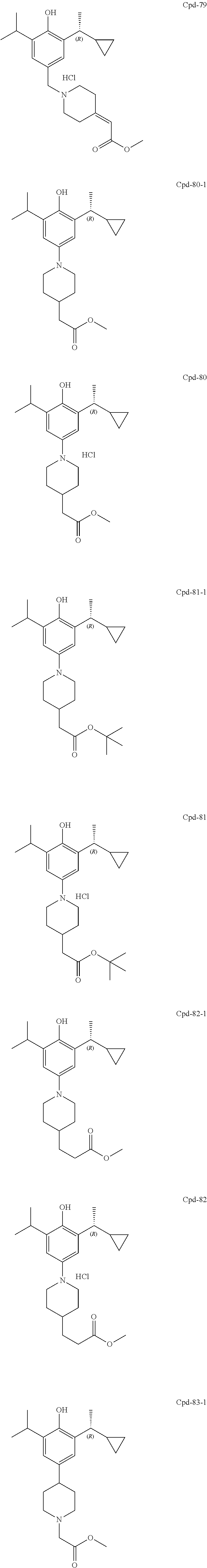

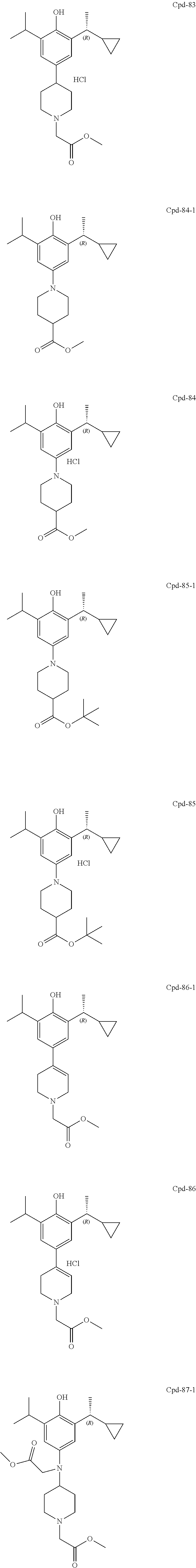

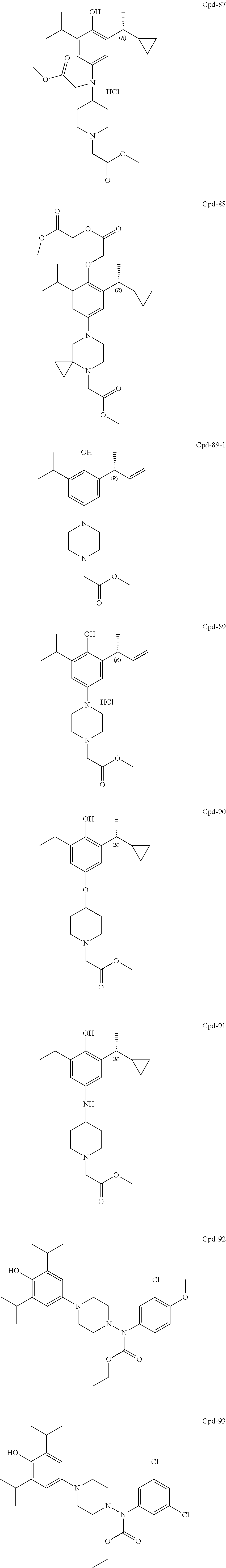

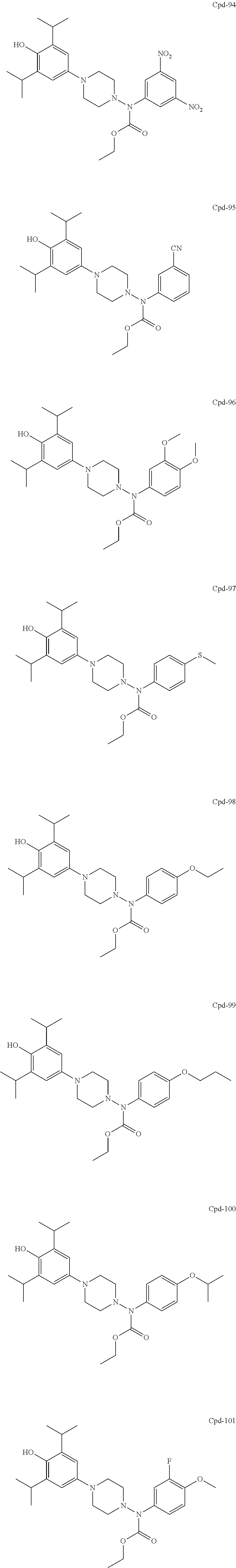

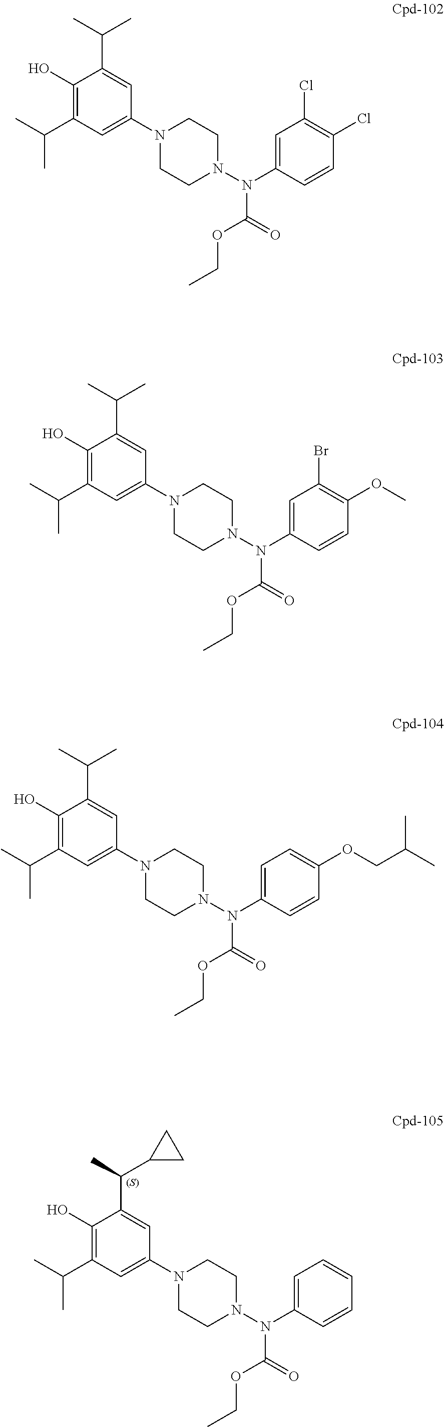

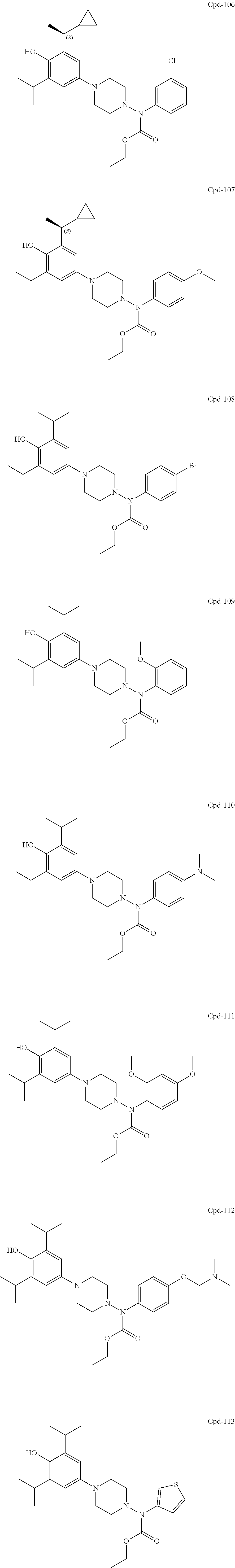

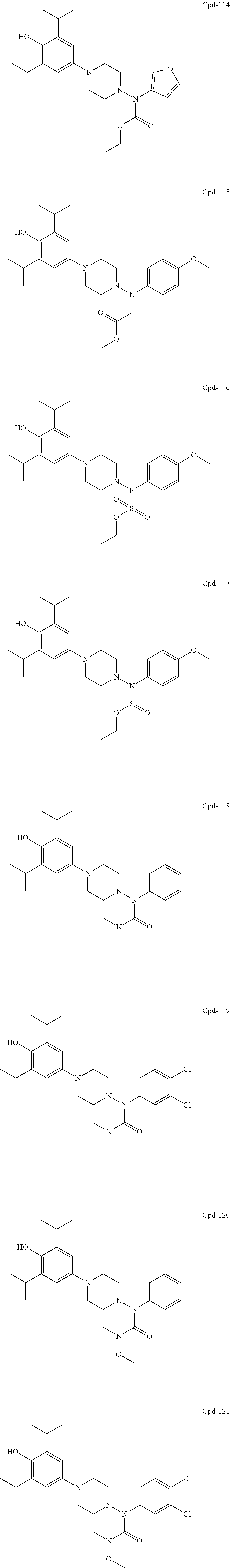

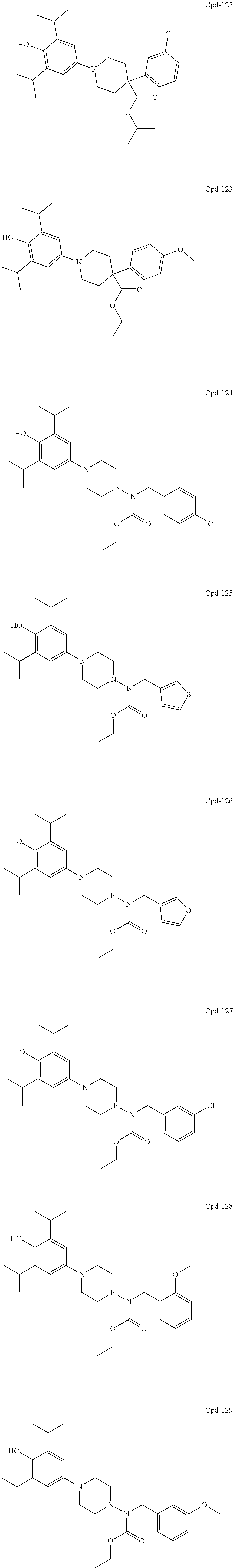

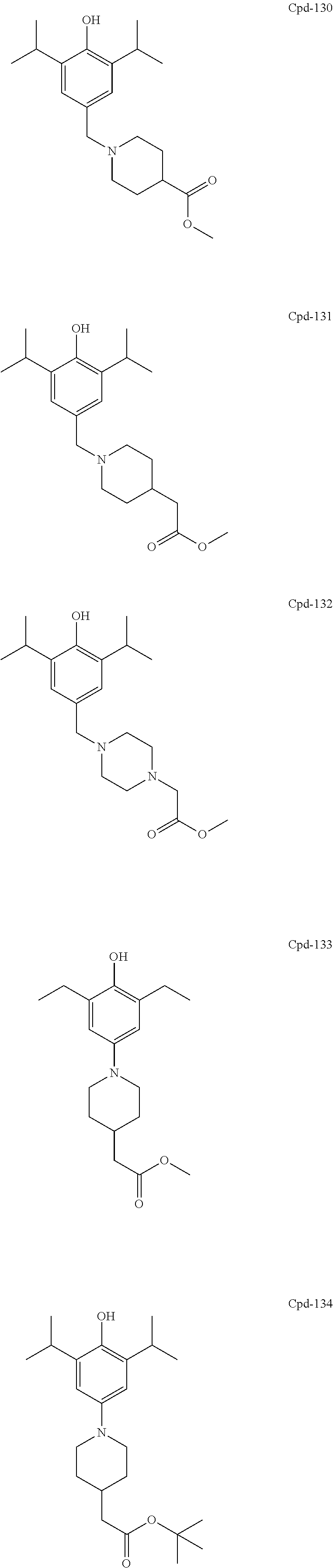

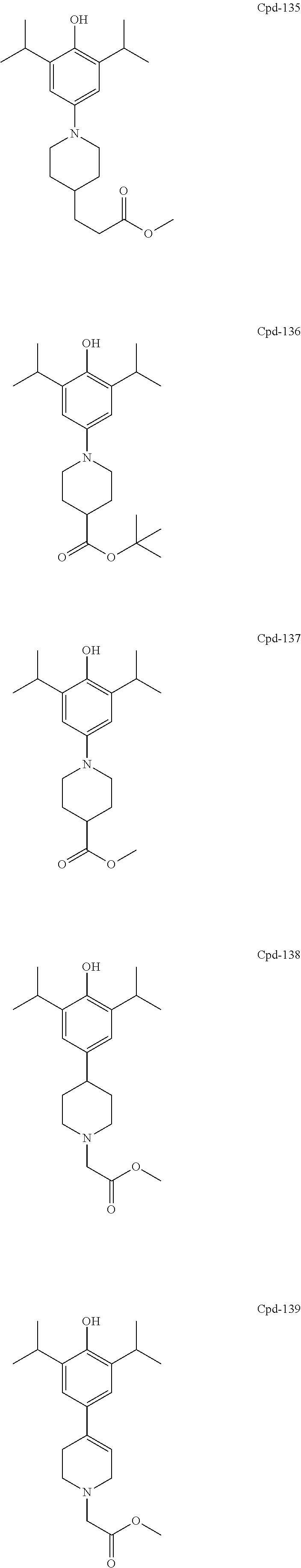

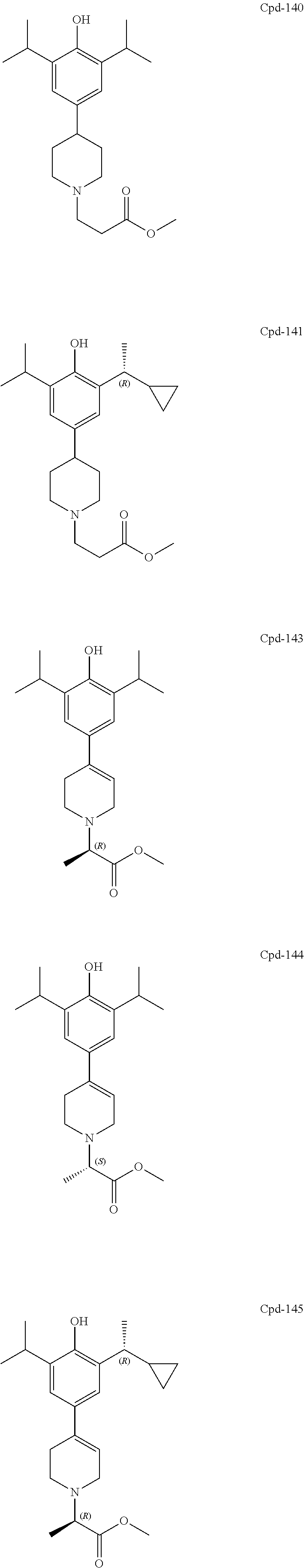

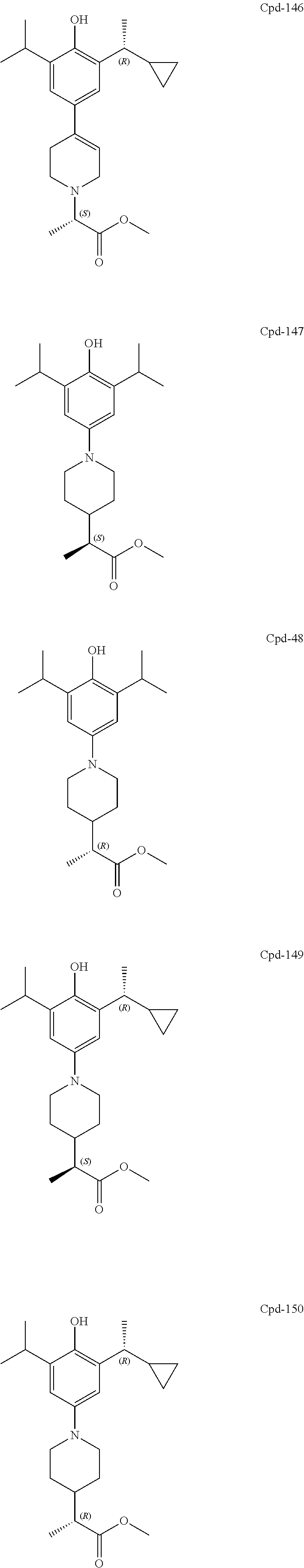

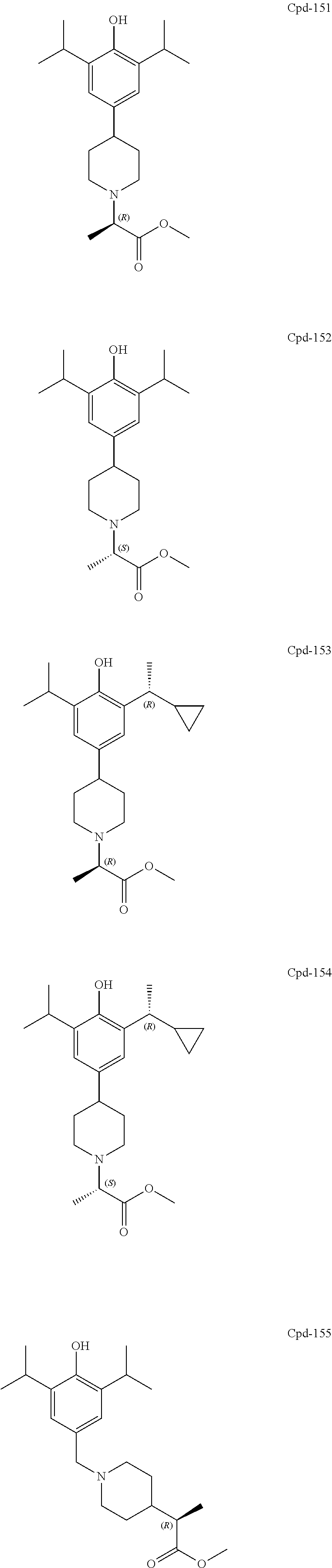

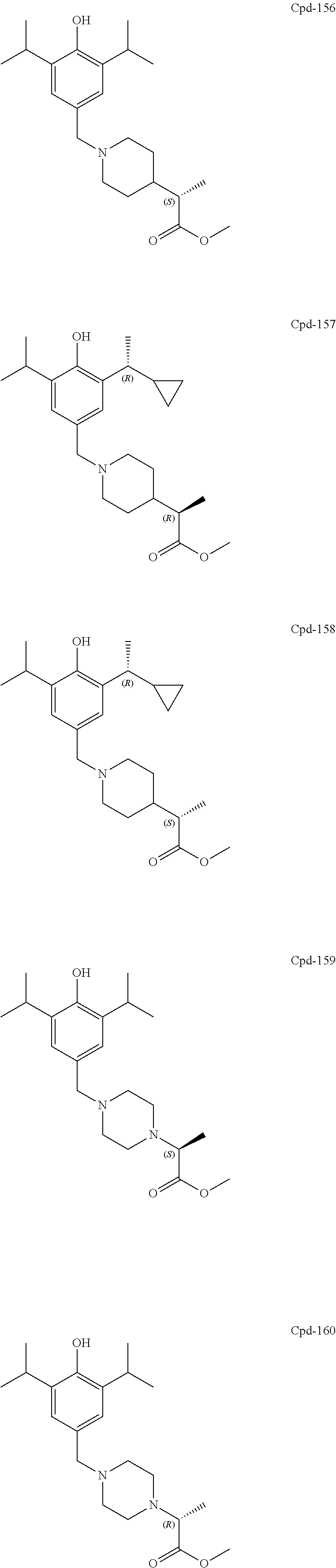

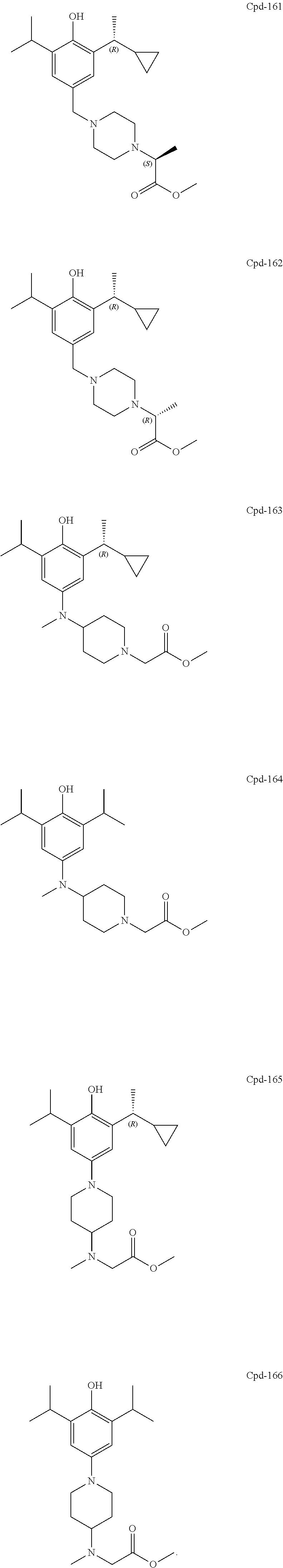

14. The compound according to claim 1, or a stereoisomer thereof, or a pharmaceutically acceptable salt thereof, or a solvate thereof, or a prodrug thereof, or a metabolite thereof, or a deuterated derivative thereof, characterized in that said pharmaceutically acceptable salt is selected from acetate, adipate, aspartate, benzoate, benzene sulfonate, bicarbonate, carbonate, bisulfate, sulfate, borate, camphorsulfonate, citrate, cyclamate, edisylate, ethane sulfonate, formate, fumarate, glucoheptonate, gluconate, glucuronate, hexafluorophosphate, hydrochloride, hydrobromide, hydroiodide, hydroxyethylsulfonate, lactate, malate, maleate, malonate, mesylate, methylsulfate, naphthylate, napsylate, nicotinate, nitrate, orotate, oxalate, palmitate, pamoate, phosphate, hydrogen phosphate, dihydrogen phosphate, pyroglutamate, saccharate, stearate, succinate, tannate, tartrate, tosylate, trifluoroacetate, xinafoate, methanesulfonate, or p-toluenesulfonate.

15. A drug, characterized in that said drug is prepared by using the compound according to claim 1, or a stereoisomer thereof, or a pharmaceutically acceptable salt thereof, or a solvate thereof, or a prodrug thereof, or a metabolite thereof, or a deuterated derivative thereof as active ingredients, with addition of pharmaceutically acceptable excipients.

16. Use of compound according to claim 1, or a stereoisomer thereof, or a pharmaceutically acceptable salt thereof, or a solvate thereof, or a prodrug thereof, or a metabolite thereof, or a deuterated derivative thereof, in preparation of a drug for inducing sedation, hypnosis, and/or anesthesia, and/or for controlling status epilepticus.
